SEQUENCE LISTING (8 sequences)

SEQ ID NO: 1 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 2 - protein, 170 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEIL

SEQ ID NO: 3 - protein, 11 aa, Synthetic construct
VSGWRLFKKIS

SEQ ID NO: 4 - protein, 11 aa, Synthetic construct
VTGYRLFEEIL

SEQ ID NO: 5 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 6 - protein, 148 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 7 - protein, 11 aa, Synthetic construct
GSMLFRVTINS

SEQ ID NO: 8 - protein, 297 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2024/0191211 A1
(43) Pub. Date: Jun. 13, 2024

(54) BIOLUMINESCENCE-TRIGGERED PHOTOCATALYTIC LABELING

(71) Applicant: Promega Corporation, Madison, WI (US)

(72) Inventors: Rachel Friedman Ohana, Madison, WI (US); Ce Shi, Madison, WI (US); Robin Hurst, Madison, WI (US); Mark A. Klein, Madison, WI (US); Jian Cao, Madison, WI (US); Thomas Kirkland, Madison, WI (US); Connor Fitzgerald, Madison, WI (US); Tetsuo Uyeda, Madison, WI (US); Thomas Machleidt, Madison, WI (US); Wenhui Zhou, Madison, WI (US); Matthew A. Larsen, Madison, WI (US); Hui Wang, Madison, WI (US); Weiwei An, Madison, WI (US); Karilyn Porter, Madison, WI (US)

(21) Appl. No.: 18/312,487

(22) Filed: May 4, 2023

Related U.S. Application Data
(60) Provisional application No. 63/338,322, filed on May 4, 2022.

(51) Int. Cl. C12N 9/02; B01J 31/22; B01J 35/39

(52) U.S. Cl. CPC C12N 9/0069; B01J 31/2295; B01J 35/39; C12Y 113/12013

(57) ABSTRACT

Provided herein are systems, methods, and compositions for bioluminescence-triggered catalysis of bioorthogonal labeling chemistries in a proximity dependent manner. In particular, provided herein are bioluminescent proteins or complexes, luminophore substrates thereof, photocatalysts or photosensitizers, activatable labels, and systems thereof, and methods of catalytically activating the activatable labels via bioluminescence-triggered catalysis.

### CROSS-REFERENCE TO RELATED APPLICATIONS

This application claims the benefit of U.S. Provisional Patent Application No. 63/338,322, filed on May 4, 2022, which is incorporated by reference herein.

### SEQUENCE LISTING

The text of the computer readable sequence listing filed herewith, titled “39793_202_SequenceListing.xml”, created May 4, 2023, having a file size of 8,882 bytes, is hereby incorporated by reference in its entirety.

### FIELD

Provided herein are systems, methods, and compositions for bioluminescence-triggered catalysis of bioorthogonal labeling chemistries in a proximity dependent manner, which can be actuated within biological systems. In particular, provided herein are bioluminescent proteins or complexes, luminophore substrates thereof, photocatalysts, activatable labels, and systems thereof, and methods for catalytically activating the activatable labels via bioluminescence-triggered catalysis.

### BACKGROUND

The need to study dynamic microenvironments, signaling pathways, and molecular processes in physiologically relevant contexts created a demand for new functional biology tools enabling such analyses in live cells and complex models in a nondestructive fashion.

### SUMMARY

Provided herein are systems, methods, and compositions for bioluminescence-triggered catalysis of bioorthogonal labeling chemistries in a proximity dependent manner. In particular, provided herein are bioluminescent proteins or complexes, luminophore substrates thereof, photocatalysts, activatable labels, and systems thereof, and methods for catalytically activating the activatable labels via bioluminescence-triggered catalysis.

In some embodiments, provided herein is light-driven photocatalysis that leverages bioluminescence as the light source and utilizing it to actuate bioorthogonal labeling of target molecules (e.g., biomacromolecules) with functional moieties for their subsequent visualization, enrichment, or manipulation. The components of such bioluminescence-driven photocatalytic systems include a bioluminescent light source (e.g., a luminophore and a luciferase or bioluminescent complex), a pair comprising a light-sensitive catalyst (photocatalyst), and an activatable label (e.g., a molecule comprising an activatable moiety and a functional moiety). In some embodiments, upon exposure to a light stimulus from the bioluminescent source, the excited catalyst engages in activation of neighboring activatable labels for subsequent generation of highly reactive intermediates that are available to undergo covalent crosslinking with biomacromolecules in the surrounding environment. Catalyst activation through absorption of visible light offers temporal control over catalytic reactivity. The use of bioluminescence to trigger photocatalysis in a proximity-dependent manner (e.g., requiring localization of the bioluminescent light source and the photocatalyst) provides a mild and minimally destructive light source, reduced phototoxicity, and efficient light delivery for triggering catalytic labelling in intact cells as well as spatial and temporal (+luminophore) control over catalyst activation, thereby increasing overall the spatiotemporal resolution of downstream covalent labeling chemistries. In some embodiments, the bioluminescent light-source and the photocatalyst are bioconjugated to induce proximity between the light source and the catalyst.

One aspect of the present technology is the use of bioluminescence, light generated by the interaction of a luminophore with a bioluminescent protein or complex of peptide(s) and/or polypeptides, to activate a photocatalyst. Other aspects of the present technology include: the activation of an activatable label by a bioluminescence-activated photocatalyst, assembling components of a bioluminescence-driven system/method through the use of one or more conjugates of the components of the systems here (e.g., via protein fusions, capture agents/elements, linkers, etc.) that drive in-cell covalent labeling chemistries using spatiotemporally arranged components to increase specificity and decrease toxicity, etc.

In one exemplary embodiment, exposure of a bioluminescent protein to an appropriate luminophore generates light that triggers local photocatalytic generation of reactive intermediates with limited diffusion radius. Those local reactive intermediates can form covalent linkage with neighboring residues for subsequent covalent modification of target molecules (e.g., biomacromolecules) with functional moieties. Such bioorthogonal labeling chemistries can be leveraged for a broad range of spatiotemporally-controlled phenotypic, proteomic, and genomic analyses including interactome and chromatin mapping, modulation of protein interactions as well as targeted visualization/enrichment/manipulation of proteins and nucleic acids.

In some embodiments, appropriate proximity between the bioluminescent protein and the photocatalyst is achieved by tethering the photocatalyst to the bioluminescent protein (directly or indirectly). In certain embodiments, the bioluminescent protein is made as a fusion with a capture agent (e.g., capture protein) and the photocatalyst is conjugated (e.g., via a linker) to a capture element. Binding of the capture agent to the capture element brings the bioluminescent protein and the photocatalyst into proximity to enable light produced by the bioluminescent protein and the luminophore to activate the photocatalyst.

In some embodiments, rather than using a bioluminescent protein, a multipart bioluminescent complex can be used as the light source for the photocatalytic system or method herein. The use of a bioluminescent complex that generates significantly enhanced light output upon complementation of two or more components (e.g., peptide(s), and/or polypeptide(s)) offers several advantages for some systems and methods herein. For example, conjugating directly or indirectly (e.g., fusing, tethering, etc.) one or more components of the bioluminescent complex to other components of the system (e.g., photocatalyst, target, etc.) ensures the proximity of that component to the bioluminescent complex upon light generation. Tethering two components of the system to separate components of the bioluminescent complex ensures the proximity of those components upon light generation by the complex. If the photocatalyst is tethered to the first component of the bioluminescent complex (e.g., LgBiT or a circularly permuted LgBiT (See, e.g., U.S. patent application Ser. No. 17/105,925; incorporated by reference in its entirety) that has high affinity for the second component of the bioluminescent complex (e.g., HiBiT), which is genetically fused to a target of interest then proximity between the photocatalyst and the second component of the bioluminescent complex is required for initiation of photocatalysis, thereby providing greater spatiotemporal control over the activation the photocatalyst and a modality agnostic approach for targeting the photocatalytic system to a site of interest (i.e., complementation and luminophore addition).

In some embodiments the bioluminescent protein or a component of the multipart bioluminescent complex is inserted at an internal position within the capture agent. In some embodiments, a position within the capture agent is selected to increase efficiency of bioluminescence activation of the catalyst through greater proximity or favorable conformation.

In some embodiments, the bioluminescent protein or a component of the multipart bioluminescent complex is circularly permuted.

Because a bioluminescent protein, or the components of a bioluminescent complex (or fusions thereof with other components of the systems herein) can be expressed within a cell or delivered into a cell, such systems offer generation of light for initiating photocatalysis within a cell.

In some embodiments, a component of a system herein (e.g., a bioluminescent protein or a component of a bioluminescent complex) is fused to a protein/peptide that results in specific localization of the component within a cell. For example, the localization protein/peptide might localize in a cellular compartment, bind to a specific protein, bind to DNA or RNA, bind to a specific nucleic acid sequence, etc. By localizing the component within a cell or linking the component to a specific cellular component, the subsequence photocatalytic labeling chemistry is similarly localized. In some embodiments, by localizing the system to a specific cellular target (e.g., protein, nucleic acid sequence, etc.), the activated label is capable of reacting with or acting upon a cellular target.

The systems and methods herein provide for bioluminescence-triggered catalysis of bioorthogonal labeling chemistries in a proximity dependent manner offering new functional biology tools to study dynamic environments and molecular process in physiologically relevant contexts, including live cells, complex cellular models, and model organisms. The technologies herein utilize a non-invasive, intrinsic light-source to activate light-sensitive catalysts, which can further engage in local generation of reactive intermediates that can form covalent linkage with neighboring biomacromolecules for their subsequent modification with functional moieties.

These bioorthogonal labeling chemistries can be leveraged for a broad range of spatiotemporally-controlled phenotypic, proteomic, and genomic analyses.

### BRIEF DESCRIPTION OF THE DRAWINGS

FIG. 1. A cartoon depiction of the bioluminescent triggered photocatalytic system utilizing a bioluminescent protein (NanoLuc) and a light sensitive photocatalyst which upon absorption of light engages in activation of an activatable label and subsequent generation of reactive intermediates that form covalent linkage with neighboring biomacromolecules.

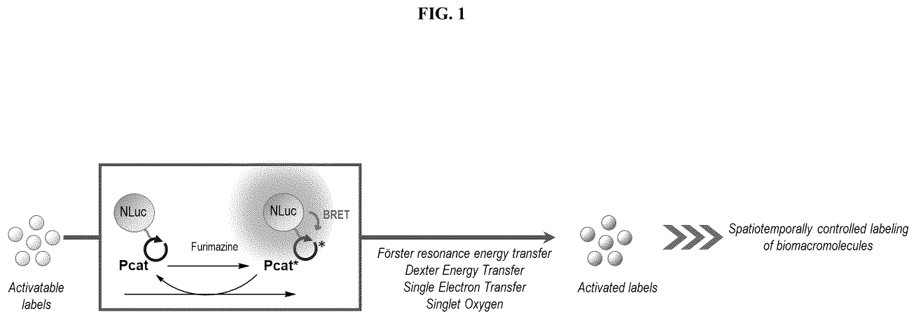

FIG. 2A-B. (A) A schematic of an activatable label comprising a photoreactive moiety and a functional moiety alongside structures of exemplary photoreactive and functional moieties. Exemplary photoreactive moieties include phenyl-trifluoro-methyl diazirine, phenylazide, and psoralen. Exemplary functional moieties include the capture reagent biotin; the click handles TCO (Trans-cyclooctene) and DBCO (Dibenzo-cyclooctene) as well as the fluorogenic dye EMA (Ethidium Monoazide Bromide). (B) Exemplary structures of reactive intermediates generated upon activation of the photoreactive moieties.

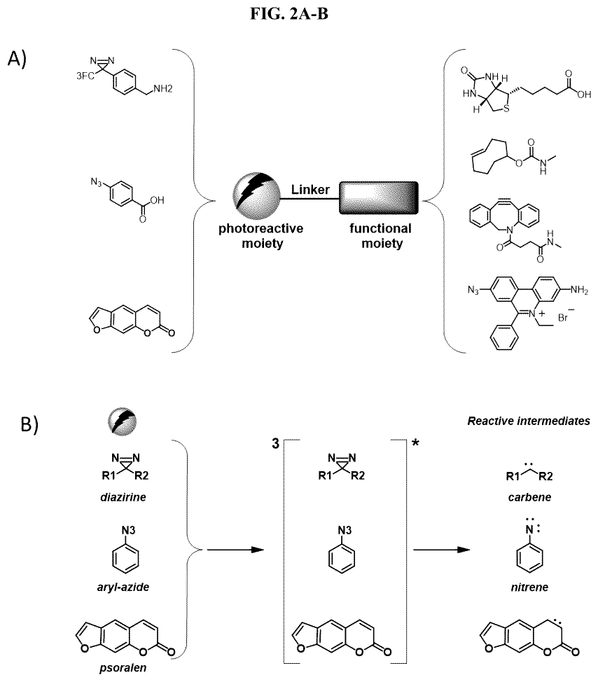

FIG. 3. A cartoon depiction of a light sensitive catalyst that is modified to enable bioconjugation and subsequent proximity to the bioluminescent light source. Exemplary catalysts include iridium-based catalyst, ruthenium-based catalyst, and Rose Bengal (organic photosensitizer). R represents an attachment motif and Linker Q represents a bioconjugation motif. Exemplary bioconjugation motifs include 2-pyridinecarboxyaldehyde (PCA) and 2-cyanobenzothiazole (CBT) linkers for direct bioconjugation as well as chloroalkane for indirect conjugation via binding to a HaloTag fusion.

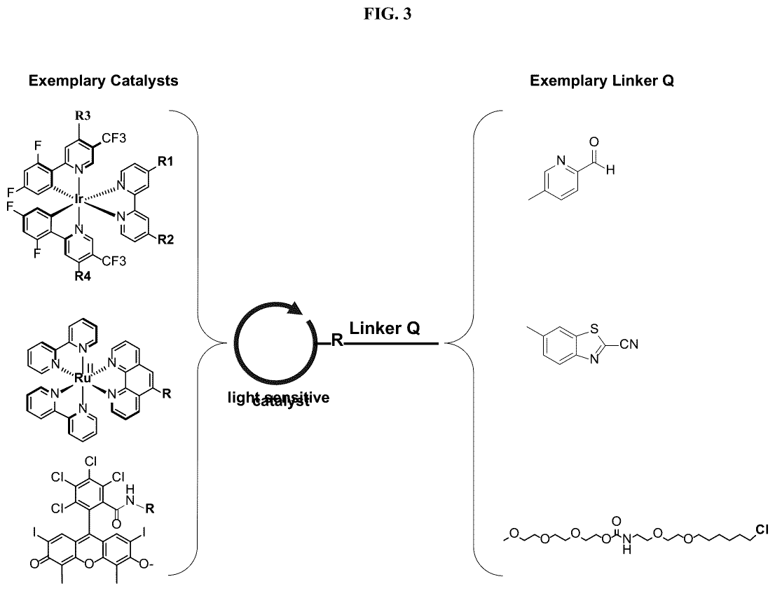

FIG. 4. Exemplary linker Qs designed for indirect conjugation via binding to a HaloTag fusion. The haloalkanes of varying lengths are designed for attachment to components of the systems herein (e.g., attachment to photocatalysts).

FIG. 5. The molecular structure of an exemplary photocatalyst linked to a HALOTAG substrate.

FIG. 6A-C. (A) A cartoon depiction of a system for localizing, inside cells, an extracellularly-added or intracellularly-assembled haloalkane-linked photocatalyst with a bioluminescent complex component (LgBiT) genetically fused to a modified dehalogenase (HALOTAG). (B) Fluorescence experiments depicted subcellular localization of a LgBiT-HaloTag fusion, which is labeled with a fluorescent Haloalkane ligand. (C) Experiment demonstrating the binding kinetics of an extracellularly added haloalkane-linked Ir-photocatalyst to a LgBiT-HaloTag fusion localized to different subcellular compartment. Results shows complete binding within 60 minutes.

FIG. 7. A cartoon depiction of a system that allows for bioluminescence-triggered spatiotemporal protein labeling in intact cells (with spatial relationships preserved). Complementation of HiBiT genetically fused to a protein of interest with LgBiT genetically fused to HaloTag and tethered to a catalyst allows localization of the catalyst, light source, and protein of interest and offers a modality agnostic approach for targeting the photocatalytic system to a site of interest.

FIG. 8. A cartoon depiction of a system for bioluminescence-triggered spatiotemporal labeling of dsDNA of interest in intact cells. Electroporation of nucleoprotein complex comprising a sgRNA and a fusion of Cas9-NanoLuc-HaloTag tethered to the catalyst allows localization of a catalyst, light source, and site of interest and offers a modality agnostic approach for targeting the photocatalytic system to a site of interest.

FIG. 9. A cartoon depiction of a system for bioluminescence-triggered generation of singlet oxygen for spatiotemporal labeling of proximal nucleic acids in intact cells. Electroporation of nucleoprotein complex comprising a sgRNA and a fusion of Cas9-NanoLuc-HaloTag tethered to a photosensitizer allows localization of the photosensitizer, light source, and site of interest and offers a modality agnostic approach for targeting the photocatalytic system to a site of interest.

FIG. 10A-C. A) Exemplary activatable labels comprising an azide group and a fluorogenic dye. B) Depiction of a system for bioluminescence-triggered spatiotemporal protein labeling with a fluorophore. Complementation of HiBiT genetically fused to a protein of interest with LgBiT genetically fused to HaloTag and tethered to a catalyst allows localization of the catalyst, light source, and site of interest. C) Depiction of a system for bioluminescence-triggered spatiotemporal labeling of dsDNA of interest in intact cells. Electroporation of nucleoprotein complex comprising a sgRNA and a fusion of Cas9-NanoLuc-HaloTag tethered to the catalyst allows localization of a catalyst, light source, and site of interest.

FIG. 11A-B. Depiction of systems for bioluminescence-triggered spatiotemporal protein labeling with a click handle, which is compatible with copper-free click ligation. These systems allow for two-step labeling with a broad range of functional moieties. Complementation of HiBiT genetically fused to a protein of interest with LgBiT genetically fused to HaloTag and tethered to a catalyst allows localization of the catalyst, light source, and site of interest. The two-step labeling depicted in A could also be applied to nucleic acids.

FIG. 12A-D. Properties of iridium (Ir) catalysts modified for increased aqueous solubility and subsequent bioconjugation. (A) Structure of Ir-catalyst with modifiable positions annotated as R1, R2, and R3. (B) Physiochemical properties of Ir-catalysts and their influence on energy transfer efficiency to diazirine. (C) and (D) Capacity of Ir-catalysts to drive LED-triggered photocatalytic labeling of a model protein (HaloTag-NanoLuc) with diazirine-biotin.

FIG. 13A-D. Influence of proximity between the Ir-catalyst and protein of interest on labeling efficiency. (A) Schematic depicting proximity driven by covalent binding of a catalyst that is conjugated to chloroalkane and HaloTag genetically fused to a protein of interest. (B) Structure of modifiable Ir-catalyst and its derivative, which is further conjugated to a chloroalkane. (C) Physiochemical properties of Ir-catalysts and their influence on energy transfer efficiency to diazirine. (D) Influence of proximity and efficiency of energy transfer to diazirine on LED-triggered photocatalytic protein labeling with diazirine-biotin.

FIG. 14A-C. Influence of physicochemical properties of Ir-catalysts as well as proximity to the bioluminescent light source on the efficiency of LED vs bioluminescence-triggered photocatalytic protein labeling. (A) Structure of modifiable Ir-catalysts and their derivatives, which are further conjugated to a chloroalkane. (B) Physiochemical properties of Ir-catalysts and their influence on energy transfer efficiencies from NanoLuc to the Ir-catalyst and from the Ir-catalyst to diazirine. (C) Influence of proximity and energy transfer efficiencies on LED vs bioluminescence-triggered photocatalytic protein labeling with diazirine-biotin.

FIG. 15A-D. Influence of chloroalkane length on catalysts' energy transfer efficiency, cell permeability and binding kinetic to HaloTag. (A) Structure of modifiable Ir-catalyst and its derivatives, which are further conjugated to a chloroalkane of different length. (B) Physiochemical properties of Ir-catalyst conjugates and their influence on energy transfer efficiency from NanoLuc to the Ir-catalyst and from the Ir-catalyst to diazirine. Influence of chloroalkane length on binding kinetic of chloroalkane-catalyst conjugates to HaloTag in either (C) cell lysate or (D) inside living cells.

FIG. 16A-B. Influence of NanoLuc HaloTag fusion orientation and chloroalkane length on the efficiency of BRET and bioluminescence-triggered photocatalytic protein labeling. (A) Influence of fusion orientation on brightness and BRET efficiency to a bound HaloTag TMR-fluorescent ligand. (B) Influence of chloroalkane length and fusion orientation on bioluminescence-triggered photocatalytic protein labeling.

FIG. 17A-C. Optimization of the bioluminescent photocatalytic complex comprising bioluminescent energy donor, chloroalkane-catalyst conjugate, and HaloTag offering the means to induce proximity between the two. (A) Scheme of the HT₁₇₈-cpNLuc-₁₇₉ chimera comprising a circularly permuted NanoLuc (i.e., cpNLuc) inserted into a HaloTag's surface loop (between residues 178-179), which is proximal to the ligand interaction site. (B) Brightness of NanoLuc-HaloTag and HT₁₇₈-cpNLuc-179 as well as subsequent BRET efficiency to a bound HaloTag TMR-fluorescent ligand. (C) Efficiency of bioluminescence-triggered photocatalytic protein labeling driven by NanoLuc-HaloTag and HT₁₇₈-cpNLuc-₁₇₉ tethered to chloroalkane-catalyst conjugates.

FIG. 18A-B. Overall optimization of the bioluminescent photocatalytic system for increased bioluminescence-triggered photocatalytic protein labeling. (A) Optimization steps resulting in an overall 900-fold increase in labeling efficiency. (B) Total filtered luminescence associated with each one of the optimization steps.

FIG. 19. Efficiencies of photocatalytic labeling triggered by either increasing LED power or bioluminescence. Analysis revealed that NanoLuc-HaloTag:Ir-9049 and HT₁₇₈-cpNLuc-₁₇₉: Ir-9049 complexes were able to drive bioluminescence-triggered protein labeling with efficiencies equivalent to 12.1 W and 55.4 W, respectively.

FIG. 20A-D. Expanding the utility of the bioluminescent photocatalytic system to include activation of other photoreactive groups. (A) Structure of phenyl-trifluoro-methyl-diazirine-biotin and phenyl-azide-biotin. (B) Absorbance profile of the two photoreactive-biotins. (C) Physiochemical properties of the two photoreactive-biotins and their influence on the capacity to undergo energy transfer events with an excited Ir-9049 catalyst. (D) Capacity of NanoLuc-HaloTag:Ir-9049 and HT₁₇₈-cpNLuc-₁₇₉: Ir-9049 to drive LED and bioluminescence-triggered photocatalytic protein labeling with phenyl-azide-biotin.

FIG. 21A-D. Fine tuning the properties of aryl-azides for increased efficiency and specificity of bioluminescence-triggered photocatalytic protein labeling. (A) Structure of aryl-azide-biotin analogs with the modification shown in gray. Physiochemical properties of aryl-azide analogs include (B) Absorbance profile and (C) Capacity to undergo energy transfer events with an excited Ir-8844 catalyst. (D) Influence of analogs' properties on efficiency and specificity of bioluminescence-triggered photocatalytic protein labeling as well as light-independent background.

FIG. 22. Fine tuning the properties of aryl-azides for increased efficiency and specificity of bioluminescence-triggered photocatalytic protein labeling. Efficiency and specificity as well as light-independent and dependent background for LED versus bioluminescence-triggered photocatalytic protein labeling for three aryl-azide biotin analogs.

FIG. 23A-D. Properties and labeling efficiencies afforded by photocatalytic complexes relying on HiBiT/LgBiT complementation (A) Complementation affinity, (B) Brightness, (C) BRET efficiency to a bound HaloTag TMR-fluorescent ligand, and (D) Photocatalytic labeling driven by complementation between FKPB-HiBiT and LgBiT-HaloTag: Ir-9049 and subsequent exposure to LED or bioluminescence.

FIG. 24A-B. Photocatalytic labeling of a proximal model protein inside living cells followed by its subsequent enrichment. (A) Structure of a cleavable phenyl-trifluoro-methyl-diazirine-biotin. (B) Western analysis of enriched model proteins (i.e., NanoLuc-HaloTag).

FIG. 25A-B. Two-step strategy coupling photocatalytic labeling of a proximal model protein with a click handle and a subsequent bioorthogonal ligation of a fluorophore. (A) Structure of phenyl-CF₃-diazirine-TCO and schematic depicting a TCO-tetrazine ligation. (B) HeLa cells expressing NanoLuc-HaloTag and subjected to LED versus bioluminescence-triggered two-step photocatalytic protein labeling with a fluorophore.

FIG. 26 A-D. Fine tuning the properties of naphthyl-azides for increased efficiency and specificity of bioluminescence-triggered photocatalytic protein labeling. (A) Structures of activatable labels comprising a photoreactive group linked to biotin as well as naphthyl-azide and quinoline-azide analogs with modifications shown in gray. (B-C) Absorbance profile for the naphthyl-azide-biotins and quinoline-azide-biotins. (D) Influence of analogs' properties on efficiency and specificity of bioluminescence-triggered photocatalytic protein labeling as well as light-dependent, light-independent, and catalyst-independent backgrounds.

FIG. 27 A-D. A screen of photoreactive groups for their capacity to undergo ruthenium-driven photocatalytic labeling. (A) Structure of ruthenium catalyst conjugated to chloroalkane. (B) Physiochemical properties of the iridium and ruthenium-catalysts conjugated to chloroalkane. (C) Structures of photoreactive groups included in the screen with modifications shown in gray. (D) Efficiencies and specificities of ruthenium-driven photocatalytic protein labeling as well as light-independent and catalyst-independent backgrounds.

FIG. 28 A-B. Ruthenium-driven photocatalytic labeling using a subset of naphthyl-azide photoreactive groups. (A) Structures of naphthyl-azide-biotin analogs with modifications shown in gray. (B) Influence of analogs' properties on efficiency and specificity of bioluminescence-triggered photocatalytic protein labeling as well as light-dependent, light-independent, and catalyst-independent backgrounds.

FIG. 29 A-B. Bioluminescence-triggered photocatalytic labeling inside living cells utilizing HT₁₇₈-cpNLuc-₁₇₉ chimera as energy donor. (A) Structures of cleavable phenyl-trifluoro-methyl-diazirine-biotin and cleavable vinyl-naphthyl-azide-biotin. (B) Western analysis for enrichment of HT₁₇₈-cpNLuc-₁₇₉ chimera following either iridium or ruthenium driven in-cell labeling over 0-60 min.

FIG. 30 A-D. Optimization of a complementation-based bioluminescent photocatalytic complex. (A) Scheme of the HT₁₇₈-cpmLgBiT-₁₇₉ chimera comprising a circularly permuted mutant LgBiT incorporating four LgTrip mutations (i.e., cpmLgBiT) inserted into a HaloTag surface loop (between residues 178-179), which is proximal to the ligand interaction site. (B) Brightness of LgBiT-HaloTag and HT₁₇₈-cpmLgBiT-₁₇₉ as well as BRET efficiencies to a bound HaloTag TMR-fluorescent ligand. (C) Cartoon depicting labeling specificity driven by complementation between HiBiT genetically fused to a protein of interest (e.g., EGFR), and HT₁₇₈-cpmLgBiT-₁₇₉ chimera coupled with BRET activation of a bound ligand (e.g., chloroalkane-conjugated to a fluorophore, chloroalkane conjugated to a light sensitive catalyst etc.). (D) Bioluminescence imaging for EGFR-HiBiT complemented with HT₁₇₈-cpmLgBiT-₁₇₉ chimera in the presence and absences of a bound HaloTag JF-549-fluorescent ligand.

FIG. 31 A-B. Utilizing HT₁₇₈-cpmLgBiT-₁₇₉ to target the bioluminescent photocatalytic system to an endogenous target that is tagged with HiBiT. (A) Structures of cleavable phenyl-trifluoro-methyl-diazirine-biotin and cleavable vinyl-naphthyl-azide-biotin. (B) Western analysis for enrichment of HT₁₇₈-cpmLgBiT-₁₇9 chimera and endogenous GAPDH-HiBiT following either iridium or ruthenium driven in-cell labeling over 0-30 min.

FIG. 32 A-E. Enrichment of proximal proteins, which were labeled inside cells by the bioluminescent photocatalytic system. (A) Cartoon depicting the localization of an EGFR-HT₁₇₈-cpNLuc-179 chimera fusion-tethered to a catalyst. (B) Bioluminescence emitted over time by an overexpressed chimera that is either unfused or genetically fused to EGFR. (C-D) Western analyses of expression as well as fluorofurimazine-dependent enrichment of EGFR-HT₁₇₈-cpNLuc-₁₇₉ chimera fusion, which was labeled inside cells. (E) Mass spectrometry analysis: summary of all proteins that were significantly enriched (≥4-fold over the no fluorofurimazine control).

FIG. 33 A-D. Expanding the utility of the bioluminescent photocatalytic system to include labeling of nucleic acids. (A) Cartoon depicting the assay for evaluating LED-triggered photocatalytic labeling of DNA. (B) Exemplary slot blot analysis. (C) Structures of the activatable labels comprising a photoreactive group linked to biotin and the different photoreactive-groups. (D) Efficiencies of LED-triggered photocatalytic labeling of DNA for the different photoreactive groups.

FIG. 34 A-B. Expanding the utility of the bioluminescent photocatalytic system to include labeling of RNA. (A) Structures of the activatable label comprising a photoreactive group linked to biotin and the different photoreactive-groups. (B) Efficiencies of LED-triggered photocatalytic labeling of RNA for the different photoreactive groups.

FIG. 35 A-B. Evaluation of a subset of photoreactive groups for their crosslinking efficiencies to DNA versus protein. (A) Slot blot analysis evaluating each photoreactive group and light-independent background as well as total (protein+DNA) and specific DNA labeling efficiencies. (B) Quantitation of slot blot analyses for specific DNA labeling over protein (%).

FIG. 36 A-B. Evaluation of a subset of photoreactive groups for their crosslinking efficiencies to RNA versus protein. (A) Slot blot analysis evaluating each photoreactive group and light-independent background as well as total (protein+RNA) and specific RNA labeling efficiencies. (B) Quantitation of slot blot analyses for specific RNA labeling over protein (%).

FIG. 37 A-B. Expanding the utility of the bioluminescent photocatalytic system to include singlet oxygen driven labeling of nucleic acids. (A) Cartoon depicting mechanisms for singlet oxygen driven labeling of DNA. (B) Efficiencies of LED-triggered singlet oxygen-dependent labeling of DNA and RNA.

FIG. 38 A-C. Strategies for targeting the bioluminescent-photocatalytic system to a specific DNA or RNA locus. (A) Cartoon depicting targeting a DNA locus by coupling a specific guide RNA and dCas9 genetically fused to the HT₁₇₈-cpNLuc-₁₇₉ chimera, which is tethered to the catalyst. The capacity to target a specific dsDNA is further demonstrated by a gel shift assay. (B) Cartoon depicting targeting an RNA locus by coupling a specific guide RNA and dCas12g1 genetically fused to the HT₁₇₈-cpNLuc-₁₇₉ chimera, which is tethered to the catalyst. The capacity to target a specific ssDNA or RNA is further demonstrated by a gel shift assay. (C) Cartoon depicting targeting an RNA/DNA locus using specific antisense oligos, which are conjugated to the Trip peptides. Upon hybridization with a specific RNA/DNA locus, the Trip peptides can complement with a HT₁₇₈-cpLgTrip-₁₇₉ chimera, which is tethered to the catalyst.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

As used herein, the term “luminescence” refers to the emission of light by a substance as a result of a chemical reaction (“chemiluminescence”) or an enzymatic reaction (“bioluminescence”).

As used herein, the term “bioluminescence” refers to production and emission of light by a reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “luminophore” refers to a chemical moiety or compound that can be placed in an excited electronic state (e.g., by a chemical or enzymatic reaction) and emits light as it returns to its electronic ground state.

As used herein, the term “imidazopyrazine luminophore” refers to a genus of luminophores including “native coelenterazine” as well as synthetic (e.g., derivative or variant) and natural analogs thereof, including furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

As used herein, the term “coelenterazine” refers to the naturally-occurring (“native”) imidazopyrazine of the structure:

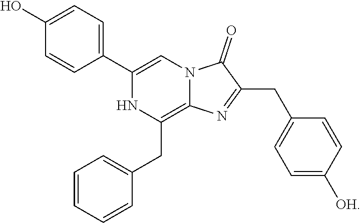 [SMILES: O=c1c(Cc2ccc(O)cc2)nc2c(Cc3ccccc3)[nH]c(-c3ccc(O)cc3)cn1-2]

As used herein, the term “furimazine” refers to the coelenterazine derivative of the structure:

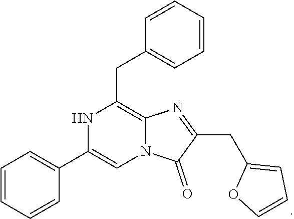 [SMILES: O=c1c(Cc2ccco2)nc2c(Cc3ccccc3)[nH]c(-c3ccccc3)cn1-2]

As used herein, the term “fluorofurimazine” refers to the furimazine derivative of the structure:

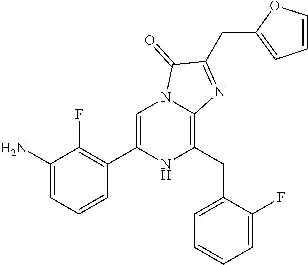 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “luciferin” refers to a compound of the structure:

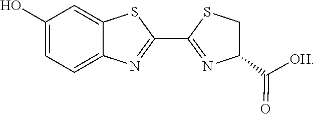 [SMILES: O=C(O)[C@H]1CSC(c2nc3ccc(O)cc3s2)=N1]

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å, depending on the degree of spectral overlap).

As used herein, the term “an Oplophorus luciferase” (“an OgLuc”) refers to a luminescent polypeptide having significant sequence identity, structural conservation, and/or the functional activity of the luciferase produce by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NANOLUC), which comprises 10 β strands (β1, β2, β3, β4, β5, β6, β7, β8, β9, β10) and utilize substrates such as coelenterazine or a coelenterazine derivative or analog to produce luminescence.

As used herein the term “complementary” refers to the characteristic of two or more structural elements (e.g., peptide, polypeptide, nucleic acid, small molecule, etc.) being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to co-localize complementary elements, to lower interaction energy for complementary, to overcome low affinity for one another, etc.

As used herein, the term “complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides and polypeptide) is formed under assay conditions such that the complex is thermodynamically favored (e.g., compared to a non-aggregated, or non-complexed, state of its component molecules). As used herein, the term “complex,” unless described as otherwise, refers to the assemblage of two or more molecules (e.g., peptides, polypeptides, or a combination thereof).

As used herein, the term “capture protein” or “capture agent” refers to a protein or other molecular entity that forms a stable covalent bond with its substrate, ligand, or other molecular element upon interaction therewith. A capture protein may be a receptor that forms a covalent bond upon binding its ligand or an enzyme that forms a covalent bond with its substrate. An example of a suitable capture protein for use in embodiments of the present invention is the HALOTAG protein described in U.S. Pat. No. 7,425,436 (herein incorporated by reference in its entirety).

As used herein, the terms “capture ligand,” “capture moiety,” or “capture element refers to a ligand, substrate, etc., that forms a covalent bond with a capture protein upon interaction therewith. An example of a suitable capture ligand for use in embodiments of the present invention is the HALOTAG ligand described, for example, in U.S. Pat. No. 7,425,436 (herein incorporated by reference in its entirety). Moieties that find use as HALOTAG ligands include haloalkane (HA) groups (e.g., chloroalkane (CA) groups). In embodiments described herein that specify an HA or CA capture ligand, other suitable capture ligands may be substituted unless otherwise specified.

As used herein, the term “activatable label” refers to a bifunctional molecule comprising an activatable moiety linked to a functional moiety. The functional moiety is a moiety that is suitable for detection or capture or subsequent ligation and serve as a label (e.g., fluorophore, chromophore, strained alkyne, haloalkane, biotin, etc.). The activatable moiety is capable of being converted from an activatable form to an activated (reactive) form by a catalyst. In some embodiments, the activatable moiety is a photoreactive group. Examples of activatable moieties include phenyl trifluoro-methyl diazirine, phenyl azide, and psoralen. The activated form of the activatable moiety is capable of forming covalent linkage with target molecules (e.g., biomacromolecules), thereby labeling them with the functional moiety.

As used herein, the term “cellular target” refers to a cellular (e.g., intracellular or surface exposed) entity (e.g., molecule, cellular compartment, complex, etc.) that can be labelled by the systems herein. Cellular targets may be biomacromolecules such as protein, polypeptide, nucleic acid (e.g., DNA or RNA), lipids, polysaccharides, or a complex comprising any of these with a polypeptide(s). A cellular target could be composed of more than one component, subunit, or polypeptide, e.g., the cellular target is a protein complex. Examples of a cellular target may include a receptor or an enzyme.

As used herein, the term “bioactive agent” refers generally to any physiologically or pharmacologically active substance or a substance suitable for detection. In some embodiments, a bioactive agent is a potential therapeutic compound (e.g., small molecule, peptide, nucleic acid, etc.) or drug-like molecule. Bioactive agents for use in embodiments described herein are not limited by size or structure.

As used herein, the term “photoreactive group,” refers to a moiety, which, upon exposure to light (e.g., a specific wavelength or wavelength range of light, etc.), forms a covalent linkage with a molecule or functional group within its immediate vicinity (e.g., within a distance range (e.g., <120 nm, <110 nm, <100 nm, <90 nm, <80 nm, <70 nm, <60 nm, <50 nm, <40 nm, <30 nm, <20 nm, <10 nm, <5 nm, <4 nm, <3 nm, <2 nm, etc.)).

As used herein, the term “photocatalyst” refers to a molecule that, upon absorption of light at an appropriate wavelength, is capable of engaging in activation of a neighboring activatable label(s) via either energy transfer or electron transfer events, thereby lowering the activation energy and/or increasing the rate of a chemical labeling reaction. In some embodiments, the excited photocatalyst is capable of regenerating itself after each energy transfer or electron transfer event, thereby repeatedly engaging in activation of neighboring activatable labels. In some embodiments, a photocatalyst that, upon absorption of light at an appropriate wavelength, is capable of engaging in energy transfer events with oxygen to generate reactive species (e.g., a proton, singlet oxygen, etc.) for subsequent chemical modification of a neighboring biomacromolecule, is referred to as a “photosensitizer.” Some embodiments herein described in conjunction with a photocatalyst may encompass or be limited to a photosensitizer.

As used herein, the term “small molecule” refers to a low molecular weight (e.g., <2000 daltons, <1000 daltons, <500 daltons) organic compound with dimensions (e.g., length, width, diameter, etc.) on the order of 1 nm. Larger structures, such as peptides, proteins, and nucleic acids, are not small molecules, although their constituent monomers (ribo- or deoxyribonucleotides, amino acids, etc.) are considered small molecules.

As used herein, the term “cell permeable” refers to a compound or moiety that is capable of effectively crossing a cell membrane that has not been synthetically permeabilized.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; Carruthers, Some Modern Methods of Organic Synthesis, 3^{rd} Edition, Cambridge University Press, Cambridge, 1987; the entire contents of each of which are incorporated herein by reference.

As used herein, the term “physiological conditions” encompasses any conditions compatible with living cells, e.g., predominantly aqueous conditions of a temperature, pH, salinity, chemical makeup, etc., that are compatible with living cells.

As used herein, the terms “conjugated” and “conjugation” refer to the covalent attachment of two molecular entities (e.g., post-synthesis and/or during synthetic production). Conjugated entities may be peptides or proteins that are “fused” by a peptide linkage, or may also include other molecular entities (e.g., nucleic acid, small molecules, etc.) connected directly or by suitable linkers.

The term “binding moiety” refers to a domain that specifically binds an antigen or epitope independently of a different epitope or antigen binding domain. A binding moiety may be an antibody, antibody fragment, a receptor domain that binds a target ligand, proteins that bind to immunoglobulins (e.g., protein A, protein G, protein A/G, protein L, protein M), a binding domain of a proteins that bind to immunoglobulins (e.g., protein A, protein G, protein A/G, protein L, protein M), oligonucleotide probe, peptide nucleic acid, DARPin, anticalin, nanobody, aptamer, affimer, a purified protein (either the analyte itself or a protein that binds to the analyte), and analyte binding domain(s) of proteins etc. Table A provides a list of exemplary binding moieties that could be used singly or in various combinations in methods, systems, and assays (e.g., immunoassays) herein.

TABLE A
Exemplary binding moieties

Protein A
Ig Binding domain of protein A
Protein G
Ig Binding domain of protein G
Protein L
Ig Binding domain of protein L
Protein M
Ig Binding domain of protein M
polyclonal antibody against analyte X
monoclonal antibody
recombinant antibody
scFv
variable light chain (V_{L}) of antibody (monoclonal,
recombinant, polyclonal) recognizing target analyte X
protein (e.g., receptor) binding domain that binds to
analyte X
(Fab) fragment
Fab′ fragment
Fv fragment
F(ab′)2 fragment
oligonucleotide probe
DARPins and other synthetic binding scaffolds (ex:
Bicycles)
peptide nucleic acid
aptamer
affimer

As used herein, the term “antibody” refers to a whole antibody molecule or a fragment thereof (e.g., fragments such as Fab, Fab′, and F(ab′)₂, variable light chain, variable heavy chain, Fv). It may be a polyclonal or monoclonal or recombinant antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope. In this regard, “affinity which is substantially higher” means affinity that is high enough to enable detection of an antigen or epitope, which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of the antigen binding region or a variable region. Antibody fragments include, but are not limited to, Fab, Fab′, F(ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises an additional constant region extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans.

As used herein, the term “biomolecule” or “biological molecule” refers to molecules and ions that are present in organisms and are essential to a biological process(es) such as cell division, morphogenesis, or development. Biomolecules include large macromolecules (or polyanions) such as proteins, carbohydrates, lipids, and nucleic acids as well as small molecules such as primary metabolites, secondary metabolites, and natural products. A more general name for this class of material is biological materials. Biomolecules are usually endogenous, but may also be exogenous. For example, pharmaceutical drugs may be natural products or semisynthetic (biopharmaceuticals), or they may be totally synthetic.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “amino” means a —NH₂ group.

As used herein, the term “halogen” or “halo” means F, Cl, Br, or I. As used herein, the term “haloalkyl” means an alkyl group, as defined herein, in which at least one hydrogen atom (e.g., one, two, three, four, five, six, seven or eight hydrogen atoms) is replaced by a halogen. In some embodiments, each hydrogen atom of the alkyl group is replaced with a halogen. Representative examples of haloalkyl include, but are not limited to, fluoromethyl, difluoromethyl, trifluoromethyl, 2,2,2-trifluoroethyl, and 3,3,3-trifluoropropyl.

As used herein, the term “heteroalkyl” means an alkyl group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. By way of example, 1, 2, or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

As used herein, the term “alkenyl” means a straight or branched hydrocarbon chain containing at least one carbon-carbon double bond. The double bond(s) may be located at any position within the hydrocarbon chain. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” means a straight or branched hydrocarbon chain containing at least one carbon-carbon triple bond. The triple bond(s) may be located at any position within the hydrocarbon chain. Representative examples of alkynyl include, but are not limited to, ethynyl, propynyl, and butynyl.

As used herein, the term “alkylene” means a divalent alkyl radical (e.g., —CH₂CH₂—). As used herein, the term “alkenylene” means a divalent alkenyl radical (e.g., —CH═CH—). As used herein, the term “alkynylene” means a divalent alkynyl radical (e.g., —C≡C—).

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” means a —NH₂ group.

As used herein, the term “aminoalkyl” means an alkyl group, as defined herein, in which at least one hydrogen atom is replaced with an amino group, as defined herein. Representative examples of aminoalkyl include, but are not limited to, aminomethyl, 2-aminoethyl, 2-aminopropyl, 3-aminopropyl, and 4-aminobutyl.

As used herein, the term “cyano” means a —CN group.

As used herein, the term “cyanoalkyl” means an alkyl group, as defined herein, in which at least one hydrogen atom is replaced with a cyano group, as defined herein. Representative examples of cyanoalkyl include, but are not limited to, cyanomethyl, 2-cyanoethyl, 2-cyanopropyl, 3-cyanopropyl, and 4-cyanobutyl.

### DETAILED DESCRIPTION

Provided herein are systems, methods, and compositions for bioluminescence-triggered catalysis of bioorthogonal chemistries in a proximity dependent manner. In particular, provided herein are bioluminescent proteins or complexes, luminophore substrates thereof, photocatalysts or photosensitizers, activatable labels, and systems thereof, and methods for catalytically activating the activatable labels via bioluminescence-triggered catalysis.

The need to study dynamic microenvironments, signaling pathways, and molecular processes in physiologically relevant contexts presents a demand for new functional, biological tools that enable such analyses in live cells and complex models in a nondestructive fashion. Bioluminescence-triggered catalysis of bioorthogonal labeling chemistries in a proximity-dependent manner offers a solution to this need by utilizing a non-invasive, intrinsic light-source to actuate bioorthogonal chemical labeling reactions in biological systems for subsequent proximity-dependent modification of biomacromolecules with functional moieties. The components for such photocatalytic systems include a bioluminescent light source (e.g., luciferase or bioluminescent complex (e.g., NanoBiT) and pairs of (1) light-sensitive catalyst (transition metal or organic dye catalyst) and (2) activatable labels. Upon luminophore substrate addition, the bioluminescent entity (e.g., NanoBiT, NanoLuc, etc.) generates light that triggers local photocatalytic generation of reactive intermediates with limited diffusion radius, which can form covalent linkage with neighboring residues for the subsequent modification of target molecules (e.g., biomacromolecules) with functional moieties. These bioorthogonal labeling chemistries can be leveraged for a broad range of spatiotemporally controlled phenotypic, proteomic, and genomic analyses including interactome and chromatin mapping, modulation of protein interactions as well as targeted visualization/enrichment/manipulation of proteins and nucleic acids.

Advantages of using bioluminescence as the light source rather than global-light radiation (e.g., LED or laser) include: using an intrinsic light source that is mild and minimally destructive; reduced phototoxicity; efficient light delivery for triggering catalysis in intact cells and complex models; local and conditional (+luminophore substrate) delivery of light for greater spatiotemporal resolution over catalyst activation and downstream labeling chemistries; and the ability to tether the light source to target molecules and/or other components of the system (e.g., the photocatalyst).

In some embodiments, provided herein are systems comprising one or more of a bioluminescent protein or structurally-complementary components of a bioluminescent complex; a luminophore, wherein the bioluminescent protein catalyzes emission of a first wavelength of light from the luminophore upon interaction therewith; a photocatalyst, wherein the photocatalyst is activated upon absorption of light of the first wavelength; and an activatable label, wherein the activatable label is converted into an activated label when in proximity to the activated photocatalyst.

In some embodiments, a bioluminescent protein or component of a bioluminescent complex is linked to a photocatalyst. In some embodiments, the linkage of the photocatalyst to the light source provides the appropriate proximity for activating the photocatalyst.

### Bioluminescent Protein or Complex

The present disclosure includes materials and methods related to bioluminescent polypeptides, bioluminescent complexes, and components thereof. In particular, light emitted from bioluminescent proteins or complexes (or from luminophores acted upon by bioluminescent proteins or complexes) is used to activate photocatalysts.

### NanoLuc

In some embodiments, systems and methods herein comprise a bioluminescent protein. In some embodiments, a bioluminescent protein is a luciferase enzyme. Suitable luciferase enzymes include those selected from the group consisting of: Photinus pyralis or North American firefly luciferase; Luciola cruciata or Japanese firefly or Genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraezeelandiae, Oplophorus typus, Oplophorus noraezelandiae, or Oplophorus spinous).

In some embodiments, the bioluminescent protein is a luciferase of Oplophorus gracilirostris, NanoLuc® luciferase (Promega Corporation; U.S. Pat. Nos. 8,557,970; 8,669,103; herein incorporated by reference in their entireties). PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides. Such polypeptides find use in embodiments herein and can be used in conjunction with the compositions, assays, devices, systems, and methods described herein. In some embodiments, compositions, assays, devices, systems, and methods provided herein comprise a bioluminescent polypeptide of SEQ ID NO: 1, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, any of the aforementioned bioluminescent proteins are linked (e.g., fused, chemically linked, etc.) to one or more other components of the assays and systems described herein (e.g., fused to a HALOTAG protein).

In some embodiments, a bioluminescent protein is a circularly permuted version of a natural or modified bioluminescent protein (See, e.g., U.S. Pat. No. 10,774,364; incorporated by reference in its entirety).

In some embodiments, systems and methods herein comprise a bioluminescent complex (e.g., two or more components (e.g., peptides and/or polypeptides) that combine through structural complementation to form a complex that is capable of activating a luminophore to emit light). In some embodiments, a luminophore emits significantly more light in the presence of the bioluminescent complex than in the presence of any one of the components alone. In some embodiments, a bioluminescent complex is formed from fragments (e.g., peptide(s) and/or polypeptide(s)) of a luciferase enzyme. In some embodiments, a bioluminescent complex is a circularly permuted version of a natural or modified bioluminescent component (e.g., formed from two fragments of a circularly permuted luciferase); See, e.g., U.S. Pat. No. 10,774,364; incorporated by reference in its entirety.

PCT Appln. Nos. PCT/US14/26354, PCT/US19/036844, and PCT/US20/62499; U.S. Pat. No. 9,797,889; U.S. patent application Ser. No. 16/439,565; and U.S. Pub. No. 2021/0262941 (each of which is herein incorporated by reference in their entirety and for all purposes) describe compositions and methods for the assembly of bioluminescent complexes; such complexes, and the peptide and polypeptide components thereof, find use in embodiments herein and can be used in conjunction with the assays and methods described herein.

In some embodiments, peptide and polypeptide components are provided for the assembly of a bioluminescent complex capable of generating luminescence in the presence of an appropriate substrate (e.g., a coelenterazine or a coelenterazine analog (e.g., furimazine, fluorofurimazine, etc.). In some embodiments, complementary polypeptide(s) and peptide(s) collectively span the length (or >75% of the length, >80% of the length, >85% of the length, >90% of the length, >95% of the length, or more) of a luciferase base sequence (or collectively comprise at least 40% sequence identity to a luciferase base sequence (e.g., >40%, >45%, >50%, >55%, >60%, >65%, >70%, >75%, >80%, >85%, >90%, >95%, or more). In some embodiments, “complementary” polypeptide(s) and peptide(s) are separate molecules that each correspond to a portion of a luciferase base sequence. Through structural complementarity, they assemble to form a bioluminescent complex. Suitable luciferase base sequences may include SEQ ID NOS: 1 or 2, or the sequences of any of the full-length luciferases listed above. In some embodiments, the bioluminescent complex comprises the NANOBIT or NANOTRIP systems (Promega; Madison, WI). In some embodiments, the peptide and/or polypeptide components of a bioluminescent complex collectively comprise at least 60% sequence identity (e.g., >60%, >65%, >70%, >75%, >80%, >85%, >90%, >95%, >99%) with SEQ ID NO: 1 and/or SEQ ID NO: 2. In some embodiments, the peptide and/or polypeptide components of the bioluminescent complex comprise HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7). In some embodiments, the peptide and/or polypeptide components of the bioluminescent complex comprise at least 60% sequence identity (e.g., >60%, >65%, >70%, >75%, >80%, >85%, >90%, >95%, >99%) with HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7). In some embodiments, the peptide and/or polypeptide components of the bioluminescent complex comprise circularly permuted variants of HIBIT (SEQ ID NO: 3), SMBIT (SEQ ID NO: 4), LGBIT (SEQ ID NO: 5; e.g., cp site 67/68 (e.g., with or without E4D, Q42M, M106K, and/or T144D substitutions)), LGTRIP (SEQ ID NO: 6; e.g., cp site 67/68, cp site 49/50, etc.), and/or SMTRIP9 (SEQ ID NO: 7)

In some embodiments, any of the aforementioned components of bioluminescent complexes are linked (e.g., fused, chemically linked, tethered, etc.) to one or more other components of the assays and systems described herein (e.g., fused to a HALOTAG protein).

There are various characteristics of the bioluminescent complexes that find use in embodiments herein that may provide advantages in certain applications. For example, a bioluminescent complex (e.g., a complex formed upon complementation of HiBiT/LgBiT) only generates light upon complementation of its component peptide/polypeptides; therefore, directly or indirectly conjugating (e.g., fusing, tethering, etc.) one or more components of the bioluminescent complex to other components of the system (e.g., photocatalyst, activatable label, target, etc.) ensures the proximity of that component to the bioluminescent complex upon light generation. Tethering of two other components of the system (e.g., photocatalyst and target binding agent) to separate components of the bioluminescent complex ensures the proximity of those components upon light generation by the complex. In some embodiments, the use of a bioluminescent complex, due to the requirement that two components come together to form the complex, provides enhanced spatiotemporal resolution through conditional activation at a specific site.

In some embodiments the bioluminescent protein or a component of the multipart bioluminescent complex is inserted in an internal position within the capture agent. In some embodiments, a position within the capture agent is selected to increase efficiency of bioluminescence activation of the catalyst through greater proximity or favorable conformation.

In some embodiments, the bioluminescent protein or a component of the multipart bioluminescent complex is circularly permuted.

### Luminophore Substrate

In some embodiments, the systems and methods herein comprise luminophore substrates that emit light upon interaction with the bioluminescent proteins and/or complexes described herein. Suitable luminophores for the bioluminescent protein or complex used in the system or method will be understood. For example, firefly luciferin, with the structure:

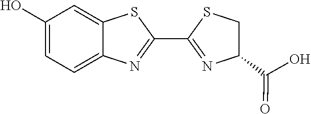 [SMILES: O=C(O)[C@H]1CSC(c2nc3ccc(O)cc3s2)=N1]

is the luciferin found in many Lampyridae species and is the substrate of beetle luciferases. Latia luciferin, with the structure:

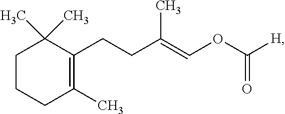

is from the freshwater snail Latia neritoides. Bacterial luciferin, with the structure:

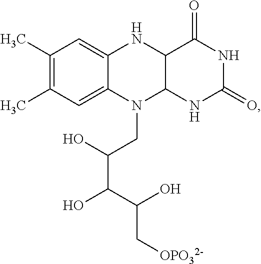

finds use as a substrate for many bacterial luciferases. Coelenterazine, of the structure:

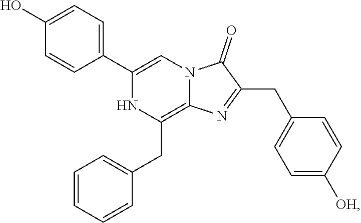 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp and is the luminophore substrate for the luciferases of those organisms. Variants and derivatives of coelenterazine, such as furimazine and fluorofurimazine, find use in embodiments herein (e.g., with Oplophorus-derived bioluminescent proteins and complexes). Other luminophore substrates include those of dinoflagellates:

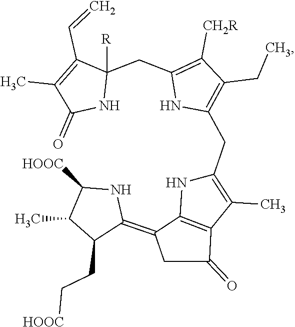

Vargulin (cypridin luciferin):

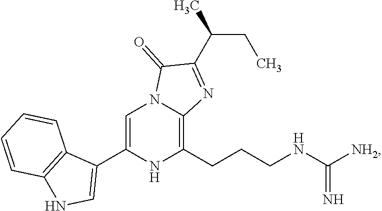 [SMILES: CC[C@H](C)c1nc2c(CCCNC(=N)N)[nH]c(-c3c[nH]c4ccccc34)cn-2c1=O]

N. nambi:

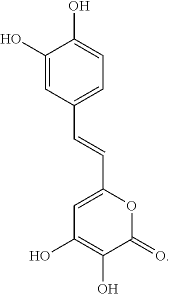 [SMILES: O=c1oc(/C=C/c2ccc(O)c(O)c2)cc(O)c1O]

Pairing of appropriate bioluminescent proteins or complexes with luminophores is understood in the field. In particular embodiments, a bioluminescent protein is provided in a system or method herein that utilizes an imidazopyrazine luminophore, such as coelenterazine, furimazine, or fluorofurimazine (U.S. application Ser. No. 16/548,214; incorporated by reference in its entirety). In some embodiments, a system or method comprises (1) an Oplophorus-derived polypeptide (e.g., NANOLUC) or components of an Oplophorus-derived bioluminescent complex (e.g., NANOBIT, NANOTRIP) and an imidazopyrazine luminophore (e.g., coelenterazine, furimazine, fluorofurimazine, etc.). In some embodiments, systems and methods herein comprise an imidazopyrazine luminophore such as native coelenterazine, furimazine, fluorofurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties.

In some embodiments, the luminophore emits light upon interaction with the bioluminescent protein or complex. In some embodiments, the luminophore emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the luminophore emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

### Photocatalysts

In some embodiments, the systems and methods herein comprise a photocatalyst that is capable of absorbing light emitted from a luminophore (upon interaction with a bioluminescent protein or complex) and subsequently activating a neighboring activatable label. Any compound or moiety capable of receiving light energy emitted from a bioluminescent protein- or complex-activated luminophore and subsequently engaging in activation of an activatable label may find use in embodiments herein. In some embodiments, the excited photocatalyst engages in activation of neighboring activatable label via Förster Resonance Energy Transfer, Dexter Energy Transfer, Single Electron Transfer, or any other suitable mechanism of energy or electron transfer.

In some embodiments, the photocatalyst is an iridium-based or ruthenium-based photocatalyst (Bevernaegie et al. ‘A Roadmap Towards Visible Light Mediated Electron Transfer Chemistry with Iridium(III) Complexes.’ ChemPhotoChem 2021, 5, 217; Day et al. Advances in Photocatalysis: A Microreview of Visible Light Mediated Ruthenium and Iridium Catalyzed Organic Transformations Org. Process Res. Dev. 2016, 20, 1156-1163; incorporated by reference in their entireties). In some embodiments, the photocatalyst is of the structure of Formula (I):

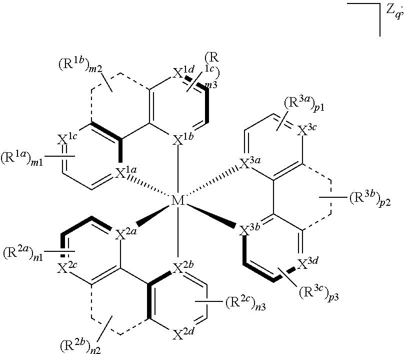

wherein:
each set of dashed lines ( [symbol] ) represents the presence or absence of a fused 6-membered ring;
M is a transition metal;
m1, m2, m3, n1, n2, n3, p1, p2, and p3 are each independently 0, 1, or 2;
R^{1a}, R^{1b}, R^{1c}, R^{2a}, R^{2b}, R^{2c}, R^{3a}, R^{3b}, and R^{3c} are each independently selected from halo, alkyl, haloalkyl, amino, heteroalkyl, and a group -Linker-Q, wherein Q is a capture element;
X^{1a}, X^{1b}, X^{2a}, X^{2b}, X^{3a}, and X^{3b} are each independently selected from N and C, wherein at least one of X^{1a} and X^{1b} is N, at least one of X^{2a} and X^{2b} is N, and at least one of X^{3a} and X^{3b} is N;
X^{1c}, X^{1d}, X^{2c}, X^{2d}, X^{3c}, and X^{3d} are each independently selected from CH and N;
Z is an anion; and
q is 0, 1, or 2.

In some embodiments, the photocatalyst comprises a transition metal selected from Ru and Ir.

In some embodiments, the photocatalyst is an iridium-based photocatalyst selected from:

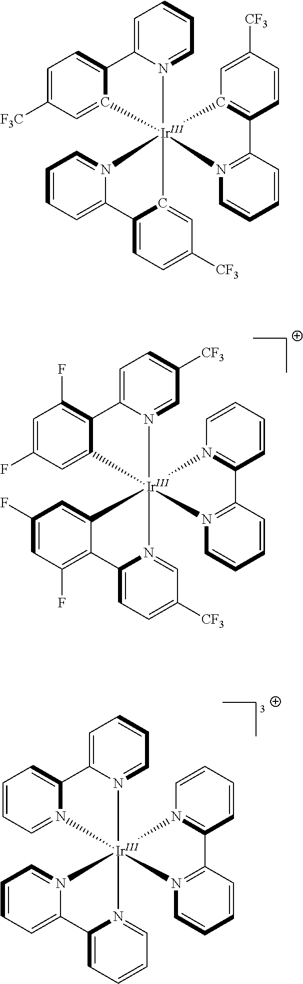

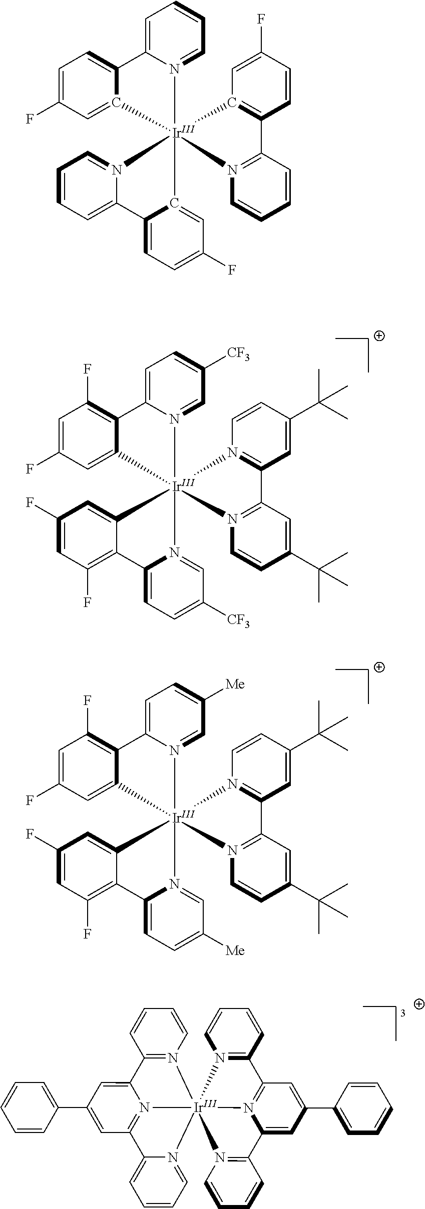

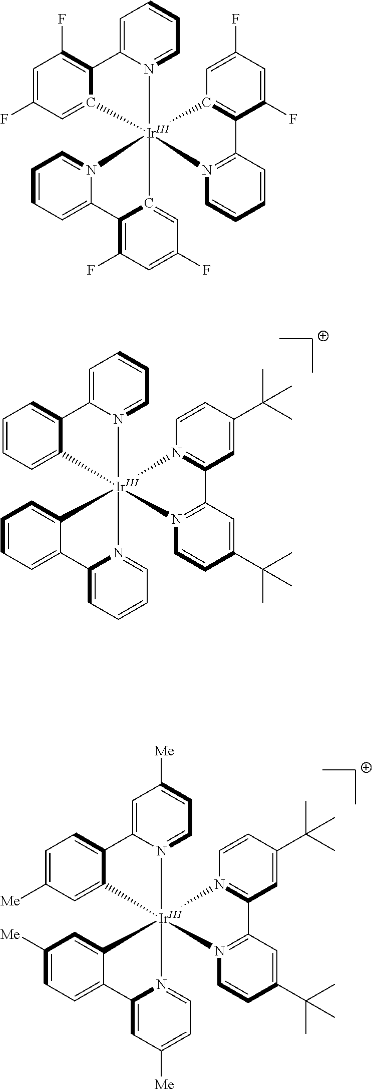

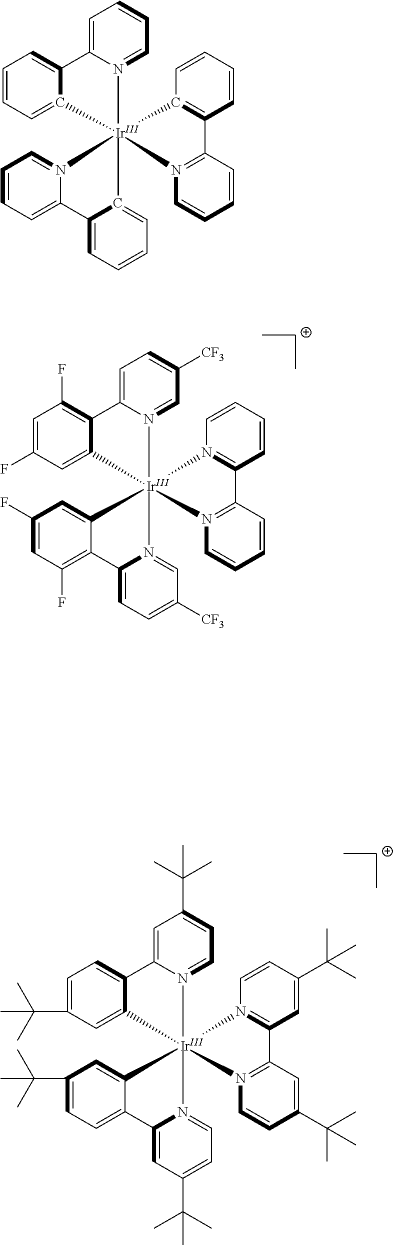

or a derivative thereof in which the compound is functionalized with at least one group -Linker-Q (wherein Q is a capture element).

In some embodiments, the photocatalyst is a ruthenium-based photocatalyst selected from:

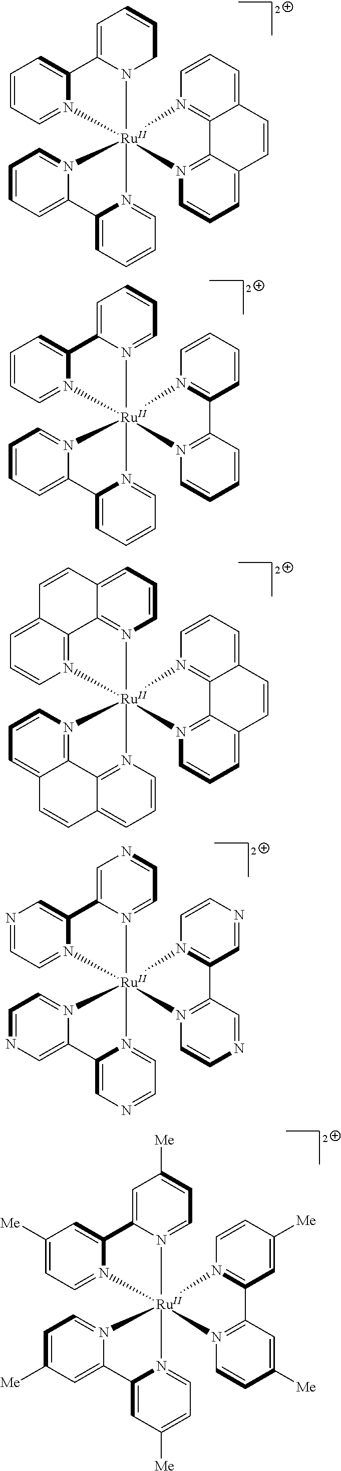

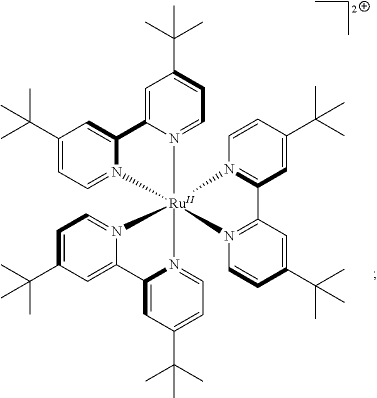

or a derivative thereof in which the compound is functionalized with at least one group -Linker-Q (wherein Q is a capture element).

In some embodiments, M is Ru. In some embodiments, M is Ir.

In some embodiments, m2, n2, and p2 are each 0 and each set of dashed lines represents the absence of a fused 6-membered ring, i.e., the compound has formula:

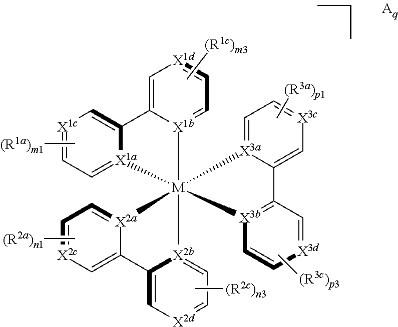

In some embodiments, X^{1a} is N, X^{1b} is C, X^{2a} is N, X^{2b} is C, X^{3a} is C, and X^{3b} is N. In some embodiments, X^{1a} is N, X^{1b} is C, X^{2a} is N, X^{2b} is C, X^{3a} is N, and X^{3b} is N.

In some embodiments, X^{1c}, X^{1d}, X^{2c}, X^{2d}, X^{3c}, and X^{3d} are each CH. In some embodiments, X^{1c}, X^{1d}, X^{2c}, X^{2d}, X^{3c}, and X^{3d} are each N.

In some embodiments, R^{1a}, R^{1b}, R^{1c}, R^{2a}, R^{2b}, R^{2c}, R^{3a}, R^{3b}, and R^{3c} are each independently selected from fluoro, methyl, tert-butyl, trifluoromethyl, and a group -Linker-Q. In some embodiments, no more than one of R^{1a}, R^{1b}, R^{1c}, R^{2a}, R^{2a}, R^{2c}, R^{3a}, R^{3b}, and R^{3c} is a group -Linker-Q.

In some embodiments, the compound comprises one group -Linker-Q, wherein Q is a capture element. In some embodiments, a capture element is an “affinity molecule,” and the corresponding capture agent is an “acceptor” (e.g., small molecule, protein, antibody, etc.) that selectively interacts with the affinity molecule. Examples of such pairs would include: an antigen as the capture element and an antibody as the capture agent, a small molecule as the capture element and a protein with high affinity for the small molecule as the capture agent (e.g., streptavidin and biotin), and the like.

In some embodiments, Q is a substrate for a dehalogenase, e.g., a haloalkane dehalogenase. Systems comprising mutant hydrolases (e.g., mutant dehalogenases) that covalently bind their substrates (e.g., haloalkyl substrates) are described, for example, in U.S. Pat. Nos. 7,238,842; 7,425,436; 7,429,472; 7,867,726; each of which is herein incorporated by reference in its entirety. For example, HALOTAG is a commercially-available modified dehalogenase enzyme that forms a stable (e.g., covalent) bond (e.g., ester bond) with its haloalkyl substrate, which finds use in embodiments herein.

In some embodiments, Q has a formula —(CH₂)ₙ—Y, wherein n is 2, 3, 4, 5, 6, 7, 8, 9, 10, 11, or 12, and Y is a halogen (i.e., F, Cl, Br, or I). In some embodiments, n is 4, 5, 6, 7, or 8, and Y is Cl. In some embodiments, n is 6 and Y is Cl, such that Q has formula —(CH₂)₆—Cl.

The linker may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

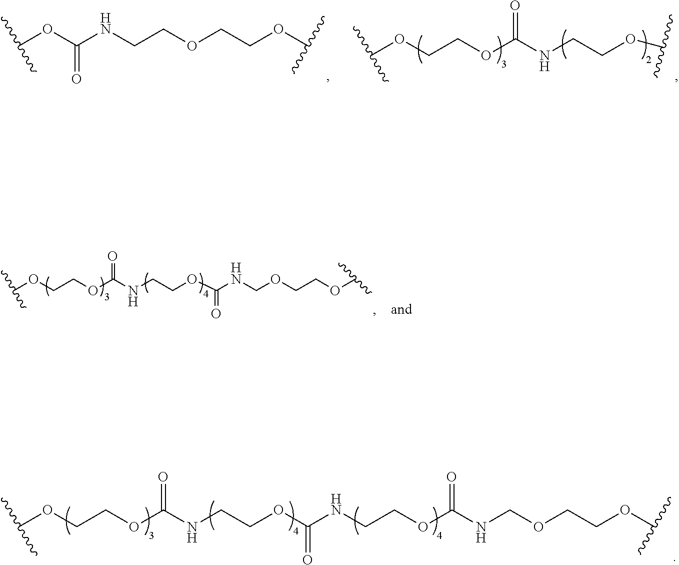

In some embodiments, q is 0, 1, or 2. Those skilled in the art will recognize that the value of q will depend on the selection of other variables and will be selected to balance the overall charge on the rest of the molecule. For example, if the overall charge of the metal-based portion of the molecule is +1, then in some embodiments, q is 1 and A is a monovalent anion (e.g., a halide or hexafluorophosphate). In some embodiments, the overall charge of the metal-based portion of the molecule is +2, then in some embodiments, q is 2 and A is a monovalent anion (e.g., a halide or hexafluorophosphate).

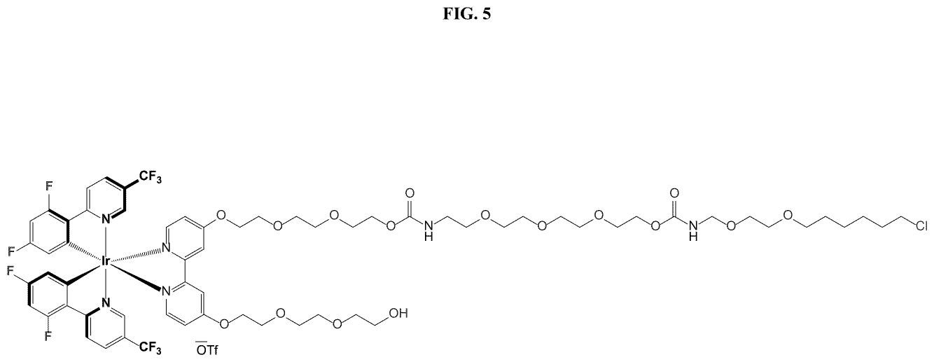

An exemplary photocatalyst linked to a HALOTAG substrate is depicted in FIG. 5. Alternative positions for attachment to the photocatalyst, other photocatalysts, different linkers and linker lengths, etc., will be understood to be within the scope herein.

In some embodiments, the photocatalyst is an organic photoredox catalyst. In some embodiments, the organic photoredox catalyst is selected from a quinone, a pyrylium, an acridinium, and a xanthene.

In some embodiments, the photocatalyst is quinone-based organic photoredox catalyst selected from:

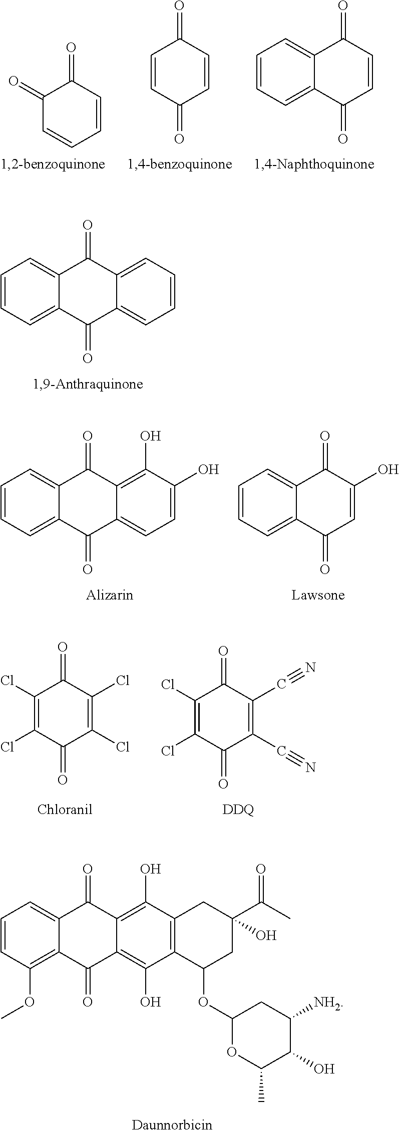

In some embodiments, the photocatalyst is pyrylium-based organic photoredox catalyst selected from:

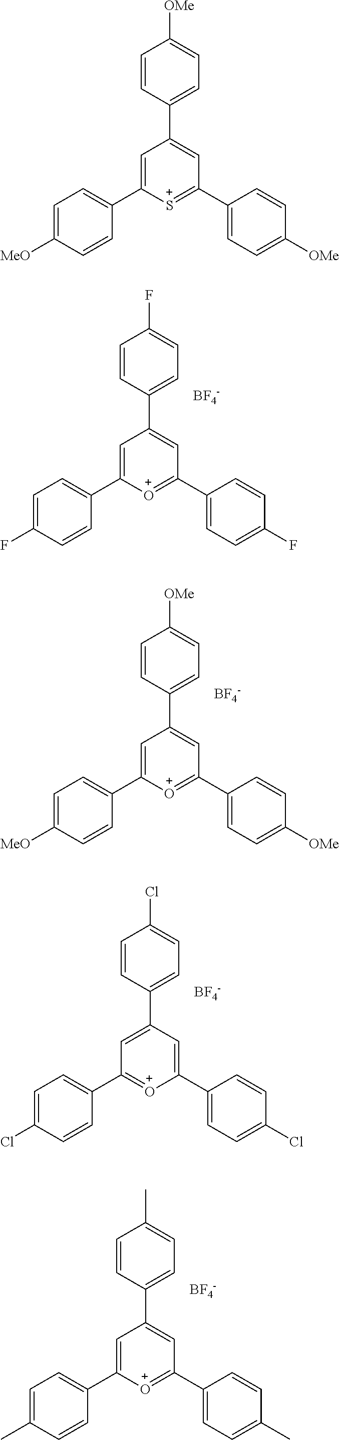

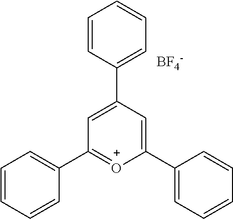

In some embodiments, the photocatalyst is acridinium-based organic photoredox catalyst selected from:

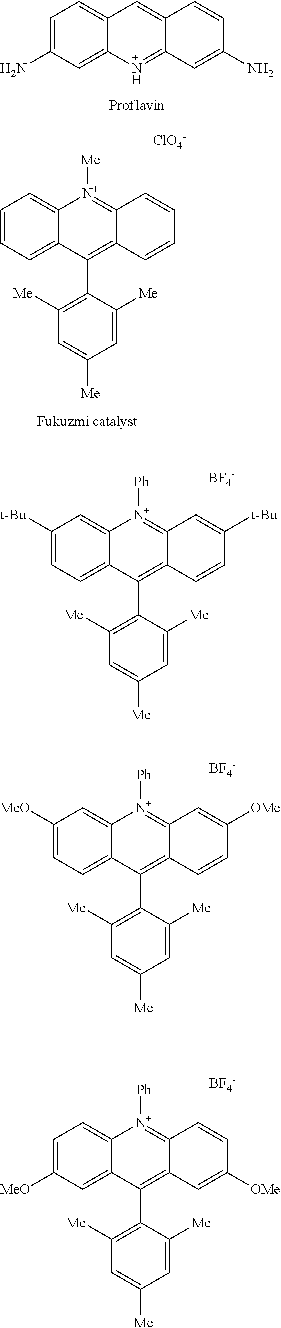

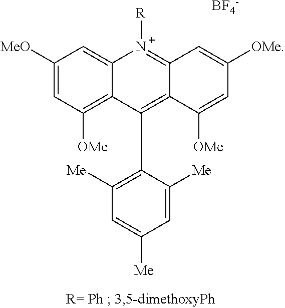

In some embodiments, the photocatalyst is xanthene-based organic photoredox catalyst selected from:

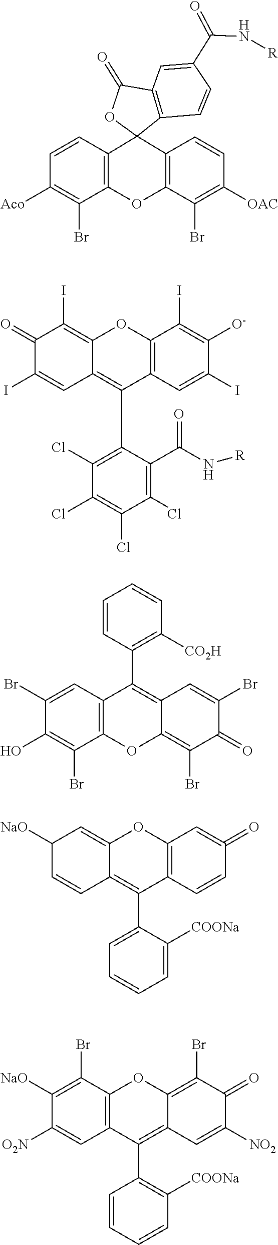

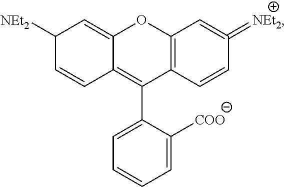

wherein R, when present represents a potential attachment site for -Linker-Q.

In some embodiments, any suitable positions in the above photocatalyst structures may find use as an attachment site for Linker-Q.

In some embodiments, the photocatalyst is thiazine-based organic photoredox catalyst selected from:

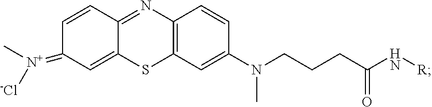

wherein R is an attachment site for Linker-Q. In some embodiments, R is an amine, a carboxyl, tert-butyl, tert-butyl-methoxy, ether, hydroxyl, PEG, etc.

In some embodiments, a photocatalyst (e.g., a quinone-based, pyrylium-based, acridinium-based, xanthene-based, or thiazine-based photoredox catalyst) is conjugated to a Linker-Q. In some embodiments, a linker (e.g., Linker-Q) is attached to the photocatalyst at any suitable position on the photocatalyst structures. In some embodiments, positions suitable for attachment of the photocatalysts are understood in the field.

### Activatable Labels

In some embodiments, systems and methods herein comprise activatable labels, which when acted upon by an excited photocatalyst, generate reactive intermediates that can form covalent linkage with neighboring biomacromolecules (e.g., attaching the activatable group to another entity, attaching a reactive moiety to another entity etc.). Embodiments herein are not limited by the mechanism of chemistry of molecular activation. In some embodiments, the activatable labels are bifunctional molecules comprising an activatable moiety and a functional moiety (e.g., detectable moiety, capture ligand, handle, etc.).

In some embodiments, the excited photocatalyst transfers energy or electrons to the activatable label. In some embodiments, the photocatalyst transfers energy to the activatable label by Forster Resonance Energy Transfer, Dexter Energy Transfer, Single Electron Transfer, Singlet oxygen, or any other suitable mechanism of energy transfer or electron transfer.

In some embodiments, the photocatalyst facilitates abstraction of a hydrogen from the activatable label. Hydrogen atom abstraction is a chemical reaction in which a hydrogen free radical is abstracted from a substrate (the activatable label) and taken on by the photocatalyst or photosensitizer. In such reactions, photoactivation of the photocatalyst or photosensitizer results in loss of a hydrogen free radical, thereby activating the photocatalyst or photosensitizer to abstract a hydrogen from the activable label, returning the photocatalyst or photosensitizer to an inactive state. Upon abstraction of the hydrogen, the activatable label is converted into an activated label.

In some embodiments, upon energy or electron transfer to the activatable label, the inert activatable moiety is converted into a reactive activated moiety.

In some embodiments, conversion of the activatable label into an activated label comprises catalyzing a redox reaction with the activatable label as a substrate for the reaction. In such embodiments, the photocatalyst or photosensitizer absorbs light and is elevated to a redox-active or excited state. The photocatalyst or photosensitizer is then capable of catalyzing a redox reaction to activate the activatable label.

In some embodiments, the activatable label comprises a photoreactive group. In some embodiments, upon activation of the photoreactive group by the exited photocatalyst, the activated label binds covalently to a target molecule. In some embodiments, the target molecule is a protein or nucleic acid.

In some embodiments, the photoreactive group comprises:

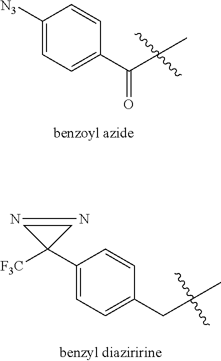

benzoyl azide, benzyl diazirine, or activatable variant thereof. In some embodiments, the activatable benzoyl azide variant is selected from:

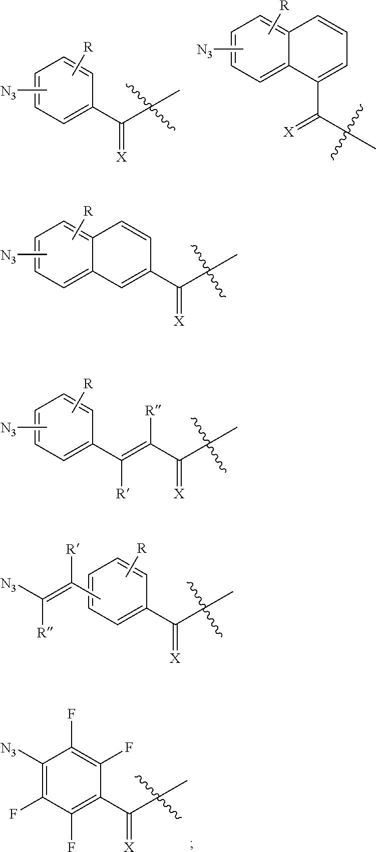

wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂; and
wherein X is O or S.

In some embodiments, the activatable benzyl diazirine variant is selected from:

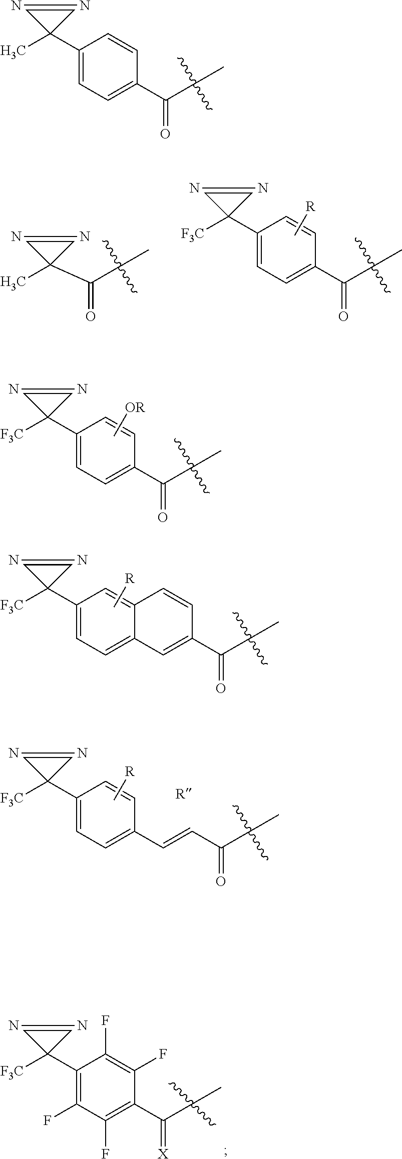

wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂.

In other embodiments, the photoreactive group comprises:

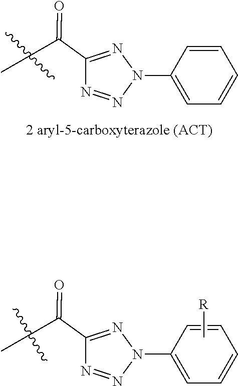

(2 aryl-5-carboxyterazole (ACT)); or an ACT derivative; wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂.

In other embodiments, the photoreactive group comprises:

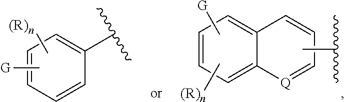

wherein:
each n is independently 1, 2, 3, or 4;
each R is independently selected from hydrogen, halo, C₁-C₄ alkyl, C₂-C₄ alkenyl, hydroxy, mercapto, amino, cyano, C₁-C₄-alkoxy, halo-C₁-C₄-alkyl, hydroxy-C₁-C₄-alkyl, amino-C₁-C₄-alkyl, mercapto-C₁-C₄-alkyl, cyano-C₁-C₄-alkyl, —C(O)—C₁-C₄-alkyl, —C(O)OH, and —C(O)NH₂;
Q is CH or N; and
   G is —N₃, —CH═CH—N₃, or

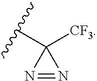 [SMILES: *C1(C(F)(F)F)N=N1]

In some embodiments, the photoreactive group comprises:

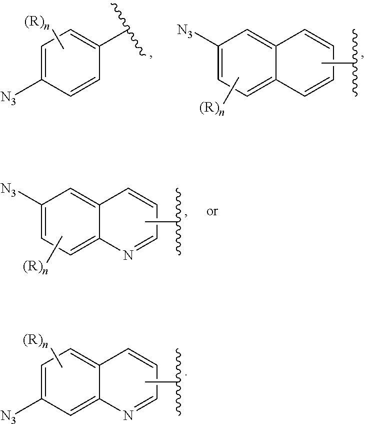

In some embodiments, the activatable label is a compound of formula (I):

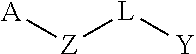

or a salt thereof, wherein:

A is the photoreactive group and is selected from:

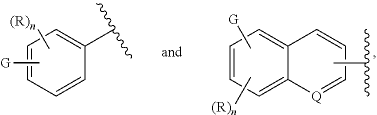

wherein:
each n is independently 1, 2, 3, or 4;
each R is independently selected from hydrogen, halo, C₁-C₄ alkyl, C₂-C₄ alkenyl, hydroxy, mercapto, amino, cyano, C₁-C₄-alkoxy, halo-C₁-C₄-alkyl, hydroxy-C₁-C₄-alkyl, amino-C₁-C₄-alkyl, mercapto-C₁-C₄-alkyl, cyano-C₁-C₄-alkyl, —C(O)—C₁-C₄-alkyl, —C(O)OH, and —C(O)NH₂;
Q is CH or N; and
G is —N₃, —CH═CH—N₃, or

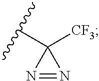 [SMILES: *C1(C(F)(F)F)N=N1]

Z is selected from —CR⁷═CR⁸—C(X)—, —C(X)—, and a bond, wherein R⁷ and R⁸ are each independently hydrogen or C₁-C₄ alkyl, and X is O or S;
L is a linker; and
Y is a functional moiety.

The group G in compounds of formula (I) or a PRG herein is or comprises an azide moiety or a diazirine moiety, which is attached to either a phenyl group or a naphthyl group. Accordingly, the compounds include aryl azide or aryl diazirine moieties which, upon exposure to light, generate reactive groups that can react with biomolecules to effect covalent modification of the biomolecule with the compound of formula (I). For example, aryl azides can undergo light-induced activation to form a reactive nitrene group, and aryl diazirines undergo light-induced activation to form a reactive carbene species.

In some embodiments, A is selected from:

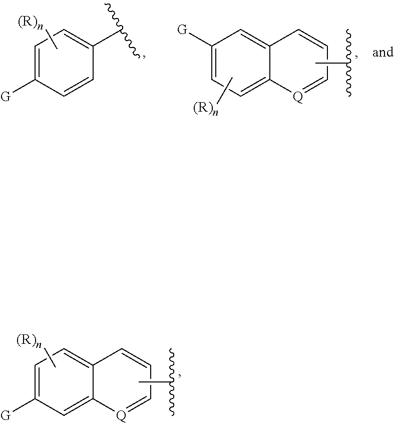

wherein G, n, R, and Q are as defined above. In some embodiments, G is —N₃. In some embodiments, G is —CH═CH—N₃. In some embodiments, G is

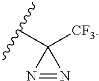 [SMILES: *C1(C(F)(F)F)N=N1]

In some embodiments, A is a group of formula:

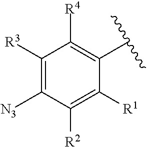

wherein R¹, R², R³, and R⁴ are each independently selected from hydrogen, halo, hydroxy, cyano, and C₁-C₄ alkoxy. In some embodiments: R¹ is hydrogen, hydroxy, or C₁-C₄ alkoxy; R² is hydrogen, halo, cyano, or C₁-C₄ alkoxy; R³ is hydrogen or halo; and R⁴ is hydrogen or halo. In some embodiments: R¹ is hydrogen, hydroxy, or methoxy; R² is hydrogen, fluoro, cyano, or methoxy; R³ is hydrogen or fluoro; and R⁴ is hydrogen.

In some embodiments, A is a group of formula:

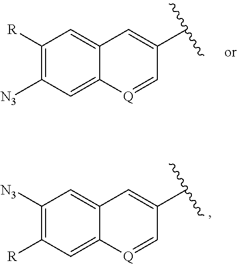

wherein R is selected from hydrogen, halo, cyano, and C₁-C₄ alkoxy. In some embodiments, R is hydrogen or cyano. In some embodiments, R is hydrogen. In some embodiments, R is cyano.

In some embodiments, A has a formula selected from:

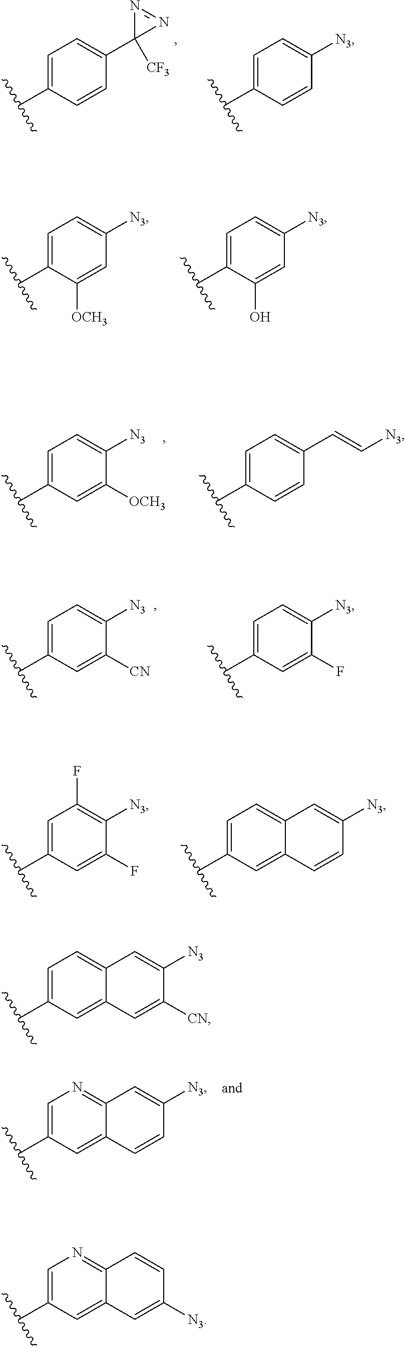

In some embodiments, Z is —CR⁷═CR⁸—C(X)—, wherein R⁷ and R⁸ are each independently hydrogen or C₁-C₄ alkyl, and X is O or S. In some embodiments, R⁷ is hydrogen. In some embodiments, R⁸ is selected from hydrogen and methyl. In some embodiments, X is O. In some embodiments, Z is —CR⁷═CH—C(O)—, wherein R⁷ is hydrogen or C₁-C₄ alkyl. In some embodiments, Z is —CR⁷═CH—C(O)—, wherein R⁷ is hydrogen or methyl. In some embodiments, Z is —C(CH₃)═CH—C(O)—. In some embodiments, Z is —CH═CH—C(O)—.

In some embodiments, Z is —C(X)—. In some embodiments, Z is —C(O)—. In some embodiments, Z is a bond.

In some embodiments, Z is selected from —C(O)— and —CR⁷═CH—C(O)—, wherein R⁷ is hydrogen or methyl.

The linker can include one or more groups independently selected from methylene (—CH₂—), ethylene (—CH═CH—), ethynylene (—C≡C—), ether (—O—), amine (—NR—) wherein R is hydrogen or an alkyl group), thioether (—S—), carbonyl (—C(O)—), thiocarbonyl (—C(S)—), sulfonyl (—S(O)₂—), arylene, heteroarylene, and heterocyclylene moieties, or any combination thereof. For example, the above moieties can be combined to form additional groups that may be included in the linker, e.g., a carbonyl group and an ether group can together provide an ester moiety (—C(O)O—); a carbonyl group and two ether groups can together provide a carbonate moiety (—OC(O)O—); a carbonyl group and an unsubstituted amine group can together provide an unsubstituted amide moiety (—C(O)NH—); a carbonyl group and two unsubstituted amine groups can together provide an unsubstituted urea moiety (—NHC(O)NH—); a carbonyl group together with an unsubstituted amine group and an ester group can provide an unsubstituted carbamate moiety (—OC(O)NH—); a carbonyl group together with a thioether and an unsubstituted amine group can provide an S-thiocarbamate moiety; a thiocarbonyl group together with an ether and an unsubstituted amine group can provide an O-thiocarbamate moiety; multiple methylene groups can together form an alkylene chain; etc. In some embodiments, the linker comprises one or more methylene, ether, ester, amide, carbamate, carbonate, urea, thioether, thioester, thioamide, thiocarbamate, thiocarbonate, thiourea, arylene, heteroarylene, or heterocyclylene moieties, or any combination thereof. In some embodiments, the linker comprises one or more —CH₂—, —O—, —C(O)O—, —C(O)NH—, —NHC(O)O—, —OC(O)O—, —NHC(O)NH—, —S—, —C(O)S—, —C(S)NH—, —NHC(S)O—, —OC(S)O—, —NHC(S)NH—, arylene, heteroarylene, or heterocyclylene moieties, or any combination thereof.

In some embodiments, the linker comprises one or more moieties selected from straight or branched chain alkylene, —O—, —NH—, —C(O)NH—, —NHC(O)O—, —NHC(O)NH—, and phenylene groups. In some embodiments, the linker comprises one or more moieties selected from straight or branched chain alkylene, —O—, and —NH— groups. In some embodiments, the linker comprises one or more ethylene glycol units (—CH₂CH₂O—).

In some embodiments, the linker has a formula:

—NHCH₂CH₂(OCH₂CH₂)ₙNH—

wherein n is 1, 2, 3, 4, 5, 6, 7, or 8. In some embodiments, n is 3, 4, 5, or 6. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

The group Y in compounds of formula (I) is a functional moiety, such as a capture element, a detectable moiety, or a reactive moiety. In some embodiments, Y is a capture element, which is a group, such as a ligand or a substrate, that forms a covalent or a non-covalent bond with a protein (a “capture protein”) upon interaction therewith. In some embodiments, the capture element is a HALOTAG ligand, which is described in, for example, in U.S. Pat. No. 7,425,436 (herein incorporated by reference in its entirety). Moieties that find use as HALOTAG ligands include haloalkane (HA) groups (e.g., chloroalkane (CA) groups). For example, in some embodiments, Y has a formula —(CH₂)ₙ-A, wherein n is 2, 3, 4, 5, 6, 7, 8, 9, 10, 11, or 12, and A is a halogen (e.g., chloro). In such embodiments, the corresponding capture protein is the HALOTAG protein, which is described in, for example, in U.S. Pat. No. 7,425,436. Another example of a capture element is biotin. For example, in some embodiments, Y has a formula:

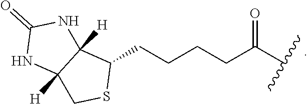 [SMILES: *C(=O)CCCC[C@@H]1SC[C@@H]2NC(=O)N[C@@H]21]

In such embodiments, the corresponding capture protein is, for example, streptavidin.

In some embodiments, Y is a detectable moiety, such as a fluorescent moiety. Suitable fluorescent functional groups include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), boron dipyrromethenes (BODIPY dyes, Invitrogen), ALEXA FLUOR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, Y comprises a fluorogenic functional group, which produces and enhanced fluorescent signal upon being associated with a target (e.g., binding of a protein to a moiety linked to the fluorogenic functional group). By producing significantly increased fluorescence (e.g., 10×, 20×, 50×, 100×, 200×, 500×, 100×, or more) upon target engagement, background signal is alleviated. Exemplary fluorogenic dyes for use in embodiments herein include the JANELIA FLUOR family of fluorophores, such as:

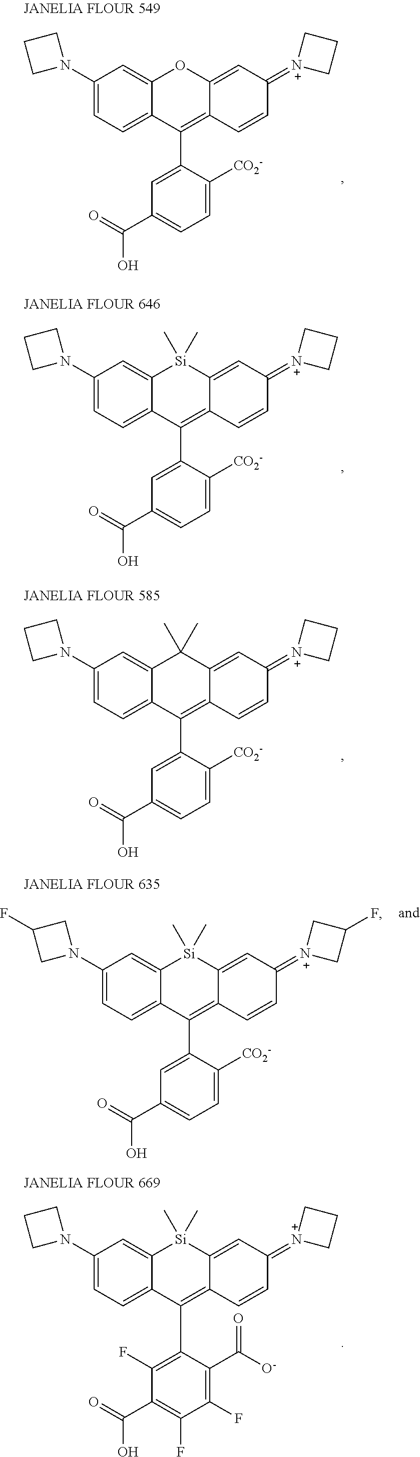

In some embodiments, Y comprises a reactive functional group, which can undergo further reaction with a corresponding reactive moiety on another molecule to effect covalent attachment. For example, in some embodiments, Y comprises a group selected from an azide, an alkyne, an alkene, or a 1,2,4,5-tetrazinyl moiety, all commonly known as “click handles” that can undergo copper-catalyzed or copper-free “click” reactions (e.g., reaction of an azide and an alkyne, reaction of an azide with a difluorinated cyclooctyne, or reaction of a 1,2,4,5-tetrazinyl group with a trans-cyclooctene moiety).

In some embodiments, the photoreactive group comprises:

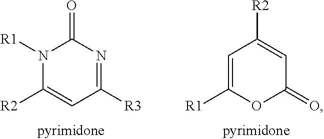

wherein one of R1-R3 is a linkage to the rest of the activatable moiety and the other two of R1-R3 are independently selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂.

In some embodiments, the photoreactive group comprises a furanocoumarin. In some embodiments, the furanocoumarin is selected from:

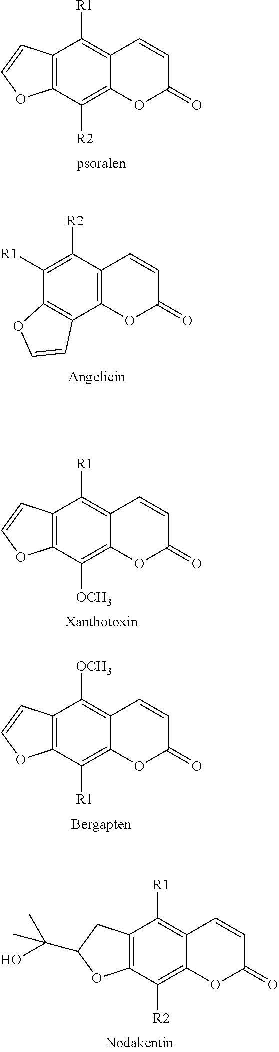

wherein one of R1-R2 is a linkage to the rest of the activatable moiety and the other R1-R2 are independently selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂.

In some embodiments, the activatable label comprises a functional moiety including a capture element (e.g., biotin, chloroalkane linker, etc.), cleavable capture element, fluorescent molecule, or a click handle (e.g., TCO, DBCO) enabling downstream enrichment, detection, or further manipulation of the labeled target molecule (e.g., biomacromolecule) via copper-free click ligation of a functional moiety. In some embodiments the fluorescent molecule is fluorogenic and selected from:

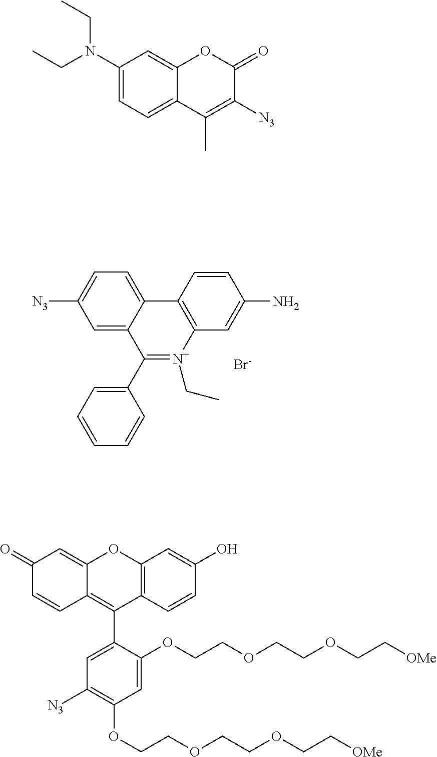 [SMILES: CCN(CC)c1ccc2c(C)c(N=[N+]=[N-])c(=O)oc2c1 ; CC[n+]1c(-c2ccccc2)c2cc(N=[N+]=[N-])ccc2c2ccc(N)cc21 ; [Br-] ; COCCOCCOCCOc1cc(OCCOCCOCCOC)c(-c2c3ccc(=O)cc-3oc3cc(O)ccc23)cc1N=[N+]=[N-]]

### Localization Elements (HALOTAG)

In some embodiments, two or more components of the systems herein are conjugated (e.g., linked, fused, etc.) to molecular elements that facilitate the localization of the components. In certain embodiments, a bioluminescent protein (or complex) and a photocatalyst are linked together, for example, via molecular localization elements connected to the bioluminescent protein (or complex) and the photocatalyst that bring the bioluminescent protein (or complex) and the photocatalyst into close enough proximity to allow light from a luminophore interacting with the bioluminescent protein (or complex) to activate the photocatalyst.

In some embodiments, the bioluminescent protein or bioluminescent complex is fused to a first molecular entity, and the photocatalyst is conjugated to a second molecular entity, wherein interaction of the first and second molecular entities places the bioluminescent protein or bioluminescent complex in sufficient proximity to the photocatalyst such that light emitted by the luminophore upon interaction with the bioluminescent protein or bioluminescent complex activates the photocatalyst. In some embodiments, the first molecular entity is a capture agent (capture protein), and the second molecular entity is a capture element.

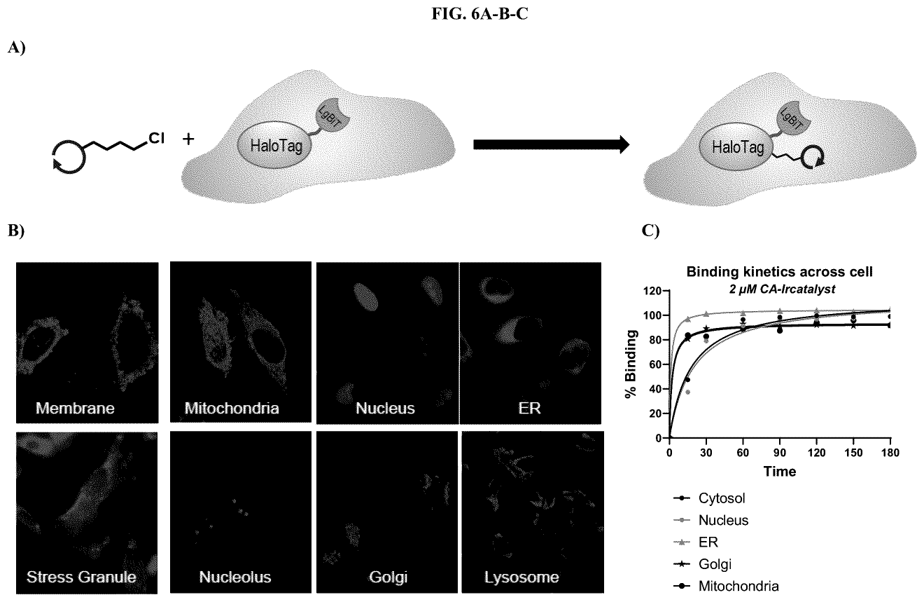

In some embodiments, the bioluminescent protein or bioluminescent complex is fused to a modified dehalogenase capable of forming a covalent bond with its substrate, and wherein the photocatalyst is conjugated to a dehalogenase substrate (See FIG. 6A). In some embodiments, binding of the modified dehalogenase to the dehalogenase substrate places the bioluminescent protein or bioluminescent complex in sufficient proximity to the photocatalyst such that light emitted by the luminophore upon interaction with the bioluminescent protein or bioluminescent complex activates the photocatalyst. In some embodiments, the minimal influence of haloalkane on cell permeability coupled with its highly specific and rapid binding of HaloTag allows for intracellular tethering of a haloalkane conjugate to HALOTAG fused to a component of the system thereby reducing the overall reliance on cellular permeability of components, and allowing localization of the system to a particular cellular compartment (FIG. 6B-C).

In some embodiments, the commercially-available HALOTAG system (Promega Corp.; Madison, WI) is utilized to link or bring together two or more components (e.g., bioluminescent protein or bioluminescent complex and photocatalyst) of the systems and methods described herein. HALOTAG is a 297-residue self-labeling polypeptide (33 kDa) derived from a bacterial hydrolase (dehalogenase) enzyme, which was modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a hydrolase with a genetically modified active site, which specifically binds to the haloalkane ligand or chloroalkane linker with an increased rate of ligand binding (Pries et al. The Journal of Biological Chemistry. 270(18):10405-11; incorporated by reference in its entirety). The reaction that forms the bond between the protein tag and chloroalkane linker is fast and essentially irreversible under physiological conditions (Waugh DS (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to a mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen DB (April 2004). Current Opinion in Chemical Biology. 8(2):150-9; incorporated by reference in its entirety).

Various HALOTAG ligands, functional groups, fusions, assays, modifications, uses, etc., are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

In some embodiments, a capture protein herein is a circularly permuted, modified dehalogenase (See, e.g., U.S. Prov. App. No. 63/338,364 and/or U.S. application Ser. No. 18/311,977; incorporated by reference in their entireties). In some embodiments, a capture protein herein is a circularly permuted, HALOTAG (cpHT) dehalogenase complexes are provided. In some embodiments, a capture protein comprises a cp variant of a polypeptide comprising at least 70% sequence identity (e.g., at least 70%, at least 75%, at least 80%, at least 85%, at least 90%, at least 95%) with SEQ ID NO: 8. In some embodiments, a capture protein comprises: (i) a first segment comprising at least 70% sequence identity (e.g., at least 70%, at least 75%, at least 80%, at least 85%, at least 90%, at least 95%) with a first portion of SEQ ID NO: 8, and (ii) a second segment comprising at least 70% sequence identity (e.g., at least 70%, at least 75%, at least 80%, at least 85%, at least 90%, at least 95%, 100%) with a second portion of SEQ ID NO: 8. In some embodiments, the first fragment and the second fragment collectively comprise amino acid sequences corresponding to at least 80% of the length of SEQ ID NO: 8 (e.g., at least 80%, at least 85%, at least 90%, at least 95%, 100%). In some embodiments, the amino acid of the polypeptide corresponding to position 297 of SEQ ID NO: 8 is peptide-bonded to the amino acid of the polypeptide corresponding to position 1 of SEQ ID NO: 8. In some embodiments, the amino acid of the polypeptide corresponding to position 297 of SEQ ID NO: 8 is connected by a linker peptide to the amino acid of the polypeptide corresponding to position 1 of SEQ ID NO: 8. In some embodiments, the linker peptide is 2 to 100 amino acids in length (e.g., 2, 3, 4, 5, 6, 7, 8, 9, 10, 15, 20, 25, 30, 35, 40, 45, 50, 55, 60, 65, 70, 75, 80, 85, 90, 95, 100, or ranges therebetween). In some embodiments, the linker peptide comprises a cleavable element (e.g., protease-cleavable site (e.g., TEV protease), chemically-cleavable site, photocleavable site, etc. In some embodiments, the capture protein is a circularly permuted variant corresponding to SEQ ID NO: 8 (e.g., having at least 70% sequence identity thereto), but having a cp site at a position corresponding to a position between positions 5 and 290 (e.g., position 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290). In some embodiments, the capture protein correspond to SEQ ID NO: 8, but with a cp site at a position corresponding to a position between positions 5 and 13 (e.g., 5, 6, 7, 8, 9, 10, 11, 12, 13, or ranges therebetween), 36 and 51 (e.g., 36, 37, 38, 39, 40, 41, 42, 43, 44, 45, 46, 47, 48, 49, 50, 11, or ranges therebetween), 63 and 72 (e.g., 63, 64, 65, 66, 67, 68, 69, 70, 71, 72, or ranges therebetween), 84 and 92 (e.g., 84, 85, 86, 87, 88, 89, 90, 91, 92, or ranges therebetween), 104 and 130 (e.g., 104, 105, 106, 107, 108, 109, 110, 111, 112, 113, 114, 115, 116, 117, 118, 119, 120, 121, 122, 123, 124, 125, 126, 127, 128, 129, 130, or ranges therebetween), 142 and 148 (e.g., 142, 143, 144, 145, 146, 147, 148, and ranges therebetween), 160 and 174 (e.g., 160, 161, 162, 163, 164, 165, 166, 167, 168, 169, 170, 171, 172, 173, 174, or ranges therebetween), 186 and 189 (e.g., 186, 187, 188, 189, or ranges therebetween), 201 and 203 (e.g., 201, 202, 203, or ranges therebetween), 221 and 229 (e.g., 221, 222, 223, 224, 225, 226, 227, 228, 229, or ranges therebetween), or 269 and 290 (e.g., 269, 270, 271, 272, 273, 274, 275, 276, 277, 278, 279, 280, 281, 282, 283, 284, 285, 286, 287, 288, 289, 290, or ranges therebetween), of SEQ ID NO: 8.

In some embodiments, a capture protein herein is a split modified dehalogenase (See, e.g., U.S. Prov. App. No. 63/338,323 and/or U.S. application Ser. No. 18/312,117; incorporated by reference in their entireties). In such embodiments, two components of a split capture protein are capable of interacting (facilitated or unfacilitated) to form a capture complex. In some embodiments, two components of a split, modified dehalogenase assemble through structural complementation into an active, modified dehalogenase complex. In some embodiments, the components of the split capture protein can be linked (e.g., fused) to different components of the systems herein (e.g., activatable label, photocatalyst, bioluminescent protein, component of a bioluminescent complex, etc.); upon assembly (facilitated or unfacilitated), the capture complex is capable of binding the capture element. In some embodiments, a split capture protein is a split HALOTAG (spHT). In some embodiments, the first and second components of a split capture protein collectively comprise at least 70% sequence similarity (e.g., at least 70%, at least 75%, at least 80%, at least 85%, at least 90%, at least 95%) with SEQ ID NO: 8. In some embodiments, the first and second components of a split capture protein collectively comprise at least 70% (e.g., at least 70%, at least 75%, at least 80%, at least 85%, at least 90%, at least 95%, 100%) sequence identity with SEQ ID NO: 8. In some embodiments, the split capture protein comprises: (i) a first fragment comprising at least 70% sequence similarity (e.g., at least 70%, at least 75%, at least 80%, at least 85%, at least 90%, at least 95%) with a first portion of SEQ ID NO: 8, and (ii) a second fragment comprising at least 70% sequence similarity (e.g., at least 70%, at least 75%, at least 80%, at least 85%, at least 90%, at least 95%, 100%) with a second portion of SEQ ID NO: 8. In some embodiments, the first fragment comprises at least 70% (e.g., at least 70%, at least 75%, at least 80%, at least 85%, at least 90%, at least 95%, 100%) sequence identity with the first portion of SEQ ID NO: 8. In some embodiments, the second fragment comprises at least 70% (e.g., at least 70%, at least 75%, at least 80%, at least 85%, at least 90%, at least 95%, 100%) sequence identity with the second portion of SEQ ID NO: 8. In some embodiments, the first fragment and the second fragment collectively comprise amino acid sequence corresponding to at least 80% of the length of SEQ ID NO: 8 (e.g., at least 80%, at least 85%, at least 90%, at least 95%, 100%). In some embodiments, the split capture protein comprises a split (“sp”) site at a position corresponding to any position between positions 5 and 290 (e.g., positions 19-34) of SEQ ID NO: 8. In some embodiments, the split capture protein comprises a sp site at a position corresponding to a position between positions 5 and 13 (e.g., 5, 6, 7, 8, 9, 10, 11, 12, 13, or ranges therebetween), 36 and 51 (e.g., 36, 37, 38, 39, 40, 41, 42, 43, 44, 45, 46, 47, 48, 49, 50, 51, or ranges therebetween), 63 and 72 (e.g., 63, 64, 65, 66, 67, 68, 69, 70, 71, 72, or ranges therebetween), 84 and 92 (e.g., 84, 85, 86, 87, 88, 89, 90, 91, 92, or ranges therebetween), 104 and 130 (e.g., 104, 105, 106, 107, 108, 109, 110, 111, 112, 113, 114, 115, 116, 117, 118, 119, 120, 121, 122, 123, 124, 125, 126, 127, 128, 129, 130, or ranges therebetween), 142 and 148 (e.g., 142, 143, 144, 145, 146, 147, 148, and ranges therebetween), 160 and 174 (e.g., 160, 161, 162, 163, 164, 165, 166, 167, 168, 169, 170, 171, 172, 173, 174, or ranges therebetween), 186 and 189 (e.g., 186, 187, 188, 189, or ranges therebetween), 201 and 203 (e.g., 201, 202, 203, or ranges therebetween), 221 and 229 (e.g., 221, 222, 223, 224, 225, 226, 227, 228, 229, or ranges therebetween), or 269 and 290 (e.g., 269, 270, 271, 272, 273, 274, 275, 276, 277, 278, 279, 280, 281, 282, 283, 284, 285, 286, 287, 288, 289, or 290 or ranges therebetween) of SEQ ID NO: 8. In some embodiments, the assembled complex of the components of the split capture protein are capable of forming a covalent bond with a haloalkane substrate. In some embodiments, a capture protein herein is a modified dehalogenase with an insertion (e.g., bioluminescent protein, component of a bioluminescent complex, circularly permuted bioluminescent protein, circularly permuted component of a bioluminescent complex, extended loop sequence, etc.) within a surface loop (See, e.g., U.S. Prov. App. No. 63/338,369 and/or U.S. application Ser. No. 18/312,441; incorporated by reference in their entireties). In some embodiments, a component of a system herein (e.g., bioluminescent protein, component of a bioluminescent complex, circularly permuted bioluminescent protein, circularly permuted component of a bioluminescent complex) is inserted between an N-terminal segment comprising at least 70% (e.g., 70%, 75%, 80%, 85%, 90%, 95%, 100%, or ranges therebetween) sequence identity with one of SEQ ID NO: 6-9 and a C-terminal segment comprising at least 70% (e.g., 70%, 75%, 80%, 85%, 90%, 95%, 100%, or ranges therebetween) sequence identity with SEQ ID NO 10-13 (e.g., SEQ ID NOS: 6/10, 7/11, 8/123, 9/13, or other combinations). In some embodiments, a component of a system herein (e.g., bioluminescent protein, component of a bioluminescent complex, circularly permuted bioluminescent protein, circularly permuted component of a bioluminescent complex) is inserted between an N-terminal segment comprising at least 70% (e.g., 70%, 75%, 80%, 85%, 90%, 95%, 100%, or ranges therebetween) sequence identity with one of SEQ ID NO: 14-20 and a C-terminal segment comprising at least 70% (e.g., 70%, 75%, 80%, 85%, 90%, 95%, 100%, or ranges therebetween) sequence identity with SEQ ID NO 21-27 (e.g., SEQ ID NOS: 14/21, 15/22, 16/23, 17/24, 18/25, 19/26, 20/27, or other combinations). In some embodiments, a component of a system herein (e.g., bioluminescent protein, component of a bioluminescent complex, circularly permuted bioluminescent protein, circularly permuted component of a bioluminescent complex) is inserted between an N-terminal segment comprising at least 70% (e.g., 70%, 75%, 80%, 85%, 90%, 95%, 100%, or ranges therebetween) sequence identity with one of SEQ ID NO: 81-85 and a C-terminal segment comprising at least 70% (e.g., 70%, 75%, 80%, 85%, 90%, 95%, 100%, or ranges therebetween) sequence identity with SEQ ID NO 86-90 (e.g., SEQ ID NOS: 81/86, 82/87, 83/88, 84/89, 85/90, or other combinations).

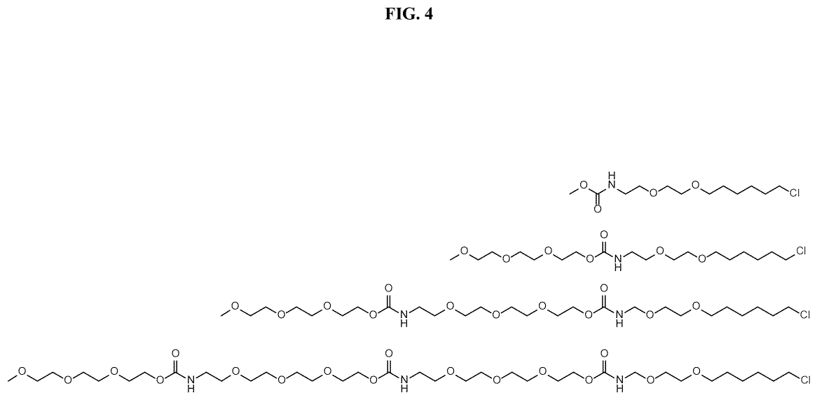

In some embodiments, a first component of the systems herein (e.g., a bioluminescent protein or component of a bioluminescent complex) is fused (e.g., expressed as a fusion) to a modified dehalogenase (e.g., HALOTAG or a variant thereof) or inserted into a surface loop of a modified dehalogenase and a second component of the systems herein (e.g., a photocatalyst) is tethered (e.g., directly or via a linker) to a dehalogenase substrate (e.g., haloalkane). For example, the structure of the photocatalyst tethered to the dehalogenase substrate is P-linker-AX, wherein P is the photocatalyst, wherein A is (CH₂)₂₋₁₂, wherein X is a halogen, and wherein the linker is a linker moiety capable of tethering P to A-X. In some embodiments, the linker is a multiatom straight or branched chain including C, N, S, or O, or a group that comprises one or more rings, e.g., saturated or unsaturated rings, such as one or more aryl rings, heteroaryl rings, or any combination thereof. In some embodiments, the linker comprises a combination of —O(CH₂)₂— —(CH₂)O—, —CH₂—, —NHC(O)O—, —OC(O)NH—, NHC(O)—, and —C(O)NH—. In some embodiments, the linker is 5 to 50 (e.g., 5, 10, 15, 20, 25, 30, 35, 40, 45, 50, or ranges therebetween) atoms in length. In some embodiments, the length of the linker for tethering the photocatalyst allows for optimization of geometry (e.g., for energy transfer). Exemplary linker-A-X groups are depicted in FIG. 4. In some embodiments, a first component of the systems herein (e.g., a bioluminescent protein or component of a bioluminescent complex) is inserted (e.g., expressed as an internal fusion) within a modified dehalogenase (e.g., HALOTAG or a variant thereof) in order to increase proximity or provide a geometry that is favorable of energy transfer to the bound catalyst (See, e.g., U.S. Prov. App. No. 63/338,369; U.S. application Ser. No. 18/312,441; incorporated by reference in their entireties). In some embodiments, the location for insertion within the modified dehalogenase (e.g., HALOTAG or a variant thereof) is selected to provide optimal proximity and geometry for the desired interactions between components, while maintaining the function or activity of the modified dehalogenase (e.g., HALOTAG or a variant thereof) and inserted component.

The scope of embodiments herein is not limited by the types of linkers available. Components and moieties thereof may be linked either directly (e.g., linker consists of a single covalent bond) or linked via a suitable linker. Embodiments are not limited to any particular linker group. A variety of linker groups are contemplated, and suitable linkers could comprise, but are not limited to, alkyl groups, methylene carbon chains, ether, polyether, alkyl amide linker, a peptide linker, a modified peptide linker, a Poly(ethylene glycol) (PEG) linker, a streptavidin-biotin or avidin-biotin linker, polyaminoacids (e.g., polylysine), functionalized PEG, polysaccharides, glycosaminoglycans, dendritic polymers (WO93/06868 and by Tomalia et al. in Angew. Chem. Int. Ed. Engl. 29:138-175 (1990), herein incorporated by reference in their entireties), PEG-chelant polymers (W94/08629, WO94/09056 and WO96/26754, herein incorporated by reference in their entireties), oligonucleotide linker, phospholipid derivatives, alkenyl chains, alkynyl chains, disulfide, or a combination thereof. In some embodiments, the linker is cleavable (e.g., enzymatically (e.g., TEV protease site), chemically, photoinduced, etc.

In some embodiments, a modified dehalogenase (e.g., HALOTAG) and dehalogenase ligand (e.g., haloalkane) are used to tether any two components of the systems and methods described herein (i.e., not limited to tethering of the bioluminescent protein or component of a bioluminescent complex to the photocatalyst). In other embodiments, the bioluminescent protein or component of a bioluminescent complex is tethered to the photocatalyst (or other components described herein) by another mechanism.

In some embodiments, a first component of a system or method herein is linked (e.g., fused) to a capture agent (e.g., capture protein) and a second component of the system or method is linked to a capture element. Binding of the capture element by the capture agent (e.g., capture protein) results in co-localization of the first component and the second component. In some embodiments, the capture agent is a modified dehalogenase, and the capture element is a haloalkane. However, other capture agent/element pairs that may find use in embodiments herein include streptavidin/biotin, antibody (or Ab fragment) and antigen, etc.

In other embodiments, components herein are connected by chemical modification/conjugation, such as by Native chemical ligation, Staudinger ligation, “traceless” Staudinger ligation, amide coupling, methods that employ activated esters, methods to target lysine, tyrosine and cysteine residues, imine bond formation (with and without ortho-boronic acid), boronic acid/diol interactions, disulfide bond formation, copper/copper free azide, diazo, and tetrazine “click” chemistry, UV promoted thiolene conjugation, diazirine photolabeling, Diels-Alder cycloaddition, metathesis reaction, Suzuki cross-coupling, 2-cyanobenzothiazole (CBT) coupling, 2-pyridinecarboxyaldehyde (PCA) coupling etc.

### Target Molecules and Localizing Elements

In some embodiments, an activated label binds to a target molecule (e.g., cellular target, protein, nucleic acid). In some embodiments, a component of the systems herein is configured to bind to, localize with, or otherwise associate with the target molecule.

In some embodiments, the bioluminescent protein or complex is conjugated to a target binding agent, wherein the target binding agent is capable of binding to the target molecule (e.g., protein, nucleic acid, or other biological molecules (e.g., lipid, sugar, etc.)). In some embodiments, the target binding agent is a protein or peptide fused directly or indirectly to the bioluminescent protein or a component of the bioluminescent complex. In some embodiments, the target molecule is a nucleic acid, and the target binding agent is capable of binding specifically or non-specifically to nucleic acids. In some embodiments, the target binding agent is a wildtype or modified Cas protein (e.g., Cas9, dCas9, dCas12, dCas13, etc.), and the target molecule is a nucleic acid that is modified by CRISPR. In some embodiments, systems further comprise a guide RNA (gRNA). In some embodiments, the target molecule is a target peptide or protein, and the target binding agent is capable of binding to the target peptide or protein. In some embodiments, the target binding agent is a small molecule or nucleic acid tethered directly or indirectly to the bioluminescent protein or a component of the bioluminescent complex.

In one set of embodiments, the bioluminescent protein, a component of the bioluminescent complex, the photocatalyst, or the activatable label is tethered to a specific ligand, nucleic acid, or a targeting protein (e.g., Cas9, dCas9, dCas12, dCas13, etc.). Exemplary targeting ligands include small molecule/drug/signaling molecule that bind specifically to the target. In some embodiments, a photocatalyst is tethered to such a small molecule/drug/signaling molecule, thereby allowing localization of the photocatalyst with a protein of interest that is fused to HiBiT or NanoLuc. Other exemplary targeting proteins/ligands include an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins. Tethering the catalyst to a binding domain that recognize a target protein allows for localization of the catalyst with a protein of interest that is already fused to HiBiT or NanoLuc. In some embodiments, approaches are used to increase proximity with the activatable label, for example using a functional moiety that has general affinity to nucleic acids; using a trifunctional molecule comprising a photoreactive moiety; a functional moiety and a recognition moiety that directly bind the target protein; etc.

In this case, complementation of HiBiT fused to a protein of interest with LgBiT-HaloTag-catalyst localize the photocatalytic system to a protein of interest. Similarly, LgBiT fused to a protein of interest will localize the protein of interest to a HiBiT-HaloTag-photocatalyst system.

### Systems

In some embodiments, two or more (e.g., 2, 3, 4, or more) of the components of the systems described herein are conjugated together. In some embodiments, one or more pairs of components of the systems described herein are conjugated together. For example, the following pairs of components may be conjugated (e.g., tethered by a linker, genetically fused, etc.): bioluminescent protein and capture protein; photocatalyst and capture ligand; bioluminescent protein and photocatalyst; component of bioluminescent complex and capture protein; component of bioluminescent complex and photocatalyst; bioluminescent protein and target molecule; components of bioluminescent complex and target molecule; bioluminescent protein and target binding agent (e.g., protein, antibody, antibody fragment, antibody-binding agent, nucleic acid, small molecule ligand, etc.); component of bioluminescent complex and target binding agent (e.g., protein, antibody, antibody fragment, antibody-binding agent, nucleic acid, small molecule ligand, etc.); component of bioluminescent complex and photocatalyst; component of bioluminescent complex and capture ligand; activatable label and capture ligand; capture protein and target binding agent (e.g., protein, antibody, antibody fragment, antibody-binding agent, nucleic acid, small molecule ligand, etc.); etc.

Components of the systems described herein may be delivered, combined, and/or produced in any suitable manners for a particular application. In embodiments in which the system resides within a cell, components may be expressed within the cell, added exogenously, and allowed to enter the cell (i.e., cell permeable components), or may be delivered to the cell. Delivery of components to the cell may be performed in any suitable delivery vehicle, such as liposomes, micelles, nanoparticles, viruses, etc. In some embodiments, components are tagged to facilitate delivery into cells (e.g., linked to a membrane translocating motif). In some embodiments, components are included to facilitate cellular uptake and/or subsequent endosomal escape. Such additional components may include modified polyethyleneimine polymers and modified poly(amidoamine) dendrimers for use in delivering biomolecules (e.g., delivery of components that cannot passively enter the cell, but cannot be expressed within the cell (e.g., LgBiT/photocatalyst direct conjugates, etc.) to cells (See, U.S. Pub. No. 2020/0399660; incorporated by reference in its entirety).

Exemplary components combinations of systems within the scope herein include:
HiBiT fused to a protein of interest; fusion of LGBIT and HALOTAG: photocatalyst conjugated to a haloalkyl HALOTAG ligand—HALOTAG binds to the haloalkyl ligand; the high affinity of HIBIT for LGBIT forms a bioluminescent complex and localizes the photocatalytic system to the protein of interest, and bioluminescence triggers the photocatalyst.
A dipeptide comprising HIBIT-TRIP9 fused to a protein of interest; fusion of LGTRIP and HALOTAG: photocatalyst conjugated to a haloalkyl HALOTAG ligand—HALOTAG binds to the haloalkyl ligand; the high affinity of HIBIT-TRIP9 for LGTRIP forms a bioluminescent complex and localizes the photocatalytic system to the protein of interest, and bioluminescence triggers the photocatalyst.
A fusion of HIBIT and an antisense oligonucleotide; LGBIT fused to HALOTAG; photocatalyst conjugated to a haloalkyl HALOTAG ligand—HALOTAG binds to the haloalkyl ligand; the high affinity of HIBIT for LGBIT forms a bioluminescent complex and hybridization of the antisense oligonucleotide to a target nucleic acid sequence localizing the photocatalytic system to a DNA/RNA of interest, and bioluminescence triggers the photocatalyst.
SMBIT fused to an antibody for a target analyte; LGBIT/HALOTAG fused to a general immunoglobulin binding moiety; photocatalyst conjugated to a haloalkyl HALOTAG ligand-HALOTAG binds to the haloalkyl ligand; binding of the antibody allows facilitated complementation and localizes the photocatalytic system to the target analyte, and bioluminescence triggers the photocatalyst.
Fusions of HIBIT and Trip9 to antisense oligonucleotides that targets the same DNA/RNA of interest; LGTRIP fused to HALOTAG: photocatalyst conjugated to a haloalkyl HALOTAG ligand—HALOTAG binds to the haloalkyl ligand; hybridization of the antisense oligonucleotides to a target nucleic acid sequence allows facilitated complementation and localizes the photocatalytic system to a DNA/RNA of interest, and bioluminescence triggers the photocatalyst.
dCas9 or dCas12g1 fused to both HaloTag and NanoLuc; photocatalyst conjugated to a haloalkyl HALOTAG ligand—HALOTAG binds to the haloalkyl ligand;
gRNA targets the photocatalytic system to a DNA/RNA of interest, and bioluminescence triggers the photocatalyst.

### Applications

In some embodiments, the systems and methods herein are utilized to carry-out various applications (e.g., within cells). A variety of functional proteomic and genomic analyses, in addition to other applications, are made possible by the advantages of the systems and methods herein. Exemplary proteomic focused applications for the systems and methods herein include: proximity-based protein labeling and subsequent detection of dynamic microenvironments, protein-protein interactions, and cell-cell interactions under relevant physiological condition; labeling a protein fused to HiBiT as well as proteins in its close vicinity with a fluorophore for downstream sorting/detection of cells expressing a HiBiT fusion; labeling a protein fused to HiBiT with a fluorophore while keeping the active site free to interact with other ligands in order to subsequently monitor protein dynamics and trafficking; protein labeling with a click-handle for subsequent attachment of diverse functional groups such as a small molecule drug, PROTAC, etc. for down-stream manipulation of a protein of interest; etc.

Exemplary genomic focused applications for the systems and methods herein include: utilizing—a photocatalytic system comprising HaloTag-NanoLuc fused to a Cas protein (e.g., Cas9, dCas9, dCas12, dCas13) and tethered to a component of the system to facilitate proximity-labeling of DNA or RNA loci targeted by the Cas protein with either biotin for enrichment and subsequent sequence analysis, a fluorophore for detection and visualization, or a click handle for subsequent attachment of diverse functionalities. Such system could be used to identify Cas9 off-target effects as well as mapping nucleic acid protein interactions.

In some embodiments, provided herein are methods of proximity-dependent activation of an activatable label within a cell, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst, wherein the capture protein forms a covalent bond with the capture ligand upon interaction therewith, and wherein the photocatalyst is activated by exposure to light of the first wavelength; and (c) an activatable label, wherein the activatable label is converted into an activated label when in proximity to the activated photocatalyst.

In some embodiments, provided herein are methods of inducing a proximity-dependent bioorthogonal chemical ligation, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst, wherein the capture protein forms a covalent bond with the capture ligand upon interaction therewith, and wherein the photocatalyst is activated by exposure to light of the first wavelength; (c) an activatable label, wherein the activatable label is converted into an activated label when in proximity to the activated photocatalyst; and (d) a target molecule, wherein the activated label form a covalent linkage with the target molecule when in proximity therewith.

In some embodiments, provided herein are methods of proximity-dependent activation of an activatable label within a cell, comprising: (a) expressing a fusion of a bioluminescent protein and a capture protein within the cell; (b) contacting the cell with a luminophore under conditions in which the luminophore enters the cell, wherein the bioluminescent protein catalyzes emission of a first wavelength of light from the luminophore upon interaction therewith; (c) contacting the cell with a conjugate of (i) a capture ligand and (ii) a photocatalyst under conditions in which the conjugate enters the cell, wherein the capture protein forms a covalent bond with the capture ligand upon interaction therewith, and wherein the photocatalyst is activated by exposure to light of the first wavelength; and (d) contacting the cell with an activatable label, wherein the activatable label is converted into an activated label when in proximity to the activated photocatalyst.

In some embodiments, provided herein are methods of inducing a proximity-dependent bioorthogonal chemical ligation with a target molecule, comprising: (a) expressing a fusion of a bioluminescent protein and a capture protein within a cell; (b) contacting the cell with a luminophore under conditions in which the luminophore enters the cell, wherein the bioluminescent protein catalyzes emission of a first wavelength of light from the luminophore upon interaction therewith; (c) contacting the cell with a conjugate of (i) a capture ligand and (ii) a photocatalyst under conditions in which the conjugate enters the cell, wherein the capture protein forms a covalent bond with the capture ligand upon interaction therewith, and wherein the photocatalyst is activated by exposure to light of the first wavelength; and (d) contacting the cell with an activatable label, wherein the activatable label is converted into an activated label when in proximity to the activated photocatalyst; wherein the activated label form a covalent linkage with the target molecule when in proximity therewith.

In some embodiments, provided herein are methods of proximity-dependent activation of a photocatalyst within a cell, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst, wherein the capture protein forms a covalent bond with the capture ligand upon interaction therewith, and wherein the photocatalyst is activated by exposure to light of the first wavelength.

In some embodiments, provided herein are methods of proximity-dependent activation of a photocatalyst within a cell, comprising: (a) expressing a fusion of a bioluminescent protein and a capture protein within the cell; (b) contacting the cell with a luminophore under conditions in which the luminophore enters the cell, wherein the bioluminescent protein catalyzes emission of a first wavelength of light from the luminophore upon interaction therewith; and (c) contacting the cell with a conjugate of (i) a capture ligand and (ii) a photocatalyst under conditions in which the conjugate enters the cell, wherein the capture protein forms a covalent bond with the capture ligand upon interaction therewith, and wherein the photocatalyst is activated by exposure to light of the first wavelength.

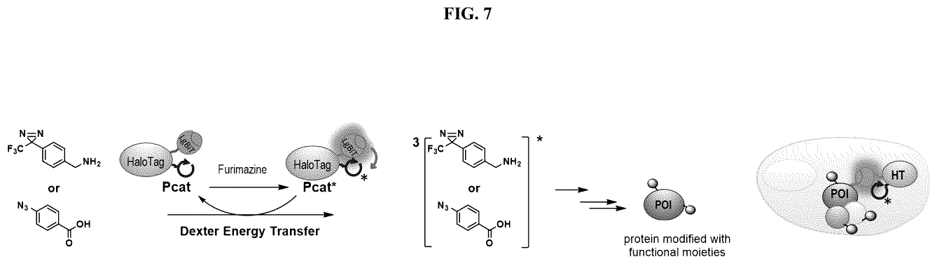

One exemplary general embodiment of the systems and methods herein is depicted in FIG. 7. In this embodiment, a first component of a bioluminescent complex (e.g., LgBiT component of NanoBiT) is conjugated (e.g., fused) to a capture protein (e.g., HALOTAG). In some versions of this embodiment, the first component of the bioluminescent complex and the capture protein are expressed as a fusion within a cell. A photocatalyst is linked to a capture ligand (e.g., comprising a haloalkane). In some embodiments, the photocatalyst linked to the capture ligand is added extracellularly and is capable of entering the cell (e.g., without permeabilizing the cell) and forming a covalent bond with the capture protein. Exposure of the first component of a bioluminescent complex to a second component of the bioluminescent complex (e.g., HiBiT) and a suitable luminophore (e.g., furimazine, fluorofurimazine, etc.) results in formation of an active bioluminescent complex and emission of light. Exposure of the photocatalyst to the light emitted from the bioluminescent complex activates the photocatalyst. The activated photocatalyst subsequently engage in energy transfer events with activatable labels within its surrounding vicinity to generate reactive intermediates that can form covalent linkage with neighboring proteins.

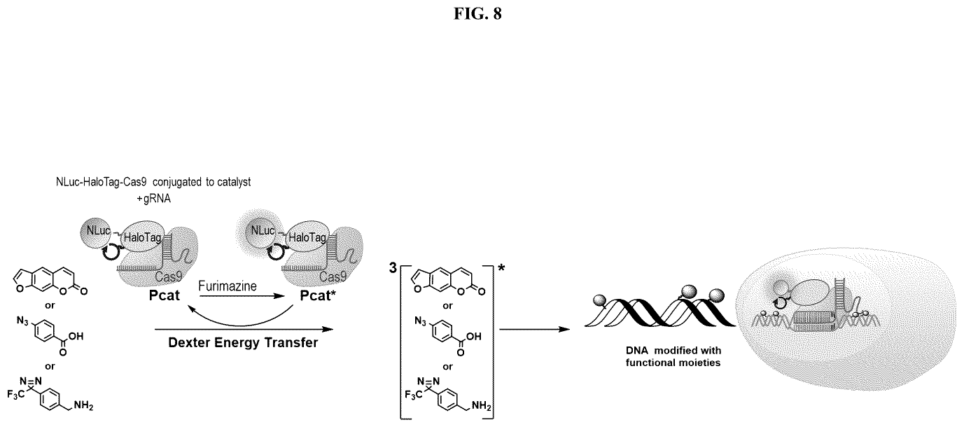

In the embodiment depicted in FIG. 7, a bioluminescent complex is utilized as the light source. In other embodiments such as depicted in FIG. 8, a bioluminescent protein is utilized in place of the bioluminescent complex. The selection of a bioluminescent complex or protein is determined based on the particular application. When applicable, embodiments described for use with one bioluminescent entity here can also find use with other bioluminescent entities herein or as understood in the field.

In some embodiments, the use of complementation to form a bioluminescent complex provides various advantages over other systems (e.g., utilizing a laser or LED as a light source) or systems herein that utilize a bioluminescent protein as the light source. For example, the use of a HiBiT/LgBiT a complementation system (or other NanoBiT-based complementation systems) as the principle light source coupled with a broad toolkit of photocatalyst/activatable label pairs offer multiple advantages within living cells or other biological systems. HiBiT is small, minimally perturbing tag, suitable for tagging endogenous target proteins. LgBiT can be tethered to a photocatalyst (e.g., via HALOTAG or another capture system), which offers a modality agnostic approach to, for example, induce proximity between the catalyst and protein of interest-tagged with HiBiT, induce proximity between the catalyst and bioluminescence source (HiBiT/LgBiT), produce greater spatiotemporal resolution through conditional activation (+Furimazine) at a specific site (HiBiT/LgBiT complementation), utilize chloroalkane chemistry to provide a convenient approach to tether the catalyst to a HaloTag-LgBiT fusion either biochemically or in cells at a specific compartment expressing the fusion, and deliver the photocatalytic system to sites of interest (e.g., intracellularly or extracellularly). The use of a bioluminescent complex (or bioluminescent protein) provides for local delivery of light of an appropriate wavelength (e.g., blue light) for catalyst activation inside intact cells or other complex models. Other embodiments herein utilize a SmBiT/LgBiT complementation system or another complementation system that requires external complementation (e.g., facilitation) to form a bioluminescent complex. Because SmBiT and LgBiT do not form an active bioluminescent complex without facilitation, the use of such components in the systems/methods herein can be used to require an additional localization event (e.g., the binding of an element conjugated (directly or indirectly) to SmBiT to an element conjugated (directly or indirectly) to LgBiT) in order to produce light to activate the photocatalyst.

In other embodiments, the use of a bioluminescent protein provides various advantages over other systems (e.g., utilizing a laser or LED as a light source) or systems herein that utilize a bioluminescent complex as the light source. For example, a bioluminescent protein (e.g., NANOLUC) provides a single-entity light source that can be expressed within cells (e.g., alone or as a fusion with other components of the systems herein). In certain embodiments herein, the enhanced simplicity/efficiency of a single entity light source is preferred over embodiments requiring complementation.

FIG. 7 depicts a system that allows for bioluminescence-triggered spatiotemporal protein labeling in intact cells (with spatial relationships preserved). Such labeling would allow for subsequent enrichment and identification by mass spectrometry or other detection/quantification analysis. Such embodiments may find use in mapping dynamic interactomes and protein complexes, mapping interactomes of memberless subcellular compartments, mapping protein translocation and secretion, etc.

FIG. 8 depicts a system for bioluminescence-triggered activation of an activatable label for covalent crosslinking with proximal dsDNA. In such embodiments, CRISPR enzymes conjugates (i.e., Cas-NanoLuc-HaloTag-catalyst) are used in combination with sgRNA to target a specific DNA locus for spatiotemporal DNA/protein labeling in intact cells (with spatial relationships preserved). Analogous systems with other protein- or nucleic acid-binding proteins conjugated to the components for the system are within the scope herein. In the CRISPR/Cas system, a guide RNA (gRNA) binds to a specific target sequence in the genomic DNA of a cell. The Cas9 enzyme binds to the complex of the gRNA and the target sequence. The Cas9 enzyme then cuts the target DNA at the targeted location. Once the DNA is cleaved, the cell's DNA repair machinery repairs the cleavage, resulting in customized changes to the target sequence. In some embodiments, by linking a bioluminescent protein (e.g., NanoLuc) or component of a bioluminescent complex to a component of a CRISPR system, and linking the photocatalyst the bioluminescent protein (e.g., NanoLuc) or component of a bioluminescent complex (e.g., directly or via HaloTag and a HaloTag ligand), the photocatalyst will be activated in proximity of the target DNA and sites of CRISPR modification can be labeled by the systems herein. Although Cas9 is the enzyme that is used most often, other enzymes (e.g., Cpf1) can also be used in CRISPR systems (and the labeling systems herein).

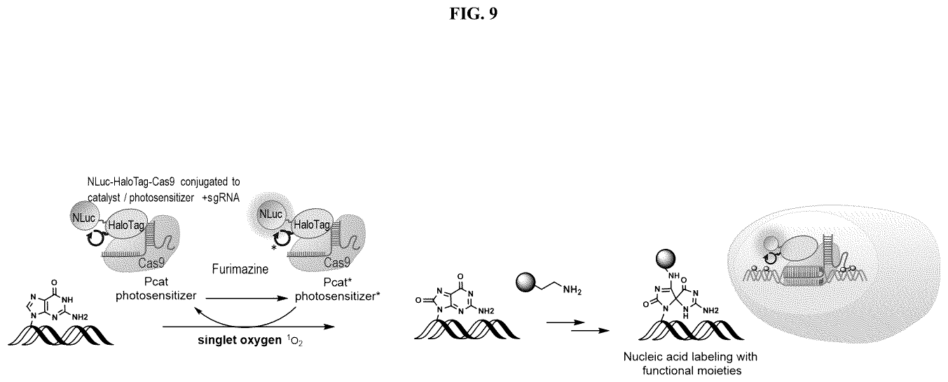

FIG. 9 depicts a system for bioluminescence-triggered generation of singlet oxygen for covalent labeling of proximal nucleic acids. CRISPR enzymes conjugates (e.g., Cas-NanoLuc-HaloTag-catalyst) are utilized in combination with sgRNA to target a specific DNA/RNA locus for spatiotemporal labeling in intact cells (with spatial relationships preserved). This exemplary system generates a shortly diffused singlet oxygen for proximal functionalization of predominantly guanine bases. Because of the lack of an external light source and the local conditional generation of singlet oxygen, issues with cytotoxicity are greatly reduced.

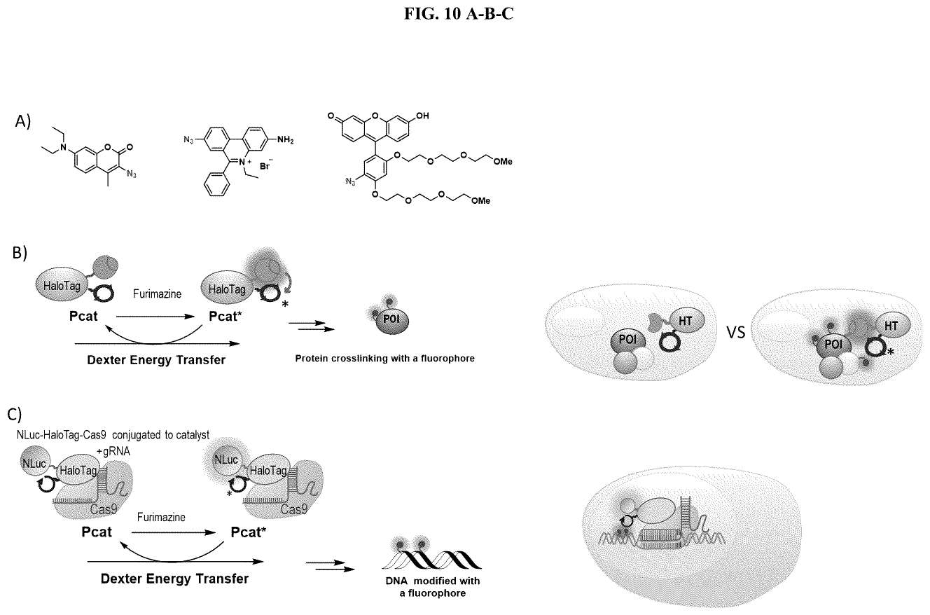

FIGS. 10 A, B, and C depict a system for bioluminescence-triggered covalent labeling of a protein genetically fused to HiBiT with a fluorogenic molecule. Such embodiments allow for converting bioluminescence to fluorescence for cell sorting applications. Means are currently unavailable for bioluminescence-based cell sorting. Systems here allow for bioluminescence-triggered activation of fluorogenic molecules. Using, for example, azide-quenched fluorogenic dyes, dye activation can be coupled with covalent protein labeling. Analogous systems (e.g., using the same EMA dye) are useful for labeling of nucleic acids. Similar approach utilizing a genetic fusion of Cas-NanoLuc-HaloTag tethered to a catalyst in combination with a sgRNA could be used for fluorescent labeling of a DNA/RNA locus of interest.

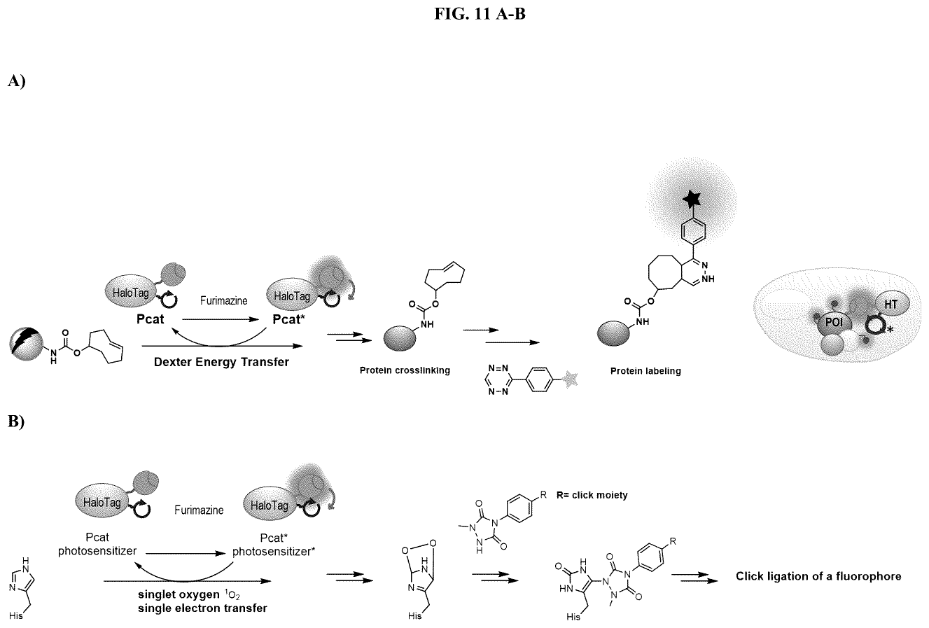

FIGS. 11A and 11B depict systems for bioluminescence-triggered proximal incorporation of a click-handle (e.g., TCO, Tetrazine, DBCO, N3), for subsequent bioorthogonal ligation of a fluorophore or other functional moieties. (A) Depicted is a two-step coupling covalent incorporation of a click handle (i.e., TCO) and subsequent labeling with a fluorophore. Such methods address potential quenching of the catalyst by the fluorophore. Similar approach could also be used to label nucleic acid of interest.

Other applications that make use of the systems, methods, and components herein are within the scope of the technology.

### EXPERIMENTAL

### Example 1

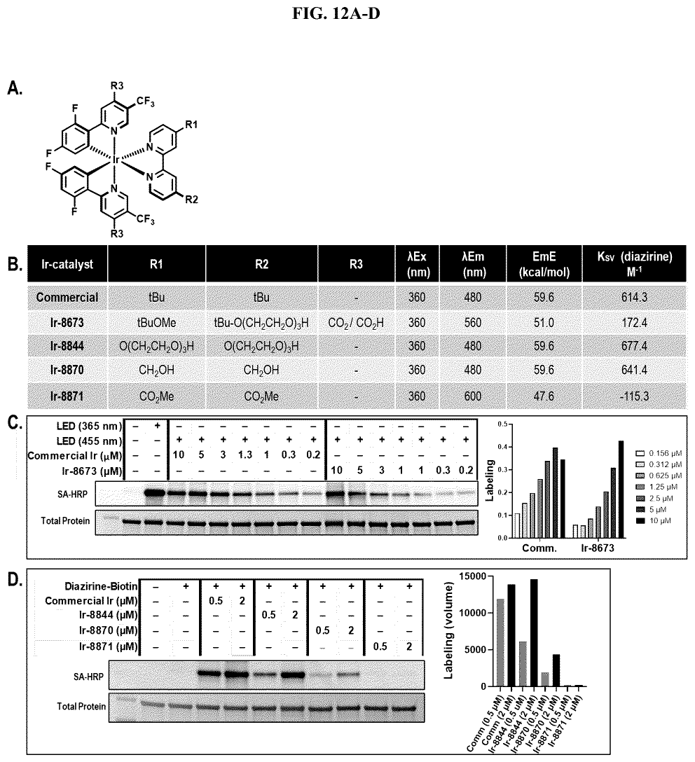

During the development of bioluminescence-triggered photocatalytic labeling described herein, experiments were conducted to evaluate the influence of modifications (R1-R3) on an iridium catalyst's physicochemical properties and subsequent capacity to drive photocatalytic labeling (FIG. 12). The model catalyst and site of modifications are shown in FIG. 12A. Structures and syntheses of these catalysts are included in Example 12.

The influences of the structural modifications described in FIG. 12B on the catalyst's physiochemical properties and subsequent capacity to undergo energy transfer events with diazirine residues were determined using the following analyses:
a. Excitation and emission profiles: the excitation and emission spectra of 200 μM catalysts in 100% DMSO were monitored on a SPARK multimode plate reader using the following setups: 380 nm excitation for emission scans and either 480 nm, 560 nm, or 600 nm emission for excitation scans.
b. Emission energies (EmE) were calculated from λ_{Em} using the Planck's equation

E(J)=(h×c)/λ
where h (Planck's constant)=6.625×10⁻³⁴ J×sec; c (speed of light)=3×10⁸ m/sec and λ(m) is λ_{Em} Energies in Joules were converted to cal/mol using the conversion of 1 J=0.239 Cal:

$$E ( \frac{cal}{mol} ) = J \times 0.239 \times Avogadro number$$

where Avogadro number=6.02214×10²³
c. Efficiencies of energy transfer from the excited catalysts to diazirine residues were driven from a Stern-Volmer quenching relationship analysis monitoring the capacity of increasing concentrations of diazirine-biotin (0-2 mM) to quench the emission of 5 μM catalysts (in TBS+0.01% BSA).

$$\frac{{I}_{f}^{0}}{{I}_{f}} = 1 + {k}_{q} {τ}_{0} [ Q ]$$

where I⁰_{f} and I_{f} are the emission intensities without or with the quencher, respectively. [Q] is the quencher concentration, k_{q} is the quencher rate coefficient and τ₀ is the lifetime of emissive excited state. The quenching rate kₛᵥ(=k_{q}τ₀) corresponds to the efficiency of energy transfer and can be determined as the slop for a plot of

$$\frac{{I}_{f}^{0}}{{I}_{f}} - 1 against [ Q ] .$$

These analyses revealed an inverse correlation between λ_{Em} and both emission energy (EmE) and efficiency of energy transfer (kₛᵥ) to diazirine residues (FIG. 12 B).

The influence of catalysts physiochemical properties on their capacity to absorb blue light and undergo energy transfer events with diazirine residues for subsequent covalent crosslinking with neighboring proteins was further evaluated through labeling efficiency of a model protein (FIGS. 12C and D). To this end, reactions comprising 3-5 M of a HaloTag-NanoLuc fusion protein, 100 μM diazirine-biotin, and varying concentrations of an iridium catalyst (0-10 μM) were assembled in TBS pH 7.5 within wells of a UV transparent 96-well plate and then irradiated using Efficiency Aggregators biophotoreactor (80 W) for 0-15 minutes at either 455 nm or 365 nm. To evaluate labeling efficiency, proteins were resolved on SDS-PAGE and transferred to a nitrocellulose membrane. Membranes were stained with Licor fluorescent total protein stain before been blocked with 5% BSA (Promega) in TBST for 1 hour at room temperature and subsequently incubated overnight at 4° C. with Streptavidin-HRP (Invitrogen) in TBST supplemented with 5% BSA. Following three washes in TBST, membranes were first scanned on the Cy5 channel to detect total protein then treated with ECL substrate (Promega W1001) and scanned on the chemiluminescence channel to detect proteins labeled with biotin. These analyses showed that photoactivation of diazirine upon 365 nm irradiation and subsequent protein labeling is catalyst independent while photoactivation upon 455 nm irradiation is catalyst dependent. Furthermore, labeling efficiency depended on catalyst concentration (concentration driven proximity between the catalyst and diazirine-biotin) as well catalyst's emission energy (EmE) and capacity to drive photoactivation of diazirine (EmE≥51 kcal/mol). Labeling efficiency was likely further influenced by other catalyst properties such as extinction coefficient (i.e., Ir-8844 provides significantly greater labeling efficiency than Ir-8870 although both have the same EmE).

### Example 2

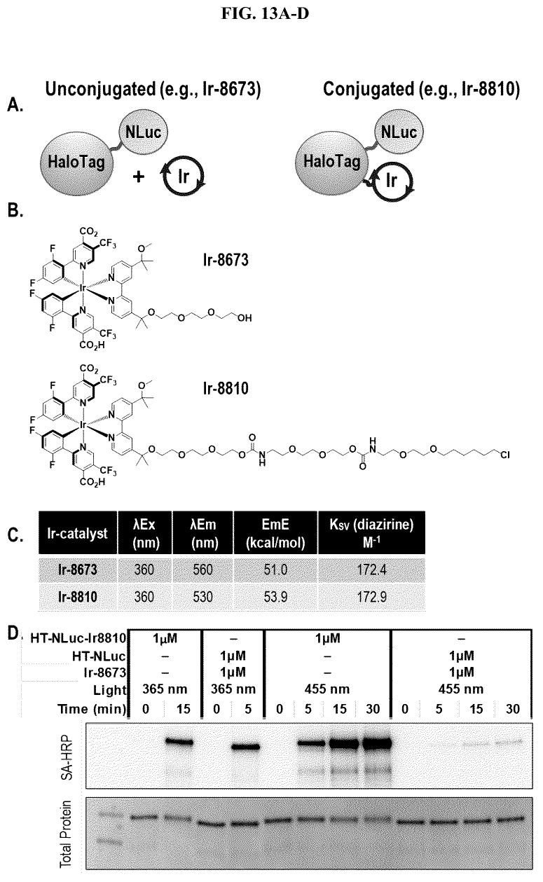

This example describes the influence of proximity between the catalyst and target protein of interest on the efficiency of LED-triggered photocatalytic protein labeling (FIG. 13). In this example, proximity between the catalyst and protein of interest is driven by covalent binding of an iridium catalyst that is conjugated to chloroalkane and HaloTag that is genetically fused to a protein of interest (e.g., NanoLuc) (FIG. 13A). The structures of the modifiable iridium catalyst and its derivative further conjugated to a chloroalkane are shown in FIG. 13B. The syntheses of these catalysts are included in Example 12.

Physiochemical properties of the iridium catalysts, and their capacities to undergo energy transfer events with diazirine-biotin, were determined as described in Example 1 and are shown in FIG. 13C. To determine the influence of proximity on labeling efficiency (FIG. 13D), reactions comprising 100 μM diazirine-biotin and 1 M of either HaloTag-NanoLuc fusion protein tethered to Ir-8810 catalyst or 1 M HaloTag-NanoLuc fusion protein+1 M Ir-8673 catalyst were assembled in TBS pH 7.5 within wells of a UV transparent 96-well plate and then irradiated using Efficiency Aggregators biophotoreactor (80 W) for 0-30 minutes at either 455 nm or 365 nm. To evaluate labeling efficiency, proteins were resolved on SDS-PAGE, transferred to a nitrocellulose membrane, and analyzed as described in Example 1. This analysis showed that labeling is dependent on radiation and subsequent activation of diazirine. Diazirine activation by 455 nm irradiation was not only catalyst dependent, but labeling efficiency was also significantly dependent on proximity between the catalyst and protein of interest. These results indicate that upon absorbance of blue light, the excited catalyst engage in energy transfer events with proximal diazirine-biotin residues to generate reactive intermediates with a very short lifetime, which can further undergo covalent crosslink with neighboring proteins before being quenched by water. As a result, labeling efficiency is directly correlated with proximity leading to greater labeling of proximal proteins.

### Example 3

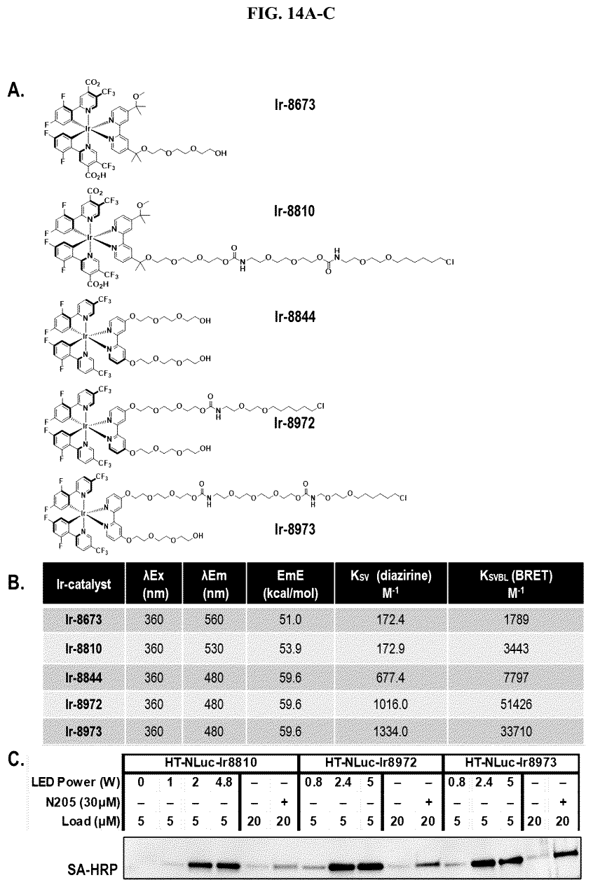

This example describes the influence of proximity between the catalyst and bioluminescent light source on the efficiency bioluminescence versus LED-triggered labeling (FIG. 14). In this example, proximity between the catalyst and bioluminescent light source (e.g., NanoLuc), which also serves as the target protein is driven by covalent binding of a catalyst conjugated to a chloroalkane and HaloTag genetically fused to NanoLuc. The structures of two modifiable catalysts and their derivatives further conjugated to chloroalkanes of different lengths are shown in FIG. 14A. The syntheses of these catalysts are included in Example 12.

Physiochemical properties of the iridium catalysts and their capacities to undergo energy transfer events with diazirine-biotin were determined as described in Example 1 and are shown in FIG. 14B. In addition, efficiency of energy transfer from NanoLuc to the catalysts was determined using a modified Stern-Volmer quenching relationship analysis monitoring the capacity of increasing concentrations of catalyst (0-125 μM) to quench the bioluminescence emission of 0.6 nM NanoLuc following the addition of 20 μM NanoGlo® Live Cell substrate in TBS+0.01% BSA (Promega Corporation, Cat. No. N205).

$$\frac{{B}^{0} - B}{B} = {k}_{s v B L} [ Q ]$$

where the quenching rate k_{SVBL} corresponds to the efficiency of energy transfer and can be determined as the slop for a plot of

$$\frac{{B}^{0} - B}{B} against [ Q ] .$$

These analyses revealed that the chloroalkane has a positive impact on the efficiencies of energy transfer, which is independent of proximity. The chloroalkane increased the efficiency of energy transfers from NanoLuc to the catalyst (2-7-fold) and from the catalyst to diazirine (0-2-fold) for Ir-8673 and Ir-8844 derivatives, respectively. This analysis also showed that Ir-8844 and its derivatives exhibit higher emission energy (EmE), which is better suited for activation of diazirine and subsequent photocatalytic labeling of proximal proteins.

To evaluate the ability of the catalysts to drive photocatalytic protein labeling upon excitation by either 455 nm radiation or bioluminescence (30 μM N205, NanoGlo® Live Cell substrate) (FIG. 14C), reactions comprising 500 μM diazirine-biotin, 0.6 μM bulking protein, and 0.06 μM HaloTag-NanoLuc tethered to either Ir-8810, Ir-8972 or Ir-8973 catalyst were assembled in TBS pH 7.5 within either wells of a UV transparent 96-well plate (LED irradiation) or white 96-well plate (bioluminescence). Plates were either irradiated for 15 minutes at 455 nm using Efficiency Aggregators biophotoreactor (0-4.8 W) or treated for 15 minutes with 30 μM NanoGlo® Live Cell substrate while control wells remained untreated. To evaluate labeling efficiency, proteins were resolved on SDS-PAGE, transferred to a nitrocellulose membrane, and analyzed as described in Example 1. This analysis demonstrated photocatalytic protein labeling triggered by either 455 nm irradiation or NanoLuc bioluminescence. HaloTag-NanoLuc tethered to Ir-8844 derivatives (i.e., Ir-8972 and Ir-8973) provided significantly higher bioluminescence-triggered labeling than HaloTag-NanoLuc tethered to Ir-8673 derivative (i.e., Ir-8810). This is attributed to the higher emission energies (EmE) and greater energy transfer efficiencies (k_{SVBL} and k_{SV}) exhibited by these catalyst-chloroalkane conjugates. In addition, Ir-8844 catalyst conjugated to a longer chloroalkane (i.e., Ir-8973) exhibited lower k_{SVBL}, but greater labeling efficiency indicating that for this configuration (HaloTag-NanoLuc), a longer chloroalkane may induce greater proximity between NanoLuc and the catalyst.

### Example 4

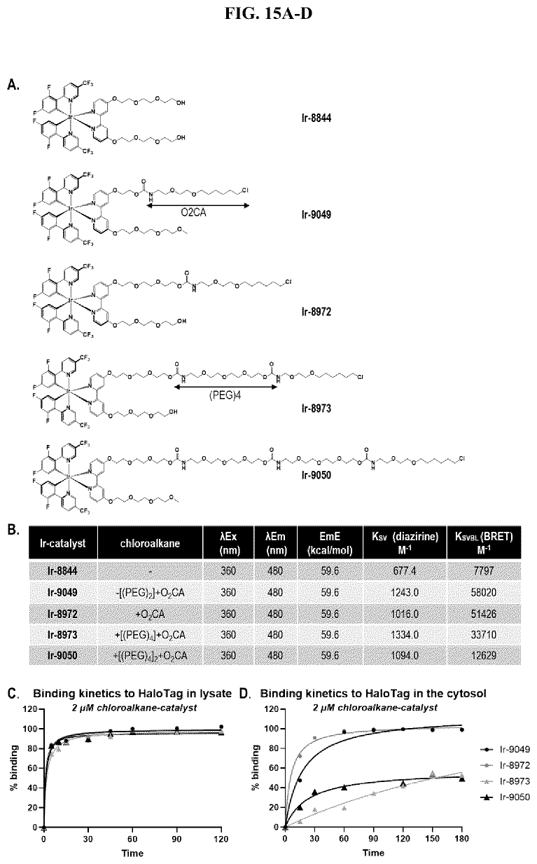

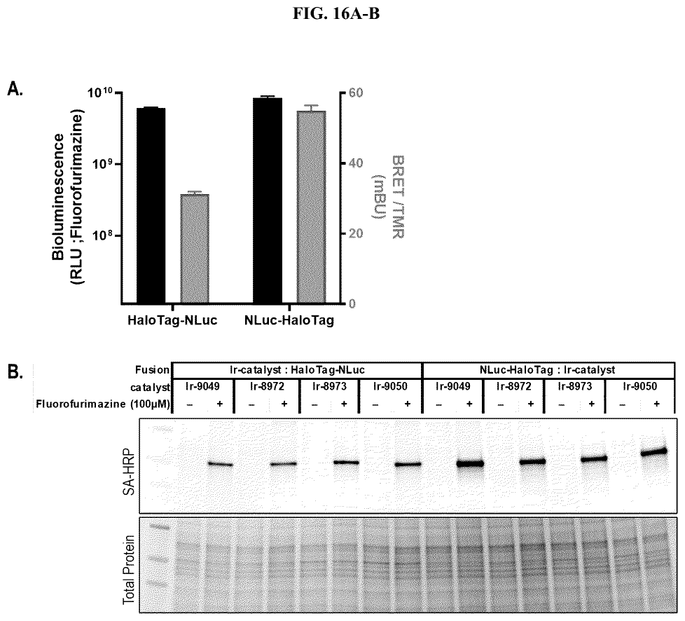

During development of the bioluminescence-triggered photocatalytic labeling described herein, experiments were conducted to evaluate the influence of the chloroalkane's length and structure on catalyst properties including, energy transfer efficiencies (NanoLuc to catalyst and catalyst to diazirine), binding kinetic to HaloTag, cellular permeability, and capacity to drive bioluminescence-triggered photocatalytic protein labeling (FIGS. 15 and 16). The structures of the modifiable catalyst Ir-8844 and its derivatives further conjugated to chloroalkanes of different lengths are shown in FIG. 15A. The syntheses of these catalysts are included in Example 12.

The influence of the chloroalkanes on the physiochemical properties of the iridium catalysts and their capacity to undergo energy transfer events were determined as described in Example 1 and 3 and are shown in FIG. 15B. These analyses revealed that the chloroalkane increased the efficiency of energy transfer from NanoLuc to the catalyst (2-7-fold) in a manner that was inversely correlated to the chloroalkane length. In addition, independent of length, the chloroalkane increased the efficiency of energy transfer from the catalyst to diazirine by 2-fold. Since the chloroalkane had no impact on catalysts' emission energy (EmE), these results indicate that the chloroalkane increased the capacity of the catalyst to absorb light in a manner that was inversely correlated with the chloroalkane length.

The chloroalkane provides the means to induce proximity between the catalyst and bioluminescence light source through covalent binding of a chloroalkane-catalyst conjugate to HaloTag genetically fused to the light source. The influence of the chloroalkane length and structure on binding kinetics to HaloTag (FIG. 15C) was evaluated by treating lysate prepared from cells expressing a HaloTag fusion protein with chloroalkane-catalyst conjugates at a final concentration of 2 M. After 0-120 minutes incubation time points, a fraction of each reaction (each containing a different chloroalkane-catalyst conjugate) was removed and treated with HaloTag TMR-fluorescent ligand (Promega) at a final concentration of 5 M. This allowed binding of the fluorescent ligand to any HaloTag fusion protein that remained unbound. The time point fractions were resolved on SDS-PAGE and scanned on a Typhoon fluorescent imager (GE healthcare). Bands were quantified using ImageQuant (GE healthcare), and binding kinetics were determined as the percent binding with time relative to time zero when no chloroalkane-catalyst conjugate was added. All chloroalkane-catalyst conjugates, regardless of the chloroalkane length, exhibited similar binding kinetic to HaloTag indicating that the length of the chloroalkane had very minimal impact on binding kinetic.

The influence of the chloroalkane length and structure on cellular permeability of catalyst conjugates was further evaluated through their binding kinetics to HaloTag inside cells (FIG. 15D). To this end, cells expressing a HaloTag fusion protein were treated with chloroalkane-catalyst conjugates at a final concentration of 2 M for 0-180 minutes before being treated for additional 15 minutes with HaloTag TMR-fluorescent ligand at a final concentration of 5 M. This allowed binding of the fluorescent ligand to any HaloTag fusion protein that remained unbound. Cells were then collected, lysed with detergent lysis buffer, and time points analyzed as described above. This analysis revealed that shorter chloroalkanes had minimal impact on cellular permeability allowing for rapid binding kinetics to HaloTag inside cells.

The efficiency of bioluminescence-triggered catalyst activation depends on the emission intensity of the luciferase energy donor, spectral overlap between luciferase emission and catalyst excitation, proximity between the luciferase and catalyst, and capacity of the complex to adopt a conformation favorable for efficient energy transfer between the two. First, the influence of the NanoLuc-HaloTag fusion orientation on the efficiency of bioluminescence resonance energy transfer (BRET) to a bound HaloTag TMR-fluorescent ligand was tested. To this end, fusions either untethered or tethered to a HaloTag TMR-fluorescent ligand were diluted in TBS+0.01% BSA to a final concentration of 6.6 nM and then treated with 10× fluorofurimazine at a final concentration of 20 M. Following 3 minutes incubation, raw luminescence (Total RLU) or filtered luminesces for donor (e.g., 450 nm/8 nm BP) and acceptor (600 nm LP) emissions, respectively were measured on a GloMax® Discover plate reader (Promega). BRET ratios were further calculated for each sample by dividing the acceptor emission value by its donor emission value. While both orientations delivered equivalent brightness one of them NanoLuc-HaloTag provided greater BRET efficiency (FIG. 16A), which is likely due to greater proximity between NanoLuc's substrate binding site and the bound fluorescent ligand.

The two fusions orientations were further tethered to chloroalkane-catalyst conjugates and compared for their capacity to drive bioluminescence-triggered photocatalytic protein labeling (FIG. 16B). Reactions comprising 100M diazirine-biotin, 0.1 mg/mL K562 cell lysate depleted of biotinylated proteins, and 60 nM HaloTag-NanoLuc or NanoLuc-HaloTag tethered to either Ir-9049, Ir-8972, Ir-8973, or Ir-9050 were assembled in TBS pH 7.5 within wells of a white 96-well plate. Bioluminescence was induced upon treatment with 10× fluorofurimazine at a final concentration of 100 M while control wells remained untreated. Following a 15 minutes incubation, samples were collected, resolved on SDS-PAGE, transferred to a nitrocellulose membrane, and analyzed as described in Example 1. In concordance with the 2-fold higher BRET efficiency observed for NanoLuc-HaloTag, all four catalysts tethered to NanoLuc-HaloTag delivered greater bioluminescence-triggered photocatalytic protein labeling than their counterparts tethered to HaloTag-NanoLuc. Furthermore, among the four chloroalkane-catalyst conjugates, NanoLuc-HaloTag fusion favored a shorter chloroalkane (i.e., Ir-9049) suggesting this orientation likely offers greater proximity between NanoLuc's substrate binding site and a bound catalyst. On the other hand, the other orientation HaloTag-NanoLuc favored a longer chloroalkane likely to mitigate reduced proximity between NanoLuc's substrate binding site and a bound catalyst.

### Example 5

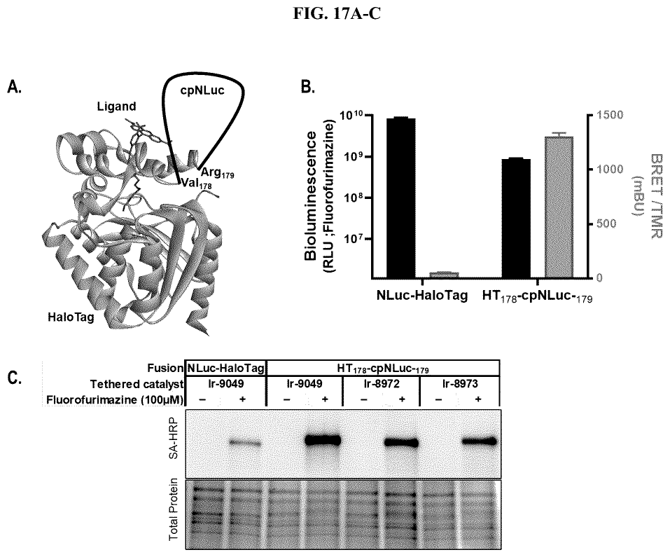

This example describes further optimization of the bioluminescent photocatalytic complex comprising a bioluminescent energy donor, chloroalkane-catalyst conjugate, and HaloTag, which offers the means to induce proximity between the two (FIG. 17). To increase proximity, a chimeric structure was engineered comprising a circularly permuted NanoLuc (e.g., cpNLuc at residues 67/68) that is inserted into a HaloTag's surface loop (between residues 178-179), which is proximal to the ligand interaction site (i.e., HT₁₇₈-cpNLuc-₁₇₉) (FIG. 17A). First, NanoLuc-HaloTag and the chimera HT₁₇₈-cpNLuc-₁₇₉ were compared for efficiency of bioluminescence resonance energy transfer (BRET) to a bound HaloTag TMR-fluorescent ligand. To this end, NanoLuc-HaloTag and HT₁₇₈-cpNLuc-₁₇₉ unconjugated or conjugated to a HaloTag TMR-fluorescent ligand were diluted in TBS+0.01% BSA to a final concentration of 6.6 nM and then treated with 10× fluorofurimazine at a final concentration of 20 μM. Following a 3 minutes incubation, raw luminescence (Total RLU) or filtered luminesces for donor (e.g., 450 nm/8 nm BP) and acceptor (600 nm LP) emissions, respectively were measured on a GloMax® Discover plate reader (Promega). BRET ratios were further calculated for each sample by dividing the acceptor emission value by its donor emission value. Although HT₁₇₈-cpNLuc-₁₇₉ was 10-fold dimmer, it provided 24-fold greater BRET efficiency (FIG. 17B) indicating that the chimeric structure was able to induce greater proximity between NanoLuc's substrate binding site and the bound fluorescent ligand or adopt a conformation favorable for energy transfer between the two or both.

HT₁₇₈-cpNLuc-₁₇₉ was further tethered to chloroalkane-catalyst conjugates and compared to NanoLuc-HaloTag:Ir-9049 for its capacity to drive bioluminescence-triggered photocatalytic protein labeling (FIG. 17C). Reactions comprising 100 μM diazirine-biotin, 0.1 mg/mL K562 cell lysate depleted of biotinylated proteins, and 60 nM conjugate were assembled in TBS pH 7.5 within wells of a white 96-well plate. Bioluminescence was induced upon treatment with 10× fluorofurimazine at a final concentration of 100 M while control wells remained untreated. Following a 15 minutes incubation, samples were collected, resolved on SDS-PAGE, transferred to a nitrocellulose membrane, and analyzed as described in Example 1. In concordance with the 24-fold higher BRET efficiency, all three catalysts tethered to HT₁₇₈-cpNLuc-₁₇₉ delivered greater photocatalytic protein labeling than NanoLuc-HaloTag:Ir-9049. Furthermore, among the three chloroalkane-catalyst conjugates, HT₁₇₈-cpNLuc-₁₇₉ also favored the shorter chloroalkane (i.e., Ir-9049). Taken together, these results indicate that the HT₁₇₈-cpNLuc-₁₇₉:Ir-9049 is well suited for catalyst activation via bioluminescence resonance energy transfer. The chimeric structure likely induces greater proximity between NanoLuc's substrate binding site and the bound catalyst or adopts a conformation favorable for energy transfer between the two or both.

### Example 6

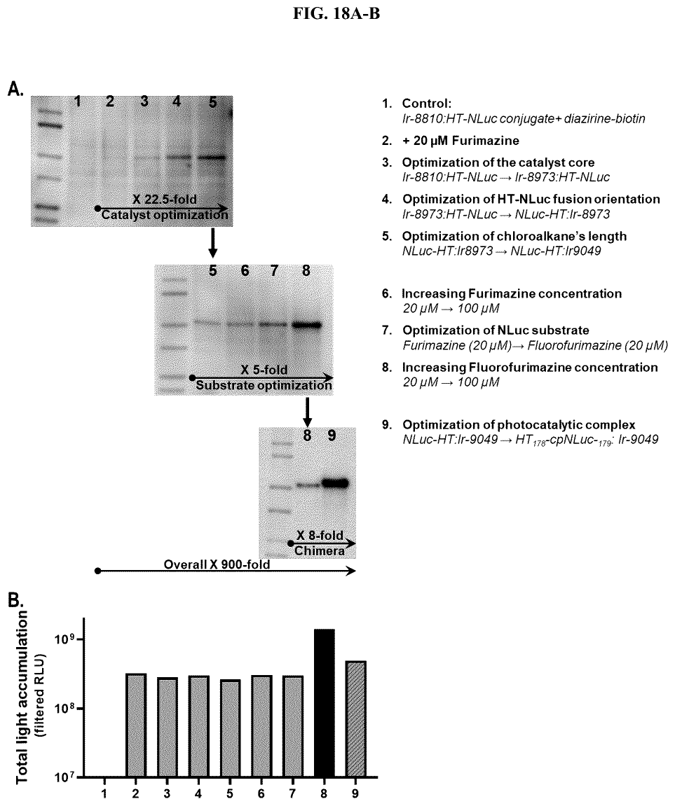

This example summarizes the optimization of the bioluminescent photocatalytic system that provided an overall 900-fold increase in bioluminescence-triggered photocatalytic protein labeling (FIG. 18). These included optimizations of the catalyst core and NanoLuc substrate as well as configuration of the bioluminescent photocatalytic complex. Reactions comprising 100 μM diazirine-biotin, 0.1 mg/mL K562 cell lysate depleted of biotinylated proteins, and 60 nM conjugate were assembled in TBS pH 7.5 within wells of a white 96-well plate. Bioluminescence was induced upon treatment with NanoLuc substrate (furimazine or fluorofurimazine) at the indicated final concentration while control wells remained untreated. Following a 20-minutes incubation, samples were collected, resolved on SDS-PAGE, transferred to a nitrocellulose membrane, and analyzed as described in Example 1 (FIG. 18A). To avoid signal saturation, accumulation of light emitted by the bioluminescent energy donor over 20 minutes incubation was monitored for each condition on a Varioskan plate reader using a filtered luminescence setup (610 nm/LP) (FIG. 18B). Optimization of the catalyst core, NanoLuc:HaloTag fusion orientation, and chloroalkane length resulted in an optimized NanoLuc-HaloTag:Ir-9049 complex, and a total 22.5-fold increase in protein labeling efficiency (condition 5). Further, exchange of the NanoLuc substrate furimazine with fluorofurimazine and increasing substrate concentration to 100 M provided a 5-fold increase in total light output and subsequently an additional 5-fold increase in protein labeling (condition 8). Finally, replacing the NanoLuc-HaloTag:Ir-9049 complex with the chimeric HT₁₇₈-cpNLuc-₁₇₉: Ir-9049 resulted in a 2.8-fold decrease in total light output, but an additional 8-fold increase in protein labeling, which is likely due to a more efficient energy transfer from NanoLuc to the catalyst.

### Example 7

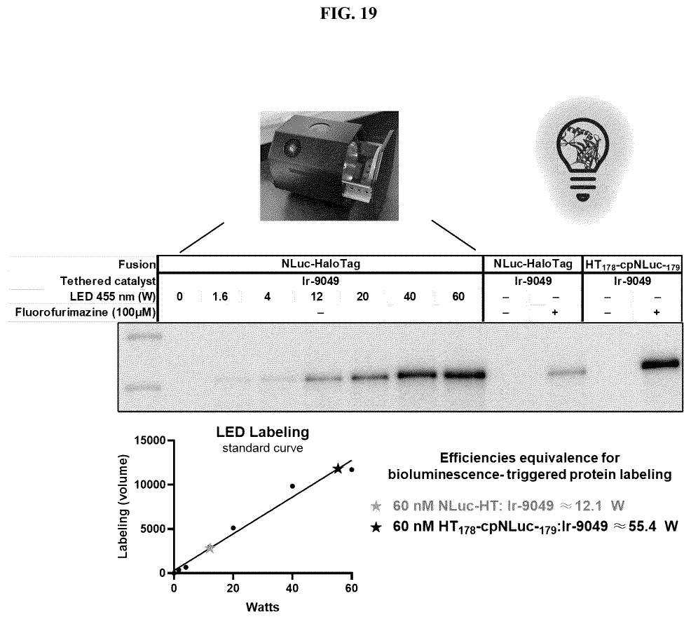

This example further demonstrates the efficiency of the optimized HT₁₇₈-cpNLuc-179: Ir-9049 bioluminescent photocatalytic complex. The efficiencies of photocatalytic labeling triggered by either increasing LED power or bioluminescence were compared (FIG. 19). Reactions comprising 100 M diazirine-biotin, 0.1 mg/mL K562 cell lysate depleted of biotinylated proteins, and 60 nM NanoLuc-HaloTag:Ir-9049 or HT₁₇₈-cpNLuc-₁₇₉: Ir-9049 were assembled in TBS pH 7.5 within either a) wells of a UV transparent 96-well plate, which was further subjected to 455 nm radiation at 0-60 W for 5 minutes (Efficiency Aggregators biophotoreactor), or b) wells of a white 96-well plate, which was further treated for 5 minutes with fluorofurimazine at a final concentration of 100 M while control wells remained untreated.

To evaluate labeling efficiency, samples were collected, resolved on SDS-PAGE, transferred to a nitrocellulose membrane, and analyzed as described in Example 1. Bands were quantified using ImageJ, and the LED power titration was further used to generate a calibration curve of labeling intensity vs watts. This analysis revealed that NanoLuc-HaloTag:Ir-9049 and HT₇₈-cpNLuc-₁₇₉:Ir-9049 complexes were able to drive bioluminescence-triggered protein labeling with efficiencies equivalent to 12.1 W and 55.4 W, respectively. This result further demonstrates the efficiency of catalyst activation by localized energy transfer vs global radiation.

### Example 8

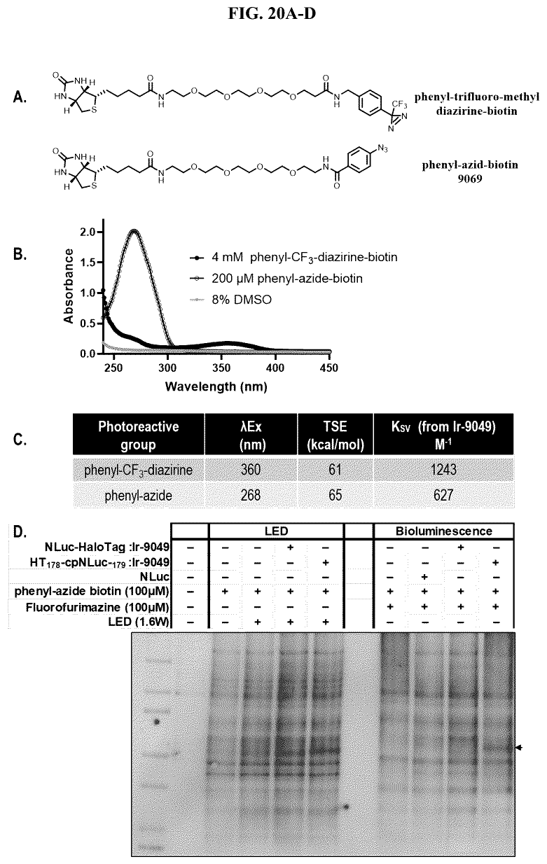

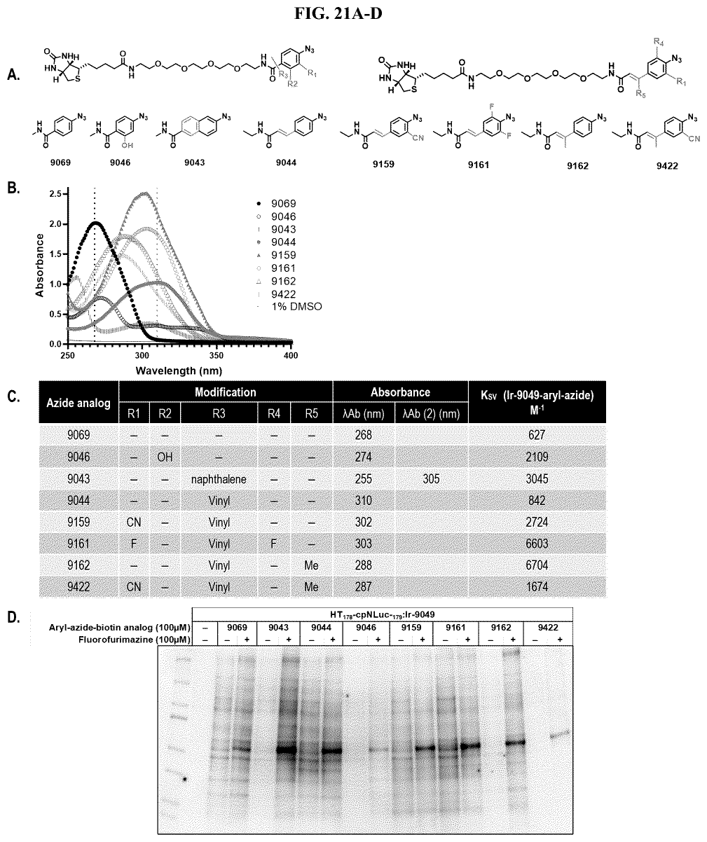

This example demonstrates the capacity to expand the utility of the bioluminescent photocatalytic system beyond diazirine to other photoreactive moieties such as aryl-azides, which are compatible with broader downstream applications. The structures of the aryl-azides biotin analogs are shown in FIGS. 20A and 21A, and their syntheses are included in Example 13.

First, the two photoreactive moieties phenyl-trifluoro-methyl diazirine and phenyl-azide were compared for their physiochemical properties and capacity to undergo energy transfer events with an excited Ir-9049 catalyst. Absorbance profiles for 4 mM phenyl-diazirine-biotin and 20-fold lower concentration of phenyl-azide-biotin (i.e., 200 M) in 8% or 1% DMSO, respectively, were monitored on a SPARK multimode plate reader (FIG. 20B). In addition, the capacity of the two photoreactive moieties to undergo energy transfer events with an excited Ir-9049 catalyst were determined as described in Example 1 (FIG. 20C). Generally, the blue shift absorbance for phenyl-azide was associated with a significantly more efficient capacity to absorb light as well as higher triplet state energy (TSE), but a decreased ability to undergo energy transfer events with an excited Ir-9049 catalyst.

The capacity of photocatalytic complexes (i.e., NanoLuc-HaloTag:Ir-9049 and HT₁₇₈-cpNLuc-₁₇₉:Ir-9049) excited by either LED or bioluminescence to undergo energy transfer events with phenyl-azide-biotin for subsequent crosslinking with proximal proteins was further investigated. Reactions comprising 100M phenyl-azide-biotin (9069), 0.1 mg/mL K562 cell lysate depleted of biotinylated proteins, and 60 nM NanoLuc, or NanoLuc-HaloTag:Ir-9049 or HT₁₇₈-cpNLuc-₁₇₉: Ir-9049 were assembled in TBS pH 7.5 within either a) wells of a UV transparent 96-well plate, which was further subjected to 455 nm radiation at 0-1.6 W for 5 minutes (Efficiency Aggregators biophotoreactor) or b) wells of a white 96-well plate, which was further treated for 20 minutes with fluorofurimazine at a final concentration of 100 M while control wells remained untreated. To evaluate labeling efficiency, samples were collected, resolved on SDS-PAGE, transferred to a nitrocellulose membrane, and analyzed as described in Example 1. Unlike diazirene, light independent as well as light-dependent background was apparent for both LED and bioluminescence. Even though photoactivation of phenyl-azide by 455 nm light alone is likely minimal, it still generates reactive intermediates with significantly longer lifetime compared to those generated upon photoactivation of diazirine. These longer-lived intermediates can diffuse further before being quenched by water resulting in overall greater labeling radius and increased light-dependent background. Catalyst activation by either LED or bioluminescence energy transfer, resulted in specific labeling of a proximal model protein (i.e., NanoLuc-HaloTag or HT₁₇₈-cpNLuc-₁₇₉), albeit with relatively high background. Notably, for bioluminescence, specific proximal labeling was only apparent for the chimeric photocatalytic complex, which enables more efficient bioluminescence-triggered catalyst activation.

Experiments were conducted during development of embodiments herein to explore structural modifications to the phenyl-azide moiety that could decrease the lifetime of its photogenerated intermediates thereby reducing light-dependent background and increasing labeling specificity. Modifications causing red shift absorbance and subsequently lower triplet state energy thereby enabling more efficient bioluminescence-triggered photocatalytic activation were also explored. Structures and physiochemical properties for a subset of aryl-azide analogs are shown in FIGS. 21A and 21B-C, respectively. Absorbance profile (200 μM in 1-2% DMSO) and capacity to undergo energy transfer events with an excited Ir-9049 were determined as described above. Generally, the analogs exhibited a range of red-shift absorbances compared to phenyl-azide-biotin 9069 (6-42 nm), which were associated with increase capacity to undergo energy transfer events (1.3-10.5-fold), indicating easier activation by blue light and photocatalytic energy transfer, respectively.

The aryl-azide-biotin analogs were further evaluated for efficiency and specificity of bioluminescence-triggered photocatalytic protein labeling as well as light-independent background (FIG. 21D). To this end, reactions comprising 100 M aryl-azide-biotin analog, 0.1 mg/mL K562 cell lysate depleted of biotinylated proteins, and 60 nM HT₁₇₈-cpNLuc-₁₇₉: Ir-9049 were assembled in TBS pH 7.5 within wells of a white 96-well plate, which was further treated for 45 minutes with fluorofurimazine at a final concentration of 100 M, while control wells remained untreated. Samples were then collected, resolved on SDS-PAGE, transferred to a nitrocellulose membrane, and analyzed as described in Example 1. Even though the longer 45 minutes incubation increased light-independent background, this background was significantly decreased by four structural modifications including: naphthene (9043), hydroxyl substitution at a meta position of the phenyl ring (9046), and a methyl substitution on the vinyl group designed to inhibit Michael addition (9162 and 9422). In addition, several substitutions to the phenyl ring increased labeling specificity to different extent likely by impeding the generation-kinetics and/or lifetime of the reactive intermediates resulting with an overall smaller labeling radius.

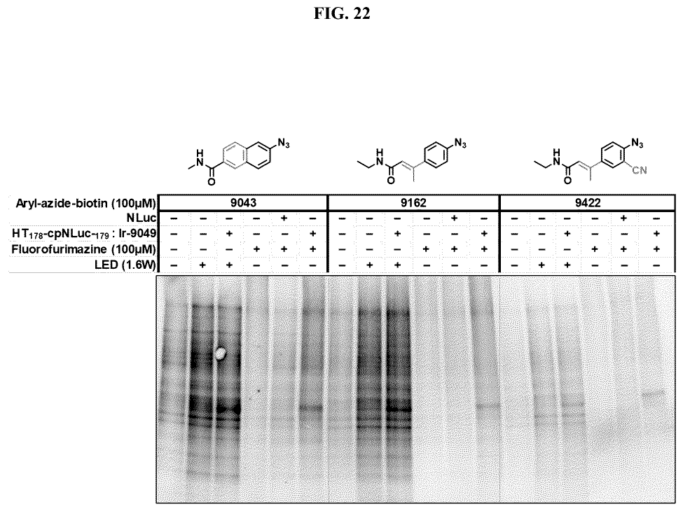

Finally, three analogs exhibiting minimal light-independent background and increasing labeling radius, 9422<9162<9043, were further evaluated for efficiency and specificity of LED versus bioluminescence triggered labeling as well as light-dependent background (FIG. 22). Reactions comprising 100 μM aryl-azide-biotin analog, 0.1 mg/mL K562 cell lysate depleted of biotinylated proteins, and either 60 nM NanoLuc or HT₁₇₈-cpNLuc-₁₇₉:Ir-9049 were assembled in TBS pH 7.5 within either a) wells of a UV transparent 96-well plate, which was further subjected to 455 nm radiation at 0-1.6 W for 10 minutes (Efficiency Aggregators biophotoreactor), or b) wells of a white 96-well plate, which was further treated for 20 minutes with fluorofurimazine at a final concentration of 100 M while control wells remained untreated. A high LED-dependent background was observed for all three analogs, which was likely due to their red shift absorbance and overall easier activation by blue light. 9162, the vinyl-phenyl-azide analog with methyl modification on the vinyl group, exhibited the highest light-dependent background, which was correlated with its 20 nm red shift absorbance and high capacity to absorb light. While the additional CN substitution to the phenyl ring (e.g., 9422) had a minimal impact on the absorbance profile, it significantly reduced light-dependent background likely by decreasing the lifetime of reactive intermediates. Subsequently, LED-triggered photocatalytic labeling of the proximal HT₁₇₈-cpNLuc-₁₇₉ was detected for all three analogs albeit with high light-dependent background. Bioluminescence, on the other hand, induced efficient catalyst-depended labeling, but with very minimal light-dependent background further demonstrating the advantage of a mild, intrinsic, localized bioluminescent light source. In addition, lifetime of photogenerated reactive intermediates resulted with a range of labeling specificities that would be useful for different applications designed to label a specific protein versus a neighboring environment.

### Example 9

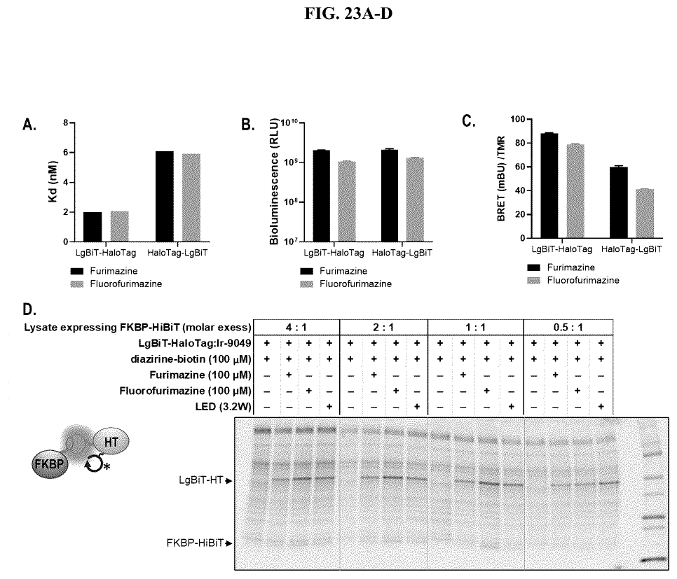

This example demonstrates the versatility offered by a bioluminescent photocatalytic complex relying on a LgBiT/HiBiT complementation reporter. Such configuration offers a modality agnostic approach for proximity between the photocatalytic complex and protein of interest genetically fused to HiBiT as well as greater spatial control over photocatalytic reactivity. First, the properties of the bioluminescent complementation reporter comprising VS-HiBiT peptide and either HaloTag-LgBiT or LgBiT-HaloTag were compared (FIG. 23 A-C). To determine complementation affinity, equal volumes of VS-HiBiT peptide serially diluted in TBS+0.01% BSA to final concentrations of 200-0 nM and HaloTag-LgBiT or LgBiT-HaloTag diluted in TBS+0.01% BSA to a final concentration of 0.2 nM were combined in wells of a white 96-well plate and mixed for 30 minutes. Following a 3-minutes treatment with either 10× furimazine or fluorofurimazine at a final concentration 20 M, bioluminescence was measured on a GloMax® Discover plate reader (Promega), and binding affinity (Kd) was derived from a saturation binding curve of luminescence against VS-HiBiT concentration (FIG. 23A). To determine brightness and BRET efficiency, HaloTag-LgBiT and LgBiT-HaloTag, either untethered or tethered to a HaloTag TMR-fluorescent ligand, were diluted in TBS+0.01% BSA to a final concentration of 12 nM, combined with equal volume of VS-HiBiT peptide diluted in TBS+0.01% BSA to a final concentration of 120 nM, and mixed for 30 minutes to allow for complementation. Following a 3-minutes treatment with either 10× furimazine or fluorofurimazine at a final concentration 20 M, raw luminescence (Total RLU; FIG. 23B) or filtered luminesces of donor (e.g., 450 nm/8 nm BP) and acceptor (600 nm LP) emissions were measured on a GloMax® Discover plate reader (Promega). BRET ratios were further calculated for each sample by dividing the acceptor emission value by its donor emission value (FIG. 23C). While both configurations had similar brightness and preference for furimazine as a substrate, the VS-HiBiT/LgBiT-HaloTag configuration exhibited 3-fold higher complementation affinity and provided 1.5-2-fold greater BRET efficiency (FIG. 23C). This was consistent with the higher BRET for NanoLuc-HaloTag fusion orientation, further suggesting that this fusion orientation offers increased proximity between the luminesce substrate binding site and a bound fluorescent ligand.

LgBiT-HaloTag was further tethered to Ir-9049 and evaluated for its capacity to drive photocatalytic protein labeling upon complementation with HiBiT genetically fused to a protein of interest (FIG. 23D). HEK293 cell lysate with estimated expression of 2000 nM FKBP-HiBiT was serially diluted into control HEK293 cell lysate to generate 4× lysate solutions with expression levels of 960 nM, 480 nM, 240 nM, and 12 nM while maintaining constant concentration of total proteins. 20 μL of the 4× serially diluted cell lysate were combined with 20 μL of a 4×LgBiT-HaloTag:Ir-9049 (i.e., 240 nM) solution in wells of a UV-transparent or white 96-well plates and mixed for 15 minutes to allow complementation. Following treatment with 10× diazirine-biotin at a final concentration of 100 M, the UV-transparent plates were subjected to 455 nm radiation at 0-3.2 W for 15 min (Efficiency Aggregators biophotoreactor), and the white plate were further treated for 30 minutes with either furimazine or fluorofurimazine at a final concentration of 100 M while control wells remained untreated. Samples were then collected, resolved on SDS-PAGE, transferred to a nitrocellulose membrane, and analyzed as described in Example 1. Light-dependent labeling of both LgBiT-HaloTag and FKBP-HiBiT were detected upon radiation or treatment with either furimazine or fluorofurimazine. Highest labeling was achieved using bioluminescence as an intrinsic light source and fluorofurimazine as the substrate even though it is not HiBiT/LgBiT's preferred substrate. This indicates that other substrate properties beside brightness such as substrate and signal stabilities over time may play a role in labeling efficiency. Furthermore, equivalent fluorofurimazine-induced labeling of both LgBiT-HaloTag and FKBP-HiBiT across 0-4-fold molar excess of HiBiT indicates that efficient complementation is driving proximal labeling of FKBP-HiBiT.

### Example 10

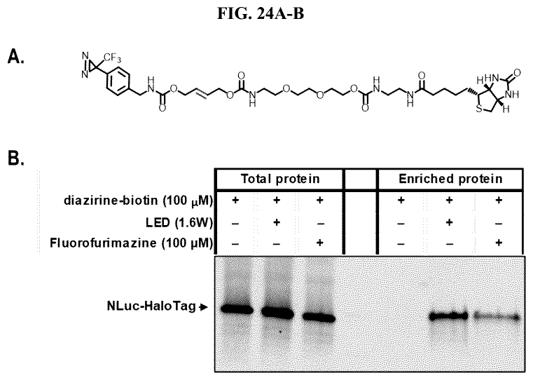

This example demonstrates the capacity to assemble a bioluminescent photocatalytic complex inside cells and utilizing it to drive labeling of proximal proteins with a cleavable-biotin for subsequent enrichment on streptavidin beads (FIG. 24). As shown above in FIG. 15, the minimal influence of chloroalkane on cell permeability of Ir-9049 coupled with its covalent, rapid, and highly specific binding to HaloTag allows conjugation to HaloTag and subsequent assembly of a bioluminescent, photocatalytic complex inside living cells. In addition, proximal labeling with a palladium cleavable-diazirine-biotin (FIG. 24A) reduces the inherent background enrichment originating from endogenously biotinylated proteins.

HeLa cells were transfected with a DNA construct encoding NanoLuc-HaloTag and plated into wells of 6-well plates at 2×10⁵ cell/mL and incubated overnight at 37° C., 5% CO₂. The next day, plates were treated with Ir-9049 catalyst at a final concentration of 2 M for 60 minutes to allow assembly of a bioluminescent, photocatalytic complex. To remove excess unreacted Ir-9049 catalyst, cells were washed twice, 15 minutes each, in HBSS buffer. The last HBSS wash was replaced with Opti-MEM media supplemented with 2% serum and 20 μM cleavable-diazirine-biotin. Following a 30-minutes incubation, plates were either exposed to 455 nm LED radiation (3.2 watts) for 15 minutes, treated with 20 μM fluorofurimazine for 45 minutes, or remained untreated (no light control). To remove excess unreacted cleavable-diazirine-biotin, cells were washed twice, 15 minutes each, in HBSS buffer. The last HBSS wash was replaced with 1 mL Mammalian Lysis Buffer (Promega) supplemented with 10-fold dilution of 10×RQ1-DNase buffer (Promega), 50-fold dilution RQ1-DNase (Promega), and 100-fold dilution Protease Inhibitor Cocktail (Promega). Following a 30-minutes incubation at room temperature with constant mixing, cell lysates were collected, and biotinylated proteins were captured on 75 μL High Capacity Magne® Streptavidin Beads (Promega) while nonspecific interactions were washed-out. Labeled proteins were then released by a 30-minutes incubation with a palladium cleavage reagent (Promega), resolved on SDS-PAGE, transferred to PVDF membrane, and subjected to Western analysis using antibodies against HaloTag (Promega). The Western blot (FIG. 24B) revealed efficient photocatalytic labeling within a complex cellular environment by either LED or bioluminescence.

### Example 11

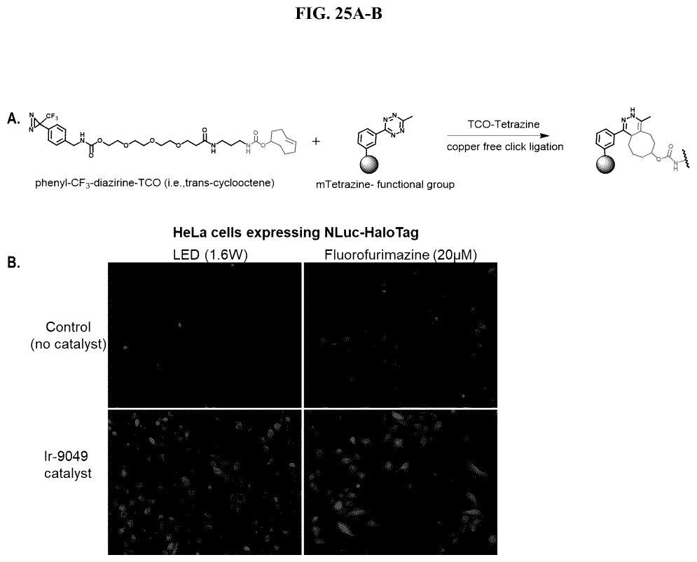

This example demonstrates the versatility of a bioluminescent, photocatalytic complex assembled inside cells and coupled with a two-steps labeling approach (FIG. 25). Proximal protein labeling with a click-handle offers flexibility to introduce diverse functionalities via a copper-free bioorthogonal ligation. In addition, this approach could minimize potential interference of functional moieties such as fluorophores with photoredox catalysis.

HeLa cells were transfected with a DNA construct encoding NanoLuc-HaloTag that was diluted 10-fold into promoterless carrier DNA, plated in flasks at 2×10⁵ cell/mL, and incubated 16-18 hours at 37° C., 5% CO₂. Next day, cells were collated, replated in 24-well plates at 2×10⁵ cell/mL, and incubated overnight at 37° C., 5% CO₂. The next day, plates were treated with either Ir-9049 catalyst or chloroalkane-biotin (control) at final concentrations of 2 M to allow assembly of bioluminescent photocatalytic complexes. To remove excess unreacted Ir-9049 catalyst or chloroalkane-biotin, cells were then washed twice, 15 minutes each, in HBSS buffer.

The last HBSS wash was replaced with Opti-MEM media supplemented with 2% serum and 20 M diazirine-TCO (trans-cyclooctene). Following a 30-minutes incubation, plates were either exposed to 455 nm LED radiation (1.6 watts) for 15 minutes or treated with 20 μM fluorofurimazine for 45 minutes. To remove excess unreacted diazirine-TCO, cells were washed twice, 15 minutes each, in HBSS buffer. The last HBSS wash was replaced with Opti-MEM media supplemented with 2% serum and 1 μM Tetrazine-Janelia-549 fluorophore conjugate (Tocris) and incubated for 15 minutes to allow for TCO-tetrazine ligation. Cells were washed for two last times before imaging on a BZ-X800 Analyzer (Keyence). Fluorescence images revealed specific LED or bioluminescence driven photocatalytic labeling with high signal over background.

### Example 12

This example describes the synthesis of the catalysts described herein.

| Catalyst | Structure | MS | Ex/nm | Em/nm |
|---|---|---|---|---|
| Ir-8673 | 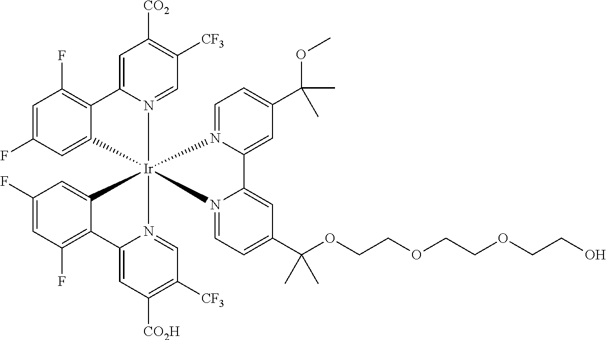 | [M + H]⁺ 1215.10 | 380 | 560 |
| Ir-8844 | 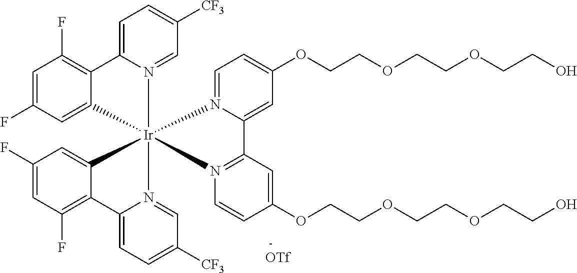 | [M]+ 1161.12 | 380 | 480 |
| Ir-8870 | 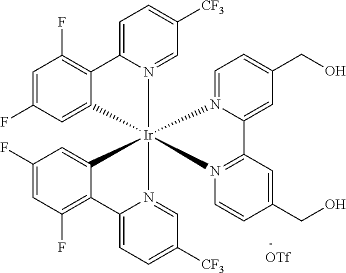 | [M]+ 924.80 | 380 | 480 |
| Ir-8871 | 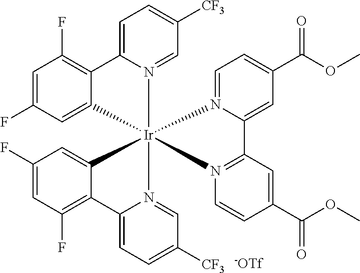 | [M + H]⁺ 980.82 | 380 | 600 |
| Ir-8810 | 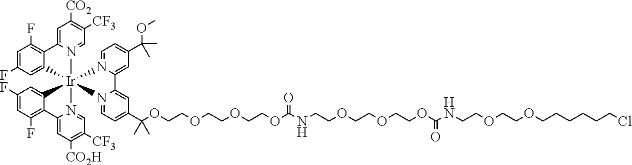 | [M + H]⁺ 1640.12 | 380 | 530 |
| Ir-8972 | 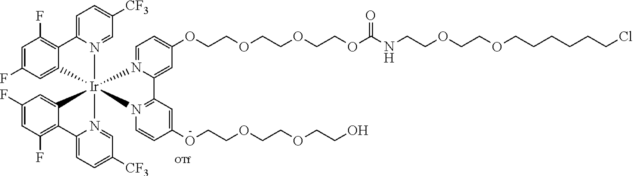 | [M]+ 1410.36 | 380 | 530 |
| Ir-8973 | 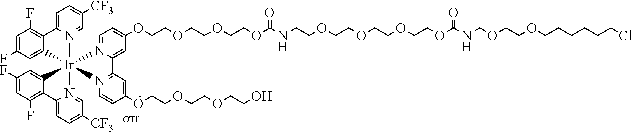 | [M]+ 1410.36 | 380 | 530 |
| Ir-9049 | 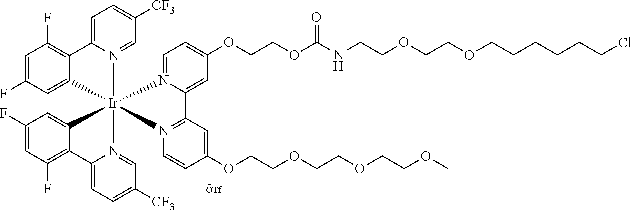 | [M]+ 1336.72 | 380 | 530 |
| Ir-9050 | 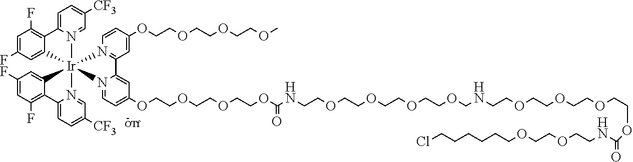 | [M]+ 1863.30 | 380 | 530 |

| Catalyst | Structure | MS | Ex/nm | Em/nm |
|---|---|---|---|---|
| Ru-8975 | 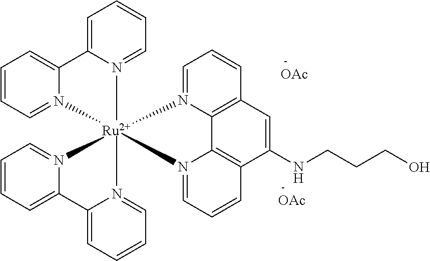 | [M]²⁺/2 333.58 | 450 | 620 |
| Ru-8974 | 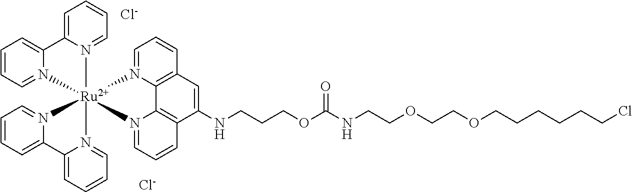 | [M]²⁺/2 458.14 | 450 | 620 |
| Ru-9003 | 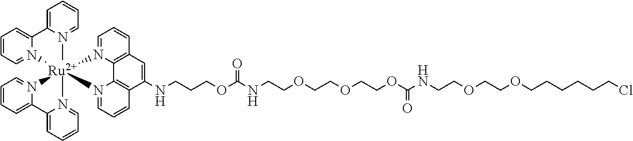 | [M]²⁺/2 545.84 | 450 | 620 |
| PS-9167 | 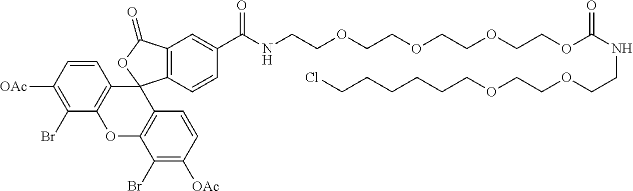 [SMILES: CC(=O)Oc1ccc2c(c1Br)Oc1c(ccc(OC(C)=O)c1Br)C21OC(=O)c2cc(C(=O)NCCOCCOCCOCCOC(=O)NCCOCCOCCCCCCCl)ccc21] | [M + H]⁺ 817.56 | 450 | 543 |

### Syntheses of Ir Catalysts:

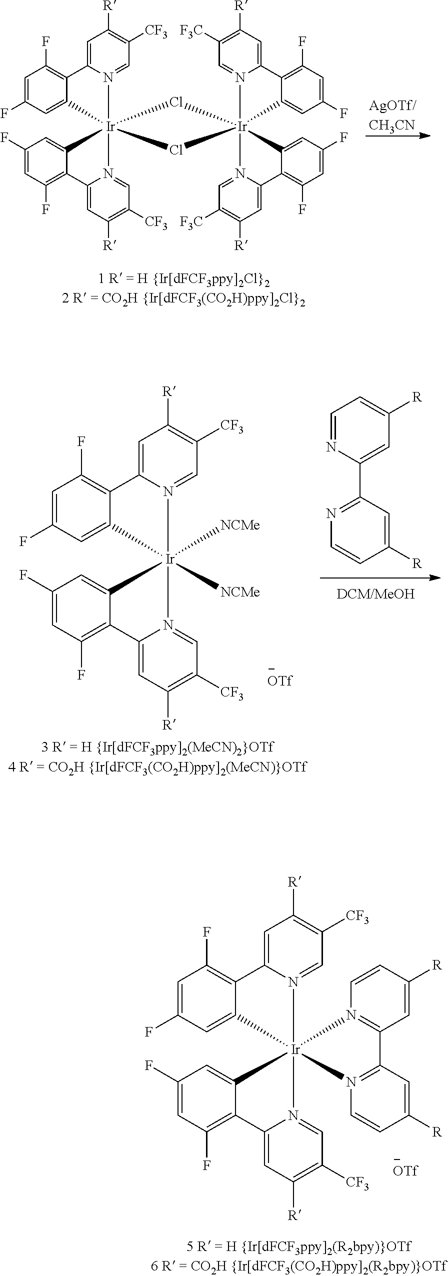

{Ir[dFCF₃ppy]₂Cl}₂ is commercially available from Strem: www.strem.com/catalog/v/77-0468/31/iridium_870987-64-7 and {Ir[dFCF₃(CO₂H)ppy]₂Cl}₂ was synthesized following literature reported procedures: Science 367, 1091-1097 (2020).

GP1: The bi-Ir—Cl complex (0.1 mmol, 1.0 equiv) was combined with AgOTf (53 mg, 0.2 mmol, 2.0 equiv) in CH₃CN (5 mL). This mixture was stirred at RT overnight in the dark. The resulting suspension was then filtered through celite and concentrated. The residue was redissolved DCM/MeOH (1/1, 10 mL), filtered through celite, and concentrated to yield the intermediate 3 or 4 as yellow film that was used without further purification.

To a solution of Intermediate 3 or 4 (0.1 mmol, 1.0 equiv) in DCM/MeOH (1/1, 2 mL), bpy was added to the reactants (0.12 mmol, 1.2 equiv). The reaction mixture was then stirred at RT for 16 h. LC-MS indicated full conversion of intermediates 3 or 4. The solution was evaporated onto celite and purified by silica gel chromatography.

Ir-8673: ¹H NMR (400 MHz, DMSO-d₆) δ 8.81 (d, J=11.2 Hz, 2H), 8.34 (s, 2H), 7.97 (t, J=5.9 Hz, 2H), 7.89 (d, J=6.0 Hz, 1H), 7.81 (d, J=5.9 Hz, 1H), 7.34 (s, 2H), 7.09 (t, J=11.0 Hz, 2H), 5.86 (d, J=8.2 Hz, 2H), 3.83-3.53 (m, 10H), 3.38 (t, J=5.2 Hz, 2H), 3.13 (s, 3H), 1.59 (s, 6H), 1.56 (s, 6H). LRMS [M+H]⁺ 1215.1.

Ir-8844: ¹H NMR (400 MHz, Methylene Chloride-d₂) δ 8.48 (d, J=8.7 Hz, 2H), 8.37 (d, J=2.5 Hz, 2H), 8.07 (d, J=8.9 Hz, 2H), 7.70 (d, J=6.2 Hz, 4H), 7.12-6.98 (m, 2H), 6.73-6.57 (m, 2H), 5.73 (dd, J=8.1, 2.2 Hz, 2H), 4.65-4.43 (m, 4H), 3.94 (d, J=4.5 Hz, 4H), 3.79-3.42 (m, 16H). LRMS [M]⁺ 1161.1.

Ir-8870: ¹H NMR (400 MHz, Methanol-d₄) δ 8.77 (s, 2H), 8.59 (d, J=9.0 Hz, 2H), 8.33 (d, J=8.8 Hz, 2H), 8.03 (d, J=5.7 Hz, 2H), 7.76 (s, 2H), 7.70 (d, J=5.7 Hz, 2H), 6.89-6.70 (m, 2H), 5.80 (d, J=8.3 Hz, 2H), 4.92 (s, 4H). LRMS [M]⁺ 924.8.

Ir-8871: ¹H NMR (400 MHz, Methanol-d₄) δ 9.32 (s, 2H), 8.60 (d, J=8.9 Hz, 2H), 8.32 (dd, J=15.8, 7.3 Hz, 4H), 8.19 (d, J=5.7 Hz, 2H), 7.78 (s, 2H), 6.86 (t, J=10.9 Hz, 2H), 5.77 (d, J=8.2 Hz, 2H), 4.08 (s, 6H). LRMS [M]⁺ 980.8.

Synthesis of bpy-1

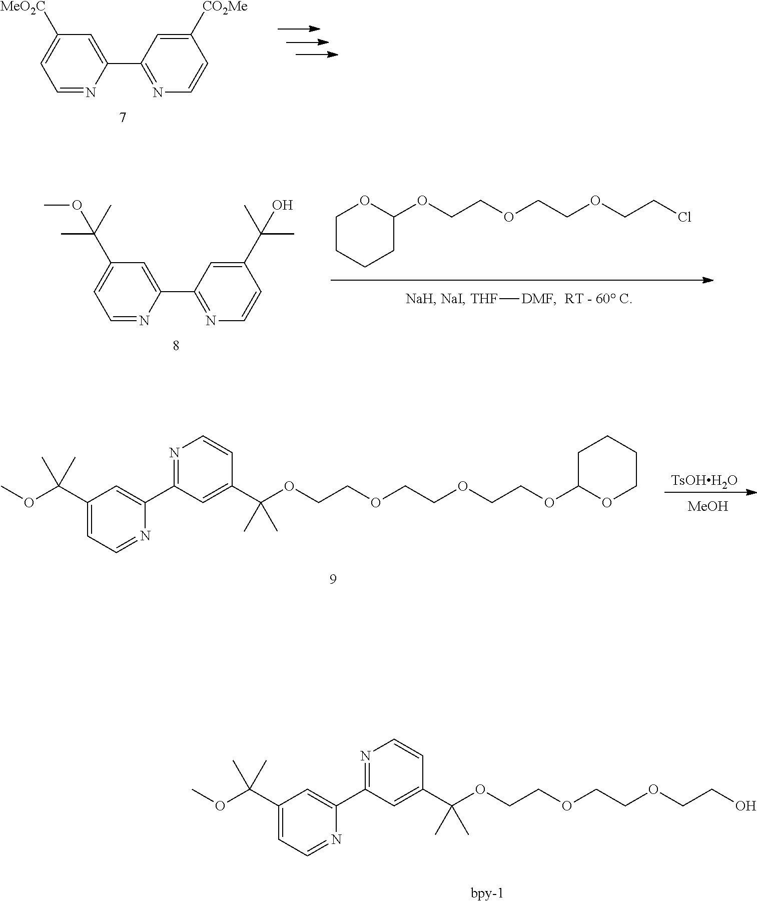

Intermediate 8 was synthesized from commercially available starting material 7 following literature procedure: Science 367, 1091-1097 (2020).

Intermediate 9: To a solution of intermediate 8 (200 mg, 0.7 mmol, 1.0 equiv) in THF (7 mL), NaH (60 wt %, 56 mg, 1.4 mmol, 2.0 equiv) was added. The mixture was stirred at RT for 30 min. To the suspension, NaI (11 mg, 0.07 mmol, 0.1 equiv) and 2-(2-(2-(2-chloroethoxy)ethoxy)ethoxy)tetrahydro-2H-pyran (350 mg, 1.4 mmol, 2.0 equiv) in DMF (3 mL) was added dropwise over 10 min. The mixture was then heated at 60° C. for 48 h. The reaction was cooled down and quenched by addition of saturated aq. NH₄Cl (10 mL). The quenched reaction was then concentrated in vacuo to remove organic solvents and extracted with EtOAc (20×3 mL). The combined organic layers were washed with H₂O (50 mL), brine (50 mL), dried over Na₂SO₄, and concentrated to afford the crude, which was used in the next step without further purification.

bpy-1: Intermediate 9 (50 mg, 0.1 mmol, 1.0 equiv) and TsOH·H₂O (19 mg, 0.1 mmol, 1.0 equiv) were dissolved in MeOH (4 mL). The solution was stirred at RT for 2 h. LC-MS indicated full conversion. The reaction was concentrated onto celite, and the desired product was isolated using silica gel chromatography. ¹H NMR (400 MHz, Chloroform-d) δ 8.64 (d, J=5.1 Hz, 2H), 8.38 (d, J=5.5 Hz, 2H), 7.41 (ddd, J=19.4, 5.0, 2.2 Hz, 2H), 3.83-3.53 (m, 10H), 3.38 (t, J=5.2 Hz, 2H), 3.13 (s, 3H), 1.59 (s, 6H), 1.56 (s, 6H). LRMS [M+H]⁺ 419.5.

Synthesis of bpy-2

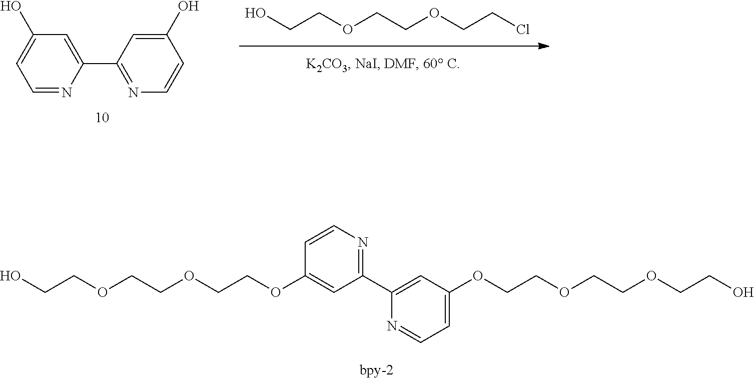 [SMILES: Oc1ccnc(-c2cc(O)ccn2)c1 ; OCCOCCOCCCl ; OCCOCCOCCOc1ccnc(-c2cc(OCCOCCOCCO)ccn2)c1]

bpy-2: To a suspension of bpy 10 (376 mg, 2.0 mmol, 1.0 equiv) and K₂CO₃ (830 mg, 6.0 mmol, 3.0 equiv) in DMF (5 mL), 2-(2-(2-chloroethoxy)ethoxy)ethan-1-ol (1.0 g, 6.0 mmol, 3.0 equiv) was added dropwise over 5 min. The mixture was heated at 60° C. for 20 h and cooled down to RT, diluted with EtOAc (100 mL), and filtrated over celite. The filtrate was concentrated in vacuo to afford the crude. The desired product, bpy-2, was isolated by silica gel chromatography. ¹H NMR (400 MHz, Methanol-d₄) δ 8.46 (dd, J=5.8, 1.8 Hz, 2H), 7.86 (d, J=2.2 Hz, 2H), 7.05 (dt, J=5.3, 2.3 Hz, 2H), 4.34 (t, J=4.2 Hz, 4H), 3.92 (p, J=2.2 Hz, 4H), 3.81-3.47 (m, 16H). LRMS [M+H]⁺ 453.3.

### Synthesis of Ir-8810

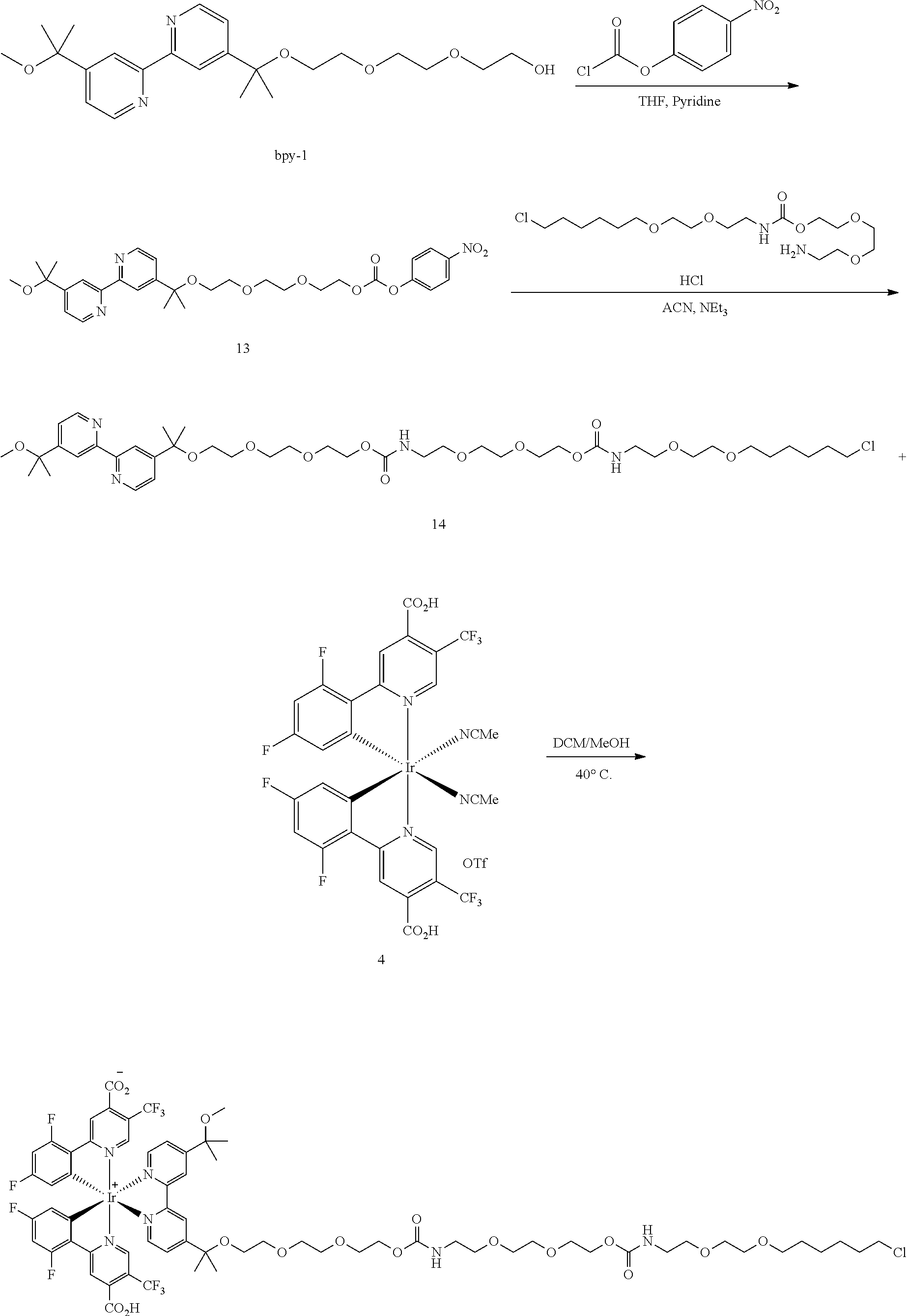

Intermediate 13: To a solution of bpy-1 (25 mg, 0.06 mmol, 1.0 equiv) in THF (4 mL), pyridine (0.5 mL) and p-nitrophenyl chloroformate (15 mg, 0.07 mmol, 1.2 equiv) was added. The solution was stirred overnight and concentrated onto celite. The desired product was purified using silica gel chromatography. ¹H NMR (400 MHz, Methylene Chloride-d₂) δ 8.67 (d, J=5.0 Hz, 2H), 8.51 (d, J=7.5 Hz, 2H), 8.38-8.20 (m, 2H), 7.61-7.32 (m, 4H), 4.45 (ddd, J=5.0, 3.4, 1.5 Hz, 2H), 3.89-3.77 (m, 2H), 3.69 (dt, J=16.6, 5.1 Hz, 6H), 3.43 (t, J=5.0 Hz, 2H), 3.17 (s, 3H), 1.63 (s, 6H), 1.60 (s, 6H). LRMS [M+H]⁺ 584.6.

Intermediate 14: To a solution of intermediate 13 (12 mg, 21 μmol, 1.0 equiv) in ACN (2 mL), NEt₃ (34 μL, 0.21 mmol, 10 equiv) and chloroalkane intermediate 14 (25 mg, 25 μmol, 1.2 equiv) was added. The reaction mixture was stirred at RT overnight and concentrated onto Celite. The desired product was isolated using silica gel chromatography. ¹H NMR (400 MHz, Chloroform-d) δ 8.68 (d, J=5.2 Hz, 2H), 8.42 (s, 2H), 7.46 (dd, J=26.0, 5.1 Hz, 2H), 5.32 (br s, 2H), 4.25 (d, J=4.9 Hz, 4H), 3.83-3.29 (m, 30H), 3.17 (s, 3H), 1.80 (p, J=6.9 Hz, 2H), 1.66 (s, 6H), 1.62 (s, 6H), 1.52-1.15 (m, 4H). LRMS [M+H]⁺ 844.5.

Ir-8810: Intermediate 4 (10.3 mg, 10 μmol, 1.0 equiv) and intermediate 14 (11 mg, 13 μmol, 1.3 equiv) was dissolved in DCM/MeOH (1/1, 2 mL). The solution was stirred at RT overnight. The desired product was isolated using prep-HPLC using 0.1% TFA in H₂O and ACN as mobile phases. LRMS [M+H]⁺ 1640.1.

### Synthesis of Ir-8972

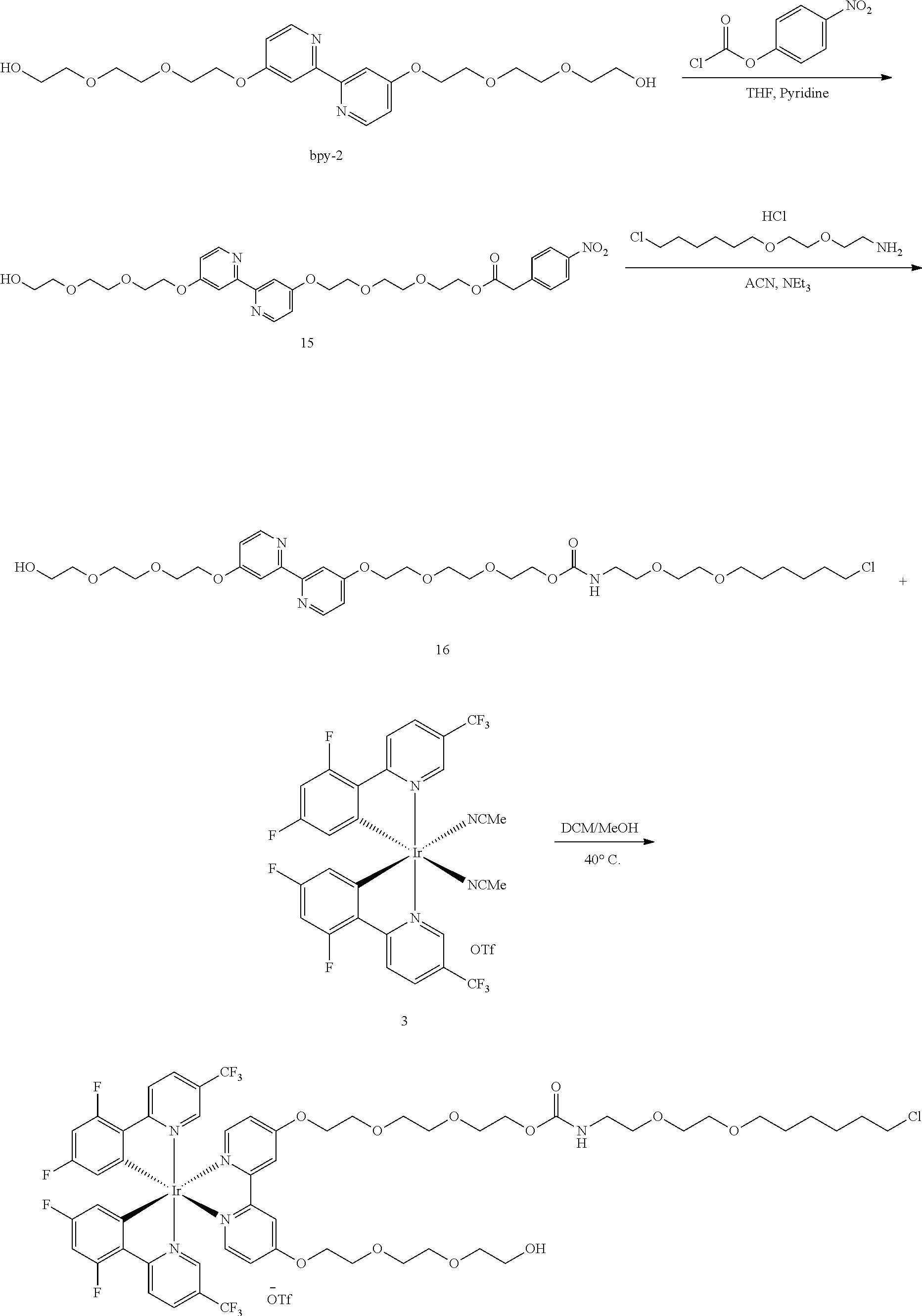

Intermediate 16: To a solution of bpy-2 (25 mg, 0.06 mmol, 1.0 equiv) in THF (4 mL), pyridine (0.5 mL) and p-nitrophenyl chloroformate (15 mg, 0.07 mmol, 1.2 equiv) was added. The solution was stirred at RT overnight. The reaction was diluted with DCM (10 mL), filtered over Celite, and the filtrate was concentrated in vacuo to afford the crude, intermediate 15, which was used in the next step without further purification.

To a solution of the crude 15 (54 mg, 87 μmol, 1.0 equiv) in ACN (2 mL), the chloroalkane amine reactive agent (15 mg, 92 μmol, 1.1 equiv) and NEt₃ (0.2 mL) was added. The solution was stirred at RT overnight and concentrated onto celite. The desired product was isolated using silica gel chromatography. ¹H NMR (400 MHz, Methanol-d₄) δ 8.47 (d, J=5.8 Hz, 2H), 7.88 (d, J=2.6 Hz, 2H), 7.07 (dd, J=5.6, 2.6 Hz, 2H), 4.35 (t, J=4.4 Hz, 4H), 4.17 (d, J=5.0 Hz, 2H), 3.93 (q, J=3.3 Hz, 4H), 3.77-3.64 (m, 12H), 3.61-3.43 (m, 12H), 3.30-3.28 (m, 2H), 1.76 (t, J=7.1 Hz, 2H), 1.66-1.30 (m, 6H). LRMS [M+H]⁺ 702.3.

Ir-8972: Intermediate 3 (10.6 mg, 11 μmol, 1.0 equiv) and intermediate 16 (8 mg, 12 μmol, 1.1 equiv) was dissolved in DCM/MeOH (1/1, 2 mL). The solution was stirred at RT overnight. The desired product was isolated using prep-HPLC using 0.1% TFA in H₂O and ACN as mobile phases. ¹H NMR (400 MHz, Methanol-d₄) δ 8.56 (d, J=9.0 Hz, 2H), 8.42-8.24 (m, 4H), 7.82 (d, J=6.3 Hz, 4H), 7.25 (d, J=6.8 Hz, 2H), 6.79 (t, J=10.9 Hz, 2H), 5.77 (d, J=8.1 Hz, 2H), 4.45 (d, J=4.7 Hz, 4H), 4.07 (s, 2H), 3.90 (d, J=4.7 Hz, 4H), 3.77-3.40 (m, 24H), 3.30-3.23 (d, J=6.1 Hz, 2H), 1.78-1.66 (m, 2H), 1.56 (t, J=7.2 Hz, 2H), 1.43-1.35 (m, 4H). LRMS [M]⁺ 1410.4.

### Synthesis of Ir-8973

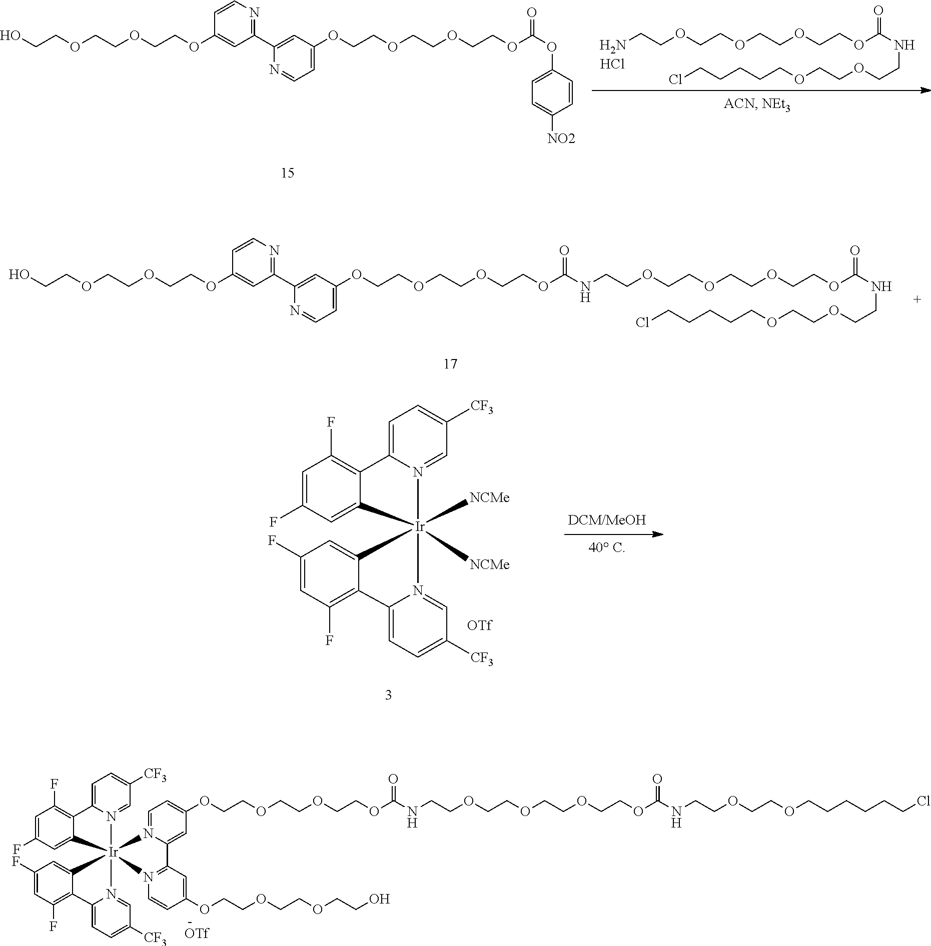

Intermediate 17: To a solution of the crude 15 (30 mg, 49 μmol, 1.0 equiv) in ACN (2 mL), the chloroalkane amine reactive agent (23 mg, 49 μmol, 1.0 equiv) and NEt₃ (0.2 mL) was added. The solution was stirred at RT overnight and concentrated onto celite. The desired product was isolated using silica gel chromatography. ¹H NMR (400 MHz, Methanol-d₄) δ 8.47 (d, J=5.8 Hz, 2H), 7.88 (t, J=2.7 Hz, 2H), 7.06 (d, J=5.7 Hz, 2H), 4.34 (d, J=4.8 Hz, 4H), 4.16 (d, J=4.9 Hz, 4H), 3.93 (h, J=2.9 Hz, 4H), 3.82-3.44 (m, 38H), 3.33-3.25 (m, 2H), 1.77 (t, J=7.3 Hz, 2H), 1.60 (t, J=7.1 Hz, 2H), 1.44 (dq, J=23.7, 7.7 Hz, 4H). LRMS [M+H]⁺ 921.4.

Ir-8973: Intermediate 3 (4.3 mg, 4.6 μmol, 1.0 equiv) and intermediate 17 (4.2 mg, 4.6 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHz, Methanol-d₄) δ 8.56 (d, J=8.9 Hz, 2H), 8.42-8.25 (m, 4H), 7.82 (d, J=4.9 Hz, 4H), 7.25 (d, J=6.4 Hz, 2H), 6.97-6.68 (m, 4H), 5.85-5.68 (m, 2H), 4.49-4.41 (m, 4H), 4.15-4.04 (m, 4H), 3.94-3.85 (m, 4H), 3.73-3.41 (m, 38H), 3.30-3.24 (m, 2H), 1.81-1.68 (m, 2H), 1.63-1.50 (m, 2H), 1.50-1.26 (m, 4H). LRMS [M+H]⁺ 1410.4.

### Synthesis of Ir-9049

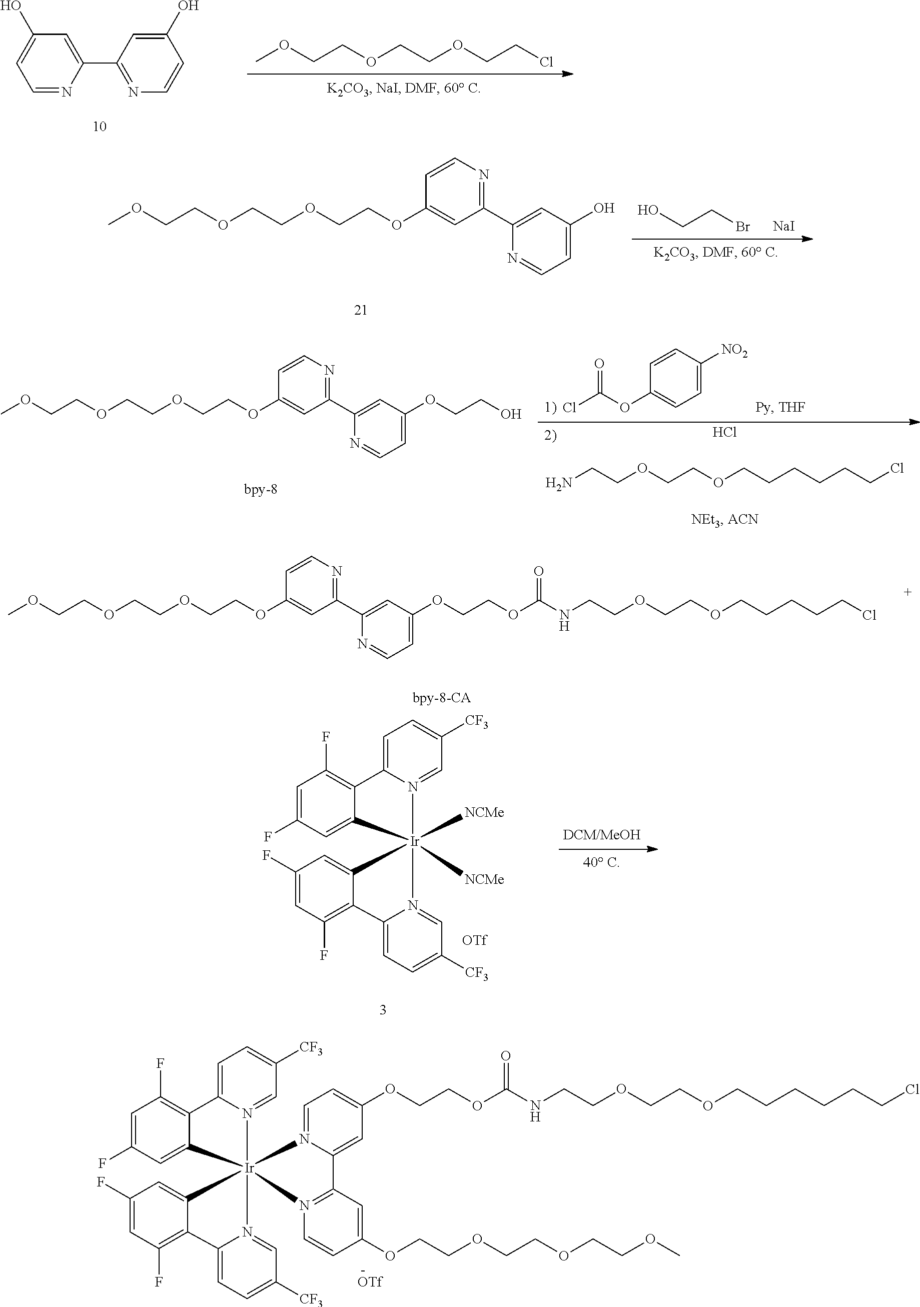

Intermediate 21: To a solution of 10 (94 mg, 0.5 mmol, 1.0 equiv) in DMF (5 mL), NaI (7.5 mg, 0.05 mmol, 0.1 equiv), 1-chloro-2-(2-(2-methoxyethoxy)ethoxy)ethane (110 mg, 0.6 mmol, 1.2 equiv), and K₂CO₃ (207 mg, 1.5 mmol, 3.0 equiv) was added. The suspension was heated at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHz, Methanol-d₄) δ 8.63 (d, J=5.9 Hz, 1H), 8.46 (dd, J=6.7, 2.1 Hz, 1H), 7.90 (s, 1H), 7.85 (s, 1H), 7.32-7.23 (m, 1H), 7.20 (d, J=6.7 Hz, 1H), 4.43 (t, J=4.6 Hz, 2H), 3.93 (p, J=2.2 Hz, 2H), 3.76-3.47 (m, 8H), 3.35 (s, 3H). LRMS [M+H]⁺ 335.4.

bpy-8: To a solution of 21 (25 mg, 75 μmol, 1.0 equiv) in DMF (5 mL), bromoethanol (47 mg, 374 μmol, 5.0 equiv), NaI (1.2 mg, 7.5 μmol, 0.1 equiv), and K₂CO₃ (31 mg, 224 μmol, 3.0 equiv) was added. The mixture was stirred at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHz, Chloroform-d) δ 8.66-8.44 (m, 2H), 8.13-7.75 (m, 2H), 7.00-6.85 (m, 2H), 6.79 (brs, 1H), 4.37-4.20 (m, 4H), 4.04-3.58 (m, 12H), 3.34 (s, 3H). LRMS [M+H]⁺ 379.4.

bpy-8-CA: To a solution of bpy-8 (16 mg, 0.04 mmol, 1.0 equiv) in THF (4 mL), pyridine (0.5 mL) and p-nitrophenyl chloroformate (10 mg, 0.05 mmol, 1.2 equiv) was added. The solution was stirred at RT overnight. The reaction was diluted with DCM (10 mL), filtered over Celite, and the filtrate was concentrated in vacuo to afford the crude, which was used in the next step without further purification.

To a solution of the crude from previous step in ACN (2 mL), the chloroalkane amine reactive agent (39 mg, 150 μmol, 3 equiv) and NEt₃ (0.2 mL) was added. The solution was stirred at RT overnight and concentrated onto celite. The desired product was isolated using silica gel chromatography. ¹H NMR (400 MHz, Methanol-d4) δ 8.48-8.46 (m, 2H), 7.95-7.74 (m, 2H), 7.17-6.92 (m, 2H), 4.47-4.25 (m, 6H), 3.95-3.42 (m, 20H), 3.35 (s, 3H), 3.29-3.17 (m, 2H), 1.77-1.50 (m, 2H), 1.63-1.50 (m, 2H), 1.47-1.21 (m, 4H). LRMS [M+H]⁺ 628.3.

Ir-9049: Intermediate 3 (14 mg, 15 μmol, 1.0 equiv) and bpy-8-CA (9.4 mg, 15 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHz, Methanol-d₄) δ 8.58 (d, J=8.9 Hz, 2H), 8.39 (s, 2H), 8.33 (d, J=8.9 Hz, 2H), 7.84 (d, J=6.9 Hz, 4H), 7.27 (s, 2H), 6.81 (t, J=10.9 Hz, 2H), 5.79 (d, J=8.0 Hz, 2H), 4.47-4.25 (m, 4H), 3.92-3.78 (m, 2H), 3.75-3.58 (m, 20H), 3.32 (s, 3H), 1.78-1.70 (m, 2H), 1.63-1.50 (m, 2H), 1.47-1.37 (m, 4H). LRMS [M+H]⁺ 1336.7.

### Synthesis of Ir-9050

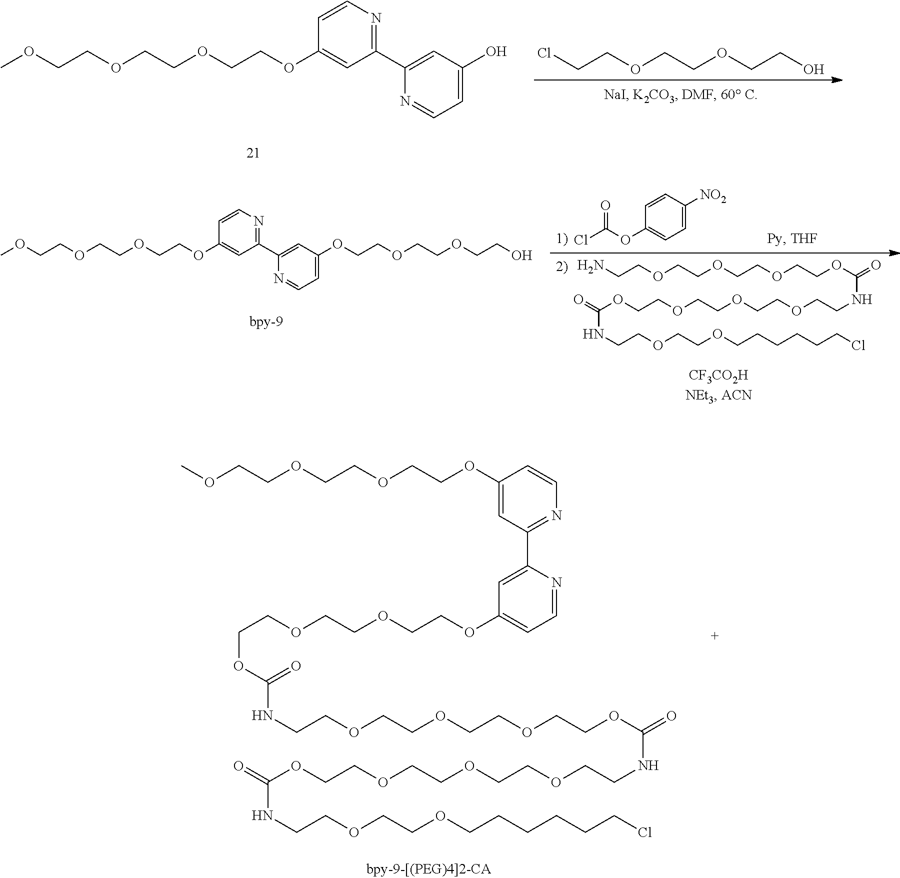

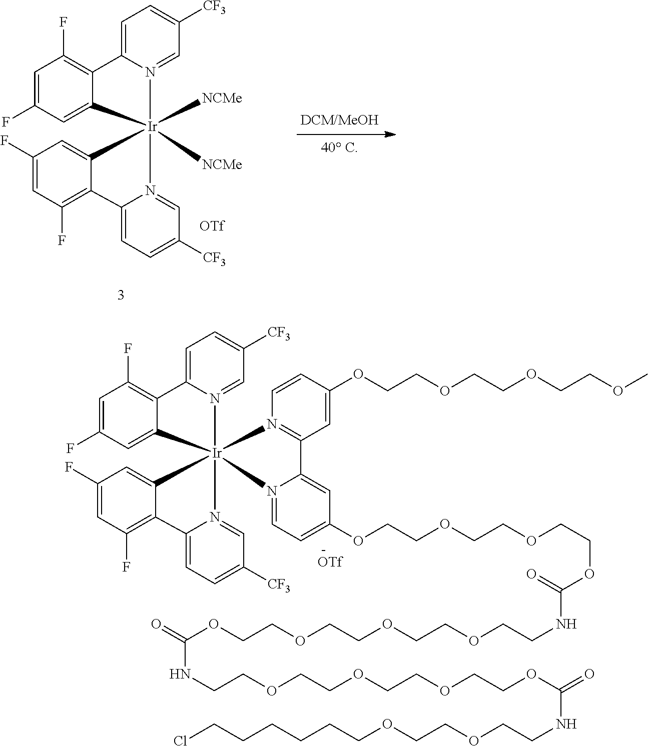

bpy-9: To a solution of 21 (25 mg, 75 μmol, 1.0 equiv) in DMF (5 mL), 2-(2-(2-chloroethoxy)ethoxy)ethan-1-ol (63 mg, 374 μmol, 5.0 equiv), NaI (1.2 mg, 7.5 μmol, 0.1 equiv), and K₂CO₃ (31 mg, 224 μmol, 3.0 equiv) was added. The mixture was stirred at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHz, Methanol-d₄) δ 8.60-8.35 (m, 2H), 7.99-7.55 (m, 2H), 7.15-6.94 (m, 2H), 4.38-4.27 (m, 4H), 3.90 (d, J=4.7 Hz, 4H), 3.76-3.47 (m, 16H), 3.30 (s, 3H). LRMS [M+H]⁺ 467.5.

bpy-9-[(PEG)4]2-CA: To a solution of bpy-9 (16 mg, 0.35 mmol, 1.0 equiv) in THF (4 mL), pyridine (0.5 mL) and p-nitrophenyl chloroformate (8.3 mg, 0.04 mmol, 1.2 equiv) was added. The solution was stirred at RT overnight. The reaction was diluted with DCM (10 mL), filtered over Celite, and the filtrate was concentrated in vacuo to afford the crude, which was used in the next step without further purification.

To a solution of the crude from previous step in ACN (2 mL), the chloroalkane amine reactive agent (54 mg, 150 μmol, 3 equiv) and NEt₃ (0.2 mL) was added. The solution was stirred at RT overnight and concentrated onto celite. The desired product was isolated using silica gel chromatography. LRMS [M+H]⁺ 1154.6.

Ir-9050: Intermediate 3 (14 mg, 15 μmol, 1.0 equiv) and bpy-9-CA (9.4 mg, 15 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHz, Methanol-d₄) δ 8.58 (d, J=8.9 Hz, 2H), 8.39 (s, 2H), 8.33 (d, J=8.9 Hz, 2H), 7.84 (d, J=6.9 Hz, 4H), 7.27 (s, 2H), 6.81 (t, J=10.9 H

### Syntheses of Ru Catalysts:

Ru-8975: The desired product was isolated as di-acetate salt. ¹H NMR (400 MHz, Methanol-d₄) δ 8.90 (d, J=8.5 Hz, 1H), 8.83-8.70 (m, 4H), 8.31 (d, J=8.5 Hz, 1H), 8.25-8.03 (m, 5H), 7.94 (dd, J=12.4, 5.6 Hz, 2H), 7.78 (s, 1H), 7.75-7.62 (m, 3H), 7.60-7.48 (m, 3H), 7.36 (d, J=6.4 Hz, 2H), 7.03 (s, 1H), 3.81 (t, J=6.0 Hz, 2H), 3.59 (t, J=7.1 Hz, 2H), 2.07 (t, J=6.6 Hz, 2H), 1.90 (s, 6H). LRMS [M]²⁺/2 333.6.

### Synthesis of Phenanthroline-1

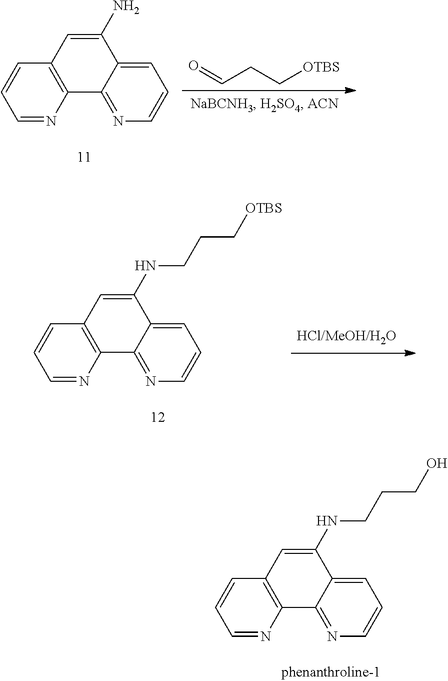

Phenanthroline-1: Phenanthroline 11 (195 mg, 1.0 mmol, 1.0 equiv), 3-((tert-butyldimethylsilyl)oxy)propanal (188 mg, 1.0 mmol, 1.0 equiv) was dissolved in ACN (10 mL) and conc. H₂SO₄ (0.1 mL) was added. The solution was stirred at RT for 30 min before NaCNBH₃ (94 mg, 1.5 mmol, 1.5 equiv) was added in one portion. The reaction mixture was then stirred at RT for additional 3 h before quenched by addition of sat. aqueous NaHCO₃ solution (1 mL). The mixture was then diluted with H₂O (30 mL) and extracted with EtOAc (30×3 mL). The combined organic layers were washed with H₂O (50 mL) and brine (50 mL), dried over Na₂SO₄, and concentrated in vacuo. The desired product 12 was purified by silica gel chromatography. ¹H NMR (400 MHz, Methanol-d₄) δ 9.18-8.92 (m, 1H), 8.80-8.51 (m, 2H), 8.12 (d, J=8.1 Hz, 1H), 7.81-7.45 (m, 2H), 6.75 (s, 1H), 3.89 (t, J=6.0 Hz, 2H), 3.48 (t, J=6.9 Hz, 2H), 2.18-1.88 (m, 2H), 0.92 (s, 9H), 0.09 (s, 6H). LRMS [M+H]⁺ 368.5.

Phenanthroline intermediate 12 (120 mg, 0.33 mmol, 1.0 equiv) was dissolved in MeOH/6N aq. HCl (1/1, 6 mL). The solution was stirred at RT for 6 h. LC-MS indicated full conversion. The desired product, phenanthroline-1, was isolated by silica gel chromatography. ¹H NMR (400 MHz, Methanol-d₄) δ 9.07 (d, J=4.2 Hz, 1H), 8.68 (dd, J=20.8, 6.3 Hz, 2H), 8.15 (d, J=8.1 Hz, 1H), 7.74 (dd, J=9.0, 4.1 Hz, 1H), 7.61-7.46 (m, 1H), 6.80 (s, 1H), 3.91-3.80 (m, 2H), 3.52 (t, J=7.1 Hz, 2H), 2.14-1.99 (m, 2H). LRMS [M+H]⁺ 254.3.

### Synthesis of Ru-8974

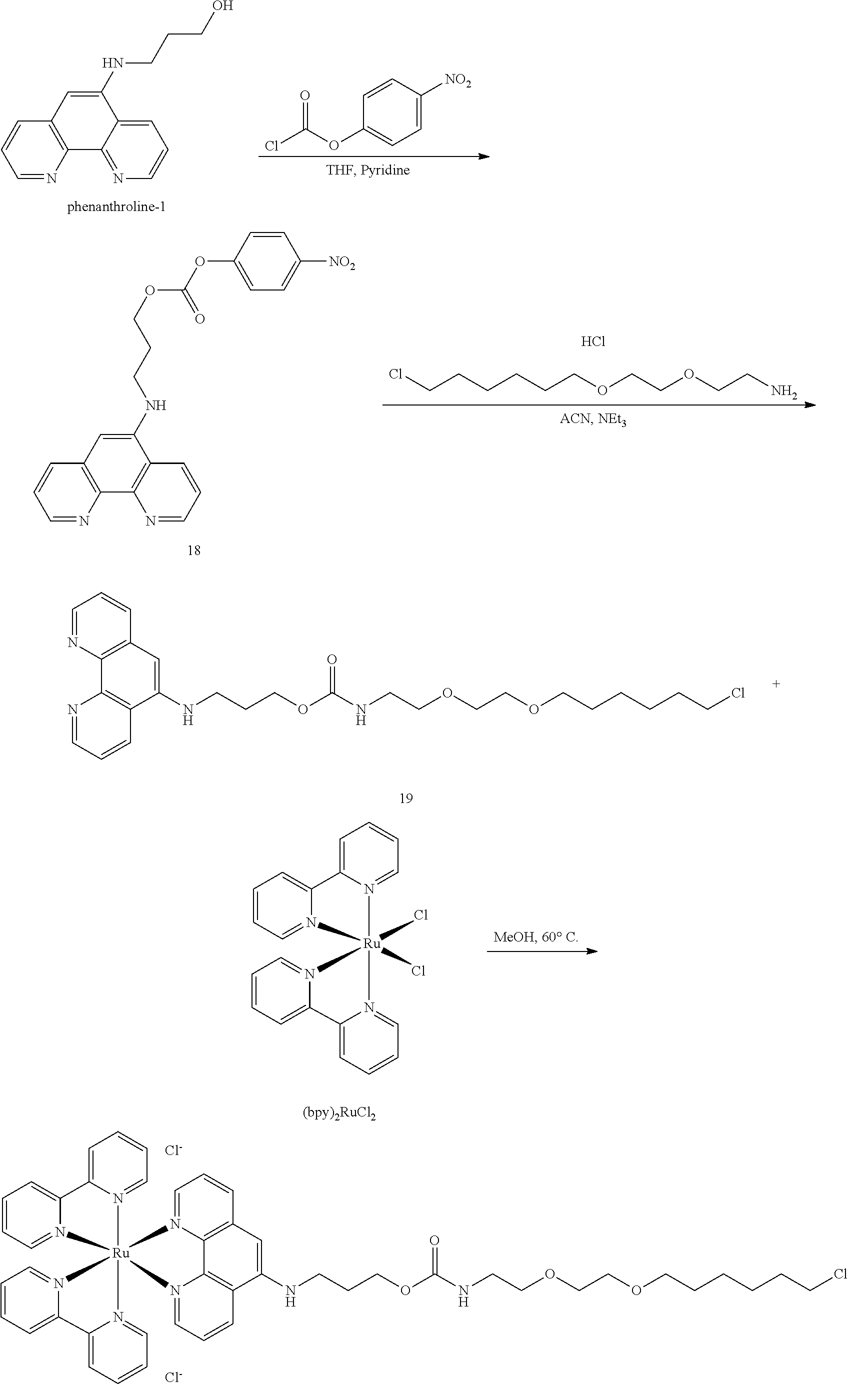

Intermediate 19: To a solution of phenanthroline-1 (16 mg, 0.06 mmol, 1.0 equiv) in THF (4 mL), pyridine (0.5 mL) and p-nitrophenyl chloroformate (13 mg, 0.06 mmol, 1.0 equiv) was added. The solution was stirred at RT overnight. The reaction was diluted with DCM (10 mL), filtered over Celite, and the filtrate was concentrated in vacuo to afford the crude, intermediate 18, which was used in the next step without further purification.

To a solution of the crude 18 (26 mg, 62 μmol, 1.0 equiv) in ACN (2 mL), the chloroalkane amine reactive agent (16 mg, 62 μmol, 1.0 equiv) and NEt₃ (0.2 mL) was added. The solution was stirred at RT overnight and concentrated onto celite. The desired product was isolated using silica gel chromatography. ¹H NMR (400 MHz, Methanol-d₄) δ 9.07 (d, J=4.3 Hz, 1H), 8.71 (t, J=7.6 Hz, 2H), 8.16 (d, J=8.1 Hz, 1H), 7.75 (dd, J=8.8, 4.1 Hz, 1H), 7.68-7.44 (m, 1H), 6.80 (s, 1H), 4.29 (t, J=6.3 Hz, 2H), 3.60-3.25 (m, 14H), 2.25-2.15 (d, J=6.6 Hz, 2H), 1.81-1.68 (m, 2H), 1.63-1.50 (m, 2H), 1.43-1.17 (m, 6H). LRMS [M+H]+ 503.3.

Ru-8974: (bpy)₂RuCl₂ (7.0 mg, 14 μmol, 1.1 equiv) and intermediate 19 (6.6 mg, 13 μmol, 1.0 equiv) was dissolved in MeOH (2 mL). The solution was stirred at 60° C. overnight. The desired product was isolated by silica column with DCM/MeOH as eluent. ¹H NMR (400 MHz, Methanol-d₄) δ 8.92 (d, J=8.6 Hz, 1H), 8.81-8.60 (m, 4H), 8.32 (d, J=8.4 Hz, 1H), 8.12 (dt, J=28.6, 9.3 Hz, 5H), 7.94 (dd, J=12.0, 5.6 Hz, 2H), 7.82-7.62 (m, 4H), 7.55 (p, J=6.6, 5.9 Hz, 3H), 7.36 (d, J=6.6 Hz, 2H), 7.04 (s, 1H), 4.36-4.15 (m, 2H), 3.67-3.41 (m, 14H), 3.30-3.17 (m, 2H), 2.25-2.15 (m, 2H), 1.81-1.68 (m, 2H), 1.63-1.50 (m, 2H), 1.43-1.17 (m, 6H). LRMS [M]²⁺/2 458.1.

### Synthesis of Ru-9003

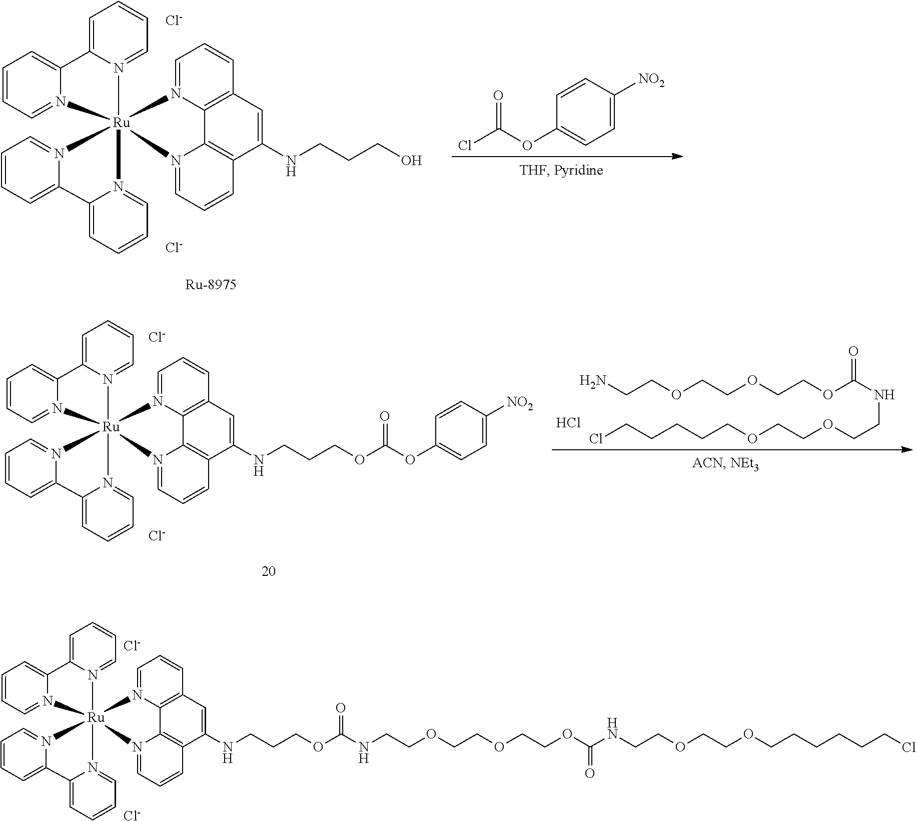

Ru-9003: To a solution of the Ru-8975 (10 mg, 13 μmol, 1.0 equiv) in ACN (2 mL), pyridine (0.5 mL) and p-nitrophenyl chloroformate (7.7 mg, 39 μmol, 3.0 equiv) was added. The solution was stirred at RT overnight. The reaction was diluted with DCM (10 mL), filtered over Celite, and the filtrate was concentrated in vacuo to afford the crude, intermediate 20, which was used in the next step without further purification.

To the solution of crude intermediate 20 was redissolved in ACN (3 mL), the chloroalkane intermediate (32 mg, 39 μmol, 3.0 equiv) and NEt₃ (0.2 mL) was added. The solution was stirred at RT overnight. The desired product was isolated by silica column with DCM/MeOH as eluent. ¹H NMR (400 MHz, Methanol-d₄) δ 8.93 (d, J=8.6 Hz, 1H), 8.81-8.66 (m, 6H), 8.33 (d, J=8.3 Hz, 1H), 8.23-8.05 (m, 7H), 7.95 (dd, J=12.0, 5.6 Hz, 3H), 7.79 (t, J=7.0 Hz, 1H), 7.68 (dd, J=15.9, 9.3 Hz, 4H), 7.63-7.51 (m, 5H), 7.38 (d, J=7.0 Hz, 3H), 7.05 (s, 1H), 4.34-4.22 (m, 2H), 4.19-4.05 (m, 2H), 3.82-3.42 (m, 22H), 3.30-3.17 (m, 2H), 2.25-2.15 (m, 2H), 1.80-1.66 (m, 2H), 1.66-1.52 (m, 2H), 1.45-1.19 (m, 4H). LRMS [M]²⁺/2 545.8. z, 2H), 5.79 (d, J=8.0 Hz, 2H), 4.49-4.41 (m, 4H), 4.15-4.04 (m, 4H), 3.94-3.85 (m, 4H), 3.79-3.47 (m, 57H), 3.24-3.15 (m, 2H), 1.90-1.67 (m, 2H), 1.67-1.47 (m, 2H), 1.46-1.28 (m, 4H). LRMS [M+H]⁺ 1863.3.

### Synthesis of Organic Catalysts:

### Synthesis of PS-9167

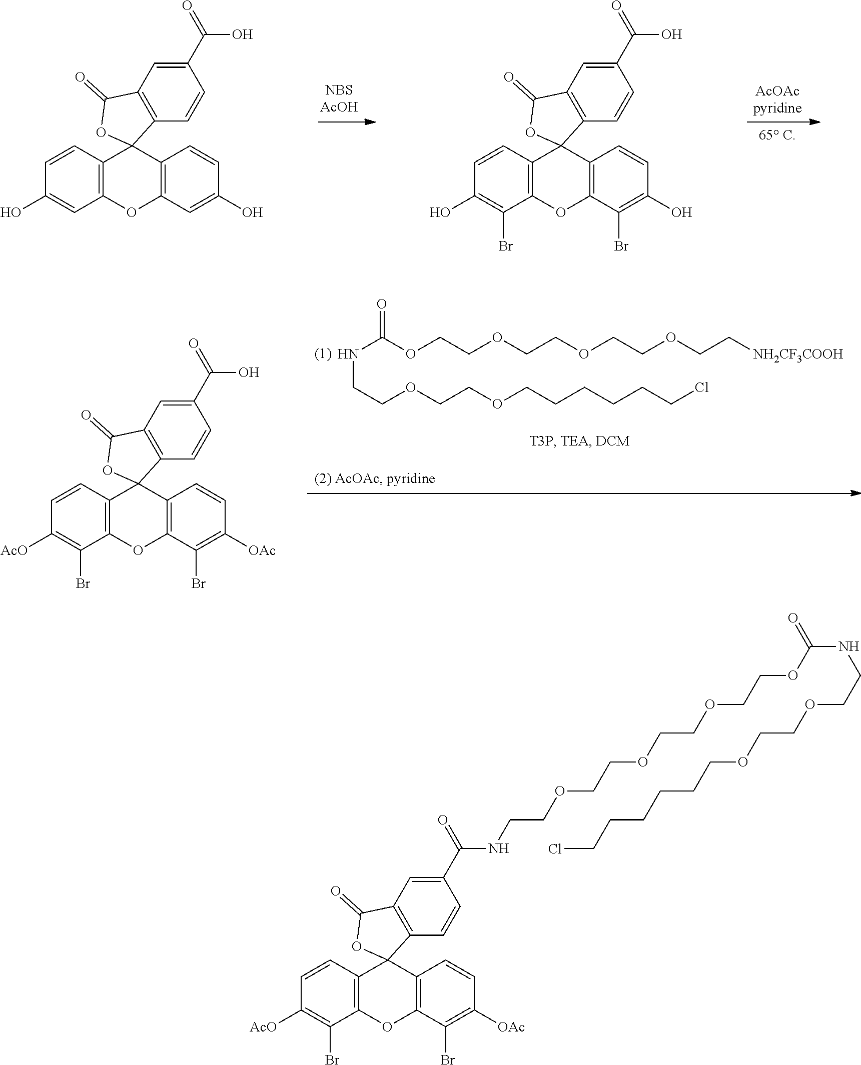

Step 1. 5-Carboxyfluorescein (1.0 g, 0.8 mmol, 1 equiv) was suspended in acetic acid (80 mL), and the mixture was heated to 60° C. NBS (354 mg, 2.0 mmol, 2.5 equiv) was dissolved separately in 20 mL acetic acid and added to the heated suspension. The addition of NBS caused the dissolution of 5-carboxyfluorescein. The reaction mixture was heated to 80° C. and stirred for another 2 h. The crude was concentrated and purified by silica gel with DCM/MeOH as eluent gives the desired product 4′,5′-dibromo-3′,6′-dihydroxy-3-oxo-3H-spiro[isobenzofuran-1,9′-xanthene]-5-carboxylic acid (DBF) (405 mg, 95%).

Step 2. DBF (400 mg, 0.75 mmol, 1 equiv) was added to 2 mL of acetic anhydride. To the mixture, 0.75 mL of dry pyridine was added. The suspension was stirred at 65° C. for 3 h until no starting material left. The mixture was concentrated and redissolved in EtOAc and washed with saturated aqueous ammonium chloride solution. The organic layer was dried with Na₂SO₄, filtered, and concentrated to afford 3′,6′-diacetoxy-4′,5′-dibromo-3-oxo-3H-spiro[isobenzofuran-1,9′-xanthene]-5-carboxylic acid (426 mg, 92%) without further purification.

Step 3. 3′,6′-diacetoxy-4′,5′-dibromo-3-oxo-3H-spiro[isobenzofuran-1,9′-xanthene]-5-carboxylic acid (85 mg, 0.14 mmol, 1 equiv) was dissolved in 2 mL DCM. DIPEA, T3P, and 2-(2-(2-(2-aminoethoxy)ethoxy)ethoxy)ethyl (2-(2-((6-chlorohexyl)oxy)ethoxy)ethyl)carbamate 2,2,2-trifluoroacetate was added into the solution. The mixture was stirred at rt overnight. The solvent was removed via a rotary evaporator, and the crude was suspended in 2 mL of anhydrous acetic anhydride. To the mixture, dry pyridine (0.5 mL) was added, and the suspension was stirred at 80° C. for 3 h to a pale-yellow solution. The mixture was concentrated down via a rotary evaporator, redissolved in ethyl acetate, and washed with saturated aqueous ammonium chloride solution. The organic layer was dried over Na₂SO₄, filtered, and concentrated down via a rotary evaporator. The pale yellow solid was purified with prep-HPLC to afford the desired product 4′,5′-dibromo-5-((26-chloro-13-oxo-3,6,9,12,17,20-hexaoxa-14-azahexacosyl)carbamoyl)-3-oxo-3H-spiro[isobenzofuran-1,9′-xanthene]-3′,6′-diyl diacetate (54 mg, 38%). LRMS: [M+H]⁺ 817.56.

### Example 13

### Compound Syntheses

This example describes the synthesis of compounds described herein, including those shown in Table 1.

TABLE 1
Compound Structures

| Cpd. No. | Structure | MS |
|---|---|---|
| 8672 | 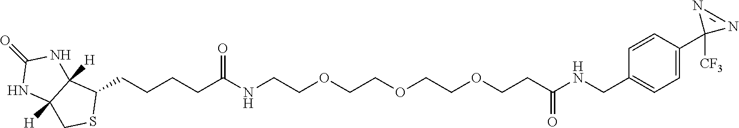 [SMILES: O=C(CCCC[C@@H]1SC[C@@H]2NC(=O)N[C@@H]21)NCCOCCOCCOCCC(=O)NCc1ccc(C2(C(F)(F)F)N=N2)cc1] | [M + H]⁺ 645.42 |
| 9107 | 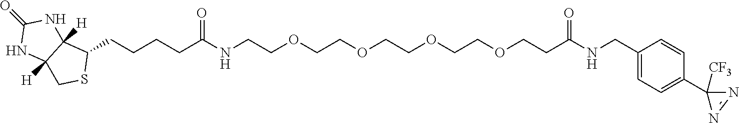 [SMILES: O=C(CCCC[C@@H]1SC[C@@H]2NC(=O)N[C@@H]21)NCCOCCOCCOCCOCCC(=O)NCc1ccc(C2(C(F)(F)F)N=N2)cc1] | [M + H]⁺ 687.31 |
| 9177 | 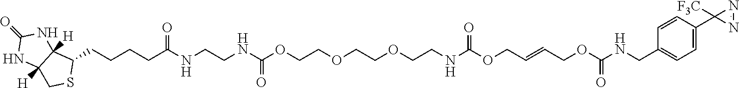 [SMILES: O=C(CCCC[C@@H]1SC[C@@H]2NC(=O)N[C@@H]21)NCCNC(=O)OCCOCCOCCNC(=O)OC/C=C/COC(=O)NCc1ccc(C2(C(F)(F)F)N=N2)cc1] | [M + H]⁺ 817.56 |
| 9069 | 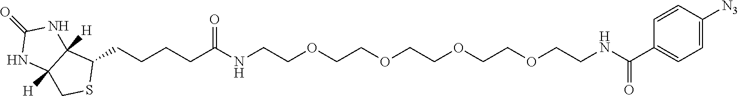 [SMILES: [N-]=[N+]=Nc1ccc(C(=O)NCCOCCOCCOCCOCCNC(=O)CCCC[C@@H]2SC[C@@H]3NC(=O)N[C@@H]32)cc1] | [M + H]⁺ 608.32 |
| 9042 | 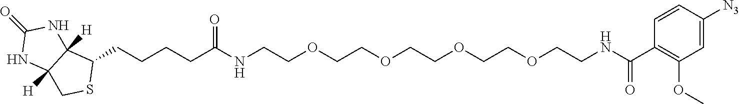 [SMILES: COc1cc(N=[N+]=[N-])ccc1C(=O)NCCOCCOCCOCCOCCNC(=O)CCCC[C@@H]1SC[C@@H]2NC(=O)N[C@@H]21] | [M + H]⁺ 638.31 |
| 9043 | 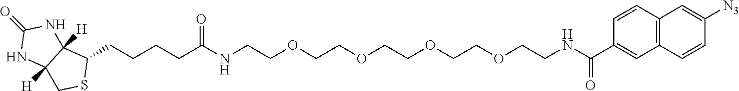 [SMILES: [N-]=[N+]=Nc1ccc2cc(C(=O)NCCOCCOCCOCCOCCNC(=O)CCCC[C@@H]3SC[C@@H]4NC(=O)N[C@@H]43)ccc2c1] | [M + H]⁺ 658.32 |
| 9044 | 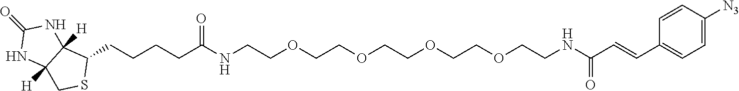 [SMILES: [N-]=[N+]=Nc1ccc(/C=C/C(=O)NCCOCCOCCOCCOCCNC(=O)CCCC[C@@H]2SC[C@@H]3NC(=O)N[C@@H]32)cc1] | [M + H]⁺ 634.28 |
| 9046 | 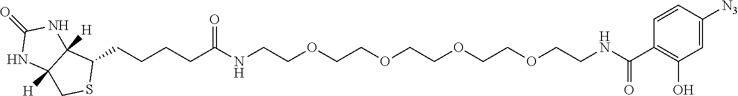 [SMILES: [N-]=[N+]=Nc1ccc(C(=O)NCCOCCOCCOCCOCCNC(=O)CCCC[C@@H]2SC[C@@H]3NC(=O)N[C@@H]32)c(O)c1] | [M + H]⁺ 624.72 |
| 9047 | 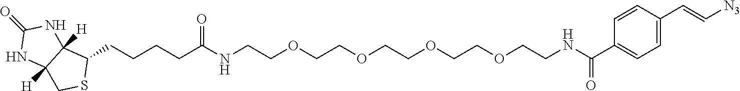 [SMILES: [N-]=[N+]=N/C=C/c1ccc(C(=O)NCCOCCOCCOCCOCCNC(=O)CCCC[C@@H]2SC[C@@H]3NC(=O)N[C@@H]32)cc1] | [M + H]⁺ 634.28 |
| 9157 | 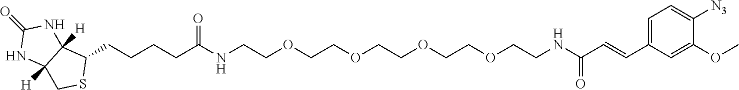 [SMILES: COc1cc(/C=C/C(=O)NCCOCCOCCOCCOCCNC(=O)CCCC[C@@H]2SC[C@@H]3NC(=O)N[C@@H]32)ccc1N=[N+]=[N-]] | [M + H]⁺ 664.28 |
| 9158 | 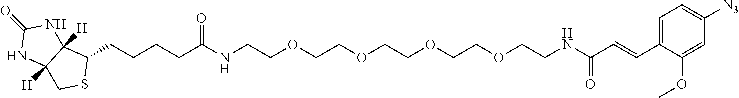 [SMILES: COc1cc(N=[N+]=[N-])ccc1/C=C/C(=O)NCCOCCOCCOCCOCCNC(=O)CCCC[C@@H]1SC[C@@H]2NC(=O)N[C@@H]21] | [M + H]⁺ 664.30 |
| 9159 | 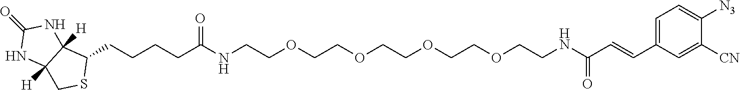 [SMILES: N#Cc1cc(/C=C/C(=O)NCCOCCOCCOCCOCCNC(=O)CCCC[C@@H]2SC[C@@H]3NC(=O)N[C@@H]32)ccc1N=[N+]=[N-]] | [M + H]⁺ 659.28 |
| 9160 | 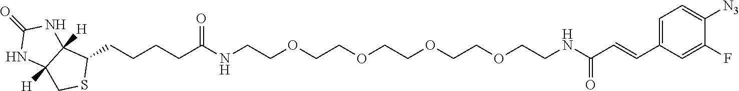 [SMILES: [N-]=[N+]=Nc1ccc(/C=C/C(=O)NCCOCCOCCOCCOCCNC(=O)CCCC[C@@H]2SC[C@@H]3NC(=O)N[C@@H]32)cc1F] | [M + H]⁺ 652.31 |
| 9161 | 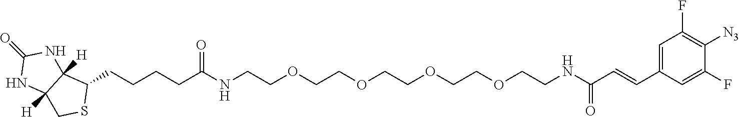 [SMILES: [N-]=[N+]=Nc1c(F)cc(/C=C/C(=O)NCCOCCOCCOCCOCCNC(=O)CCCC[C@@H]2SC[C@@H]3NC(=O)N[C@@H]32)cc1F] | [M + H]⁺ 670.28 |
| 9162 | 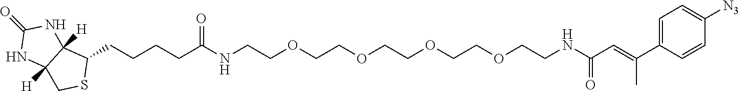 [SMILES: C/C(=C\C(=O)NCCOCCOCCOCCOCCNC(=O)CCCC[C@@H]1SC[C@@H]2NC(=O)N[C@@H]21)c1ccc(N=[N+]=[N-])cc1] | [M + H]⁺ 648.32 |
| 9422 | 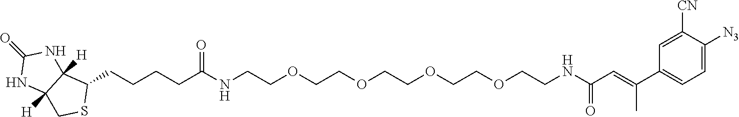 [SMILES: C/C(=C\C(=O)NCCOCCOCCOCCOCCNC(=O)CCCC[C@@H]1SC[C@@H]2NC(=O)N[C@@H]21)c1ccc(N=[N+]=[N-])c(C#N)c1] | [M + H]⁺ 673.34 |
| 9140 | 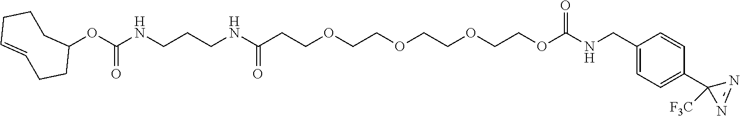 [SMILES: O=C(CCOCCOCCOCCOC(=O)NCc1ccc(C2(C(F)(F)F)N=N2)cc1)NCCCNC(=O)OC1CC/C=C/CCC1] | [M + H]⁺ 672.89 |
| 9086 | 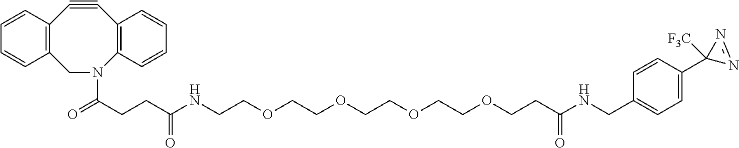 [SMILES: O=C(CCC(=O)N1Cc2ccccc2C#Cc2ccccc21)NCCOCCOCCOCCOCCC(=O)NCc1ccc(C2(C(F)(F)F)N=N2)cc1] | [M + H]⁺ 750.78 |
| 9476 | 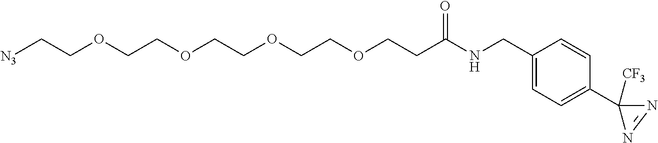 [SMILES: [N-]=[N+]=NCCOCCOCCOCCOCCC(=O)NCc1ccc(C2(C(F)(F)F)N=N2)cc1] | [M + H]⁺ 509.68 |
| 9421 | 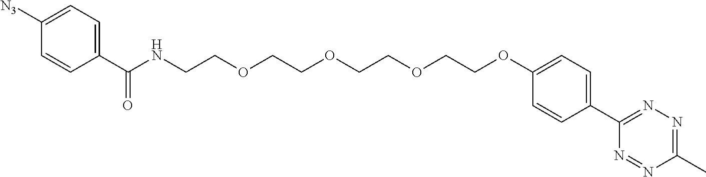 [SMILES: Cc1nnc(-c2ccc(OCCOCCOCCOCCNC(=O)c3ccc(N=[N+]=[N-])cc3)cc2)nn1] | [M + Na]⁺ 511.21 |
| 9582 | 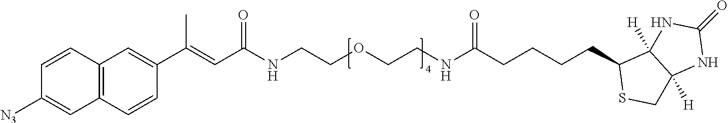 | [M + H]⁺ 698 |
| 9595 | 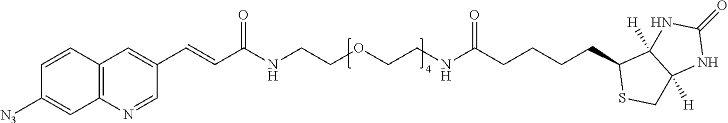 | [M + H]⁺ 685 |
| 9917 | 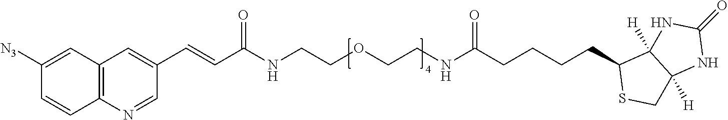 | [M + H]⁺ 685 |
| 9599 | 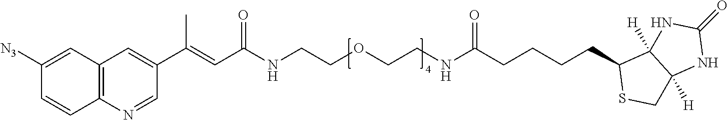 | [M + H]⁺ 699 |
| 9615 | 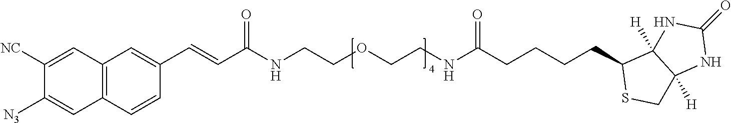 | [M + H]⁺ 709 |
| 9616 | 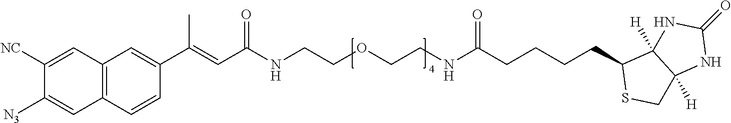 | [M + H]⁺ 723 |

### Syntheses of Diazirine-Biotin Analogs:

### Synthesis of Compound 8672

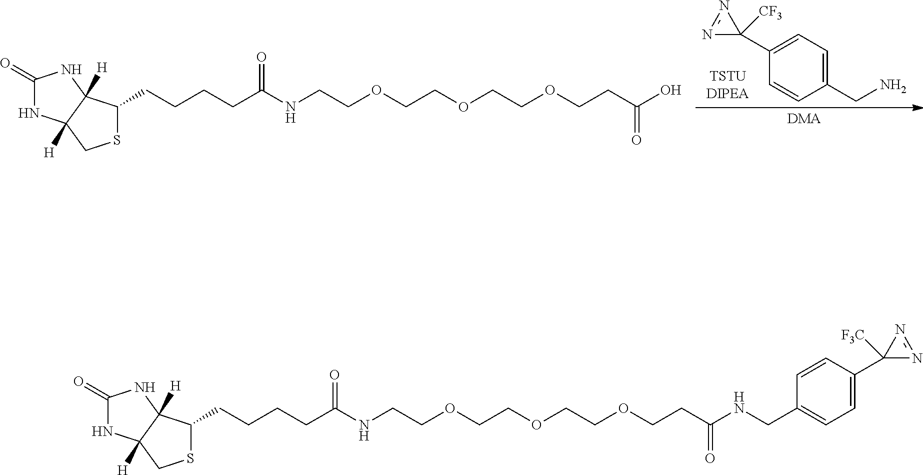

(4-(3-(trifluoromethyl)-3H-diazirin-3-yl)phenyl)methanamine (25.2 mg, 0.10 mmol, 1.0 equiv) was dissolved in 2 ml DMA. To this solution, Biotin-PEG3-COOH (49.2 mg, 0.11 mmol, 1.1 equiv), TSTU (36.1 mg, 0.12 mmol, 1.2 equiv), and DIPEA (43.6 μL, 0.25 mmol, 2.5 equiv) were added, and the reaction was allowed to stir at rt overnight. The crude was concentrated and purified through silica gel column with DCM/MeOH to afford the desired product, N-(3-oxo-1-(4-(3-(trifluoromethyl)-3H-diazirin-3-yl)phenyl)-6,9,12-trioxa-2-azatetradecan-14-yl)-5-((3aS,4S,6aR)-2-oxohexahydro-1H-thieno[3,4-d]imidazol-4-yl)pentanamide. LCMS: [M+H]⁺ 645.42.

### Synthesis of Compound 9107

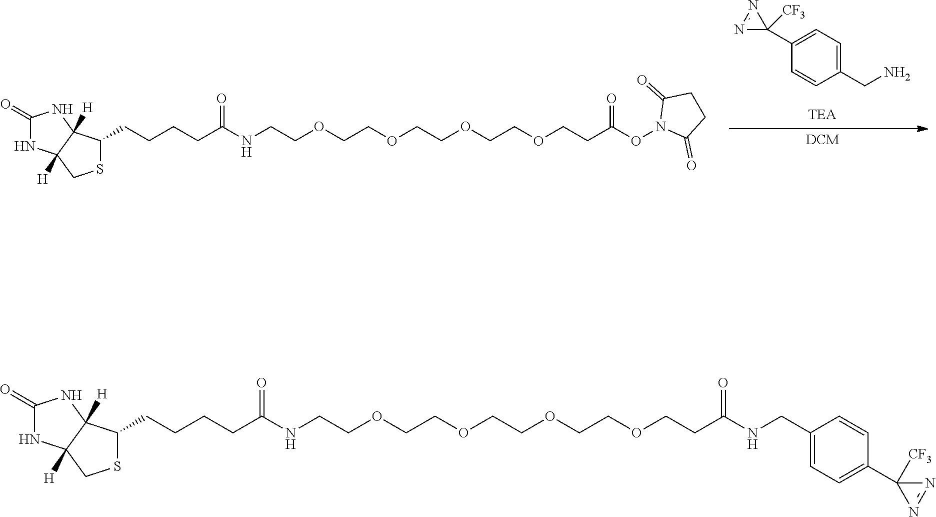

Biotin-PEG4-NHS (440 mg, 0.75 mmol, 1.0 equiv) was dissolved in 10 ml DCM. To this solution, (4-(3-(trifluoromethyl)-3H-diazirin-3-yl)phenyl)methanamine (193 mg, 0.90 mmol, 1.2 equiv) and TEA (313 μL, 2.24 mmol, 3.0 equiv) were added, and the reaction was allowed to stir at rt overnight. The crude was concentrated and purified through silica gel column with DCM/MeOH to afford the desired product, 1-(5-((3aS,4S,6aR)-2-oxohexahydro-1H-thieno[3,4-d]imidazol-4-yl)pentanamido)-N-(4-(3-(trifluoromethyl)-3H-diazirin-3-yl)benzyl)-3,6,9,12-tetraoxapentadecan-15-amide (506 mg, 98%). LCMS: [M−H]⁺ 687.31.

### Synthesis of Compound 9177

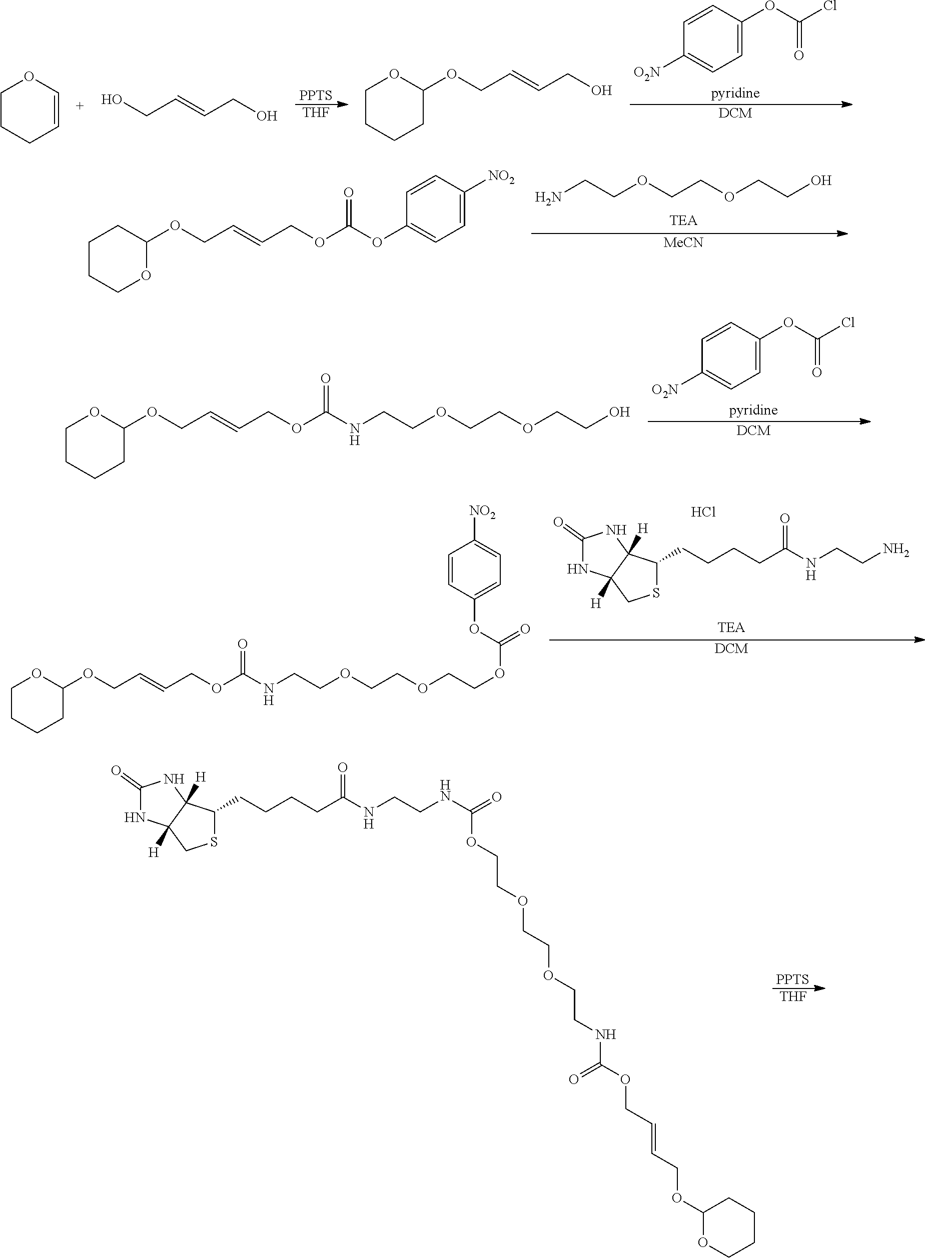

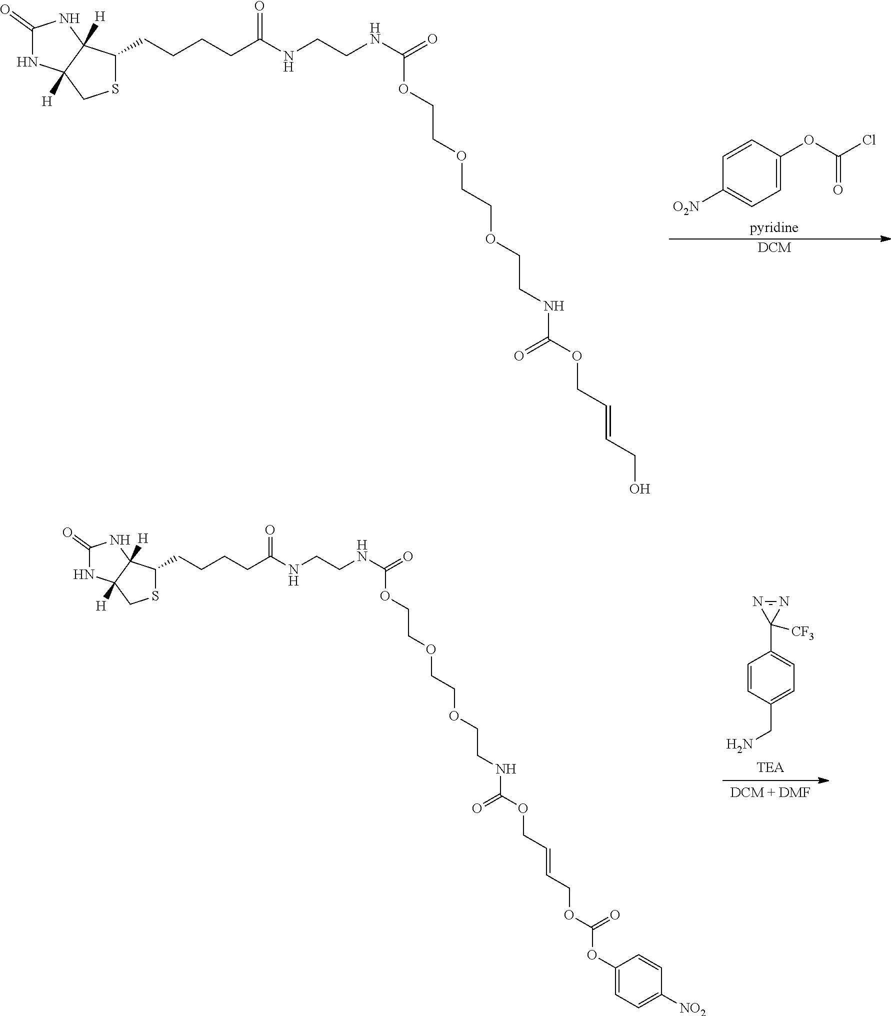

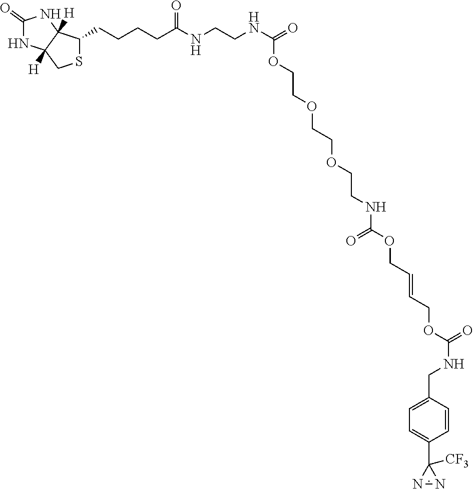 [SMILES: O=C(CCCC[C@@H]1SC[C@@H]2NC(=O)N[C@@H]21)NCCNC(=O)OCCOCCOCCNC(=O)OC/C=C/COC(=O)NCc1ccc(C2(C(F)(F)F)N=N2)cc1]

Steps 1-4 to generate (E)-4-((tetrahydro-2H-pyran-2-yl)oxy)but-2-en-1-yl (2-(2-(2-(((4-nitrophenoxy)carbonyl)oxy)ethoxy)ethoxy)ethyl)carbamate were performed according to a published literature procedure (ACS Chem. Biol. 2016, 11, 9, 2608-2617).

Step 5. To a solution of (E)-4-((tetrahydro-2H-pyran-2-yl)oxy)but-2-en-1-yl (2-(2-(2-(((4-nitrophenoxy)carbonyl)oxy)ethoxy)ethoxy)ethyl)carbamate (570 mg, 1.11 mmol, 1 equiv) and N-(2-aminoethyl)-5-((3aS,4S,6aR)-2-oxohexahydro-1H-thieno[3,4-d]imidazol-4-yl)pentanamide hydrochloride (395 mg, 1.22 mmol, 1.1 equiv), triethylamine (466 μL, 3.34 mmol, 3 equiv) was added. The resulting solution was stirred at rt for 20 h, at which point TLC analysis indicated complete consumption of the starting materials. The solvent was removed under vacuum, and the residue was purified by silica gel chromatography using MeOH/DCM) to provide desired product (E)-4-((tetrahydro-2H-pyran-2-yl)oxy)but-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (680 mg, 93%).

Step 6. (E)-4-((tetrahydro-2H-pyran-2-yl)oxy)but-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (900 mg, 1.36 mmol, 1 equiv) was dissolved in 50 mL of EtOH in RBF. To this solution, PPTS (34.2 mg, 136 μmol) was added. The resulting solution was heated at 50° C. for 1 h. The solvent was removed under vacuum, and the residue was purified by silica gel chromatography with MeOH/DCM to provide product (E)-4-hydroxybut-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (654 mg, 83%).

Step 7. (E)-4-hydroxybut-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (640 mg, 1.11 equiv, 1 equiv) was dissolved in DCM. P-nitrophenylchloroformate (269 mg, 1.33 mmol, 1.2 equiv) and pyridine (146 μL, 1.81 mmol, 1.5 equiv) were added. The resulting solution was left stirred at rt for 20 hours. Solvent was removed via rotary evaporation, and the crude was loaded directly onto silica gel column and purified by flash chromatography to afford (E)-4-(((4-nitrophenoxy)carbonyl)oxy)but-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (450 mg, 55%).

Step 8. To a solution of (E)-4-(((4-nitrophenoxy)carbonyl)oxy)but-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (27 mg, 36.5 μmol, 1 equiv) in 3 mL of DCM and 1 mL of DMF, (4-(3-(trifluoromethyl)-3H-diazirin-3-yl)phenyl)methanamine hydrochloride (9.5 mg, 37.8 μmol, 1.04 equiv) and triethyl amine (15.3 μL, 109.3 μmol, 3 equiv) were added. The mixture was allowed to stir at rt overnight. Solvent was removed via rotary evaporation, and the crude was loaded directly onto silica gel column and purified by flash chromatography to afford (E)-4-(((4-(3-(trifluoromethyl)-3H-diazirin-3-yl)benzyl)carbamoyl)oxy)but-2-en-1-yl (10,15-dioxo-19-((3aS,4S,6aR)-2-oxohexahydro-1H-thieno[3,4-d]imidazol-4-yl)-3,6,9-trioxa-11,14-diazanonadecyl)carbamate (16 mg, 54%). LRMS: [M+H]⁺ 817.56.

### Syntheses of Aryl-Azide Precursor Compounds

### Synthesis of Aryl-Azide 1:

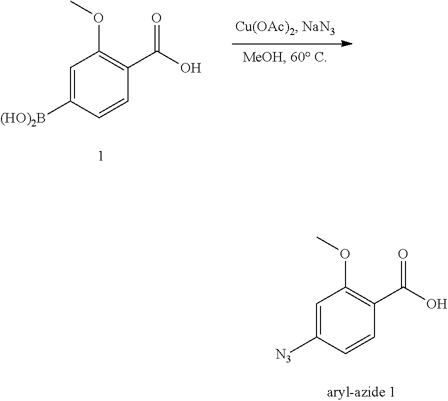

Aryl-azide 1: To a solution of aryl boric acid reagent 1 (98 mg, 0.5 mmol, 1.0 equiv) in MeOH (5 mL), Cu(OAc)₂ (9.1 mg, 0.05 mmol, 0.1 equiv) and NaN₃ (33 mg, 0.5 mmol, 1.0 equiv) was added in one portion. The solution was heated at 60° C. for 3 h. LC-MS indicated full conversion. The reaction was diluted with EtOAc (50 mL) then quenched by addition of sat. aq. NH₄Cl (10 mL). The aqueous layer was extracted with EtOAc (10×3 mL). The combined organic layer was washed with H₂O (30 mL) and brine (30 mL) and dried over Na₂SO₄ and concentrated in vacuo. The desired product was purified by silica gel chromatography. ¹H NMR (400 MHz, Acetonitrile-d₃) δ 7.44 (d, J=8.0 Hz, 1H), 6.75-6.47 (m, 2H), 3.81 (s, 3H). LRMS [M−H]⁻ 192.04.

Synthesis of aryl-azide 2:

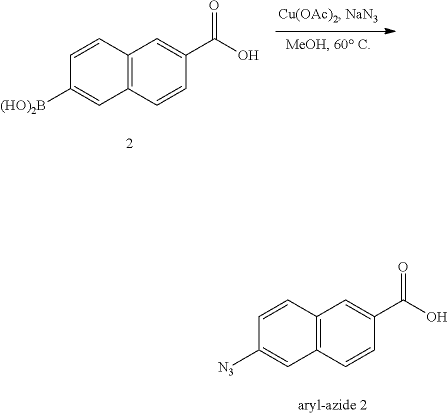 [SMILES: O=C(O)c1ccc2cc(B(O)O)ccc2c1 ; [N-]=[N+]=Nc1ccc2cc(C(=O)O)ccc2c1]

Aryl-azide 2: To a solution of aryl boric acid reagent 2 (108 mg, 0.5 mmol, 1.0 equiv) in MeOH (5 mL), Cu(OAc)₂ (9.1 mg, 0.05 mmol, 0.1 equiv) and NaN₃ (33 mg, 0.5 mmol, 1.0 equiv) was added in one portion. The solution was heated at 60° C. for 3 h. LC-MS indicated full conversion. The reaction was diluted with EtOAc (50 mL) then quenched by addition of sat. aq. NH₄Cl (10 mL). The aqueous layer was extracted with EtOAc (10×3 mL). The combined organic layer was washed with H₂O (30 mL) and brine (30 mL) and dried over Na₂SO₄ and concentrated in vacuo. The desired product was purified by silica gel chromatography. ¹H NMR (400 MHz, Methanol-d₄) δ 8.58 (s, 1H), 8.04 (t, J=7.7 Hz, 2H), 7.88 (d, J=8.7 Hz, 1H), 7.60 (s, 1H), 7.27 (d, J=8.8 Hz, 1H). LRMS [M−H]⁻ 212.05.

Synthesis of aryl-azide 3:

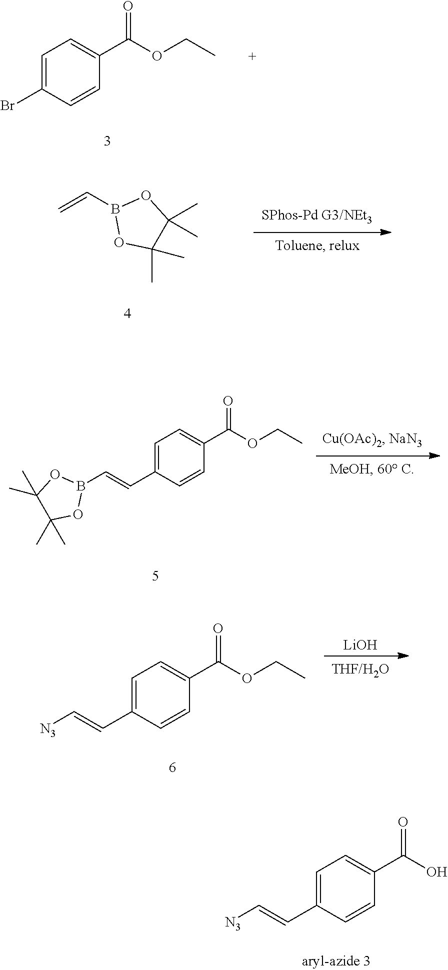

Intermediate 5: To a suspension of aryl bromide 3 (229 mg, 1.0 mmol, 1.0 equiv), vinylBpin 4 (154 mg, 1.0 mmol, 1.0 equiv) and SPhos-Pd G3 (15 mg, 0.02 mmol, 0.02 equiv) in Toluene (5 mL) under N₂, NEt₃ (0.68 mL, 5.0 mmol, 5.0 equiv) was added. The reaction mixture was stirred at RT for 15 min and heated up to 120° C. for 16 h. After cooling down to RT, the reaction was diluted with Et₂O/DCM (1/1, 50 mL), filtered over celite, and concentrated in vacuo. The crude was redissolved in DCM (50 mL) and filtered over a small pad of silica gel. The filtrate was concentrated to afford the crude, which was used in the next step without further purification. LRMS [M+H]⁺ 303.17.

Intermediate 6: To a solution of intermediate 5 (63 mg, 0.2 mmol, 1.0 equiv) in MeOH (4 mL), Cu(OAc)₂ (3.8 mg, 0.02 mmol, 0.1 equiv) and NaN₃ (14 mg, 0.2 mmol, 1.0 equiv) was added in one portion. The solution was heated at 60° C. for 3 h. LC-MS indicated full conversion. The reaction was diluted with EtOAc (50 mL), then quenched by addition of sat. aq. NH₄Cl (10 mL). The aqueous layer was extracted with EtOAc (10×3 mL). The combined organic layer was washed with H₂O (30 mL) and brine (30 mL), dried over Na₂SO₄, and concentrated in vacuo.

The desired product was purified by silica gel chromatography. ¹H NMR (400 MHz, Chloroform-d) δ 8.00-7.94 (m, 2H), 7.36-7.29 (m, 2H), 6.75 (dd, J=13.8, 2.0 Hz, 1H), 6.29 (d, J=13.8 Hz, 1H), 4.37 (q, J=7.1 Hz, 2H), 1.39 (t, J=7.1 Hz, 3H). LRMS [M+H]⁺ 218.09.

Aryl-azide 3: To a solution of intermediate 6 (25 mg, 0.12 mmol, 1.0 equiv) in THF (4 mL), LiOH (14 mg) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude, which was used in the next step without further purification. LRMS [M−H]⁻ 188.05.

Synthesis of aryl-azide 4:

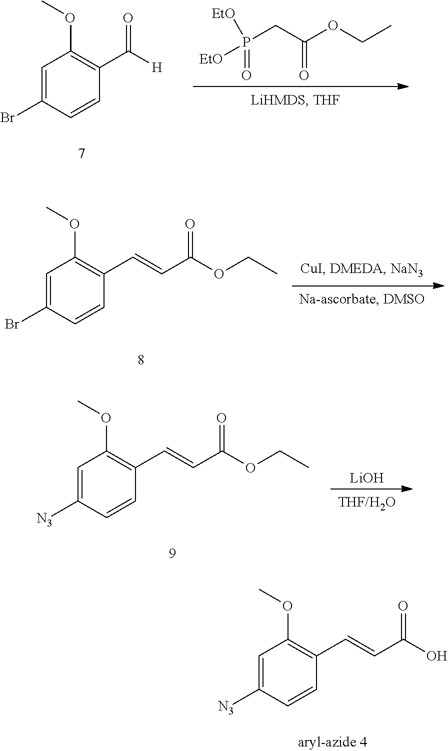

Intermediate 8: To a solution of phosphonate reagent (0.6 mL, 3.0 mmol, 1.0 equiv), LiHMDS solution (3.3 mL, 1.0 N, 3.3 mmol, 1.1 equiv) was added dropwise over 10 min. The mixture was stirred for 30 min at RT before addition of aldehyde intermediate 7 in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄, concentrated in vacuo to afford the crude. The desired product was isolated using silica gel chromatography. ¹H NMR (400 MHz, Chloroform-d) δ 7.88 (dt, J=16.1, 1.5 Hz, 1H), 7.35 (dt, J=8.4, 1.7 Hz, 1H), 7.15-6.98 (m, 2H), 6.50 (dt, J=16.2, 1.7 Hz, 1H), 4.26 (q, J=7.3 Hz, 2H), 3.89 (s, 3H), 1.33 (t, J=7.3 Hz, 3H). LRMS [M+H]⁺ 285.01.

Intermediate 9: Intermediate 8 (285 mg, 1.0 mmol, 1.0 equiv), CuI (9.5 mg, 0.05 mmol, 0.05 equiv), and Na-ascorbate (20 mg, 0.1 mmol, 0.1 equiv) were charged into a vial purged with N₂. To the mixture, DMSO (5 mL) and DMEDA (17 μL, 0.15 mmol, 0.15 equiv) was added. The mixture was then stirred at RT for 15 min before NaN₃ (98 mg, 1.5 mmol, 1.5 equiv) was added. The reaction was heated at 100° C. for 16 h. The reaction was then stirred at RT for 16 h and quenched by addition of sat. aq. NH₄Cl (20 mL). The quenched reaction was extracted with EtOAc (50×3 mL). The combined organic layer was dried over Na₂SO₄ and concentrated in vacuo to afford the crude. The desired product was isolated using silica gel chromatography. ¹H NMR (400 MHz, Chloroform-d) δ 7.90 (d, J=16.1 Hz, 1H), 7.48 (d, J=8.3 Hz, 1H), 6.74-6.60 (m, 1H), 6.56-6.38 (m, 2H), 4.25 (q, J=7.2 Hz, 2H), 3.88 (s, 3H), 1.33 (t, J=7.2 Hz, 3H). LRMS [M+H]⁺ 248.10.

Aryl-azide 4: To a solution of intermediate 9 (170 mg, 0.69 mmol, 1.0 equiv) in THF (4 mL), LiOH (32 mg, 1.38 mmol, 2.0 equiv) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude. The product was isolated using silica gel chromatography. ¹H NMR (400 MHz, Methanol-d₄) δ 7.89 (d, J=16.2, 1H), 7.60 (d, J=8.4 Hz, 1H), 6.87-6.60 (m, 2H), 6.47 (dd, J=16.1 Hz, 1H), 3.91 (s, 3H). LRMS [M−H]⁻ 218.06.

Synthesis of aryl-azide 5:

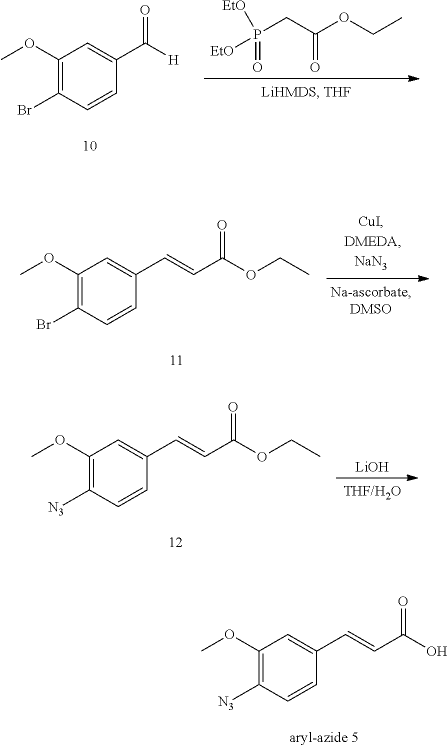

Intermediate 11: The desired product was synthesized analogously following procedures for intermediate 8 using aldehyde 10. ¹H NMR (400 MHz, Chloroform-d) δ 7.61 (d, J=16.0 Hz, 1H), 7.54 (d, J=8.1 Hz, 1H), 7.00 (d, J=7.3 Hz, 2H), 6.43 (d, J=16.0 Hz, 1H), 4.27 (q, J=7.1 Hz, 2H), 3.92 (s, 3H), 1.34 (t, J=7.1 Hz, 3H). LRMS [M+H]⁺ 285.01.

Intermediate 12: The desired product was synthesized analogously following procedures for intermediate 9. ¹H NMR (400 MHz, Chloroform-d) δ 7.49 (d, J=16.0 Hz, 1H), 7.23-7.16 (m, 2H), 7.05-6.92 (m, 1H), 6.63 (d, J=6.0 Hz, 1H), 4.27 (q, J=7.1 Hz, 2H), 3.92 (s, 3H), 1.34 (t, J=7.1 Hz, 3H). LRMS [M+H]⁺ 248.10.

Aryl-azide 5: The desired product was synthesized analogously following procedures for intermediate aryl azide 4. ¹H NMR (400 MHz, Chloroform-d) δ 7.48 (d, J=16.0 Hz, 1H), 7.25-7.16 (m, 2H), 7.10-6.95 (m, 1H), 6.63 (d, J=6.0 Hz, 1H), 3.97 (s, 3H). LRMS [M−H]⁻ 218.06.

Synthesis of aryl-azide 6:

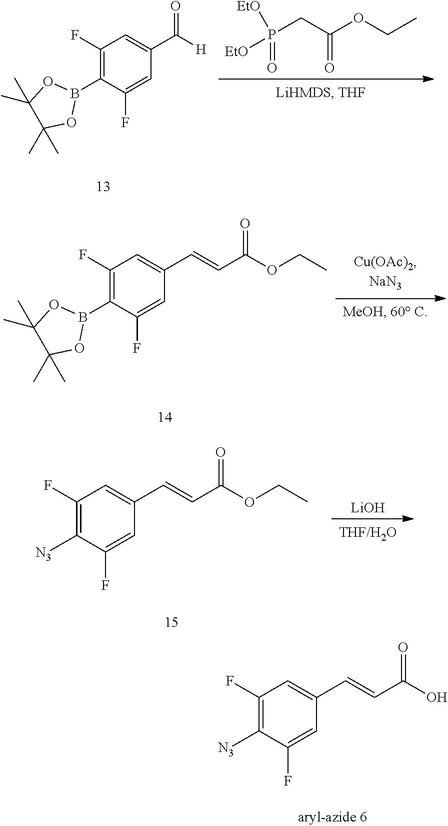

Intermediate 14: To a solution of phosphonate reagent (0.32 mL, 1.7 mmol, 1.1 equiv), LiHMDS solution (1.7 mL, 1.0 N, 1.7 mmol, 1.1 equiv) was added dropwise over 10 min. The mixture was stirred for 30 min at RT before addition of aldehyde intermediate 13 in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄, concentrated in vacuo to afford the crude. The desired product was isolated using silica gel chromatography. ¹H NMR (400 MHz, Chloroform-d) δ 7.57 (d, J=16.0 Hz, 1H), 7.01 (d, J=7.5 Hz, 2H), 6.46 (d, J=16.0 Hz, 1H), 4.36-4.20 (q, J=7.1 Hz, 2H), 1.35 (t, J=7.1 Hz, 3H). LRMS [M+H]⁺ 339.16.

Intermediate 15: To a solution of intermediate 14 (73 mg, 0.22 mmol, 1.0 equiv) in MeOH (4 mL), Cu(OAc)2 (4.0 mg, 0.022 mmol, 0.1 equiv) and NaN₃ (14 mg, 0.22 mmol, 1.0 equiv) was added. The reaction was heated at 60° C. for 30 min. LC-MS indicated full conversion. The reaction was diluted with EtOAc (50 mL) then quenched by addition of sat. aq. NH₄Cl (10 mL). The aqueous layer was extracted with EtOAc (10×3 mL). The combined organic layer was washed with H₂O (30 mL) and brine (30 mL), dried over Na₂SO₄, and concentrated in vacuo. The desired product was purified by silica gel chromatography and isolated as a mixture with proto-deboronated side product. ¹H NMR (400 MHz, Chloroform-d) δ 7.50 (d, J=15.9 Hz, 1H), 7.08 (d, J=8.7 Hz, 2H), 6.35 (d, J=15.9 Hz, 1H), 4.32-4.23 (q, J=7.2 Hz, 2H), 1.34 (t, J=7.2 Hz, 3H). LRMS [M+H]⁺ 254.07.

Aryl-azide 6: To a solution of intermediate 15 (30 mg, 0.12 mmol, 1.0 equiv) in THF (4 mL), LiOH (80 mg, 2.0 mmol, 17 equiv) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude. The desired product was isolated using silica gel chromatography. ¹H NMR (400 MHz, Methanol-d₄) δ 7.54 (d, J=15.9 Hz, 1H), 7.34 (d, J=9.2 Hz, 2H), 6.49 (d, J=16.0 Hz, 1H). LRMS [M−H]⁻ 224.03.

Synthesis of aryl-azide 7:

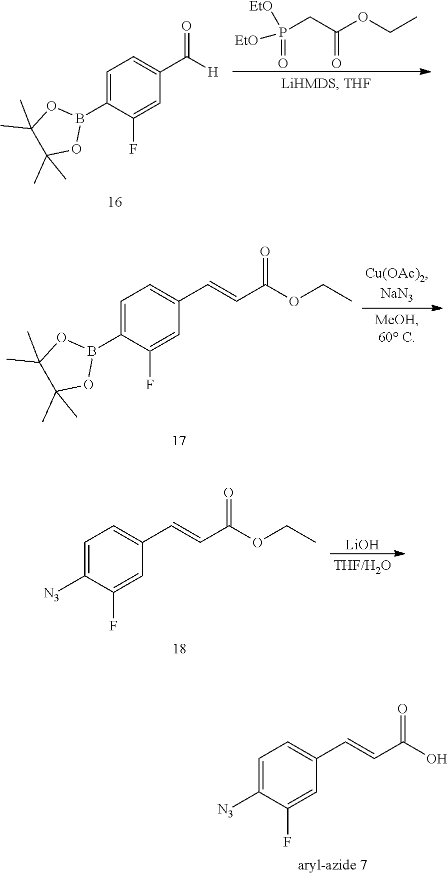

Intermediate 17: The desired product was synthesized analogously following procedures for intermediate 14 using aldehyde 16. ¹H NMR (400 MHz, Chloroform-d) δ 7.76 (dd, J=7.7, 6.2 Hz, 1H), 7.64 (d, J=15.9 Hz, 1H), 7.30 (dd, J=7.7, 1.5 Hz, 1H), 7.19 (dd, J=10.1, 1.6 Hz, 1H), 6.48 (d, J=16.0 Hz, 1H), 4.28 (q, J=7.1 Hz, 2H), 1.48-1.26 (t, J=7.1 Hz, 3H). LRMS [M+H]⁺ 321.17.

Intermediate 18: The desired product was synthesized analogously following procedures for intermediate 15. The desired product was purified by silica gel chromatography and isolated as a mixture with proto-deboronated side product. LRMS [M+H]⁺ 236.18.

Aryl-azide 7: The desired product was synthesized analogously following procedures for aryl azide 6. ¹H NMR (400 MHz, Methanol-d₄) δ 7.61 (d, J=15.9 Hz, 1H), 7.53-7.41 (m, 2H), 7.23 (d, J=8.4 Hz, 1H), 6.49 (d, J=15.6 Hz, 1H). LRMS [M−H]⁻ 206.04.

Synthesis of aryl-azide 8:

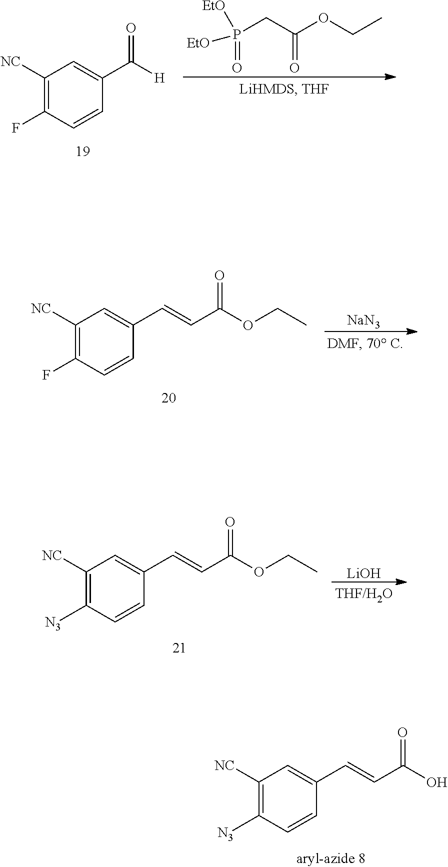

Intermediate 20: To a solution of phosphonate reagent (0.57 mL, 2.9 mmol, 1.0 equiv), LiHMDS solution (3.1 mL, 1.0 N, 3.1 mmol, 1.1 equiv) was added dropwise over 10 min. The mixture was stirred for 30 min at RT before addition of aldehyde intermediate 19 in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄ and concentrated in vacuo to afford the crude. The desired product was isolated using silica gel chromatography. ¹H NMR (400 MHz, Chloroform-d) δ 7.83-7.71 (m, 2H), 7.60 (d, J=16.0 Hz, 1H), 7.26 (t, J=8.5 Hz, 1H), 6.42 (d, J=16.0 Hz, 1H), 4.28 (q, J=7.1 Hz, 2H), 1.34 (t, J=7.1 Hz, 3H). LRMS [M+H]⁺ 220.1.

Intermediate 21: To a solution of intermediate 20 (149 mg, 0.68 mmol, 1.0 equiv) in DMF (3 mL), NaN₃ (66 mg, 1.0 mmol, 1.5 equiv) was added in one portion. The mixture was heated at 70° C. overnight. The reaction was cooled down, diluted with EtOAc (50 mL), and poured onto crashed ice. The After partition, the aqueous layer, was extracted with EtOAc (20×3 mL). The combined organic layer was washed with H₂O (50 mL) and brine (50 mL), dried over Na₂SO₄, and concentrated in vacuo. The desired product was isolated using silica gel chromatography. ¹H NMR (400 MHz, Chloroform-d) δ 7.79-7.70 (m, 2H), 7.58 (d, J=16.0 Hz, 1H), 7.28 (d, J=9.2 Hz, 1H), 6.42 (d, J=16.0 Hz, 1H), 4.28 (q, J=7.1 Hz, 2H), 1.34 (t, J=7.1 Hz, 3H). LRMS [M+H]⁺ 243.1.

Aryl-azide 8: To a solution of intermediate 21 (25 mg, 0.10 mmol, 1.0 equiv) in THF (4 mL), LiOH (40 mg, 1.0 mmol, 10 equiv) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude. The desired product was isolated using silica gel chromatography. ¹H NMR (400 MHz, Methanol-d₄) δ 7.96 (d, J=11.4 Hz, 2H), 7.63 (d, J=16.0 Hz, 1H), 7.57-7.44 (m, 1H), 6.66-6.44 (m, 1H). LRMS [M−H]⁻ 213.04.

Synthesis of aryl-azide 9

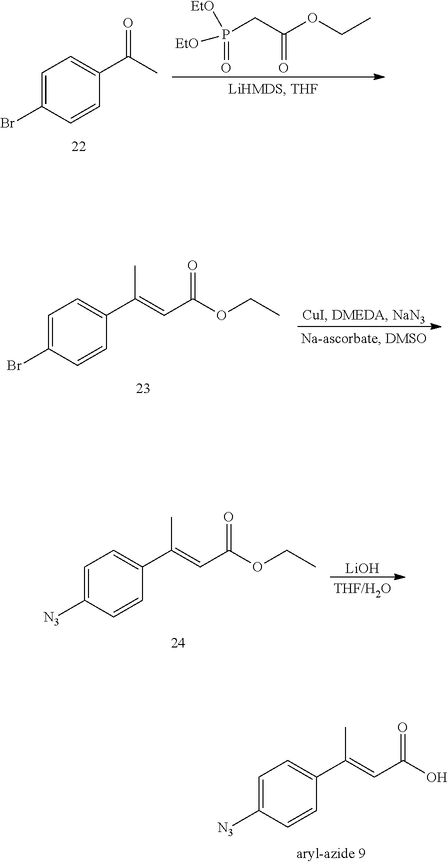

Intermediate 23: To a solution of phosphonate reagent (0.57 mL, 2.9 mmol, 1.0 equiv), LiHMDS solution (3.1 mL, 1.0 N, 3.1 mmol, 1.1 equiv) was added dropwise over 10 min. The mixture was stirred for 30 min at RT before addition of aldehyde intermediate 22 in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄ and concentrated in vacuo to afford the crude. The desired product was isolated using silica gel chromatography. ¹H NMR (400 MHz, Chloroform-d) δ 7.50 (d, J=8.3 Hz, 2H), 7.34 (d, J=8.2 Hz, 2H), 6.11 (t, J=1.3 Hz, 1H), 4.21 (q, J=7.1 Hz, 2H), 2.54 (t, J=1.1 Hz, 3H), 1.31 (t, J=7.1 Hz, 3H). LRMS [M+H]⁺ 269.02.

Intermediate 24: Intermediate 23 (110 mg, 0.41 mmol, 1.0 equiv), CuI (7.8 mg, 0.04 mmol, 0.1 equiv), and Na-ascorbate (8.0 mg, 0.04 mmol, 0.1 equiv) were charged into a vial purged with N₂. To the mixture, DMSO (5 mL) and DMEDA (6.6 μL, 0.06 mmol, 0.15 equiv) was added. The mixture was then stirred at RT for 15 min before NaN₃ (53 mg, 0.82 mmol, 2.0 equiv) was added. The reaction was heated at 100° C. for 16 h. The reaction was then stirred at RT for 16 h and quenched by addition of sat. aq. NH₄Cl (20 mL). The quenched reaction was extracted with EtOAc (50×3 mL). The combined organic layer was dried over Na₂SO₄ and concentrated in vacuo to afford the crude. The desired product was isolated using silica gel chromatography. ¹H NMR (400 MHz, Chloroform-d) δ 7.62-7.45 (m, 2H), 7.13-6.95 (m, 2H), 6.24-6.04 (m, 1H), 4.24 (q, J=7.1 Hz, 2H), 2.58 (d, J=1.3 Hz, 3H), 1.34 (t, J=7.1 Hz, 3H). LRMS [M+H]⁺ 232.11.

Aryl-azide 9: To a solution of intermediate 24 (30 mg, 0.12 mmol, 1.0 equiv) in THF (4 mL), LiOH (80 mg, 2.0 mmol, 17 equiv) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude. The desired product was isolated using silica gel chromatography. ¹H NMR (400 MHz, Acetonitrile-d₃) δ 7.64 (d, J=7.8 Hz, 2H), 7.12 (d, J=7.8 Hz, 2H), 6.17 (s, 1H), 2.54 (s, 3H). [M−H]⁻ 202.04.

### Synthesis of A-1 (E)-3-(6-azidonaphthalen-2-yl)acrylic acid

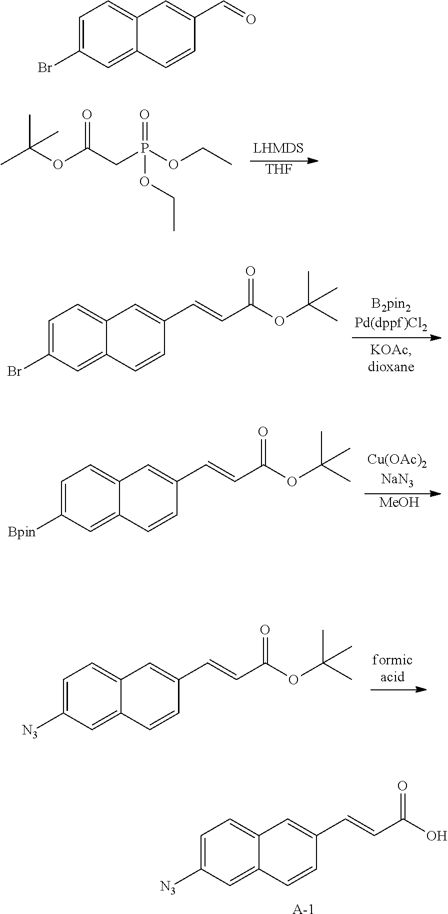

Step 1: To a 20 mL vial, the tert-butyl 2-(diethoxyphosphoryl)acetate (0.475 mL, 2.13 mmol) and THF (6 mL) was added. The mixture was stirred under nitrogen, and 1M LHMDS in THF (2.13 mL, 2.13 mmol) added dropwise over 5 min. To the mixture, 6-bromo-2-naphthaldehyde (500 mg, 2.13 mmol) was added over 1 min. After 20 min, the mixture was adsorbed to Celite and purified by silica gel chromatography with 0-30% EtOAc in heptane as eluent to afford tert-butyl (E)-3-(6-bromonaphthalen-2-yl)acrylate. LRMS [M+H—C₄H₄]⁺ 277.

Step 2: To a 20 mL vial, tert-butyl (E)-3-(6-bromonaphthalen-2-yl)acrylate (200 mg, 0.600 mmol), Pd(dppf)Cl₂ (22.0 mg, 0.030 mmol), B₂pin₂ (183 mg, 0.720 mmol), potassium acetate (118 mg, 1.20 mmol), and dioxane (4 mL) were added. The mixture was degassed with nitrogen for 1 min. The mixture was stirred and heated at 100° C. for 1 h. The mixture was cooled to RT. The mixture was diluted in EtOAc and filtered through Celite. The solvents were evaporated to afford tert-butyl (E)-3-(6-(4,4,5,5-tetramethyl-1,3,2-dioxaborolan-2-yl)naphthalen-2-yl)acrylate, which was taken forward to the next step without further purification. LRMS [M+H—C₄H₄]⁺325.

Step 3: To a 20 mL vial, tert-butyl (E)-3-(6-(4,4,5,5-tetramethyl-1,3,2-dioxaborolan-2-yl)naphthalen-2-yl)acrylate (225 mg, 0.592 mmol), NaN₃ (57.8 mg, 0.889 mmol), Cu(OAc)₂ (215 mg, 1.18 mmol), and MeOH (3 mL) was added. The mixture was vigorously at 65° C. for 90 min. The mixture was diluted with EtOAc and washed with 10% ammonia. The organic layer was dried over sodium sulfate, filtered, and the solvents were evaporated. The residue was purified by silica gel chromatography with 0-30% EtOAc in heptane as eluent to afford tert-butyl (E)-3-(6-azidonaphthalen-2-yl)acrylate. LRMS [M+H—C₄H₄]⁺240.

Step 4: To a 20 mL vial, tert-butyl (E)-3-(6-azidonaphthalen-2-yl)acrylate (100 mg, 0.339 mmol), DCM (2 mL), and formic acid (1 mL) were added. The mixture was stirred for 14 h. Solids precipitated. The solids were collected by filtration and washed with DCM to afford A-1 (E)-3-(6-azidonaphthalen-2-yl)acrylic acid. LRMS [M−H]⁻ 238.

### Synthesis of A-2 (E)-3-(6-azidonaphthalen-2-yl)but-2-enoic acid

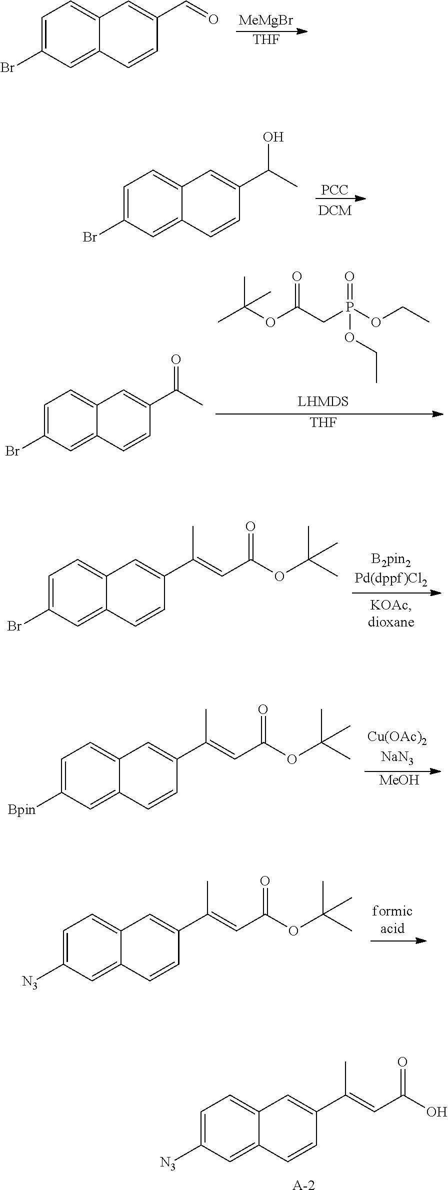

Step 1: To a 20 mL vial, 6-bromo-2-naphthaldehyde (500 mg, 2.13 mmol) and THF (10 mL) was added. The mixture was stirred under nitrogen at 0° C. To the mixture, a 1.4 M solution of MeMgBr (1.82 mL, 2.55 mmol) was added over 5 min. The mixture was stirred for 10 min. The mixture was quenched with saturated ammonium chloride (˜0.5 mL) and filtered through Celite. The organic layer was diluted in diethyl ether and washed with water. The organic layer was dried over magnesium sulfate, filtered, and the solvents were evaporated. The residue was purified by silica gel chromatography with 0-30% EtOAc in heptane as eluent to afford 1-(6-bromonaphthalen-2-yl)ethan-1-ol. LRMS [M+H—H₂O]⁺233.

Step 2: To a 100 mL flask, PCC (1.09 g, 5.07 mmol), Celite (2.5 g), and DCM (20 mL) was added. To the stirring mixture, 1-(6-bromonaphthalen-2-yl)ethan-1-ol (424 mg, 1.69 mmol) was added. The mixture was stirred at room temperature for 1 h. The mixture was filtered through Celite, washing with DCM. The solvents of the filtrate were evaporated. The residue was purified by silica gel chromatography with 0-30% EtOAc in heptane as eluent to afford 1-(6-bromonaphthalen-2-yl)ethan-1-one. LRMS [M+H]⁺ 249.

Step 3: To a 20 mL vial, the tert-butyl 2-(diethoxyphosphoryl)acetate (0.307 mL, 1.38 mmol) and THF (5 mL) was added. The mixture was stirred under nitrogen. To the mixture, 1M LHMDS in THF (1.38 mL, 1.38 mmol) was added dropwise over 5 min. To the mixture, a solution of 1-(6-bromonaphthalen-2-yl)ethan-1-one (343 mg, 1.38 mmol) in THF was added dropwise over 5 min. After 5 min, the vial was sealed then stirred and heated at 70° C. for 3 h. The mixture was adsorbed to Celite and purified by silica gel chromatography with 0-20% EtOAc in heptane as eluent to afford tert-butyl (E)-3-(6-bromonaphthalen-2-yl)but-2-enoate. LRMS [M+H—C₄H₄]⁺ 291.

Step 4: To a 20 mL vial, tert-butyl (E)-3-(6-bromonaphthalen-2-yl)but-2-enoate (350 mg, 1.01 mmol), Pd(dppf)Cl₂ (73.8 mg, 0.101 mmol), B₂pin₂ (307 mg, 1.21 mmol), potassium acetate (198 mg, 2.02 mmol), and dioxane (5 mL) were added. The mixture was degassed with nitrogen for 1 min. The mixture was stirred and heated at 120° C. for 2.5 h. The mixture was cooled to RT. The mixture was diluted in EtOAc and filtered through Celite. The solvents were evaporated to afford tert-butyl (E)-3-(6-(4,4,5,5-tetramethyl-1,3,2-dioxaborolan-2-yl)naphthalen-2-yl)but-2-enoate which was taken forward to the next step without further purification. LRMS [M+H—C₄H₄]⁺ 339.

Step 5: To a 20 mL vial, tert-butyl (E)-3-(6-(4,4,5,5-tetramethyl-1,3,2-dioxaborolan-2-yl)naphthalen-2-yl)but-2-enoate (145 mg, 0.368 mmol), NaN₃ (35.9 mg, 0.552 mmol), Cu(OAc)₂ (134 mg, 0.736 mmol), and MeOH (8 mL) was added. The mixture was vigorously at 65° C. for 90 min. The mixture was diluted with EtOAc and washed with 10% ammonia. The organic layer was dried over sodium sulfate, filtered, and the solvents were evaporated. The residue was purified by silica gel chromatography with 0-50% EtOAc in heptane as eluent to afford tert-butyl (E)-3-(6-azidonaphthalen-2-yl)but-2-enoate. LRMS [M+H—C₄H₄]⁺ 254.

Step 6: To a 20 mL vial, tert-butyl (E)-3-(6-azidonaphthalen-2-yl)but-2-enoate (30.0 mg, 0.0970 mmol), DCM (2 mL), and formic acid (1 mL) was added. The mixture was stirred for 14 h. The solvents were evaporated to afford A-2 (E)-3-(6-azidonaphthalen-2-yl)but-2-enoic acid. LRMS [M−H]⁻ 252.

### Synthesis of A-3 (E)-3-(7-azidoquinolin-3-yl)acrylic acid

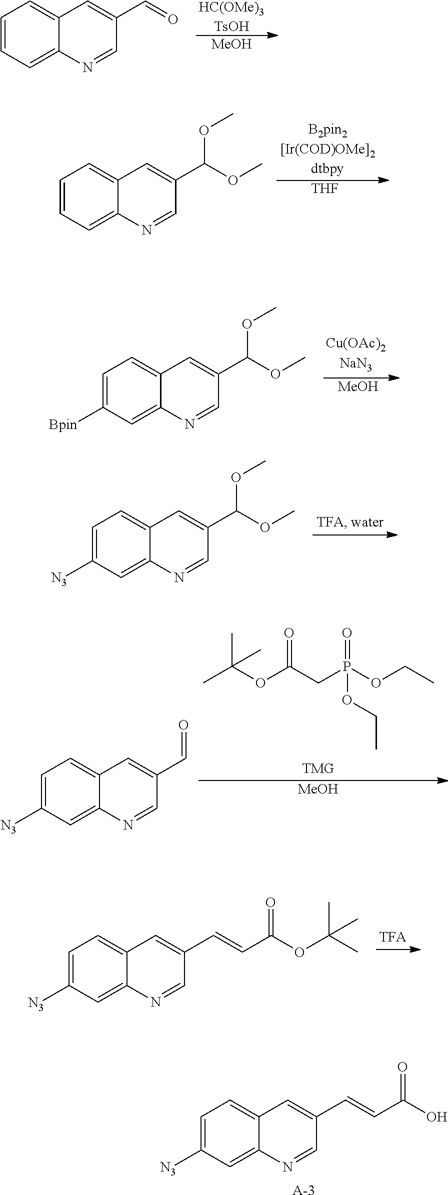

Step 1: To a 100 mL round bottom flask, quinoline-3-carbaldehyde (2.00 g, 12.7 mmol), p-toluenesulfonic acid monohydrate (219 mg, 1.27 mmol), MeOH (15 mL), and the trtimethylorthoformate (13.9 mL, 127 mmol) was added. The mixture was stirred and heated at 70° C. for 3 hours. The mixture was concentrated and purified by silica gel chromatography with 0-70% EtOAc in heptane as eluent to afford 3-(dimethoxymethyl)quinoline. LRMS [M+H]⁺ 204.

Step 2: To a 20 mL vial, 3-(dimethoxymethyl)quinoline (500 mg, 2.46 mmol), [Ir(COD)OMe]2 (81.5 mg, 0.123 mmol), B₂pin₂ (937 mg, 3.69 mmol), and 4,4′-di-tert-butylbipyridine (dtbpy, 66.0 mg, 0.246 mmol) was added. The vial was purged with nitrogen. To the mixture, dry, THF (5 mL) was added. The mixture was sparged with nitrogen for 1 min. The mixture was stirred at RT for 14 h. The solvents was evaporated to afford crude 3-(dimethoxymethyl)-7-(4,4,5,5-tetramethyl-1,3,2-dioxaborolan-2-yl)quinoline, which was taken to the next step without purification. LRMS [M+H]⁺ 330.

Step 3: To a 20 mL vial, 3-(dimethoxymethyl)-7-(4,4,5,5-tetramethyl-1,3,2-dioxaborolan-2-yl)quinoline (810 mg, 2.46 mmol), NaN₃ (240 mg, 3.69 mmol), Cu(OAc)₂ (894 mg, 4.62 mmol), and MeOH (8 mL) was added. The mixture was stirred vigorously at 55° C. for 90 min. The mixture was diluted with EtOAc and washed with 10% ammonia. The organic layer was dried over sodium sulfate, filtered, and the solvents were evaporated. The residue was purified by silica gel chromatography with 0-40% EtOAc in heptane as eluent to afford 7-azido-3-(dimethoxymethyl)quinoline. LRMS [M+H]⁺ 245.

Step 4: To a 20 mL vial, 7-azido-3-(dimethoxymethyl)quinoline (26.9 mg, 0.110 mmol), TFA (1 mL), and water (0.1 mL) was added. The mixture was stirred for 10 min. The solvents were evaporated to afford 7-azidoquinoline-3-carbaldehyde. LRMS [M+H]⁺ 199.

Step 5: To a 20 mL vial, tert-butyl 2-(diethoxyphosphoryl)acetate (0.0278 mL, 0.110 mmol), 7-azidoquinoline-3-carbaldehyde (21.8 mg, 0.110 mmol), and MeOH (1 mL) was added. To the stirring mixture, tetramethylguanidine (TMG, 0.055 mL, 0.441 mmol) was added dropwise. After 20 min, the solvents were evaporated, and the residue was purified by silica gel chromatography with 0-50% EtOAc in heptane as eluent to afford tert-butyl (E)-3-(7-azidoquinolin-3-yl)acrylate. LRMS [M+H]⁺ 297.

Step 6: To a 20 mL vial, tert-butyl (E)-3-(7-azidoquinolin-3-yl)acrylate (23.7 mg, 0.0800 mmol) and TFA (1 mL) was added. The mixture was stirred for 15 min. The solvents were evaporated to afford A-3 (E)-3-(7-azidoquinolin-3-yl)acrylic acid. LRMS [M+H]⁺ 241.

### Synthesis of A-4 (E)-3-(6-azidoquinolin-3-yl)acrylic acid

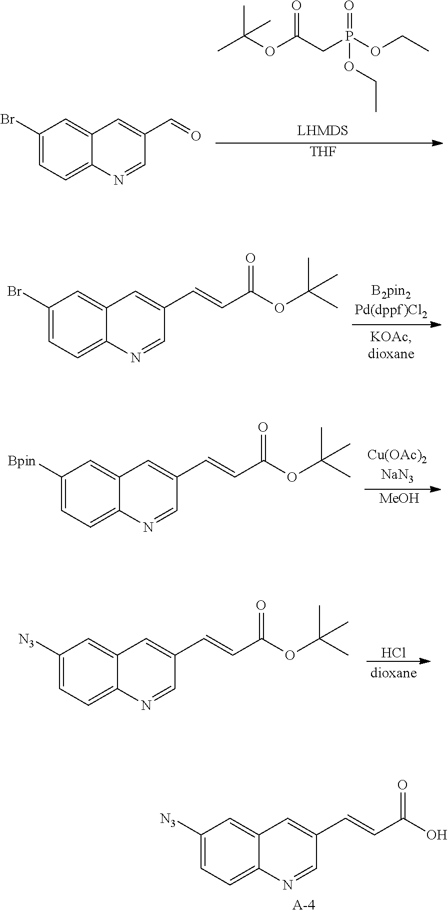

Step 1: To a 20 mL vial, the tert-butyl 2-(diethoxyphosphoryl)acetate (0.378 mL, 1.69 mmol) and THF (6 mL) was added. The mixture was stirred under nitrogen. To the mixture, 1M LHMDS in THF (1.69 mL, 1.69 mmol) was added dropwise over 5 min. To the mixture, 6-bromoquinoline-3-carbaldehyde (Cheng, Yuan et al. WO2011063233 A1) (400 mg, 1.69 mmol) was added over 1 min. After 20 min, the mixture was adsorbed to Celite and purified by silica gel chromatography with 0-50% EtOAc in heptane as eluent to afford tert-butyl (E)-3-(6-bromoquinolin-3-yl)acrylate. LRMS [M+H]⁺ 334.

Step 2: To a 20 mL vial, tert-butyl (E)-3-(6-bromoquinolin-3-yl)acrylate (64.0 mg, 0.191 mmol), Pd(dppf)Cl₂ (7.0 mg, 0.0096 mmol), B₂pin₂ (58.4 mg, 0.230 mmol), potassium acetate (37.6 mg, 0.383 mmol), and dioxane (4 mL) were added. The mixture was degassed with nitrogen for 1 min. The mixture was stirred and heated at 100° C. for 2 h. The mixture was cooled to RT. The mixture was diluted in EtOAc and filtered through Celite. The solvents were evaporated to afford tert-butyl (E)-3-(6-(4,4,5,5-tetramethyl-1,3,2-dioxaborolan-2-yl)quinolin-3-yl)acrylate, which was taken forward to the next step without further purification. LRMS [M+H]⁺ 382.

Step 3: To a 20 mL vial, tert-butyl (E)-3-(6-(4,4,5,5-tetramethyl-1,3,2-dioxaborolan-2-yl)quinolin-3-yl)acrylate (73.0 mg, 0.191 mmol), NaN₃ (18.7 mg, 0.287 mmol), Cu(OAc)₂ (69.6 mg, 0.383 mmol), and MeOH (3 mL) was added. The mixture was vigorously at 65° C. for 90 min. The mixture was diluted with EtOAc and washed with 10% ammonia. The organic layer was dried over sodium sulfate, filtered, and the solvents were evaporated. The residue was purified by silica gel chromatography with 0-50% EtOAc in heptane as eluent to afford tert-butyl (E)-3-(6-azidoquinolin-3-yl)acrylate. LRMS [M+H]⁺ 297.

Step 4: To a 20 mL vial, tert-butyl (E)-3-(6-azidoquinolin-3-yl)acrylate and 4 M HCl in dioxane (2 mL) was added. The mixture was stirred and heated at 70° C. for 2 h. The solvents were concentrated to afford A-4 (E)-3-(6-azidoquinolin-3-yl)acrylic acid. LRMS [M+H]⁺ 241.

### Synthesis of A-5 (E)-3-(6-azidoquinolin-3-yl)but-2-enoic acid

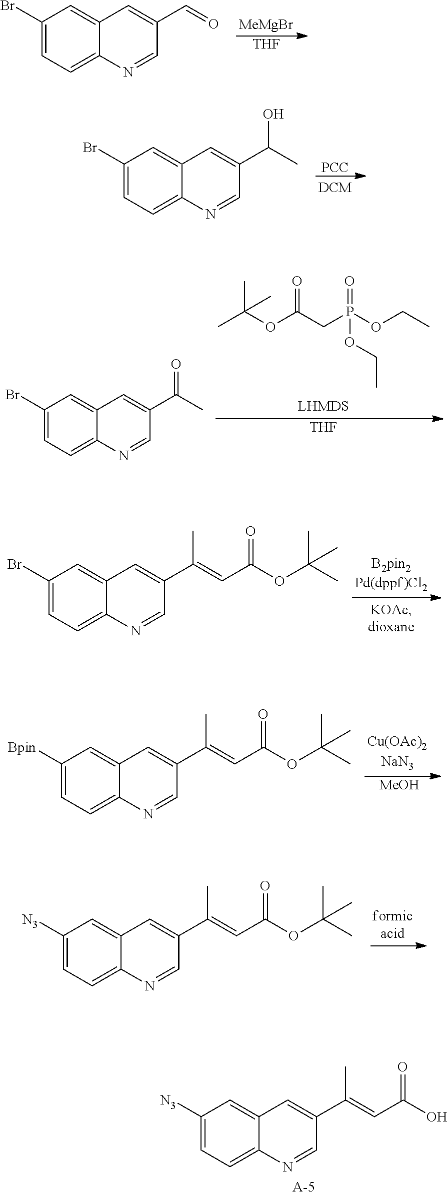

Step 1: To a 20 mL vial, 6-bromoquinoline-3-carbaldehyde (Cheng, Yuan et al. WO2011063233 A1) (334 mg, 1.42 mmol) and THF (8 mL) was added. The mixture was stirred under nitrogen at 0° C. To the mixture was added a 1.4 M solution of MeMgBr (1.52 mL, 2.12 mmol) over 5 min. The mixture was stirred for 10 min. The mixture was quenched with saturated ammonium chloride (1 mL). The mixture was diluted with EtOAc and filtered through Celite. The filtrate was concentrated, and the residue was purified by silica gel chromatography with 0-70% EtOAc in heptane as eluent to afford 1-(6-bromoquinolin-3-yl)ethan-1-ol. LRMS [M+H]⁺ 252.

Step 2: To a 20 mL vial, 1-(6-bromoquinolin-3-yl)ethan-1-ol (260 mg, 1.03 mmol), PCC (668 g, 3.10 mmol), Celite (1 g), and DCM (6 mL) was added. The mixture was stirred at room temperature for 2 h. The mixture was filtered through Celite, washing with DCM. The solvents of the filtrate were evaporated. The residue was purified by silica gel chromatography with 0-70% EtOAc in heptane as eluent to afford 1-(6-bromoquinolin-3-yl)ethan-1-one. LRMS [M+H]⁺ 250.

Step 3: To a 20 mL vial, the tert-butyl 2-(diethoxyphosphoryl)acetate (0.185 mL, 0.829 mmol) and THF (6 mL) was added. The mixture was stirred under nitrogen. To the mixture, 1 M LHMDS in THF (1.13 mL, 1.13 mmol) was added dropwise over 5 min. To the mixture, a solution of 1-(6-bromoquinolin-3-yl)ethan-1-one (188 mg, 0.753 mmol) in THF was added dropwise over 5 min. The mixture was stirred for 20 min. The mixture was adsorbed to Celite and purified by silica gel chromatography with 0-50% EtOAc in heptane as eluent to afford tert-butyl (E)-3-(6-bromoquinolin-3-yl)but-2-enoate. LRMS [M+H]⁺ 348.

Step 4: To a 20 mL vial, tert-butyl (E)-3-(6-bromoquinolin-3-yl)but-2-enoate (55.0 mg, 0.158 mmol), Pd(dppf)Cl₂ (5.8 mg, 0.0079 mmol), B₂pin₂ (48.1 mg, 0.190 mmol), potassium acetate (31.0 mg, 0.316 mmol), and dioxane (1 mL) were added. The mixture was degassed with nitrogen for 1 min. The mixture was stirred and heated at 100° C. for 2 h. The mixture was cooled to RT. The mixture was diluted in EtOAc and filtered through Celite. The solvents were evaporated to afford tert-butyl (E)-3-(6-(4,4,5,5-tetramethyl-1,3,2-dioxaborolan-2-yl)quinolin-3-yl)but-2-enoate, which was taken forward to the next step without further purification. LRMS [M+H]⁺ 396.

Step 5: To a 20 mL vial, tert-butyl (E)-3-(6-(4,4,5,5-tetramethyl-1,3,2-dioxaborolan-2-yl)quinolin-3-yl)but-2-enoate (62.4 mg, 0.158 mmol), NaN₃ (15.4 mg, 0.237 mmol), Cu(OAc)₂ (57.3 mg, 0.316 mmol), and MeOH (3 mL) was added. The mixture was vigorously at 65° C. for 90 min. The mixture was diluted with EtOAc and washed with 10% ammonia. The organic layer was dried over sodium sulfate, filtered, and the solvents were evaporated. The residue was purified by silica gel chromatography with 0-50% EtOAc in heptane as eluent to afford tert-butyl (E)-3-(6-azidoquinolin-3-yl)but-2-enoate. LRMS [M+H]⁺ 311.

Step 6: To a 20 mL vial, tert-butyl (E)-3-(6-azidoquinolin-3-yl)but-2-enoate (43.5 mg, 0.140 mmol) and 4 M HCl in dioxane (2 mL) was added. The mixture was stirred and heated at 70° C. for 2 h. The solvents were evaporated to afford A-5 (E)-3-(6-azidoquinolin-3-yl)but-2-enoic acid. LRMS [M+H]⁺ 255.

### Synthesis of A-6 (E)-3-(6-azido-7-cyanonaphthalen-2-yl)acrylic acid

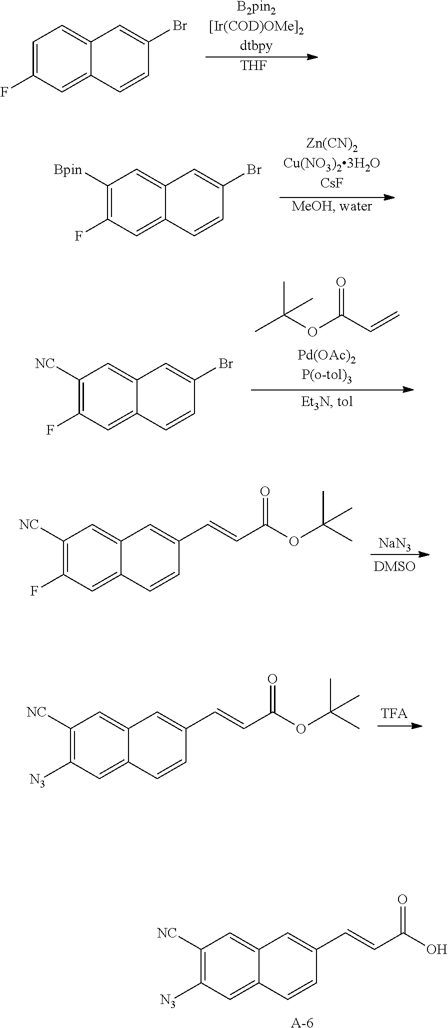

Step 1: To a 100 mL flask, 2-bromo-6-fluoronaphthalene (1.00 g, 4.44 mmol), [Ir(COD)OMe]2 (147 mg, 0.222 mmol), B₂pin₂ (1.69 g, 6.66 mmol), and 4,4′-di-tert-butylbipyridine (dtbpy, 119 mg, 0.444 mmol) was added. The vial was purged with nitrogen. To the mixture, dry THF (10 mL) was added. The mixture was sparged with nitrogen for 1 min. The mixture was stirred at RT for 14 h. The solvents were evaporated. The residue was purified by silica gel chromatography to afford 2-(7-bromo-3-fluoronaphthalen-2-yl)-4,4,5,5-tetramethyl-1,3,2-dioxaborolane. ¹H NMR (400 MHz, DMSO-d₆) δ 8.54 (d, J=2.2 Hz, 1H), 8.38 (dd, J=4.3, 2.2 Hz, 2H), 8.33 (d, J=6.0 Hz, 1H), 8.21 (d, J=11.6 Hz, 1H), 8.03 (d, J=2.2 Hz, 1H), 7.89 (d, J=8.8 Hz, 1H), 7.76-7.65 (m, 2H), 1.39 (s, 12H).

Step 2: To a 100 mL flask, 2-(7-bromo-3-fluoronaphthalen-2-yl)-4,4,5,5-tetramethyl-1,3,2-dioxaborolane (1.18 g, 3.35 mmol), Cu(NO₃)₂·3H₂O (1.62 g, 6.70 mmol), Zn(CN)₂ (1.18 g, 10.1 mmol), CsF (509 mg, 3.35 mmol), MeOH (20 mL), and water (8 mL) were added. The reaction was stirred and heated at reflux for 1 hr. The mixture was cooled to RT. The mixture was diluted in EtOAc and washed with 1M aqueous ammonia. The organic layer was dried over sodium sulfate, filtered, and the solvents of the filtrate were evaporated. The residue was purified by silica gel chromatography with 0-20% EtOAc in heptane to afford 7-bromo-3-fluoro-2-naphthonitrile. ¹H NMR (400 MHz, DMSO-d₆) δ 8.71 (d, J=6.6 Hz, 1H), 8.39 (d, J=1.9 Hz, 1H), 8.11 (d, J=10.6 Hz, 1H), 8.02 (d, J=8.9 Hz, 1H), 7.91 (dd, J=8.8, 2.0 Hz, 1H).

Step 3: To a 20 mL vial, 7-bromo-3-fluoro-2-naphthonitrile (199 mg, 0.797 mmol), t-butyl acrylate (0.146 mL, 0.996 mmol), triethylamine (0.222 mL, 1.59 mmol), Pd(OAc)₂ (1.8 mg, 0.0080 mmol), tri-(o-tolyl)phosphine (9.7 mg, 0.032 mmol), and toluene (8 mL) was added. The mixture was purged with nitrogen for 1 min. The mixture was stirred and heated at 100° C. under nitrogen for 3 h. The solvents were evaporated, and the residue was purified by silica gel chromatography with 0-20% EtOAc in heptane to afford tert-butyl (E)-3-(7-cyano-6-fluoronaphthalen-2-yl)acrylate. LRMS [M+H+MeCN]⁺ 339.

Step 4: To a 20 mL vial, the tert-butyl (E)-3-(7-cyano-6-fluoronaphthalen-2-yl)acrylate (64.7 mg, 0.218 mmol), NaN₃ (15.5 mg, 0.239 mmol), and DMSO (1 mL) was added. The mixture was stirred and heated at 100° C. for 2 h. The mixture was diluted in 1:2 EtOAc/Et₂O (12 mL) and filtered through Celite, rinsing with Et₂O. The filtrate was washed with water (3×10 mL). The organic layer was dried over sodium sulfate, filtered, and the solvents of the filtrate were evaporated. The mixture was purified by silica gel chromatography with 0-30% EtOAc in heptane as eluent to afford the tert-butyl (E)-3-(6-azido-7-cyanonaphthalen-2-yl)acrylate. LRMS [M+H—N₂]⁺ 293.

Step 5: To a 20 mL vial, tert-butyl (E)-3-(6-azido-7-cyanonaphthalen-2-yl)acrylate (37.4 mg, 0.117 mmol) and TFA (1 mL) was added. The mixture was stirred for 15 min. The solvents were evaporated to afford A-6 (E)-3-(6-azido-7-cyanonaphthalen-2-yl)acrylic acid. LRMS [M−H]⁻ 263.

### Synthesis of A-7 (E)-3-(6-azido-7-cyanonaphthalen-2-yl)but-2-enoic acid

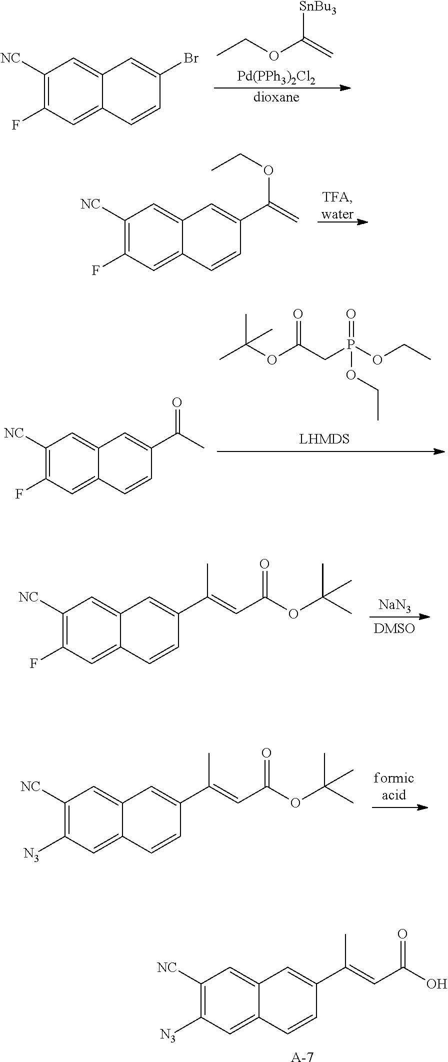

Step 1: To a solution of 7-bromo-3-fluoro-2-naphthonitrile (from Step 2 of A-6) (266 mg, 1.07 mmol) in dioxane (2 mL), tributyl(1-ethoxyvinyl)tin (0.396 mL, 1.17 mmol), and Pd(PPh₃)₂Cl₂ (37.4 mg, 0.0533 mmol) was added. The mixture was purged with nitrogen for 2 min. The mixture was stirred and heated at 130° C. for 30 min. The mixture was cooled to RT, and the mixture was diluted in EtOAc and filtered through Celite. The solvents of the filtrate were evaporated. The residue was purified by silica gel chromatography with 0-30% EtOAc in heptane as eluent to afford 7-(1-ethoxyvinyl)-3-fluoro-2-naphthonitrile. LRMS [M+H]⁺ 242.

Step 2: To a 20 mL vial, 7-(1-ethoxyvinyl)-3-fluoro-2-naphthonitrile (154 mg, 0.637 mmol) and 10% v/v water in TFA (2 mL) was added and stirred for 10 mins. The solvents were evaporated to afford 7-acetyl-3-fluoro-2-naphthonitrile.

Step 3: To a 20 mL vial, the tert-butyl 2-(diethoxyphosphoryl)acetate (0.152 mL, 0.679 mmol) and THF (3 mL) was added. The mixture was stirred under nitrogen. To the mixture, 1M LHMDS in THF (0.679 mL, 0.679 mmol) was added dropwise over 5 min. To the mixture, a solution of 7-acetyl-3-fluoro-2-naphthonitrile (145 mg, 0.679 mmol) in THF was added dropwise over 5 min. After 5 min, the vial was sealed then stirred and heated at 70° C. for 14 h. The mixture was adsorbed to Celite and purified by silica gel chromatography with 0-30% EtOAc in heptane as eluent to afford tert-butyl (E)-3-(7-cyano-6-fluoronaphthalen-2-yl)but-2-enoate. LRMS [M+H+MeCN]⁺ 353.

Step 4: To a 20 mL vial, the tert-butyl (E)-3-(7-cyano-6-fluoronaphthalen-2-yl)but-2-enoate (40.5 mg, 0.130 mmol), NaN₃ (9.3 mg, 0.14 mmol), and DMSO (1 mL) was added. The mixture was stirred and heated at 100° C. for 2 h. The mixture was diluted in 1:2 EtOAc/Et₂O (12 mL) and filtered through Celite, rinsing with Et₂O. The filtrate was washed with water (3×10 mL). The organic layer was dried over sodium sulfate, filtered, and the solvents of the filtrate were evaporated. The mixture was purified by silica gel chromatography with 0-30% EtOAc in heptane as eluent to afford the tert-butyl (E)-3-(6-azido-7-cyanonaphthalen-2-yl)but-2-enoate. LRMS [M+H—N₂]⁺ 307.

Step 5: To a 20 mL vial, tert-butyl (E)-3-(6-azido-7-cyanonaphthalen-2-yl)but-2-enoate (13.5 mg, 0.0404 mmol) and formic acid (1 mL) was added. The mixture was stirred and heated at 40° C. for 15 min. The solvents were evaporated to afford A-7 (E)-3-(6-azido-7-cyanonaphthalen-2-yl)but-2-enoic acid. LRMS [M−H]⁻ 277.

### Synthesis of A-8:

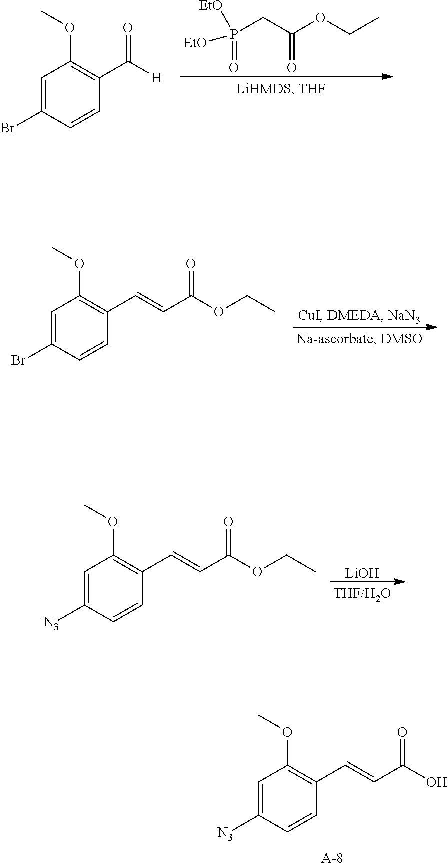

Step 1: To a solution of ethyl 2-(diethoxyphosphoryl)acetate (0.60 mL, 3.0 mmol), 1M LHMDS solution in THF (3.3 mL, 3.3 mmol) was added dropwise over 10 min. The mixture was stirred for 30 min at RT before addition of 4-bromo-2-methoxybenzaldehyde (645 mg, 3.00 mmol) in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄, concentrated in vacuo to afford the crude. Ethyl (E)-3-(4-bromo-3-methoxyphenyl)acrylate was isolated using silica gel chromatography. LRMS [M+H]⁺ 285.

Step 2: Ethyl (E)-3-(4-bromo-3-methoxyphenyl)acrylate (285 mg, 1.00 mmol), CuI (9.5 mg, 0.050 mmol), and Na-ascorbate (20 mg, 0.10 mmol) were charged into a vial purged with N₂. To the mixture, DMSO (5 mL) and DMEDA (17 μL, 0.15 mmol) were added. The mixture was then stirred at RT for 15 min before NaN₃ (98 mg, 1.5 mmol) was added. The reaction was heated at 100° C. for 16 h. The reaction was then stirred at RT for 16 h and quenched by addition of sat. aq. NH₄Cl (20 mL). The quenched reaction was extracted with EtOAc (50×3 mL). The combined organic layer was dried over Na₂SO₄ and concentrated in vacuo to afford the crude. Ethyl (E)-3-(4-azido-2-methoxyphenyl)acrylate was isolated using silica gel chromatography. LRMS [M+H]⁺ 248.

Step 3: To a solution of ethyl (E)-3-(4-azido-2-methoxyphenyl)acrylate (170 mg, 0.690 mmol) in THF (4 mL), LiOH (32.0 mg, 1.38 mmol) pre-dissolved in H₂O (2 mL) was added. The reaction mixture was stirred at RT for 3 h. The reaction was concentrated in vacuo and diluted with H₂O (20 mL). The aqueous suspension was adjusted to pH 4 and extracted with EtOAc (20×3 mL). The combined organic layer was washed with H₂O (30 mL) and brine (30 mL), dried over Na₂SO₄, and concentrated in vacuo to afford the crude. A-8 was isolated using silica gel chromatography. LRMS [M−H]⁻ 218.

### Synthesis of A-9

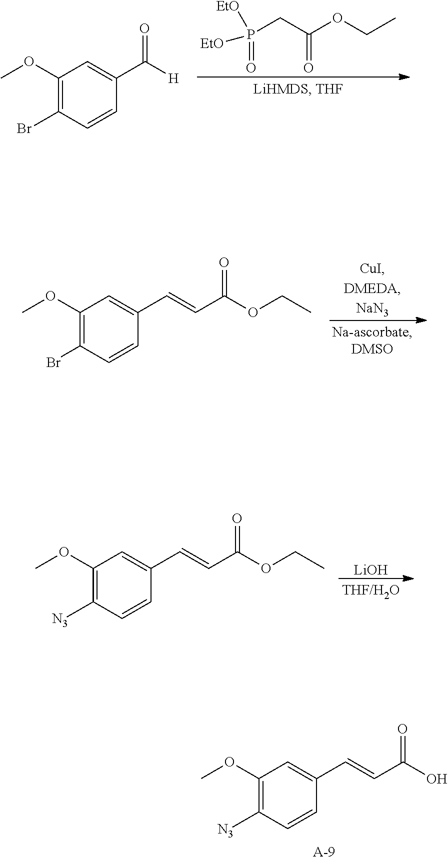

In analogy to the synthesis of A-8, 4-bromo-3-methoxybenzaldehyde was converted in three steps to A-9. LRMS [M−H]⁻ 218.

### Synthesis of A-10

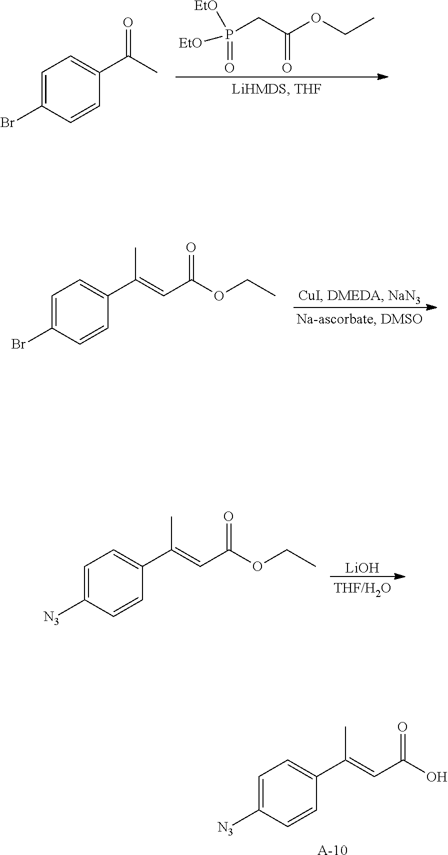

In analogy to the synthesis of A-8, 1-(4-bromophenyl)ethan-1-one was converted in three steps to A-10. LRMS [M−H]⁻ 202.

### Synthesis of A-11

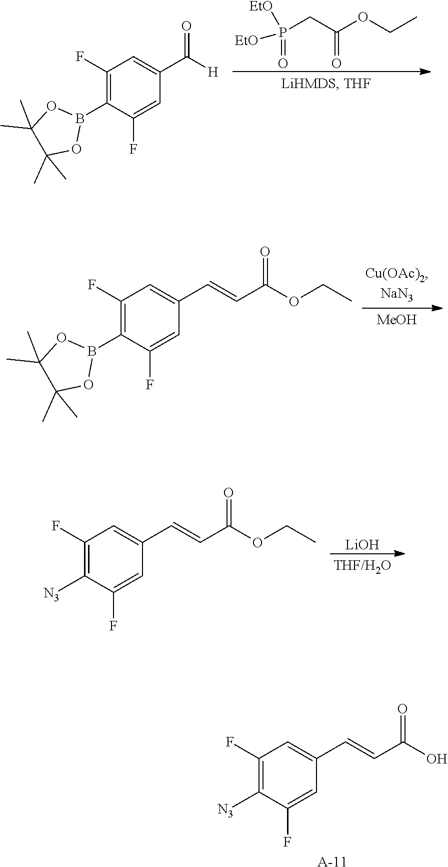

Step 1: To a solution of ethyl 2-(diethoxyphosphoryl)acetate (0.32 mL, 1.7 mmol), 1M LHMDS solution in THF (1.7 mL, 1.7 mmol) was added dropwise over 10 min. The mixture was stirred for 30 min at RT before addition of 3,5-difluoro-4-(4,4,5,5-tetramethyl-1,3,2-dioxaborolan-2-yl)benzaldehyde (402 mg, 1.50 mmol) in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄, concentrated in vacuo to afford the crude. Ethyl (E)-3-(3,5-difluoro-4-(4,4,5,5-tetramethyl-1,3,2-dioxaborolan-2-yl)phenyl)acrylate was isolated using silica gel chromatography. LRMS [M+H]⁺ 339.

Step 2: To a solution of ethyl (E)-3-(3,5-difluoro-4-(4,4,5,5-tetramethyl-1,3,2-dioxaborolan-2-yl)phenyl)acrylate (73 mg, 0.22 mmol) in MeOH (4 mL), Cu(OAc)₂ (4.0 mg, 0.022 mmol) and NaN₃ (14 mg, 0.22 mmol) was added. The reaction was heated at 60° C. for 30 min. The reaction was diluted with EtOAc (50 mL) then quenched by addition of sat. aq. NH₄Cl (10 mL). The aqueous layer was extracted with EtOAc (10×3 mL). The combined organic layer was washed with H₂O (30 mL) and brine (30 mL), dried over Na₂SO₄, and concentrated in vacuo. The desired product was purified by silica gel chromatography to afford ethyl (E)-3-(4-azido-3,5-difluorophenyl)acrylate. LRMS [M+H]⁺ 254.

Step 3: To a solution of ethyl (E)-3-(4-azido-3,5-difluorophenyl)acrylate (30 mg, 0.12 mmol) in THF (4 mL), LiOH (80 mg, 2.0 mmol) pre-dissolved in H₂O (2 mL) was added. The reaction mixture was stirred at RT for 3 h. The reaction was concentrated in vacuo and diluted with H₂O (20 mL). The aqueous suspension was adjusted to pH 4 and extracted with EtOAc (3×20 mL). The combined organic layer was washed with H₂O (30 mL) and brine (30 mL), dried over Na₂SO₄, and concentrated in vacuo to afford the crude. A-11 was isolated using silica gel chromatography. LRMS [M−H]⁻ 224.

### Synthesis of A-12

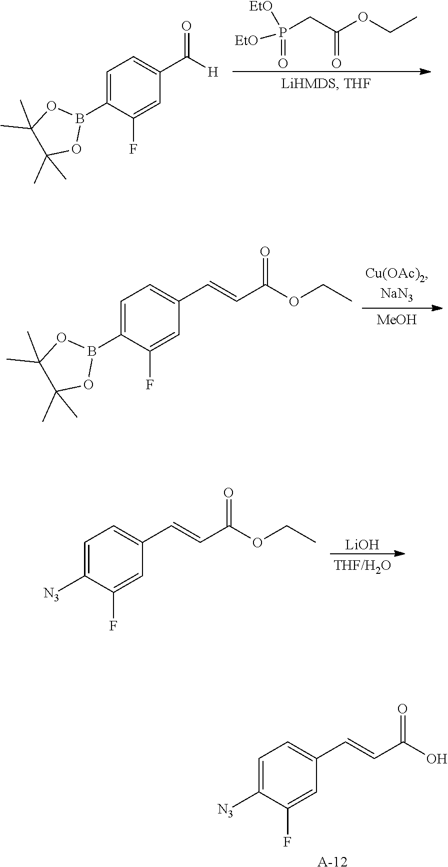

In analogy to the synthesis of A-11, 3-fluoro-4-(4,4,5,5-tetramethyl-1,3,2-dioxaborolan-2-yl)benzaldehyde was converted in three steps to A-12. LRMS [M−H]⁻ 206.

### Synthesis of A-13

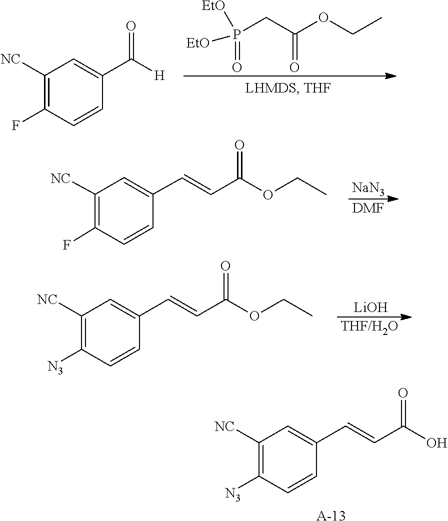

Step 1: To a solution of ethyl 2-(diethoxyphosphoryl)acetate (0.57 mL, 2.9 mmol), 1M LHMDS solution in THF (3.1 mL, 3.1 mmol) was added dropwise over 10 min. The mixture was stirred for 30 min at RT before addition of 2-fluoro-5-formylbenzonitrile (425 mg, 2.85 mmol) in one portion. The reaction was then stirred at RT for 16 h and quenched by addition of sat. aq. NH₄Cl (20 mL). The quenched reaction was extracted with EtOAc (30×3 mL). The combined organic layer was dried over Na₂SO₄ and concentrated in vacuo to afford the crude. Ethyl (E)-3-(3-cyano-4-fluorophenyl)acrylate was isolated using silica gel chromatography. LRMS [M+H]⁺ 220.

Step 2: To a solution of Ethyl (E)-3-(3-cyano-4-fluorophenyl)acrylate (149 mg, 0.68 mmol) in DMF (3 mL), NaN₃ (66 mg, 1.0 mmol) was added in one portion. The mixture was heated at 70° C. overnight. The reaction was cooled down, diluted with EtOAc (50 mL), and poured onto crushed ice. After partitioning in a separatory funnel, the aqueous layer was extracted with EtOAc (3×20 mL). The combined organic layer was washed with H₂O (50 mL) and brine (50 mL), dried over Na₂SO₄, and concentrated in vacuo. Ethyl (E)-3-(4-azido-3-cyanophenyl)acrylate was isolated using silica gel chromatography. LRMS [M+H]⁺ 243.

Step 3: To a solution of ethyl (E)-3-(4-azido-3-cyanophenyl)acrylate (25 mg, 0.10 mmol) in THF (4 mL), LiOH (40 mg, 1.0 mmol) pre-dissolved in H₂O (2 mL) was added. The reaction mixture was stirred at RT for 3 h. The reaction was concentrated in vacuo and diluted with H₂O (20 mL). The aqueous suspension was adjusted to pH 4 and extracted with EtOAc (3×20 mL). The combined organic layer was washed with H₂O (30 mL) and brine (30 mL), dried over Na₂SO₄, and concentrated in vacuo to afford the crude. A-13 was isolated using silica gel chromatography. LRMS [M−H]⁻ 213.

### Synthesis of A-14

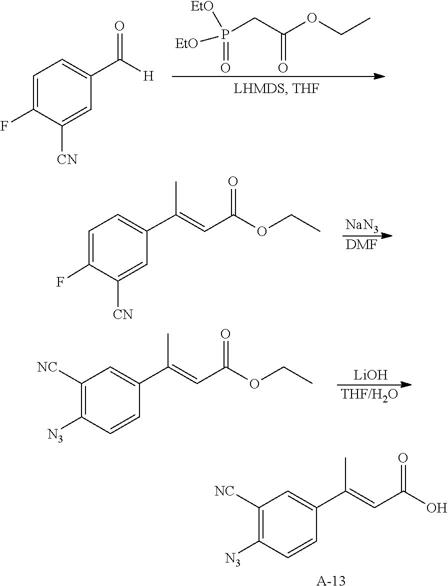

In analogy to the synthesis of A-13, 5-acetyl-2-fluorobenzonitrile was converted in three steps to A-14. LRMS [M−H]⁻ 227.

### Synthesis of Biotin Compounds

### Synthesis of 9069

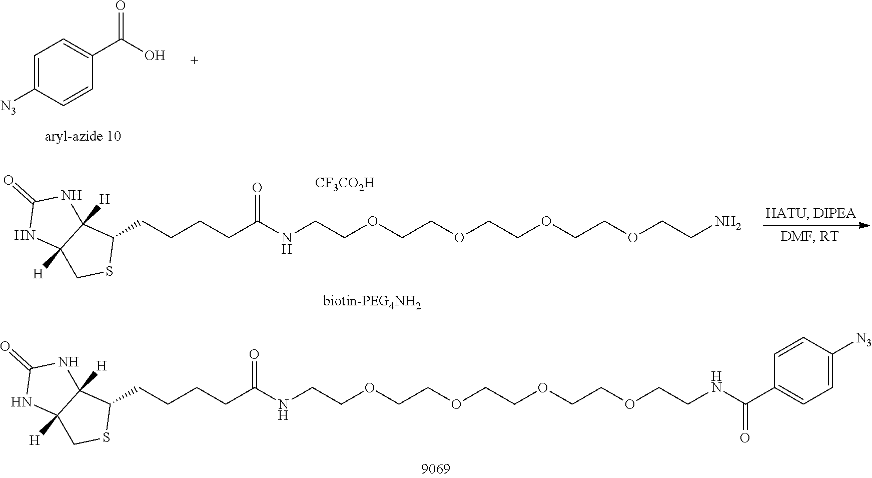 [SMILES: [N-]=[N+]=Nc1ccc(C(=O)O)cc1 ; NCCOCCOCCOCCOCCNC(=O)CCCC[C@@H]1SC[C@@H]2NC(=O)N[C@@H]21 ; "CF3CO2H" ; [N-]=[N+]=Nc1ccc(C(=O)NCCOCCOCCOCCOCCNC(=O)CCCC[C@@H]2SC[C@@H]3NC(=O)N[C@@H]32)cc1]

4-Azidobenzoic acid (aryl-azide 10, 7.8 mg, 0.048 mmol, 1.0 equiv), biotin-PEG₄-NH₂ (45 mg, 0.097 mmol, 2.0 equiv), HATU (37 mg, 0.097 mmol, 2.0 equiv), and DIPEA (84 μL, 0.48 mmol, 10 equiv) were dissolved in DMF (2 mL), and the solution was stirred at RT overnight. The reaction was quenched by addition of ACN/0.1% TFA in H₂O (1/1, 3.0 mL). The desired product was purified by reverse phase HPLC using ACN and 0.1% TFA in H₂O as mobile phases.

### Synthesis of 9042

The desired product was synthesized analogously following procedures for 9069 using aryl-azide 1. ¹H NMR (400 MHz, Chloroform-d) δ 8.30-8.04 (m, 2H), 6.78 (d, J=8.4 Hz, 1H), 6.24 (br s, 1H), 5.64 (br s, 1H), 4.63-4.49 (m, 1H), 4.44-4.26 (m, 1H), 3.96 (s, 3H), 3.75-3.39 (m, 20H), 3.13 (d, J=21.2 Hz, 2H), 2.96-2.87 (m, 1H), 2.74 (d, J=12.9 Hz, 1H), 2.23 (t, J=7.1 Hz, 2H), 1.57-1.37 (m, 6H). LRMS [M+H]⁺ 638.31.

### Synthesis of 9043

The desired product was synthesized analogously following procedures for 9069 using aryl-azide 2. ¹H NMR (400 MHz, Methanol-d₄) δ 8.40 (s, 1H), 8.03 (d, J=8.8 Hz, 1H), 7.96-7.86 (m, 2H), 7.62 (s, 1H), 7.30 (d, J=8.9 Hz, 1H), 4.56-4.40 (m, 1H), 4.32-4.20 (m, 1H), 3.77-3.66 (m, 20H), 3.22-3.07 (m, 1H), 2.92 (dd, J=13.1, 4.8 Hz, 1H), 2.75-2.60 (m, 2H), 2.20 (t, J=7.4 Hz, 2H), 1.80-1.45 (m, 6H). LRMS [M+H]⁺ 658.32.

### Synthesis of 9046

The desired product was synthesized analogously following procedures for 9069 using commercially available 4-azido-2-hydroxybenzoic acid. ¹H NMR (400 MHz, Methanol-d₄) δ 7.80 (dd, J=8.3, 2.1 Hz, 1H), 6.63-6.53 (m, 2H), 4.56-4.40 (m, 1H), 4.32-4.20 (m, 1H), 3.70-3.47 (m, 20H), 3.22-3.07 (m, 1H), 2.91 (dd, J=12.8, 4.8 Hz, 1H), 2.69 (d, J=12.7 Hz, 1H), 2.20 (t, J=7.3 Hz, 3H), 1.80-1.50 (m, 4H), 1.46-1.38 (m, 2H). LRMS [M+H]⁺ 624.72.

### Synthesis of 9044

The desired product was synthesized analogously following procedures for 9069 using commercially available (E)-3-(4-azidophenyl)acrylic acid. ¹H NMR (400 MHz, Methanol-d₄) δ 7.61 (d, J=8.1 Hz, 2H), 7.52 (d, J=15.7 Hz, 1H), 7.12 (d, J=8.1 Hz, 2H), 6.62 (d, J=15.5 Hz, 1H), 4.56-4.40 (m, 1H), 4.32-4.20 (m, 1H), 3.74-3.46 (m, 20H), 3.22-3.07 (m, 1H), 2.93 (dd, J=12.7, 5.0 Hz, 1H), 2.71 (d, J=12.7 Hz, 1H), 2.22 (t, J=7.4 Hz, 2H), 1.88-1.57 (m, 4H), 1.53-1.38 (m, 2H). LRMS [M+H]⁺ 634.28.

### Synthesis of 9047

The desired product was synthesized analogously following procedures for 9069 using aryl-azide 3. ¹H NMR (400 MHz, Chloroform-d) δ 7.80 (d, J=7.9 Hz, 2H), 7.35 (br s, 1H), 7.33 (d, J=7.9 Hz, 2H), 6.73 (d, J=14.1 Hz, 1H), 6.27 (d, J=13.8 Hz, 1H), 5.97 (s, 1H), 5.01 (s, 1H), 4.63-4.49 (m, 1H), 4.44-4.26 (m, 1H), 3.71-3.38 (m, 20H), 3.16-3.09 (m, 1H), 2.91 (dd, J=13.3, 4.9 Hz, 1H), 2.71 (d, J=12.9 Hz, 1H), 2.20 (t, J=7.1 Hz, 2H), 1.80-1.45 (m, 6H). LRMS [M+H]⁺ 634.28.

### Synthesis of 9157

The desired product was synthesized analogously following procedures for 9069 using aryl-azide 5. ¹H NMR (400 MHz, Methanol-d₄) δ 7.50 (d, J=15.7 Hz, 1H), 7.27-7.11 (m, 2H), 7.05-6.92 (m, 1H), 6.63 (d, J=15.8 Hz, 1H), 4.56-4.40 (m, 1H), 4.32-4.20 (m, 1H), 3.94 (s, 3H), 3.74-3.46 (m, 20H), 3.22-3.07 (m, 1H), 3.02-2.86 (m, 1H), 2.71 (d, J=12.8 Hz, 1H), 2.22 (t, J=7.7 Hz, 2H), 1.88-1.57 (m, 4H), 1.53-1.38 (m, 2H). LRMS [M+H]⁺ 664.28.

### Synthesis of 9158

The desired product was synthesized analogously following procedures for 9069 using aryl-azide 4. ¹H NMR (400 MHz, Methanol-d₄) δ 7.76 (d, J=15.9 Hz, 1H), 7.56 (d, J=8.2 Hz, 1H), 6.80-6.58 (m, 3H), 4.52-4.40 (m, 1H), 4.32-4.22 (m, 1H), 3.90 (s, 3H), 3.74-3.46 (m, 20H), 3.22-3.09 (m, 1H), 2.91 (dd, J=12.8, 5.0 Hz, 1H), 2.69 (d, J=12.7 Hz, 1H), 2.20 (t, J=7.4 Hz, 2H), 1.88-1.57 (m, 4H), 1.53-1.38 (m, 2H). LRMS [M+H]⁺ 664.30.

### Synthesis of 9159

The desired product was synthesized analogously following procedures for 9069 using aryl-azide 8. ¹H NMR (400 MHz, Methanol-d₄) δ 8.00-7.81 (m, 2H), 7.57-7.44 (m, 2H), 6.77-6.63 (m, 1H), 4.52-4.40 (m, 1H), 4.32-4.22 (m, 1H), 3.74-3.46 (m, 20H), 3.24-3.16 (m, 1H), 2.94 (dd, J=12.8, 5.0 Hz, 1H), 2.72 (d, J=12.8 Hz, 1H), 2.22 (t, J=7.5 Hz, 2H), 1.88-1.57 (m, 4H), 1.53-1.38 (m, 2H). LRMS [M+H]⁺ 659.28.

### Synthesis of 9160

The desired product was synthesized analogously following procedures for 9069 using aryl-azide 7. ¹H NMR (400 MHz, Methanol-d₄) δ 7.52-7.33 (m, 3H), 7.19 (t, J=8.5 Hz, 1H), 6.61 (d, J=15.9 Hz, 1H), 4.52-4.40 (m, 1H), 4.32-4.22 (m, 1H), 3.74-3.56 (m, 16H), 3.53-3.43 (m, 4H), 3.22-3.07 (m, 1H), 2.91 (d, J=12.7 Hz, 1H), 2.69 (d, J=13.0 Hz, 1H), 2.20 (t, J=7.5 Hz, 2H), 1.88-1.57 (m, 4H), 1.53-1.38 (m, 2H). LRMS [M+H]⁺ 652.31.

### Synthesis of 9161

The desired product was synthesized analogously following procedures for 9069 using aryl-azide 6. ¹H NMR (400 MHz, Methanol-d₄) δ 7.41 (d, J=15.7 Hz, 1H), 7.28 (d, J=9.3 Hz, 2H), 6.62 (d, J=15.7 Hz, 1H), 4.55-4.40 (m, 1H), 4.32-4.22 (m, 1H), 3.76-3.57 (m, 16H), 3.53-3.43 (m, 4H), 3.24-3.12 (m, 1H), 2.91 (dd, J=12.9, 4.9 Hz, 1H), 2.69 (d, J=12.8 Hz, 1H), 2.20 (t, J=7.4 Hz, 2H), 1.88-1.57 (m, 4H), 1.53-1.38 (m, 2H). LRMS [M+H]⁺ 670.28.

### Synthesis of 9162

The desired product was synthesized analogously following procedures for 9069 using aryl-azide 9. ¹H NMR (400 MHz, Methanol-d₄) δ 7.57 (dd, J=8.5 Hz, 2H), 7.17-7.01 (d, J=8.5, 2H), 6.25 (s, 1H), 4.59-4.41 (m, 1H), 4.32-4.22 (m, 1H), 3.76-3.43 (m, 20H), 3.24-3.12 (m, 1H), 2.92 (d, J=4.5 Hz, 1H), 2.71 (d, J=12.7 Hz, 1H), 2.51 (s, 3H), 2.22 (t, J=7.3 Hz, 2H), 1.88-1.57 (m, 4H), 1.53-1.38 (m, 2H). LRMS [M+H]⁺ 648.32.

### Synthesis of 9422

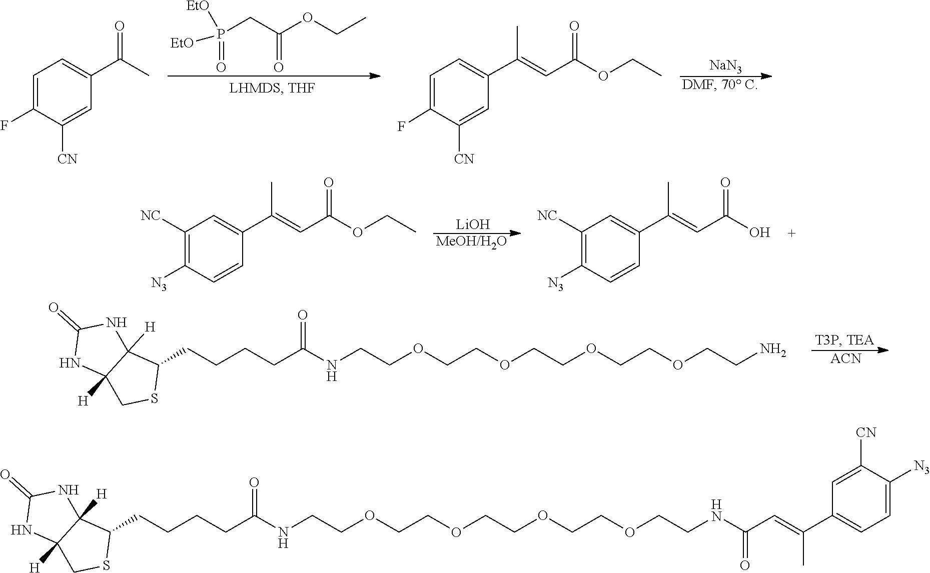

Step 1: To a solution of ethyl 2-(diethoxyphosphoryl)acetate (1.1 mL, 5.6 mmol, 1.3 equiv), LiHMDS solution (6.4 mL, 1.0 N, 6.4 mmol, 1.5 equiv) was added dropwise over 10 min. The mixture was stirred for 30 min at RT before addition of 5-acetyl-2-fluorobenzonitrile (700 mg, 4.3 mmol, 1 equiv). The reaction was then stirred at RT for overnight and quenched by addition of sat. aq. NH₄Cl (20 mL). The quenched reaction was extracted with EtOAc (30×3 mL). The combined organic layer was dried over Na₂SO₄ and concentrated in vacuo to afford the crude. The desired product, ethyl (E)-3-(3-cyano-4-fluorophenyl)but-2-enoate, was isolated using silica gel chromatography (538 mg, 54%).

Step 2: To a solution of ethyl (E)-3-(3-cyano-4-fluorophenyl)but-2-enoate (265 mg, 1.14 mmol, 1.0 equiv) in DMF (5 mL), NaN₃ (111 mg, 1.70 mmol, 1.5 equiv) was added in one portion. The mixture was heated at 70° C. overnight. The reaction was cooled down, diluted with EtOAc (50 mL), and poured onto crashed ice. The After partition, the aqueous layer, was extracted with EtOAc (30×3 mL). The combined organic layer was washed with H₂O (30 mL) and brine (30 mL), dried over Na₂SO₄, and concentrated in vacuo. The crude was concentrated and purified through silica gel column with DCM/MeOH to afford the desired product, 4 ethyl (E)-3-(4-azido-3-cyanophenyl)but-2-enoate (121 mg, 42%).

Step 3: To a solution of 4 ethyl (E)-3-(4-azido-3-cyanophenyl)but-2-enoate (50 mg, 0.20 mmol, 1.0 equiv) in MeOH (2 mL), LiOH (9.3 mg, 0.39 mmol, 2 equiv) pre-dissolved in H₂O (2 mL) was added. The reaction mixture was stirred at RT for 3 h. LC-MS indicated full conversion. The reaction was concentrated in vacuo to remove the volatile and diluted with H₂O (20 mL). The aqueous suspension was pH adjusted to 4 and extracted with EtOAc (20×3 mL). The combined organic layer was washed with H₂O (30 mL) and brine (30 mL), dried over Na₂SO₄, and concentrated in vacuo to afford the crude. The desired product was isolated using silica gel chromatography.

Step 4: The product from step 3 (50 mg. 0.22 mmol, 1 equiv) was dissolved in ACN. To this solution, biotin-PEG4-amine (111 mg, 0.24 mmol, 1.1 equiv), TEA (92 μL, 0.66 mmol, 3 equiv), and T3P (261 μL, 0.44 mmol, 2 equiv) were added, and the reaction was allowed to stir at rt overnight. The crude was concentrated and purified through silica gel column with DCM/MeOH to afford the desired product, 4-azido-N-(2-(2-(2-(2 N-((E)-18-(4-azido-3-cyanophenyl)-16-oxo-3,6,9,12-tetraoxa-15-azanonadec-17-en-1-yl)-5-((3aS,4S,6aR)-2-oxohexahydro-1H-thieno[3,4-d]imidazol-4-yl)pentanamide (112 mg, 76%). LRMS: [M+H]⁺ 673.34.

### Synthesis of Compound 9575

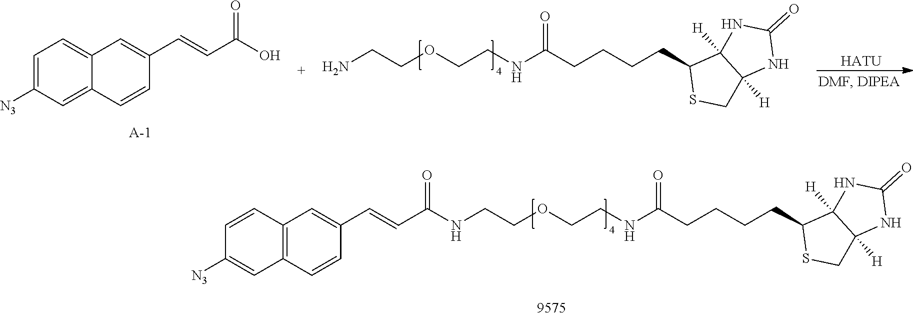

To a 20 mL vial, A-1, biotin-PEG-4-amine, DMF (1 mL), and DIPEA (0.161 mL, 0.920 mmol) was added. To the mixture, HATU (69.9 mg, 0.184 mmol) was added. The mixture was purified by RP HPLC (MeCN/water w/ 0.1% TFA) to afford compound 9575. LRMS [M+H]⁺ 684. ¹H NMR (400 MHz, DMSO-d₆) δ 8.22 (t, J=5.7 Hz, 1H), 8.07 (s, 1H), 8.00 (d, J=8.8 Hz, 1H), 7.94 (d, J=8.7 Hz, 1H), 7.83 (t, J=5.7 Hz, 1H), 7.74 (dd, J=8.7, 1.7 Hz, 1H), 7.70 (d, J=2.2 Hz, 1H), 7.57 (d, J=15.7 Hz, 1H), 7.31 (dd, J=8.7, 2.3 Hz, 1H), 6.79 (d, J=15.7 Hz, 1H), 6.42 (s, 1H), 6.36 (s, 1H), 4.30 (dd, J=7.8, 5.0 Hz, 1H), 4.12 (dd, J=7.8, 4.4 Hz, 1H), 3.56-3.45 (m, 16H), 3.40-3.35 (m, 4H), 3.12-3.03 (m, 1H), 2.81 (dd, J=12.5, 5.1 Hz, 1H), 2.57 (d, J=12.4 Hz, 1H), 2.06 (t, J=7.4 Hz, 2H), 1.67-1.40 (m, 4H), 1.37-1.22 (m, 3H).

### Synthesis of Additional Biotin Compounds

Compounds 9582, 9595, 9917, 9559, 9615, and 9616 were synthesized in a manner analogous to the synthesis of 9575 from the corresponding aryl azide intermediate (or commercially available aryl azide) and biotin-PEG4-amine. Mass spec data is provided in Table 1 shown above.

### Syntheses of Activatable Labels Comprising a Click Handle or Caged Fluorophore

### Synthesis of 9140

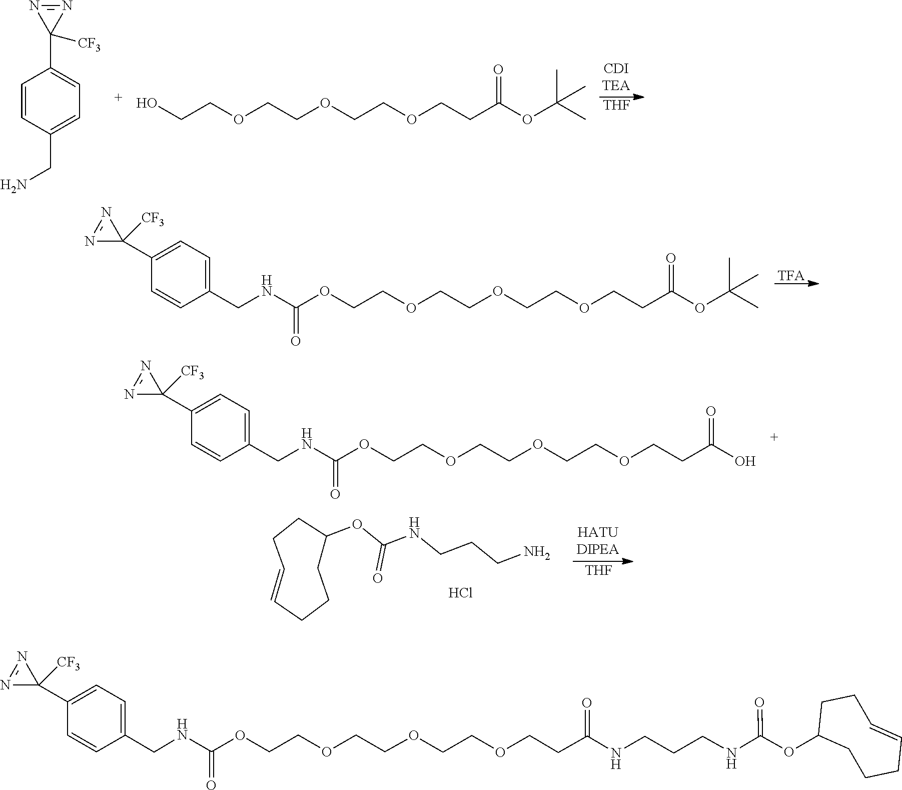

Step 1. Tert-butyl 3-(2-(2-(2-hydroxyethoxy)ethoxy)ethoxy)propanoate (200.0 mg, 0.72 mmol, 1 equiv) was dissolved in 5 mL THF at 0° C. To the solution, CDI (174.8 mg, 1.08 mmol, 1.5 equiv) was added by keeping the reaction in an ice bath. The reaction was allowed to stir for 1 h. Afterward, (4-(3-(trifluoromethyl)-3H-diazirin-3-yl)phenyl)methanamine (309.2 mg, 1.44 mmol, 2.0 equiv) and TEA (300.9 μL, 2.16 mmol, 3 equiv) was added into the reaction mixture, and the reaction was continued for another 12 h. The crude was concentrated and purified by silica gel purification using Heptane/EtOAc as eluents to afford the desired product tert-butyl 3-oxo-1-(4-(3-(trifluoromethyl)-3H-diazirin-3-yl)phenyl)-4,7,10,13-tetraoxa-2-azahexadecan-16-oate (305.0 mg, 82%).

Step 2. Tert-butyl 3-oxo-1-(4-(3-(trifluoromethyl)-3H-diazirin-3-yl)phenyl)-4,7,10,13-tetraoxa-2-azahexadecan-16-oate (220 mg) was dissolved in 2 mL DCM. To this solution, 1 ml TFA was added, and the reaction was allowed to stir at rt for an hour till LC-MS indicates no starting material left. The desired product, 3-oxo-1-(4-(3-(trifluoromethyl)-3H-diazirin-3-yl)phenyl)-4,7,10,13-tetraoxa-2-azahexadecan-16-oic acid (159 mg) was obtained without further purification.

Step 3. 3-oxo-1-(4-(3-(trifluoromethyl)-3H-diazirin-3-yl)phenyl)-4,7,10,13-tetraoxa-2-azahexadecan-16-oic acid (60.0 mg, 0.13 mmol, 1 equiv) was dissolved in 5 mL THF. To this solution, HATU (73.8 mg, 0.19 mmol, 1.5 equiv), DIPEA (67.6 μL, 0.39 mmol, 3 equiv), and (E)-cyclooct-4-en-1-yl (3-aminopropyl)carbamate hydrochloride (34.0 mg, 0.13 mmol, 1 equiv) were added, and the reaction was allowed to stir at rt for overnight. The silica gel purification using DCM/MeOH was performed to deliver final product, (E)-1-(cyclooct-4-en-1-yloxy)-1,7-dioxo-10,13,16-trioxa-2,6-diazaoctadecan-18-yl (4-(3-(trifluoromethyl)-3H-diazirin-3-yl)benzyl)carbamate (9140) (67 mg, 77%). LRMS: [M+H]⁺ 672.89.

### Synthesis of 9086

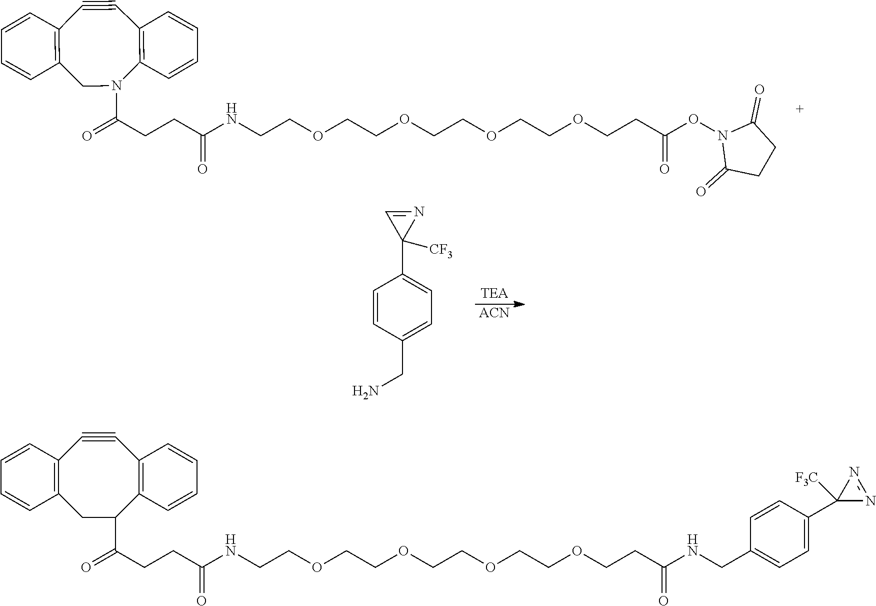

Dibenzocyclooctyne-PEG4-N-hydroxysuccinimidyl ester (100 mg, 0.15 mmol, 1 equiv) was dissolved in 5 ml ACN. To this solution, TEA (64.5 μL, 0.46 mmol, 3 equiv) and (4-(3-(trifluoromethyl)-3H-diazirin-3-yl)phenyl) methenamine hydrochloride (36.4 mg, 0.17 mmol, 1.1 equiv) were added, and the reaction was allowed to stir at rt for 1 h. The crude was concentrated and purified through silica gel column with DCM/MeOH to afford the desired product, Dibenzocyclooctyne-PEG4-phenyl diazirine (67 mg, 77%). LRMS: [M+H]⁺ 750.78.

### Synthesis of 9421

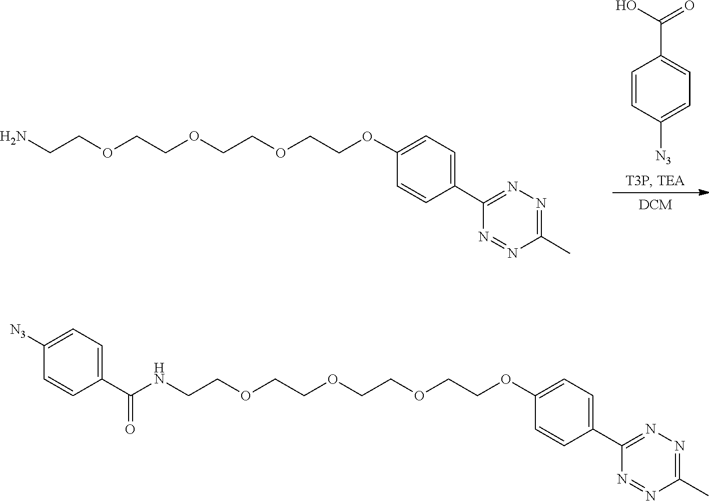 [SMILES: Cc1nnc(-c2ccc(OCCOCCOCCOCCN)cc2)nn1 ; [N-]=[N+]=Nc1ccc(C(=O)O)cc1 ; Cc1nnc(-c2ccc(OCCOCCOCCOCCNC(=O)c3ccc(N=[N+]=[N-])cc3)cc2)nn1]

2-(2-(2-(2-(4-(6-methyl-1,2,4,5-tetrazin-3-yl)phenoxy)ethoxy)ethoxy)ethoxy)ethan-1-amine (50 mg, 0.14 mmol, 1 equiv) was dissolved in 5 ml DCM. To this solution, 4-azidobenzoic acid (25 mg, 0.15 mmol, 1.1 equiv), TEA (58 μL, 0.41 mmol, 3 equiv), and T3P (123 μL, 0.21 mmol, 2 equiv) were added, and the reaction was allowed to stir at rt overnight. The crude was concentrated and purified through silica gel column with DCM/MeOH to afford the desired product, 4-azido-N-(2-(2-(2-(2-(4-(6-methyl-1,2,4,5-tetrazin-3-yl)phenoxy)ethoxy)ethoxy)ethoxy)ethyl)benzamide (56 mg, 80%). LRMS: [M+H]⁺ 509.68.

### Synthesis of 9476

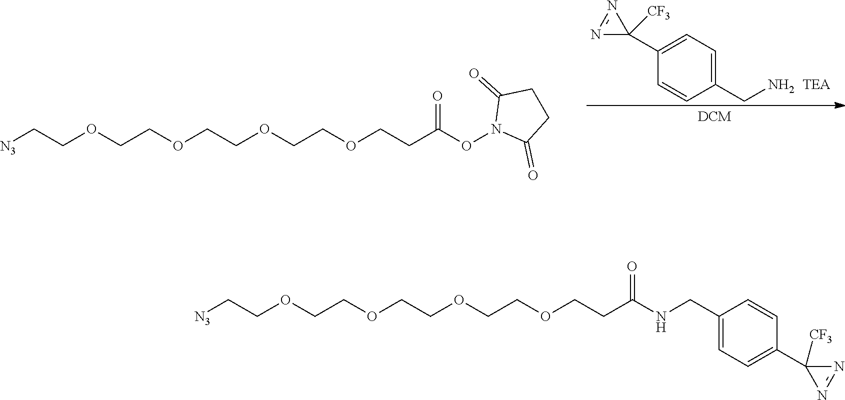 [SMILES: [N-]=[N+]=NCCOCCOCCOCCOCCC(=O)ON1C(=O)CCC1=O ; NCc1ccc(C2(C(F)(F)F)N=N2)cc1 ; [N-]=[N+]=NCCOCCOCCOCCOCCC(=O)NCc1ccc(C2(C(F)(F)F)N=N2)cc1]

To a solution of Azido-PEG4-NHS ester (80 mg, 206 μmol, 1 equiv) in DCM (5 mL), (4-(3-(trifluoromethyl)-3H-diazirin-3-yl)phenyl)methanamine (49 mg, 227 μmol, 1.1 equiv) and triethylamine (0.2 mL) were added. The mixture was stirred for 2 h at RT and concentrated in vacuo to afford the crude. The desired product, 1-azido-N-(4-(3-(trifluoromethyl)-3H-diazirin-3-yl)benzyl)-3,6,9,12-tetraoxapentadecan-15-amide, was isolated using silica gel chromatography (87 mg, 86%). LRMS: [M+Na]⁺ 511.21.

### Example 14

This example describes the syntheses of additional activatable labels and catalysts described herein.

### Synthesis of Compound 9578

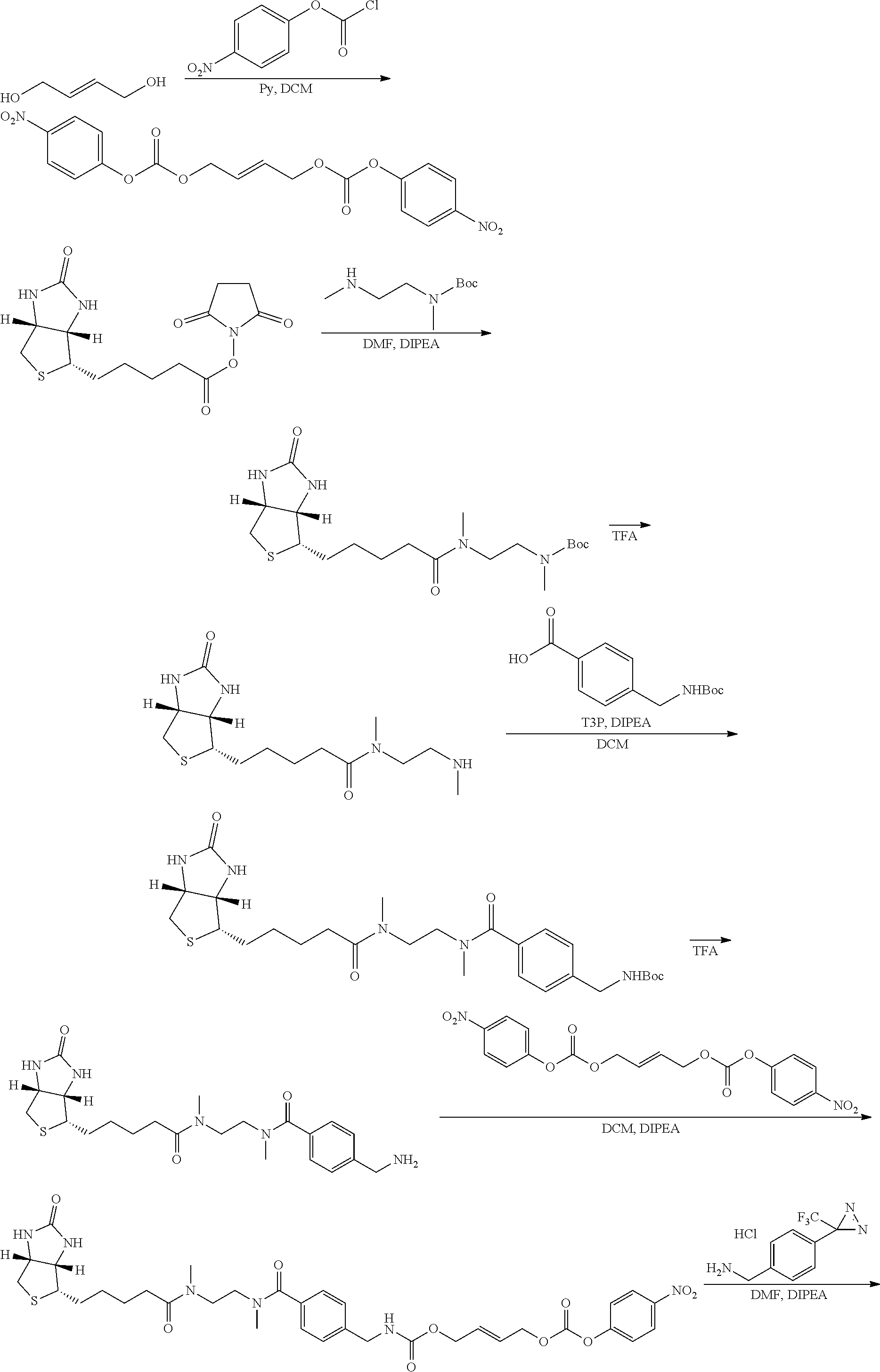

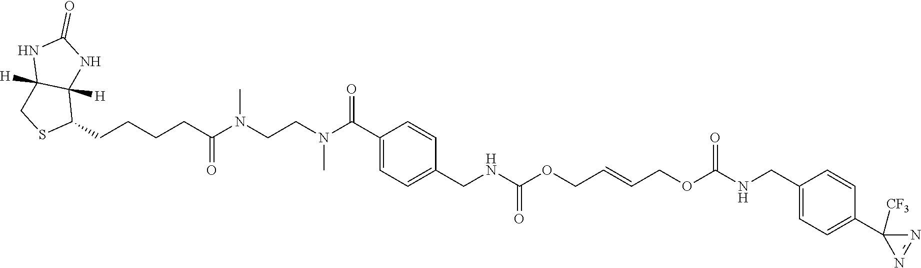 [SMILES: CN(CCN(C)C(=O)c1ccc(CNC(=O)OC/C=C/COC(=O)NCc2ccc(C3(C(F)(F)F)N=N3)cc2)cc1)C(=O)CCCC[C@@H]1SC[C@@H]2NC(=O)N[C@@H]21]

Step 1: To a 100 mL flask was added (E)-but-2-ene-1,4-diol (2.64 g, 30.0 mmol), 4-nitrophenylchloroformate (18.1 g, 89.9 mmol), and DCM (50 mL). The mixture was cooled to 0° C. To the mixture, pyridine (12.1 mL, 150 mmol) was added. After 3 h, solids precipitated. The solids were collected by filtration and washed with DCM to afford the (E)-but-2-ene-1,4-diyl bis(4-nitrophenyl) bis(carbonate). ¹H NMR (400 MHz, DMSO-d₆) δ 8.36-8.28 (m, 4H), 7.59 (d, J=9.1 Hz, 4H), 6.08 (t, J=2.9 Hz, 2H), 4.86-4.81 (m, 4H).

Step 2: To a 100 mL flask, the Biotin-NHS ester (2.90 g, 8.49 mmol), DMF (8 mL), and DIPEA (2.97 mL, 17.0 mmol) was added. The mixture was stirred for 18 h. The solvents were evaporated, and the residue was purified by silica gel chromatography with 0-10% MeOH in DCM to afford tert-butylmethyl(2-(N-methyl-5-((3aS,4S,6aR)-2-oxohexahydro-1H-thieno[3,4-d]imidazol-4-yl)pentanamido)ethyl)carbamate. LRMS [M+H]⁺ 415.

Step 3: To a 100 mL flask, tert-butylmethyl(2-(N-methyl-5-((3aS,4S,6aR)-2-oxohexahydro-1H-thieno[3,4-d]imidazol-4-yl)pentanamido)ethyl)carbamate (3.50 g, 8.44 mmol) and TFA (5 mL) was added. The mixture was stirred for 30 min. The solvents were evaporated, and the residue was co-evaporated with toluene three times to afford the N-methyl-N-(2-(methylamino)ethyl)-5-((3aS,4S,6aR)-2-oxohexahydro-1H-thieno[3,4-d]imidazol-4-yl)pentanamide. LRMS [M+H]⁺ 315.

Step 4: To a 20 mL vial, 4-(((tert-butoxycarbonyl)amino)methyl)benzoic acid (400 mg, 1.59 mmol), N-methyl-N-(2-(methylamino)ethyl)-5-((3aS,4S,6aR)-2-oxohexahydro-1H-thieno[3,4-d]imidazol-4-yl)pentanamide (600 mg, 1.91 mmol), DIPEA (1.11 mL), and DCM (4 mL) was added. To the mixture, T3P (50% in MeCN, 1.46 mL, 2.39 mmol) was added. The mixture was stirred for 1 h. The mixture was purified by silica gel chromatography with 0-10% MeOH in DCM as eluent to afford the product, which contained significant amounts of DIPEA-TFA salts. The material was triturated vigorously with Et₂O two times. Each time, the ether was decanted off leaving an oily solid behind. The solid was then dried under vacuum to afford tert-butyl (4-(methyl(2-(N-methyl-5-((3aR,4R,6aS)-2-oxohexahydro-1H-thieno[3,4-d]imidazol-4-yl)pentanamido)ethyl)carbamoyl)-benzyl)carbamate. LRMS [M+H]⁺ 548.

Step 5: To a 20 mL vial, tert-butyl (4-(methyl(2-(N-methyl-5-((3aR,4R,6aS)-2-oxohexahydro-1H-thieno[3,4-d]imidazol-4-yl)pentanamido)ethyl)carbamoyl)-benzyl)carbamate (488 mg, 0.891 mmol) and TFA (3 mL) was added. The mixture was stirred for 15 min. The solvents were evaporated, and the residue was co-evaporated with toluene 2 times to afford 4-(aminomethyl)-N-methyl-N-(2-(N-methyl-5-((3aR,4R,6aS)-2-oxohexahydro-1H-thieno[3,4-d]imidazol-4-yl)pentanamido)ethyl)benzamide. LRMS [M+H]⁺ 448.

Step 6: To a 100 mL flask, 4-(aminomethyl)-N-methyl-N-(2-(N-methyl-5-((3aR,4R,6aS)-2-oxohexahydro-1H-thieno[3,4-d]imidazol-4-yl)pentanamido)ethyl)benzamide (398 mg, 0.889 mmol), (E)-but-2-ene-1,4-diyl bis(4-nitrophenyl) bis(carbonate) (from Step 1, 744 mg, 1.78 mmol), and DMF (10 mL) was added. The slurry was stirred. To the mixture, DIPEA (1.24 mL, 7.11 mmol) was added. After 30 min, the mixture was concentrated. The residue was diluted in DCM and filtered. The filtrate was loaded onto a silica gel column and purified with 0-15% MeOH in DCM as eluent. The product residue was triturated three times with Et₂O, pouring off the supernatant away from the oily solid that formed each time. The remaining solid was dried under vacuum to afford (E)-4-(((4-nitrophenoxy)carbonyl)oxy)but-2-en-1-yl (4-(methyl(2-(N-methyl-5-((3aR,4R,6aS)-2-oxohexahydro-1H-thieno[3,4-d]imidazol-4-yl)pentanamido)ethyl)carbamoyl)benzyl)carbamate. LRMS [M+H]⁺ 727.

Step 7: To a 20 mL vial, (4-(3-(trifluoromethyl)-3H-diazirin-3-yl)phenyl)methanamine hydrochloride (40.0 mg, 0.159 mmol), (E)-4-(((4-nitrophenoxy)carbonyl)oxy)but-2-en-1-yl (4-(methyl(2-(N-methyl-5-((3aR,4R,6aS)-2-oxohexahydro-1H-thieno[3,4-d]imidazol-4-yl)pentanamido)ethyl)carbamoyl)benzyl)carbamate (116 mg, 0.159 mmol), DMF (4 mL), and DIPEA (0.278 mL, 1.59 mmol) was added. The mixture was stirred for 2 hours. The mixture was purified by RP HPLC (MeCN/water w/ 0.1% TFA) to afford compound 9578. LRMS [M+H]⁺ 803. ¹H NMR (400 MHz, DMSO-d₆) δ 7.92-7.75 (m, 2H), 7.39 (d, J=8.2 Hz, 2H), 7.34-7.23 (m, 6H), 6.43 (s, 2H), 5.83 (d, J=3.7 Hz, 2H), 4.50 (t, J=3.4 Hz, 4H), 4.30 (t, J=6.3 Hz, 2H), 4.22 (d, J=6.1 Hz, 4H), 4.17-4.08 (m, 2H), 3.58 (d, J=12.6 Hz, 3H), 3.37 (s, 2H), 3.10 (td, J=7.4, 4.8 Hz, 1H), 3.01 (s, 1H), 2.95 (d, J=16.8 Hz, 1H), 2.88 (s, 2H), 2.86-2.80 (m, 1H), 2.68 (s, 1H), 2.58 (d, J=12.4 Hz, 1H), 2.34 (d, J=7.2 Hz, 1H), 2.23 (q, J=15.7, 11.6 Hz, 2H), 1.66-1.29 (m, 6H).

### Synthesis of Compound 9643

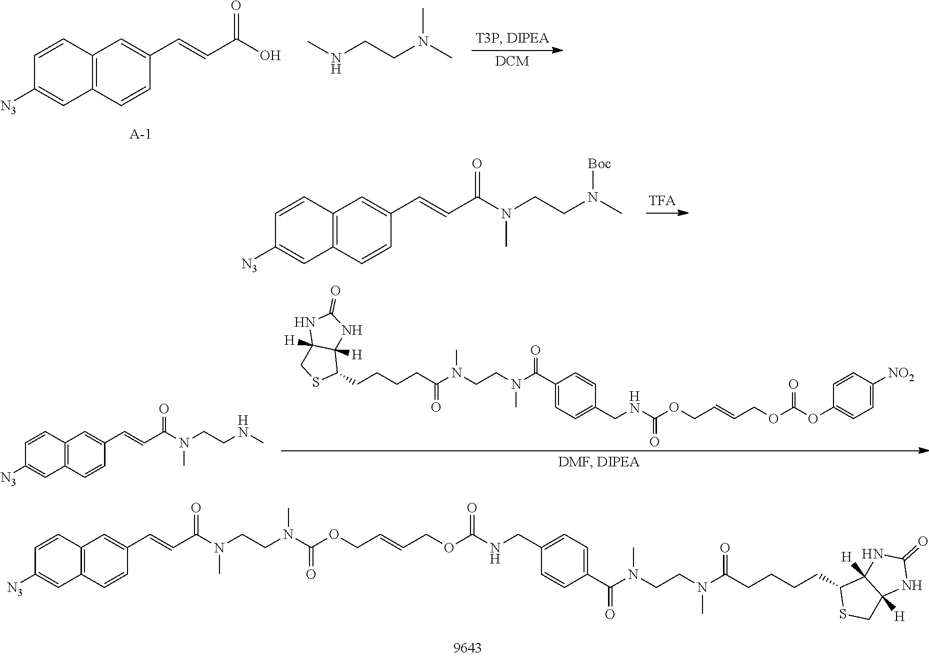

Step 1: To a 20 mL vial, A-1 (232 μmg, 0.971 mmol), tert-butyl methyl(2-(methylamino)ethyl)carbamate (366 mg, 1.94 mmol), DIPEA (0.509 mL, 2.91 mmol), and DCM (4 mL) was added. To the mixture, T3P (50% in MeCN, 0.891 mL, 1.46 mmol) was added. The mixture was stirred for 10 min. The mixture was purified by silica gel chromatography with 0-10% MeOH in DCM to afford tert-butyl (E)-(2-(3-(6-azidonaphthalen-2-yl)-N-methylacrylamido)ethyl)-(methyl)carbamate. LRMS [M+H]⁺ 410.

Step 2: To a 20 μmL vial, tert-butyl (E)-(2-(3-(6-azidonaphthalen-2-yl)-N-methylacrylamido)ethyl)-(methyl)carbamate (60.0 mg, 0.146 mmol) and formic acid (2 mL). was added. The mixture was stirred for 2 hours. The solvents were evaporated to afford the (E)-3-(6-azidonaphthalen-2-yl)-N-methyl-N-(2-(methylamino)ethyl)acrylamide. LRMS [M+H]⁺ 310.

Step 3: To a 20 μmL vial, (E)-3-(6-azidonaphthalen-2-yl)-N-methyl-N-(2-(methylamino)ethyl)acrylamide (45.3 μmg, 0.146 μmmol), (E)-4-(((4-nitrophenoxy)carbonyl)oxy)but-2-en-1-yl (4-(methyl(2-(N-methyl-5-((3aR,4R,6aS)-2-oxohexahydro-1H-thieno[3,4-d]imidazol-4-yl)pentanamido)ethyl)carbamoyl)benzyl)carbamate (from Step 6 of 9578 106 mg, 0.146 mmol), DMF (4 mL), and DIPEA (0.256 mL, 1.46 mmol) was added. The mixture was stirred for 2 hours. The mixture was purified by RP HPLC (MeCN/water w/ 0.1% TFA) to afford compound 9643. LRMS [M+H]⁺ 897. ¹H NMR (400 MHz, DMSO-d₆) δ 8.23 (t, J=5.9 Hz, 1H), 8.08 (s, 1H), 8.00 (d, J=8.8 Hz, 1H), 7.94 (d, J=8.7 Hz, 1H), 7.81 (q, J=6.2, 5.3 Hz, 1H), 7.75 (d, J=8.7 Hz, 1H), 7.70 (d, J=2.3 Hz, 1H), 7.57 (d, J=15.7 Hz, 1H), 7.39-7.25 (m, 6H), 6.72 (d, J=15.8 Hz, 1H), 6.39 (d, J=27.5 Hz, 2H), 5.83 (d, J=3.0 Hz, 2H), 4.59-4.40 (m, 4H), 4.30 (t, J=6.6 Hz, 1H), 4.22 (d, J=5.9 Hz, 2H), 4.12 (q, J=7.7, 6.5 Hz, 1H), 3.38 (br s, 12H), 3.26 (q, J=6.3 Hz, 2H), 3.12 (q, J=6.9, 6.4 Hz, 2H), 3.00 (s, 1H), 2.95 (d, J=15.9 Hz, 1H), 2.87 (s, 1H), 2.82 (dd, J=11.7, 4.5 Hz, 1H), 2.58 (d, J=12.3 Hz, 1H), 2.21 (d, J=20.6 Hz, 2H), 1.67-1.28 (m, 6H).

### Synthesis of Compound 9679

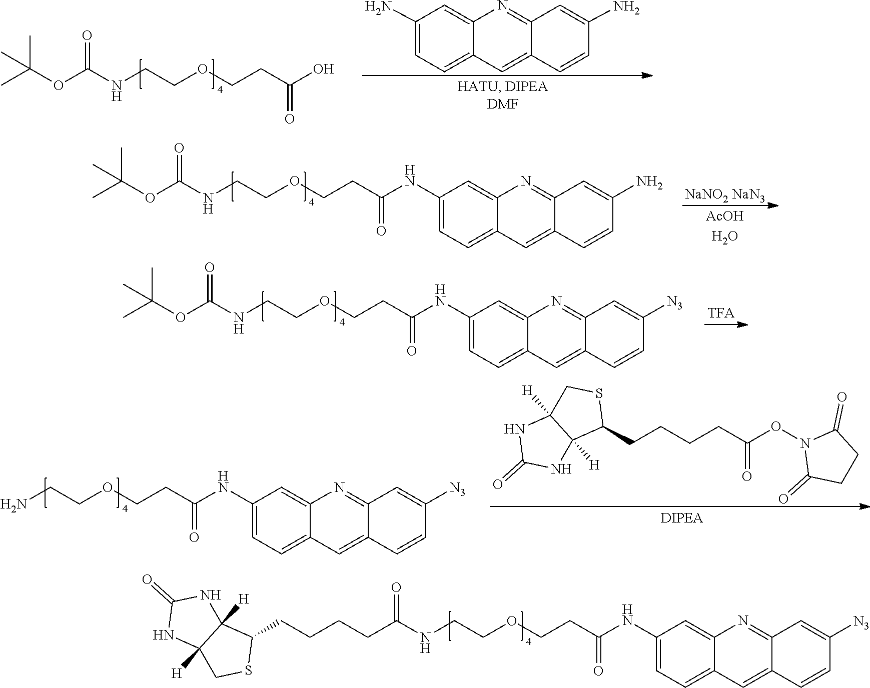

Step 1: To a 20 mL vial, Boc-15-amino-4,7,10,13-tetraoxapentadecanoic acid (250.0 mg, 0.68 mmol) was added and dissolved with 6 mL anhydrous DMF. HATU (390.2 mg, 1.03 mmol) and DIPEA (357.5 uL, 2.05 mmol) were added to the vial at room temperature and stir for 30 minutes. Acridine-3,6-diamine (286.3 mg, 1.37 mmol) was added to the reaction mixture and stirred at room temperature. After 1.5 hours, the reaction mixture was diluted with another 5 mL DMF and purified by RP HPLC (MeCN/water w/ 0.1% TFA) to afford tert-butyl (15-((6-aminoacridin-3-yl)amino)-15-oxo-3,6,9,12-tetraoxapentadecyl)carbamate. LRMS [M+H]⁺ 557.

Step 2: To a 20 mL vial, tert-butyl (15-((6-aminoacridin-3-yl)amino)-15-oxo-3,6,9,12-tetraoxapentadecyl)carbamate (139.9 mg, 0.25 mmol) was added and dissolved with 4.5 mL water with the addition of 3 mL AcOH. NaNO₂ (29.5 mg, 0.43 mmol) was dissolved with 1 mL water and added to the reaction at 0° C. After 5 min, NaN₃ (32.7 mg, 0.50 mmol) was dissolved with 1 mL water and added to the reaction at 0° C. dropwise. Bubbles were observed. When the bubbles disappeared, the temperature was raised to room temperature. After 30 min, the pH of the aqueous layer was adjusted to 7 with NaHCO₃. The reaction was quenched with the addition of brine and extracted with 30 mL EtOAc three times. The organic layer was collected, and the crude product was purified by RP HPLC (MeCN/water w/ 0.1% TFA) to afford tert-butyl (15-((6-azidoacridin-3-yl)amino)-15-oxo-3,6,9,12-tetraoxapentadecyl)carbamate. LRMS [M+H]⁺ 583.

Step 3: To a 20 mL vial, tert-butyl (15-((6-azidoacridin-3-yl)amino)-15-oxo-3,6,9,12-tetraoxapentadecyl)carbamate (93.0 mg, 0.16 mmol) was added and dissolved with 2.4 mL DCM with the addition of 0.6 mL TFA at 0° C. After 30 min, the reaction mixture was diluted with DMF and purified by RP HPLC (MeCN/water w/ 0.1% TFA) to afford 1-amino-N-(6-azidoacridin-3-yl)-3,6,9,12-tetraoxapentadecan-15-amide. LRMS [M+H]⁺ 483.

Step 4: To a 20 mL vial, 1-amino-N-(6-azidoacridin-3-yl)-3,6,9,12-tetraoxapentadecan-15-amide (86.7 mg, 0.18 mmol) was dissolved with 3 mL anhydrous DMF. DIPEA (94 uL, 0.54 mmol) was added to the reaction at RT. Biotin-NHS (92.0 mg, 0.27 mmol) was added to the reaction. After 10 minutes, the reaction mixture was diluted with DMF and purified by RP HPLC (MeCN/water w/ 0.1% TFA) compound 9679. LRMS [M+H]⁺ 709. ¹H NMR (400 MHz, DMSO-d₆) δ 10.95 (s, 1H), 9.56 (s, 1H), 8.84 (s, 1H), 8.42 (d, J=9.0 Hz, 1H), 8.34 (d, J=9.1 Hz, 1H), 7.81 (t, J=5.7 Hz, 1H), 7.73 (dd, J=9.1, 1.9 Hz, 1H), 7.71 (d, J=2.1 Hz, 1H), 7.57 (dd, J=9.0, 2.2 Hz, 1H), 6.42 (s, 1H), 6.37 (s, 1H), 4.30 (dd, J=7.8, 4.9 Hz, 1H), 4.12 (dd, J=7.8, 4.4 Hz, 1H), 3.79 (t, J=6.1 Hz, 2H), 3.57-3.46 (m, 16H), 3.35 (t, J=5.9 Hz, 2H), 3.08 (m, 1H), 2.81 (dd, J=12.4, 5.1 Hz, 1H), 2.75 (t, J=6.1 Hz, 2H), 2.05 (t, J=7.4 Hz, 2H), 1.66-1.54 (m, 1H), 1.53-1.39 (m, 2H), 1.36-1.20 (m, 2H).

### Synthesis of Compound 9680

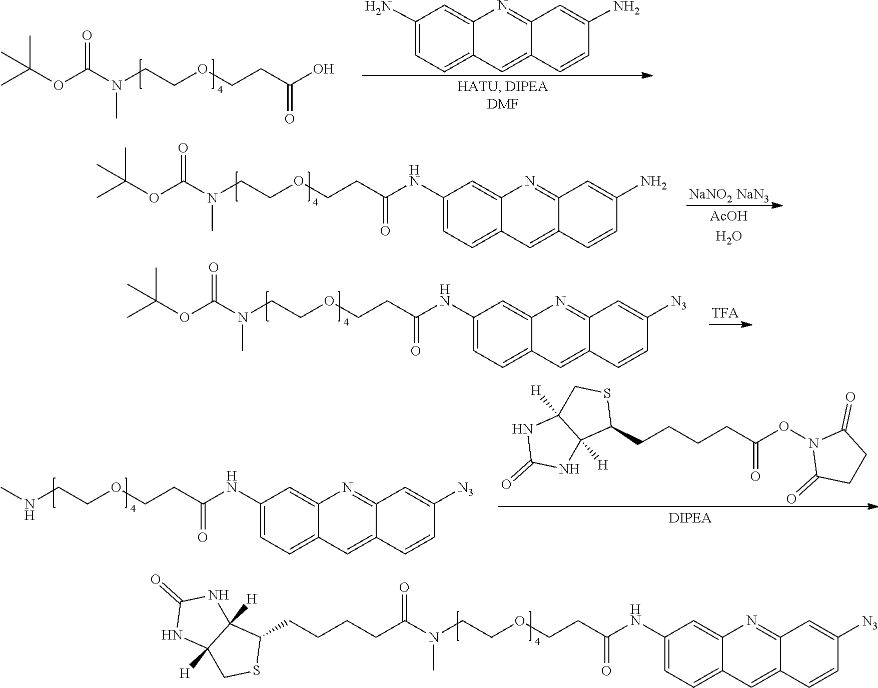

The synthesis of compound 9680 was conducted in analogy to that of 9679 from the appropriate starting material, affording 9680. LRMS [M+H]⁺ 723.

### Synthesis of PS-9850

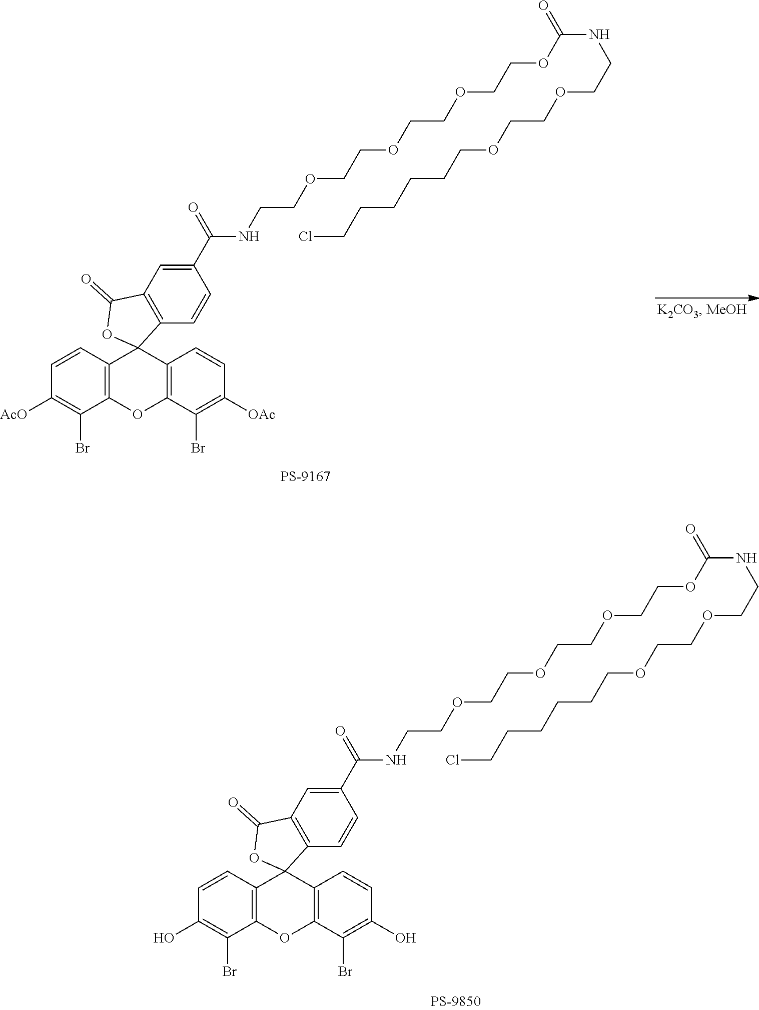 [SMILES: CC(=O)Oc1ccc2c(c1Br)Oc1c(ccc(OC(C)=O)c1Br)C21OC(=O)c2cc(C(=O)NCCOCCOCCOCCOC(=O)NCCOCCOCCCCCCCl)ccc21 ; O=C(NCCOCCOCCCCCCCl)OCCOCCOCCOCCNC(=O)c1ccc2c(c1)C(=O)OC21c2ccc(O)c(Br)c2Oc2c1ccc(O)c2Br]

To a 20 mL vial, PS-9167 (15.9 mg, 0.0152 mmol), K₂CO₃ (20.0 mg, 0.145 mmol) and MeOH (1 mL) was added. The mixture was stirred for 20 min. The mixture was diluted with DMF and purified by RP HPLC (MeCN/water w/ 0.1% TFA) to afford PS-9850. LRMS [M+H]⁺ 957. ¹H NMR (400 MHz, DMF-d₇) δ 11.34 (s, 2H), 8.98 (t, J=5.4 Hz, 1H), 8.60 (s, 1H), 8.43 (dd, J=8.0, 1.6 Hz, 1H), 7.67 (d, J=8.1 Hz, 1H), 6.93 (d, J=8.8 Hz, 2H), 6.79 (d, J=8.7 Hz, 2H), 4.12 (t, J=4.9 Hz, 2H), 3.71 (t, J=5.8 Hz, 2H), 3.68-3.59 (m, 12H), 3.56 (t, J=7.0 Hz, 2H), 3.52 (m, 5H), 3.43 (t, J=6.5 Hz, 2H), 3.26 (q, J=6.0 Hz, 2H), 1.76 (p, J=6.9 Hz, 2H), 1.54 (p, J=6.8 Hz, 2H), 1.48-1.31 (m, 4H).

### Example 15

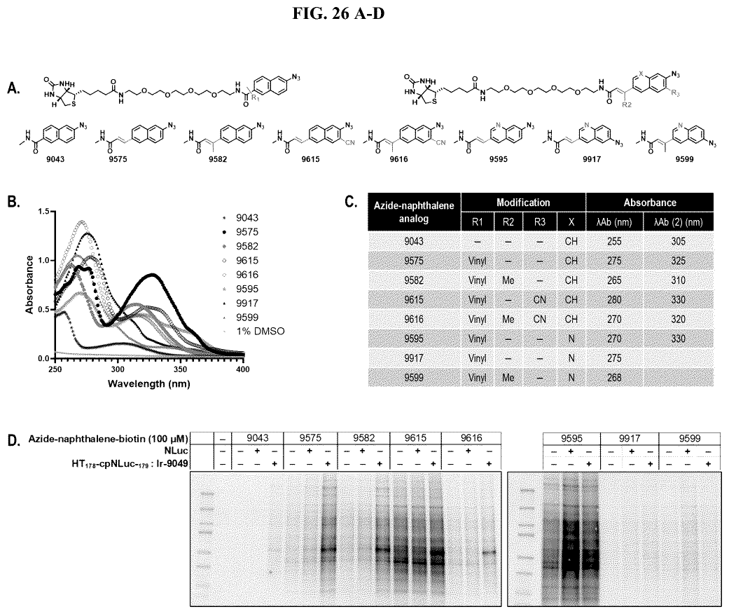

This example demonstrates the capacity to further expand the toolkit of the photoreactive groups, which are compatible with the bioluminescent photocatalytic system to include vinyl-naphthyl-azides. The structures of the vinyl-naphthyl-azide-biotins and vinyl-quinoline-azide-biotins are shown in FIG. 26A, and their syntheses are included in Example 13. Absorbance profiles for 200 μM activatable labels in 2% DMSO were monitored on a SPARK multimode plate reader (FIG. 26 B-C). Generally, the substitutions onto the naphthyl resulted in a range of red-shifted absorbances relative to the two absorbance peaks of parental naphthyl-azide 9043 (λmax=255 and 305 nm) suggesting higher capacity to absorb light and subsequently easier activation by bioluminescence-triggered photocatalytic energy transfer. The naphthyl-azide analogs were further evaluated for their capacity to undergo bioluminescence-triggered photocatalytic protein labeling as well as for their light-dependent, light-independent, and catalyst-independent backgrounds (FIG. 26D). To this end, reactions comprising 100 μM naphthyl-azide biotin analog, 0.1 mg/mL K562 lysate depleted of biotinylated proteins, and 60 nM HT₁₇₈-cpNLuc-₁₇₉: Ir-9049 conjugate were assembled in TBS pH 7.5 within wells of a white, 96-well plate. Control reactions either didn't include the HT₁₇₈-cpNLuc-₁₇₉:Ir-9049 conjugate (light-independent and catalyst-independent background) or exchanged the HT₁₇₈-cpNLuc-₁₇₉: Ir-9049 conjugate with NanoLuc (no catalyst; light-dependent background). Bioluminescence was induced upon treatment with 100 μM fluorofurimazine for 20 minutes. To evaluate labeling efficiencies, samples were collected, resolved on SDS-PAGE, transferred to nitrocellulose membranes, and analyzed as described in Example 1. Western analyses revealed that the red-shifted absorbance associated with the vinyl substitution resulted with increased labeling efficiencies relative to parental naphthyl-azide 9043 without any significant increase in light-dependent and light-independent backgrounds except for 9615. Furthermore, a methyl substitution on the vinyl group designed to inhibit Michael addition reduced background, especially for 9615, and increased labeling specificity. This reduced background was generally associated with a blue-shifted absorbance relative to the equivalent vinyl-naphthyl-azide analog.

The absorbance profile and capacity to undergo bioluminescence-triggered protein labeling as well as light-dependent and light-independent backgrounds for the quinolone-azide analogs were strongly influenced by the position of the azide. The position associated with 15 nm and 25 nm red shifted absorbances (i.e., 9595) exhibited very high background, while the position that resulted in a single blue-shifted absorbance peak (i.e., 9917 and 9599) exhibited poor protein labeling.

### Example 16

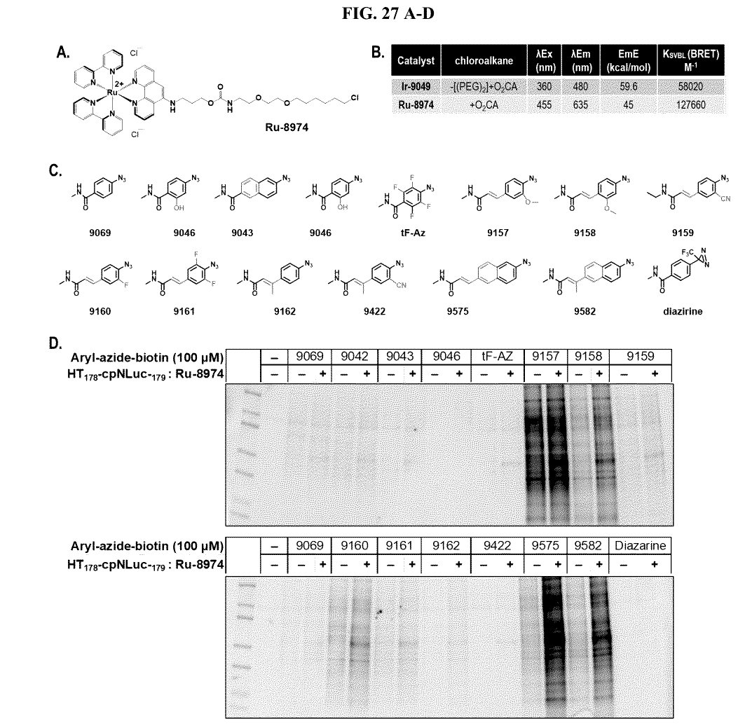

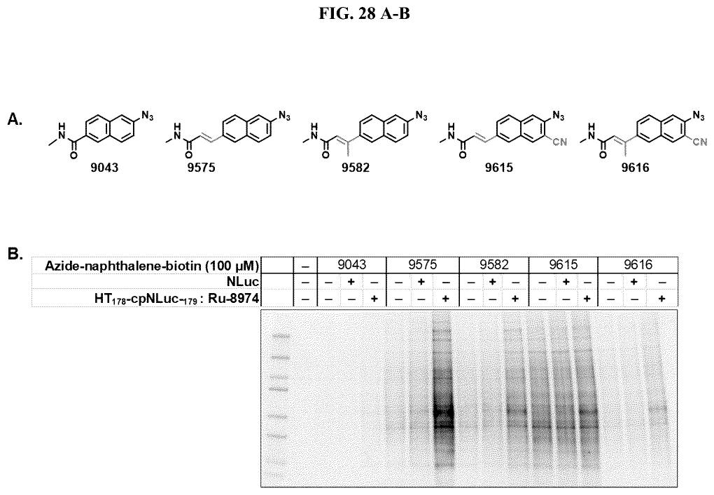

During development of the bioluminescence-triggered photocatalytic labeling described herein, experiments were conducted to evaluate the capacity of a ruthenium catalyst to drive bioluminescence-triggered photocatalytic protein labeling (FIGS. 27-28). The structure of the chloroalkane-conjugated ruthenium catalyst Ru-8974 is shown in FIG. 27A, and its synthesis is included in Example 12. The physiochemical properties of the iridium and ruthenium catalysts, and their capacity to undergo activation by bioluminescence energy transfer, were determined as described in Examples 1 and 3 and are shown in FIG. 27B. These analyses revealed that ruthenium's red-shifted excitation and greater overlap with NanoLuc emission resulted in a significantly higher energy transfer efficiency from NanoLuc to the catalyst. At the same time, its red-shifted emission resulted in a lower Emission Energy (EmE) compared to the iridium catalyst as well as a subsequently lower capacity to undergo energy transfer events with the activatable labels.

The capacity of the ruthenium catalyst to drive bioluminescence-triggered photocatalytic protein labeling was further evaluated for a subset of photoreactive groups (FIGS. 27D and 28B). Structures of the photoreactive groups included in the screen are shown in FIGS. 27C and 28A, and their syntheses are included in Example 13. To this end, reactions comprising 100 μM activatable label, 0.1 mg/mL K562 lysate depleted of biotinylated proteins, and 60 nM HT₁₇₈-cpNLuc-₁₇₉:Ru-8974 conjugate were assembled in TBS pH 7.5 within wells of a white, 96-well plate. Control reactions either didn't include the HT₁₇₈-cpNLuc-₁₇₉:Ru-8974 conjugate (light-independent and catalyst-independent background) or exchanged the HT₁₇₈-cpNLuc-₁₇₉:Ru-8974 conjugate with NanoLuc (no catalyst; light-dependent background). Bioluminescence was induced upon treatment with 100 μM fluorofurimazine for 20 minutes. To evaluate labeling efficiencies, samples were collected, resolved on SDS-PAGE, transferred to nitrocellulose membranes, and analyzed as described in Example 1. Western analyses revealed that ruthenium could drive bioluminescence-triggered photocatalytic protein labeling with several activatable labels albeit with a broad range of efficiencies and light-dependent and light-independent backgrounds. Several substitutions increased labeling specificity to different extents likely by impeding the generation-kinetics and/or lifetime of the reactive intermediates resulting with an overall smaller labeling radius.

### Example 17

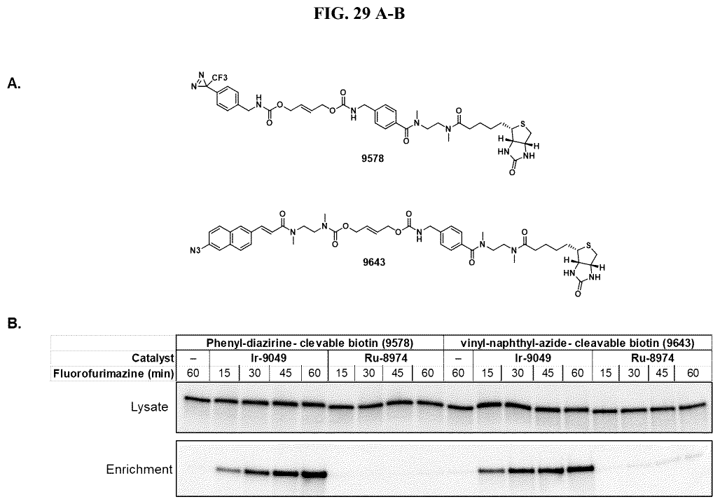

This example demonstrates the capacity to assemble bioluminescent photocatalytic complexes inside cells for subsequent protein labeling and enrichment (FIG. 29). Structures of the activatable labels comprising either phenyl-trifluoro-methyl diazirine or vinyl-naphthyl-azide linked to palladium cleavable biotin are shown in FIG. 29A, and their syntheses are included in Example 13 Briefly, HeLa cells were transfected with a DNA construct encoding HT₁₇₈-cpNLuc-₁₇₉, plated into wells of 6-well plates at 2×10⁵ cell/mL, and incubated overnight at 37° C., 5% CO₂. The next day, plates were treated with Ir-9049 or Ru-8974 catalysts at a final concentration of 3 M for 90 minutes to allow assembly of the bioluminescent photocatalytic complex. To remove excess unreacted catalyst, cells were washed twice, 15 minutes each, in HBSS buffer. The last HBSS wash was replaced with Opti-MEM media supplemented with 2% serum and 20 μM cleavable-activatable label. Following 30-minutes incubation, plates were treated with 20 μM fluorofurimazine for up to 60-minutes while control cells remained untreated. To remove excess unreacted activatable-labels, cells were washed twice, 15 minutes each, in HBSS buffer. The last HBSS wash was replaced with 1 mL Mammalian Lysis Buffer (Promega) supplemented with 10-fold dilution of 10×RQ1-DNase buffer (Promega), 50-fold dilution RQ1-DNase (Promega), and 100-fold dilution Protease Inhibitor Cocktail (Promega). Following 30-minutes incubation at room temperature with constant mixing, cell lysates were collected, and biotinylated proteins were captured on 75 μL High Capacity Magne® Streptavidin Beads (Promega) while nonspecific interactions were washed-out. Labeled proteins were then released by a 30-minutes incubation with a palladium cleavage reagent (Promega), resolved on SDS-PAGE, transferred to PVDF membrane, and subjected to Western analysis using antibody against HaloTag (Promega). The Western blots (FIG. 29B) revealed significantly more efficient Iridium driven photocatalytic labeling and enrichment of the chimera with either diazirine or vinyl-naphthyl azide.

### Example 18

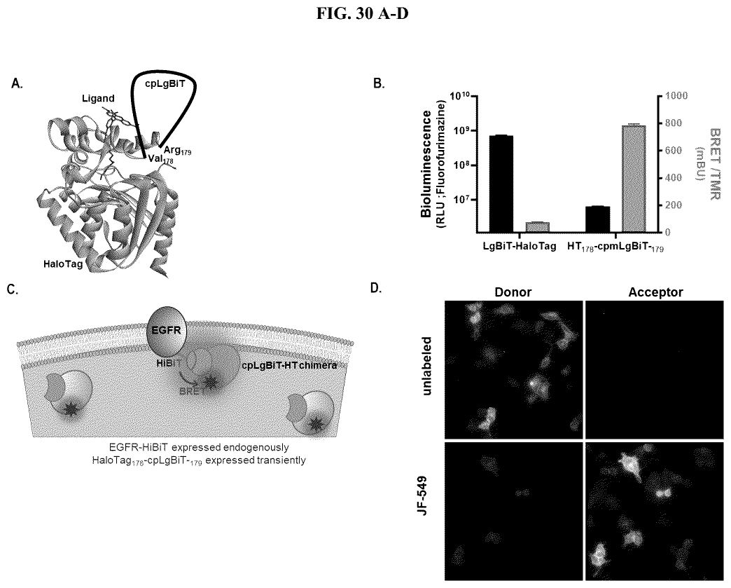

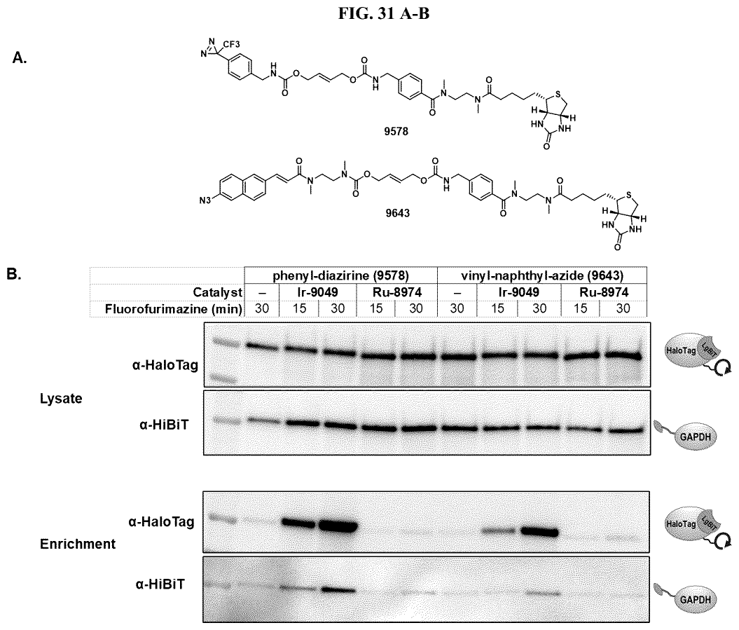

This example describes further optimization of a complementation-based bioluminescent photocatalytic complex and utilizing it as the means for targeting the photocatalytic system to an endogenous HiBiT-tagged target (FIGS. 30 and 31). To increase the efficiency of bioluminescence energy transfer to abound catalyst, a chimeric structure comprising a circularly permuted LgBiT mutant incorporating 4 mutations from LgTrip (E4D, Q42M, M106K, T144D (i.e., cpmLgBiT circularly permutated at residues 67/68)) that is inserted into a HaloTag's surface loop (between residues 178-179), which is proximal to the ligand interaction site (i.e., HT₁₇₈-cpmLgBiT-₁₇₉) (FIG. 30A), was engineered. First, the chimera was compared to a simple LgBiT-HaloTag fusion for its brightness and efficiency of bioluminescence resonance energy transfer (BRET) to a bound HaloTag TMR-fluorescent ligand. To this end, the LgBiT-HaloTag fusion and chimera were diluted in TBS+0.01% BSA to a final concentration of 13 nM and allowed to complement for 60 minutes with an equal volume of 130 nM of VS-HiBiT peptide. Following complementation, reactions were either further incubated with 10× HaloTag-TMR ligand at a final concentration of 300 nM or remained untreated. Upon treatment with 10× fluorofurimazine at a final concentration of 20 μM, raw luminescence (Total RLU) or filtered luminescence for donor (e.g., 450 nm/8 nm BP) and acceptor (600 nm LP) emissions were measured on a GloMax® Discover plate reader (Promega). BRET ratios were further calculated for each sample by dividing the acceptor emission value by its donor emission value. Although HT₁₇₈-cpmLgBiT-₁₇₉ was 100-fold dimmer, it provided 10-fold greater BRET efficiency (FIG. 30B) indicating that the chimeric structure was able to induce greater proximity between the luminogenic substrate binding site and the bound fluorescent ligand or adopt a conformation favorable for energy transfer between the two or both.

To demonstrate the specificity driven by a system coupling complementation of the bioluminescent energy donor with BRET activation of the bound ligand (e.g. chloroalkane-conjugated to a fluorophore, chloroalkane-conjugated to a light sensitive catalyst etc.), cells expressing an endogenous HiBiT-tagged EGFR, which is localized to the cellular membrane, were transfected with DNA encoding HT₁₇₈-cpmLgBiT-₁₇₉ chimera, plated into 35 mm glass bottom dishes (MatTek Corporation), and incubated for 24 hour in a tissue culture incubator. Next day, cells were either remained unlabeled or were labeled for 1 hour with HaloTag JF-549-fluorescent ligand at a final concentration of 1 M. Following two washes, 15 minutes each, cells were treated with fluorofurimazine at a final concentration of 20 μM and imaged on the Olympus LV200 bioluminescence microscope (Olympus). A suitable field of view was identified based on imaging of total luminescent signal of the donor. To image BRET events, images of donor and acceptor emissions were acquired sequentially using a 460/80 bandpass filter and a 590 nm long-pass filter, respectively. Imaging analysis revealed total luminescent and BRET signals were localized to the cellular membrane as well as highly efficient energy transfer to the fluorescent acceptor.

The capacity of a complementation-based bioluminescent photocatalytic system to drive labeling of an endogenous HiBiT-tagged GAPDH was further evaluated (FIG. 31). Structures of the activatable labels comprising either phenyl-trifluoro-methyl diazirine or vinyl-naphthyl-azide linked to palladium cleavable biotin are shown in FIG. 31A. Briefly, HeLa cells expressing an endogenous HiBiT-tagged GAPDH were transfected with a DNA construct encoding HT₁₇₈-cpmLgBiT-₁₇₉, plated into wells of 6-well plates at 2×10⁵ cell/mL, and incubated overnight at 37° C., 5% CO₂. The next day, plates were treated with either Ir-9049 or Ru-8974 catalysts at a final concentration of 3 μM for 90 minutes to allow assembly of the bioluminescent photocatalytic complex. Bioluminescence-triggered labeling, and enrichments were carried out as described above in Example 17. Enriched proteins were resolved on SDS-PAGE, transferred to PVDF membranes, and subjected to Western analysis using antibodies against either HaloTag (Promega) or HiBiT (Promega). The Western blots (FIG. 31B) revealed significantly more efficient Iridium driven photocatalytic labeling and enrichment of both the chimera and endogenous HiBiT-GAPDH with either diazirine or vinyl-naphthyl azide.

### Example 19

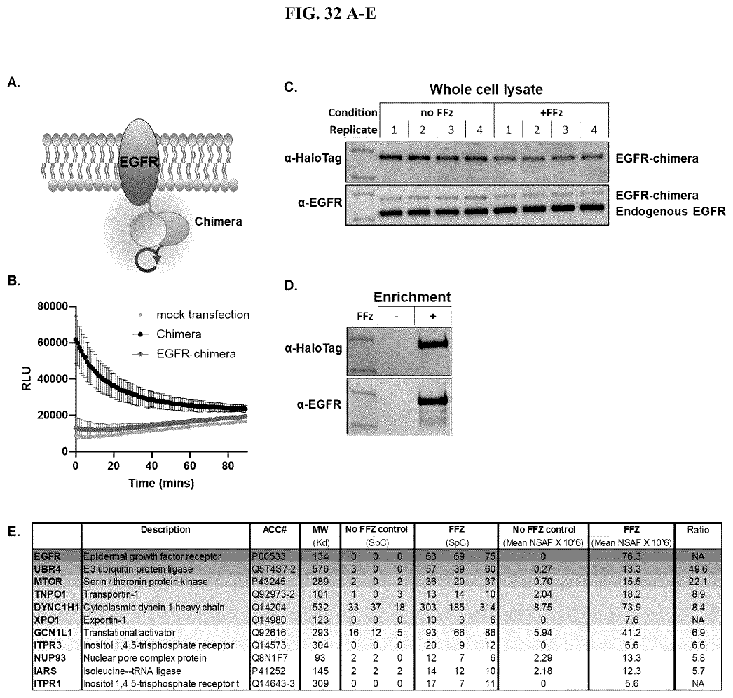

During development of the bioluminescence-triggered photocatalytic labeling described herein, experiments were conducted to evaluate the capacity to drive intracellular labeling of neighboring proteins (FIG. 32). This evaluation made use of a construct encoding a genetic fusion of EGFR-HT₁₇₈-cpNLuc-₁₇₉, which is localized to the cellular membrane (FIG. 32A). Total luminescence emitted over time from cells expressing either the chimera alone or the EGFR-HT₁₇₈-cpNLuc-₁₇₉ fusion showed that the fusion is significantly dimmer (FIG. 32B).

Cells expressing the chimera were further evaluated for their capacity to drive fluorofurimazine-dependent labeling of neighboring proteins. To this end, HeLa cells were transfected with the DNA construct encoding EGFR-HT₁₇₈-cpNLuc-₁₇₉ fusion, plated into 10 cm dishes at 1×10⁶ cells/dish, and incubated overnight at 37° C., 5% CO₂. The next day, plates were treated with Ir-9049 catalyst at a final concentration of 3 μM for 90 minutes to allow assembly of the bioluminescent photocatalytic complex. Following two washes to remove excess unreacted catalyst, cells were treated with 20 M cleavable-diazirine-biotin for 30-minutes. Bioluminescence was initiated upon treatment with 20 μM fluorofurimazine while control cells remained untreated. Following 60-minutes incubation, cells were washed to remove excess unreacted cleavable diazirine-biotin and then collected into 10 mM MOPS buffer pH 7.4 supplemented with 100-fold dilution of Protease Inhibitor Cocktail (Promega). Following sonication, lysates were supplemented with DDM at a final concentration of 1% and NaCl at a final concentration of 150 mM, incubated for 60 minutes with constant mixing, and then briefly centrifuged to remove cellular debris. Small fraction of lysates from replicates, which were either treated or untreated with fluorofurimazine, were subjected to Western analysis using antibodies against HaloTag (Promega) or EGFR (Cell Signaling) to verify equivalent processing across replicates. This analysis also revealed significantly lower expression of the over expressed EGFR-HT₁₇₈-cpNLuc-₁₇₉ fusion relative to the endogenous EGFR (FIG. 32C). Labeled proteins were further enriched by overnight capture onto 120 μL High Capacity Magne® Streptavidin Beads (Promega). Next day, nonspecific interactions were washed-out, and labeled proteins were released by a 45-minutes incubation with a palladium cleavage reagent (Promega). Western analyses of eluted proteins using antibodies against HaloTag (Promega) and EGFR (Cell Signaling) revealed fluorofurimazine-dependent enrichment of the chimera despite its relatively low expression and dim luminescence (FIG. 32D).

To detect enrichment of neighboring proteins, lysates were further subjected to mass spectrometry analysis. To this end, lysates were incubated for 30 minutes with silica HMBC beads (Promega) in LCMS grade acetonitrile at a final concentration of 80% for 30 minutes. Following three washes in 80% ethanol, captured proteins were subjected to 30 minutes reduction followed by 30 minutes alkylation and then overnight on-beads digestion with Trypsin-LysC. Tryptic peptides were removed, quenched with 5% formic acid, desalted, and subjected to mass spectrometry analysis. This analysis revealed not only significant fluorofurimazine-dependent enrichment of EGFR, but also significant enrichment of other membrane localized proteins as well as proteins associated with degradation suggesting the EGFR-chimera is continuously phosphorylated and degraded (FIG. 32 E).

### Example 20

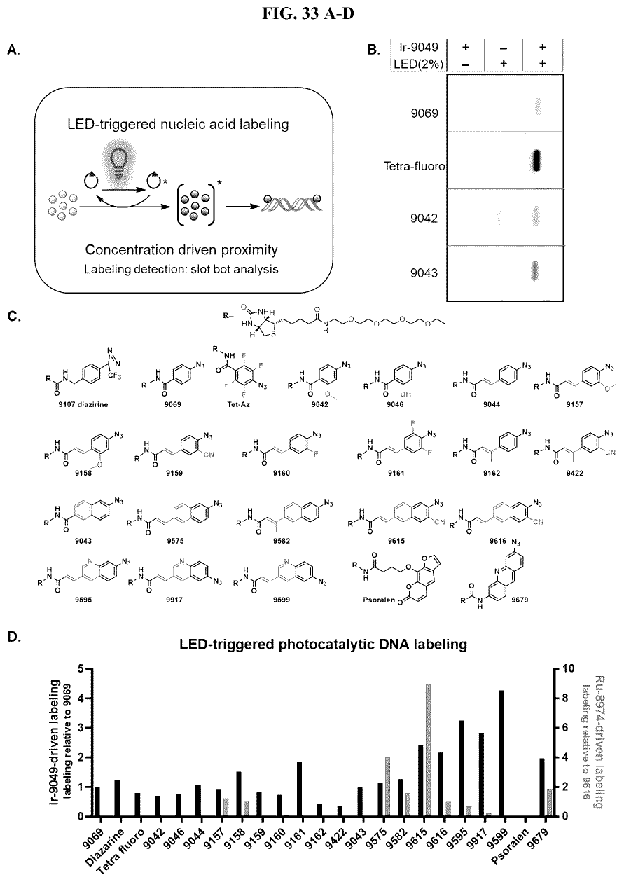

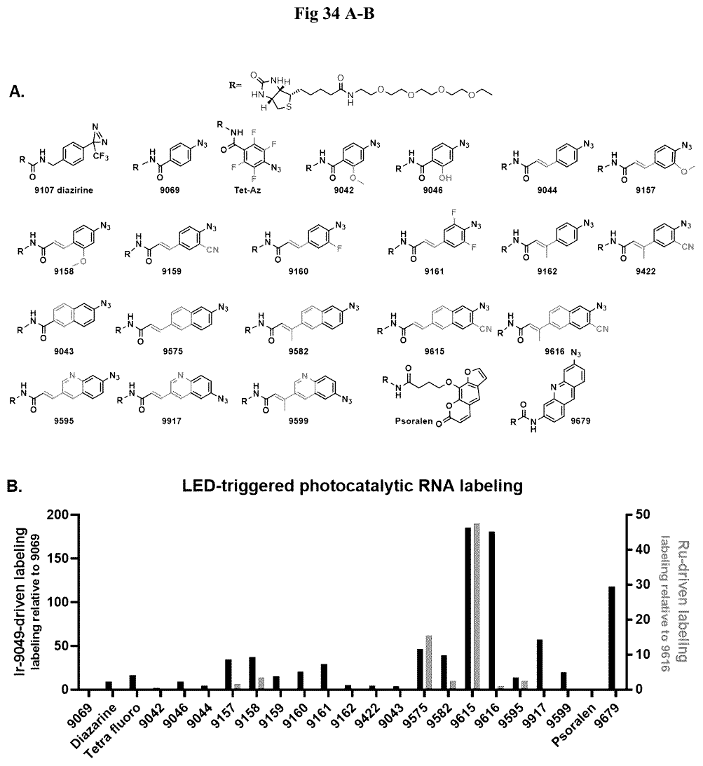

During development of the bioluminescence-triggered photocatalytic labeling described herein, experiments were conducted to evaluate the capacity of the activatable labels to undergo covalent crosslinking with proximal nucleic acids (FIGS. 33-34). Structures of the evaluated photoreactive groups are shown in FIGS. 33C and 34A, and their syntheses are described in Example 13.

Briefly, experimental and control reactions comprising either (1) 0.1 μg/L DNA or RNA markers (Promega) and 50 μM activatable label or (2) 0.1 μg/μL DNA or RNA markers (Promega), 50 μM activatable label, and 100 μM catalyst (Ir-9049 or Ru-8974) were assembled in TE buffer pH 7.4 within wells of UV transparent, 96-well plates. Plates remained in the dark (control) or irradiated at 455 nm (2% LED; ˜1.6 W) for 20 minutes using Efficiency Aggregators biophotoreactor. To evaluate covalent crosslinking efficiencies, reactions were cleaned from non-crosslinked activatable labels using Zeba column (ThermoFisher) before being spotted on nitrocellulose membranes using a slot blot apparatus. Membranes were blocked with 5% BSA (Promega) in TBST for 1 hour at room temperature and then incubated overnight at 4° C. with anti-biotin antibody (Invitrogen) in TBST. Next day, membranes were washed three times with TBST and then incubated for an hour with a secondary anti-goat-HRP-antibody (Jackson laboratories). Following three washes in TBST, membranes were treated with ECL substrate (Promega) and scanned on the chemiluminescence channel to detect DNA or RNA labeled with biotin. Bands volumes quantitated using Image J software were normalized as follows: Ir-9049 driven DNA or RNA-labeling efficiencies were derived from normalization to labeling using 9069 as the activatable label, while Ru-8974 driven DNA- or RNA-labeling efficiencies were derived from normalization to labeling using 9616 as the activatable label. These analyses revealed generally more efficient iridium driven activation and crosslinking to DNA (FIG. 33D) and RNA (FIG. 34B). In addition, several vinyl-naphthyl-azides and vinyl-quinoline-azides exhibited relatively high labeling efficiencies.

### Example 21

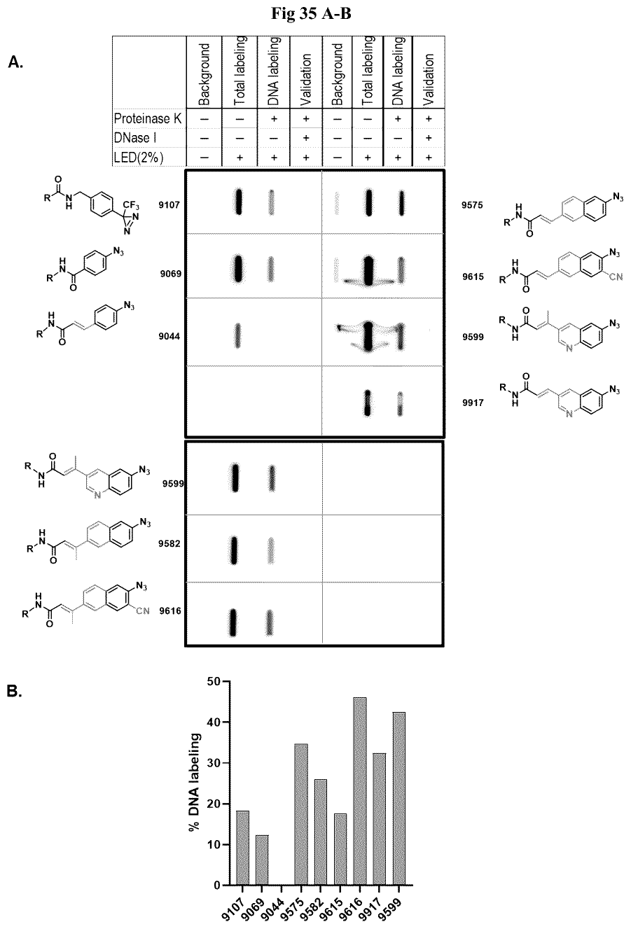

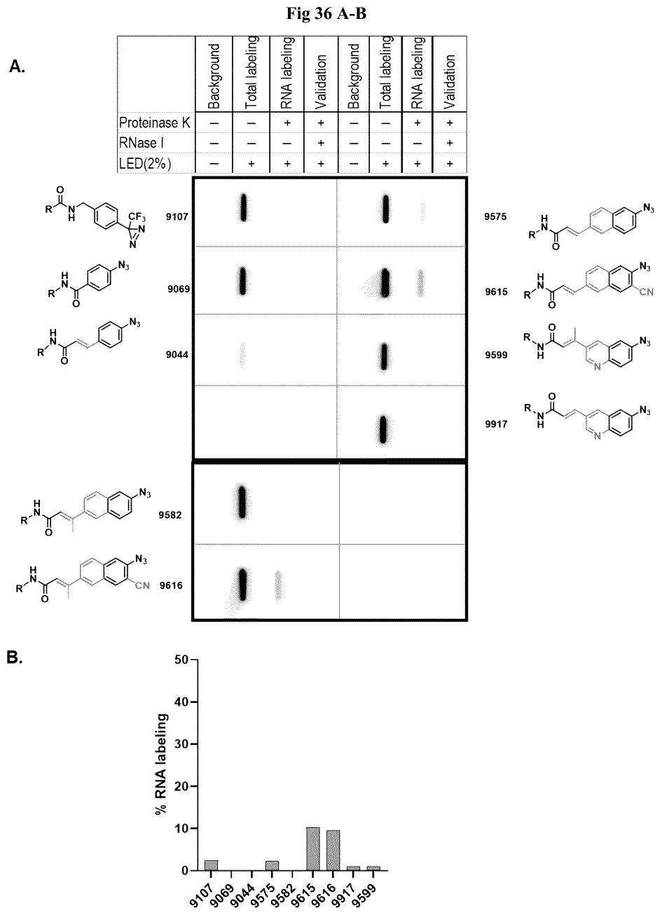

Since the activatable labels were shown to crosslink to both proteins and nucleic acids, experiments were further conducted to evaluate their preference for crosslinking to DNA or RNA versus protein (FIGS. 35-36). Structures of a subset of photoreactive groups included in these evaluations are shown in FIGS. 35A and 36A alongside the slot blot analyses while their syntheses are described in Example 13.

Briefly, for each activable label, four replicate reactions (3 experimental and one no light control) comprising 1 pmol of DNA or RNA, 1 pmol of acetylated BSA, 50 μM activatable label, and 100 μM iridium catalyst (Ir-9049) were assembled in TE buffer pH 7.4 within wells of UV transparent, 96-well plates. Plates were remained in the dark (control) or irradiated at 455 nm (2% LED; ^{˜}1.6 W) for 20 minutes using Efficiency Aggregators biophotoreactor. To evaluate efficiencies of total labeling (protein+nucleic acid) as well as specific nucleic acids labeling, non-crosslinked activatable labels were removed from all reactions using Zeba column (ThermoFisher). The control reactions as well as replicates design to evaluate total labeling (protein+nucleic acid) were not subjected to additional treatment. To evaluate % of nucleic acids labeling form total, two replicates were further subjected to proteinase K digestion followed by DNA or RNA cleanup using either Wizard columns (Promega) or Zymo RNA columns (Zymo), respectively. To further verify the signal is indeed originating from either DNA or RNA labeling, one of the proteinase K reactions was further subjected to DNase I or RNase I digestion, and an additional clean up, respectively. The four reactions for each activatable label were spotted on nitrocellulose membranes using a slot blot apparatus. Membranes were then blocked with 5% BSA (Promega) in TBST and subjected to Western analysis using anti-biotin antibody as describes in example 20. Bands volumes quantitated using Image J software were used to determine the % of DNA labeling (FIG. 35 B) or % of RNA labeling (FIG. 36 B) out of the total labeling of protein and nucleic acids. These analyses revealed that several vinyl-naphthyl-azides and quinoline-azides exhibited 30-46% specific DNA labeling while only two vinyl-naphthyl-azides incorporating an additional CN substitution exhibited 10% specific RNA labeling.

### Example 22

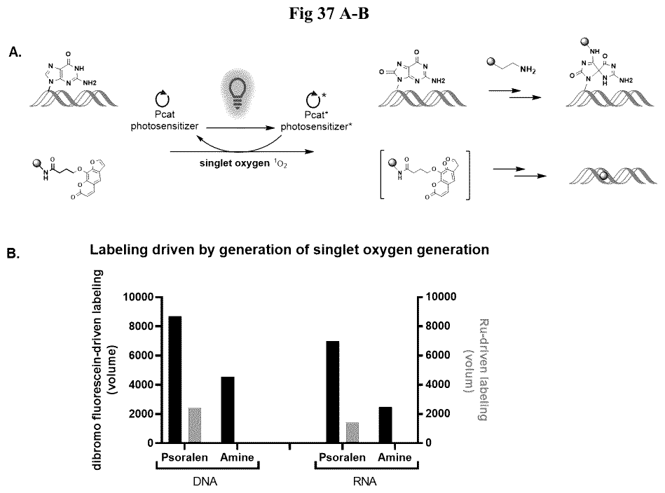

During development of the bioluminescence-triggered photocatalytic labeling described herein, experiments were conducted to evaluate the capacity to drive labeling of nucleic acids via generation of singlet oxygen, which can facilitate functionalization of predominantly guanine bases for subsequent interaction with an amine-biotin or can oxidize furanocoumarins (e.g., psoralen) to facilitate their subsequent crosslinking with nucleic acids (FIG. 37A).

The capacity of two singlet oxygen generators, Ru-8974 and di-bromo-fluorescein (PS-9850), to drive singlet oxygen-dependent labeling was evaluated as described in Example 20. Quantitation of the slot blot analyses revealed nucleic acid labeling via singlet oxygen generation albeit at lower efficiencies compared to those shown in Example 20 for Ir-9049 driven photocatalytic activation of vinyl-naphthyl-azides and vinyl-quinoline-azides (FIG. 37 B).

### Example 23

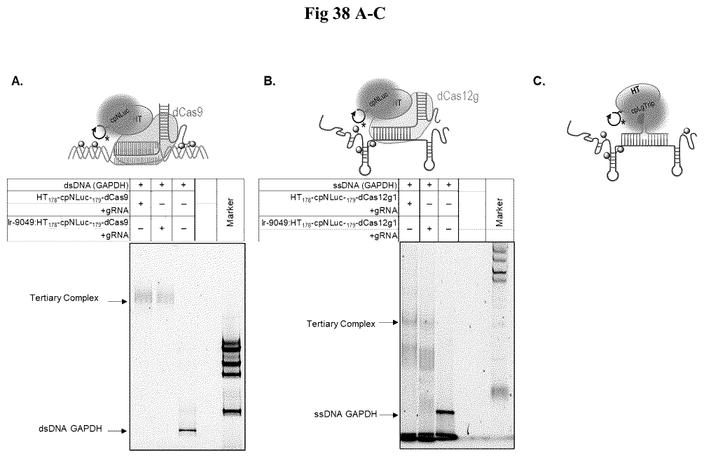

This example describes several strategies for targeting a specific nucleic acid sequence including the use of 1) a specific guide RNA in combination with a dCas-HT₁₇₈-cpNLuc-₁₇₉ chimera fusion, which is tethered to a chloroalkane-conjugated catalyst or the use of 2) specific antisense oligos conjugated to Trip peptides, which upon hybridization with a specific RNA/DNA locus can undergo facilitated complementation with a HT₁₇₈-cpLgTrip-₁₇₉ chimera that is tethered to the catalyst (FIG. 38). The capacity to target a specific dsDNA or ssDNA using a guide RNA in combination with a dCas-HT₁₇₈-cpNLuc-₁₇₉ chimera fusion was further evaluated using a gel shift assay. To this end, nucleoprotein complexes comprising 1 μM guide RNA and 100 nM dCas9-HT₁₇₈-cpNLuc-₁₇₉ or 100 nM dCas12g1-HT₁₇₈-cpNLuc-₁₇₉ were assembled and then incubated with either a specific dsDNA or ssDNA, respectively for 30 min at 37° C. Complexes as well as dsDNA or ssDNA alone were resolved on native agarose gels and then stained with either gel red (dsDNA) or SYBR green (ssDNA). Formation of tertiary complexes with the specific dsDNA/ssDNA were verified via a gel shift in the nucleic acid migration.

1. A system comprising:
(a) (i) a bioluminescent protein having at least 70% sequence identity with SEQ ID NO: 1 or a circularly permuted variant thereof or (ii) structurally-complementary components of a bioluminescent complex wherein the structurally-complementary components collectively comprise at least 70% sequence identity with SEQ ID NO: 2;
(b) a coelenterazine luminophore or a derivative thereof, wherein the bioluminescent protein or complex catalyzes emission of a first wavelength of light from the luminophore upon interaction therewith;
(c) a photocatalyst, wherein the photocatalyst is activated by exposure to light of the first wavelength; and
(d) an activatable label, wherein the activatable label is converted into an activated label when in proximity to the activated photocatalyst.

2-19. (canceled)

20. The system of claim 1, wherein the coelenterazine luminophore is furimazine or fluorofurimazine.

21. (canceled)

22. The system of claim 1, wherein the photocatalyst is of the structure of Formula (I): 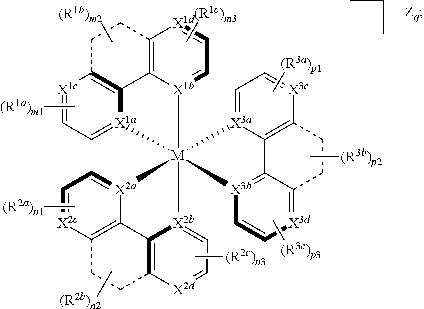
wherein:
each set of dashed lines ( [symbol] ) represents the presence or absence of a fused 6-membered ring;
M is a transition metal;
m1, m2, m3, n1, n2, n3, p1, p2, and p3 are each independently 0, 1, or 2;
R^{1a}, R^{1b}, R^{1c}, R^{2a}, R^{2b}, R^{2c}, R^{3a}, R^{3b}, and R^{3c} are each independently selected from halo, alkyl, haloalkyl, amino, heteroalkyl, and a group -Linker-Q, wherein Q is a capture element;
X^{1a}, X^{1b}, X^{2a}, X^{2b}, X^{3a}, and X^{3b} are each independently selected from N and C, wherein at least one of X^{1a} and X^{1b} is N, at least one of X^{2a} and X^{2b} is N, and at least one of X^{3a} and X^{3b} is N;
X^{1c}, X^{1d}, X^{2c}, X^{2d}, X^{3c}, and X^{3d} are each independently selected from CH and N;
Z is an anion; and
q is 0, 1, or 2.

23. (canceled)

24. The system of claim 22, wherein the photocatalyst is an iridium-based photocatalyst selected from: 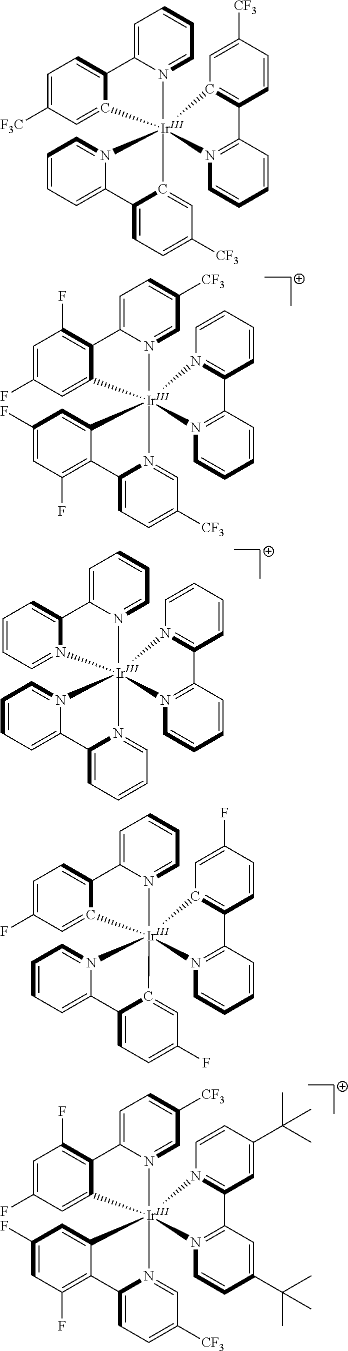 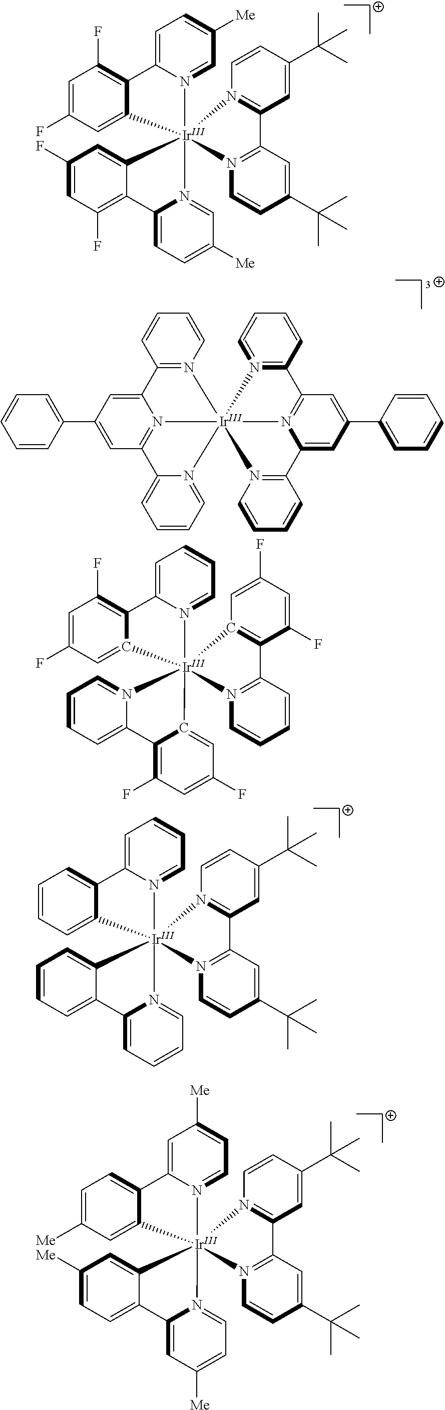 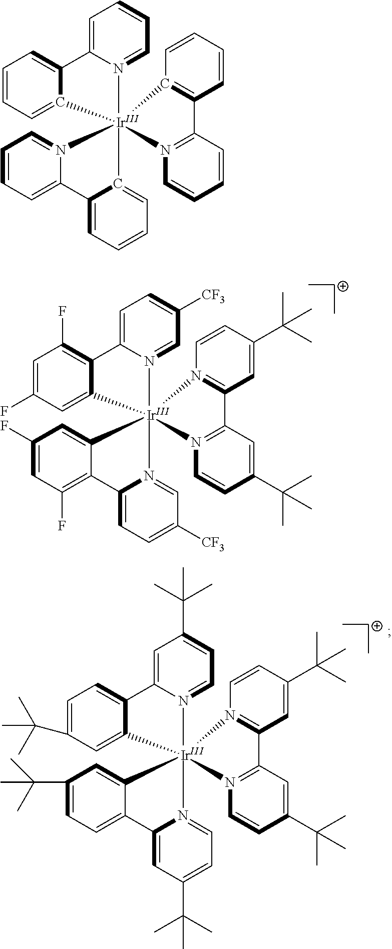
or a ruthenium-based photocatalyst selected from: 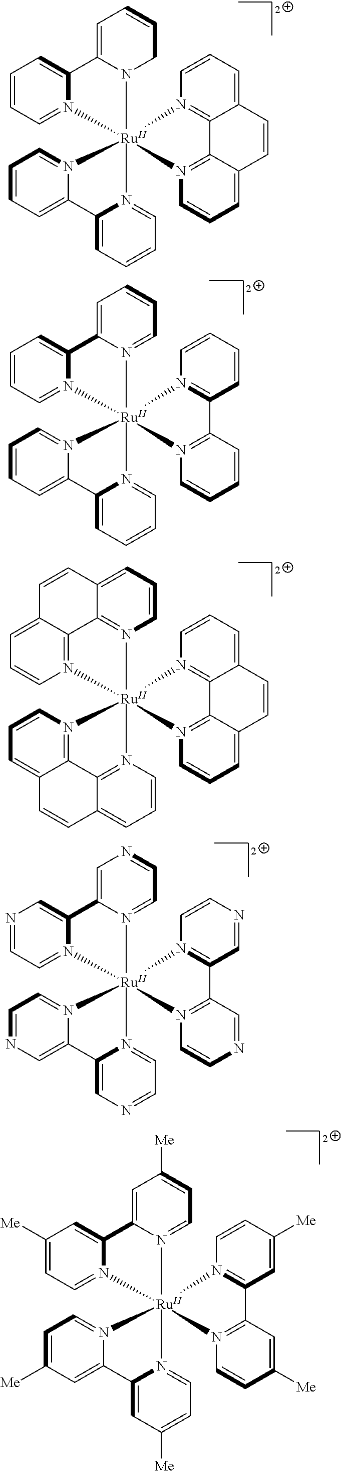 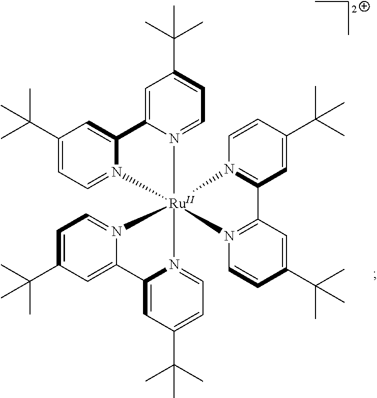
or a derivative thereof in which the compound is functionalized with at least one group -Linker-Q, wherein Q is a capture element.

25. (canceled)

26. The system of claim 22, wherein the photocatalyst is of the formula:

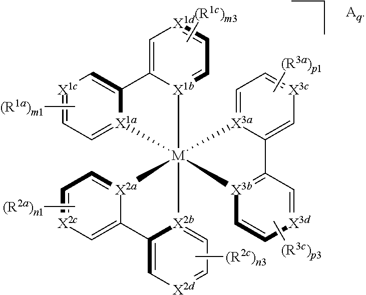

27. The system of claim 22, wherein one of R^{1a}, R^{1b}, R^{1c}, R^{2a}, R^{2b}, R^{2c}, R^{3a}, R^{3b} and R^{3c} is a group -Linker-Q, wherein Q is a haloalkane capture element.

28-29. (canceled)

30. The system of claim 27, wherein the Linker comprises ester (—C(O)O—), amide (—C(O)NH—), carbamate (—NHC(O)O—), urea (—NHC(O)NH—), phenylene, straight or branched chain alkylene, oligo- or poly-ethylene glycol (—(CH₂CH₂O)ₓ—), or combinations thereof.

31. (canceled)

32. The system of claim 30, wherein the Linker is selected from:

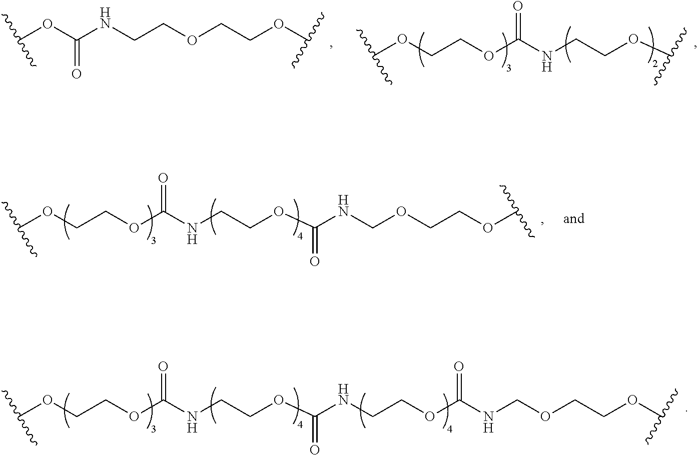

33. (canceled)

34. The system of claim 1, wherein the photocatalyst is an organic photoredox catalyst, wherein the organic photoredox catalyst is selected from a quinone, a pyrylium, an acridinium, and a xanthene.

35. The system of claim 34, wherein the organic photoredox catalyst is a quinone selected from:

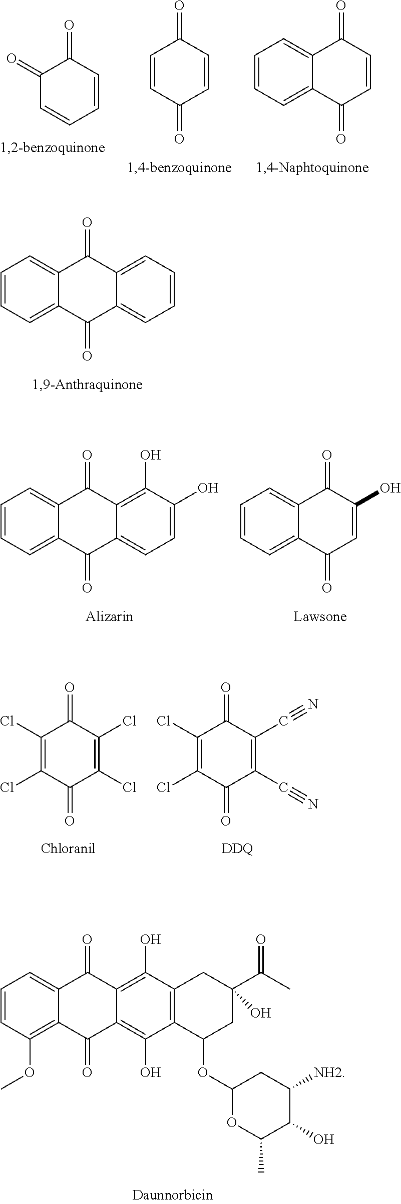

36. The system of claim 34, wherein the organic photoredox catalyst is a pyrylium selected from:

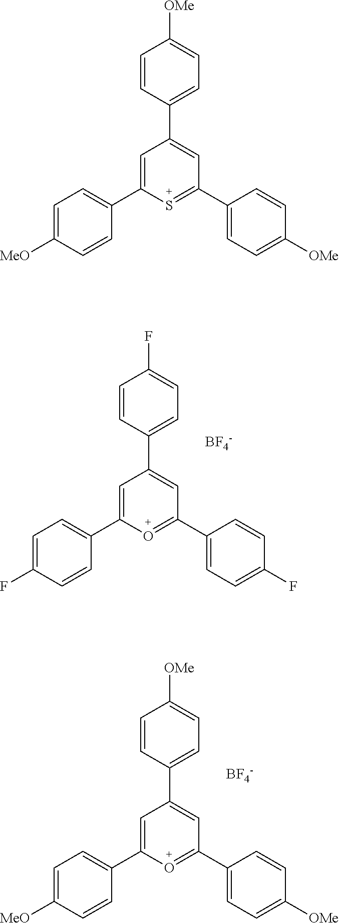

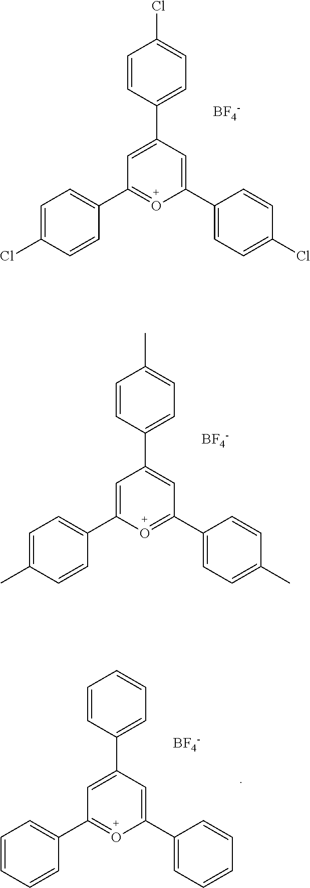

37. The system of claim 34, wherein the organic photoredox catalyst is an acridinium selected from:

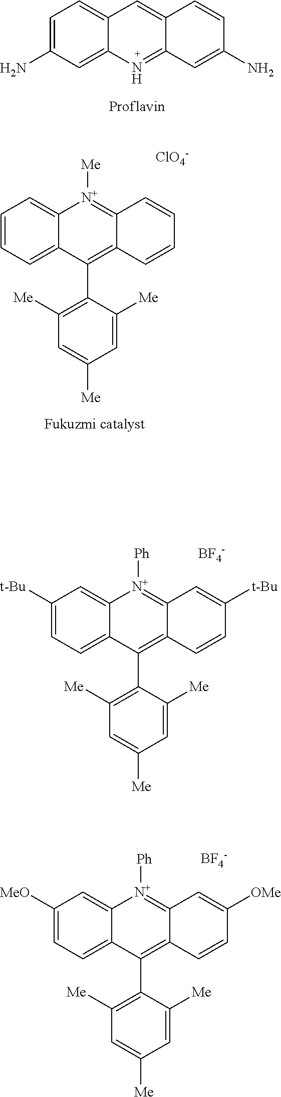

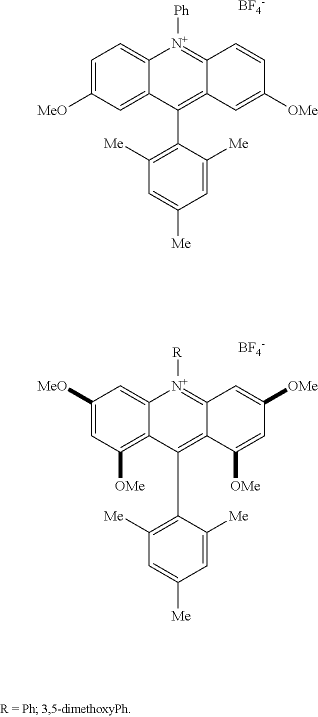

38. The system of claim 34, wherein the organic photoredox catalyst is a xanthene selected from:

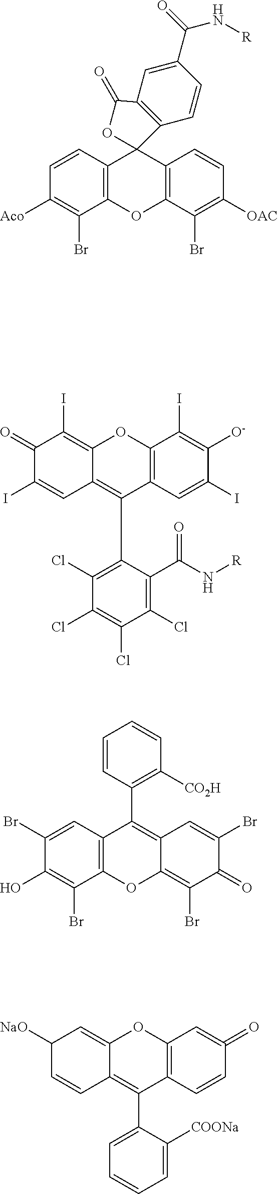

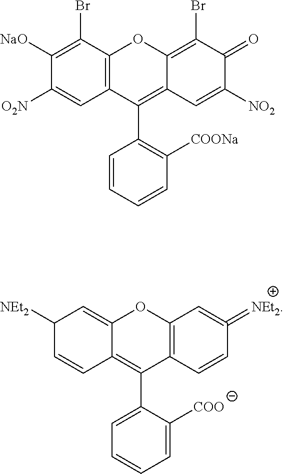

39. The system of claim 34, wherein the organic photoredox catalyst is a thiazine-based organic photoredox catalyst.

40-44. (canceled)

45. The system of claim 1, wherein the activatable label comprises a fluorogenic dye, capture agent, cleavable capture agents as well as click handle.

46. The system of claim 1, wherein the activatable label comprises a photoreactive group and a functional group.

47. The system of claim 46, wherein upon activation of the photoreactive group by the photocatalyst or photosensitizer, the activated label binds covalently to a target molecule.

48. The system of claim 47, further comprising a target molecule that the activated label is capable of reacting with.

49-50. (canceled)

51. The system of claim 46, wherein the photoreactive group comprises:
(a) an activatable benzoyl azide variant is selected from: 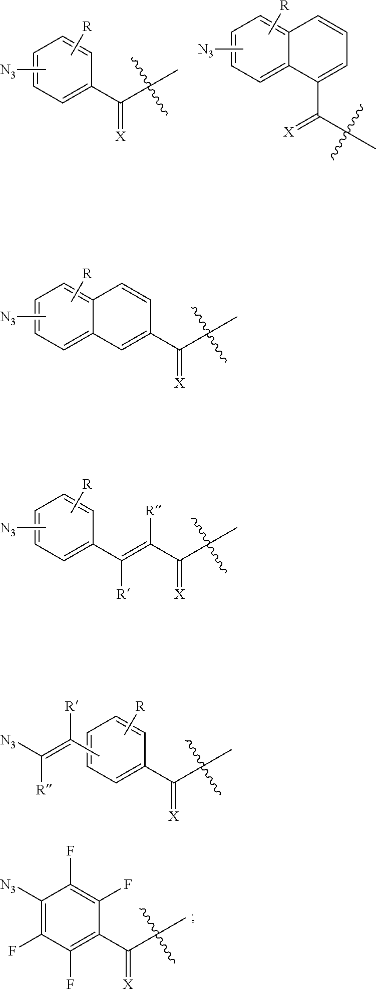
wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂; and
wherein X is O or S;
(b) an activatable benzyl diazirine variant is selected from: 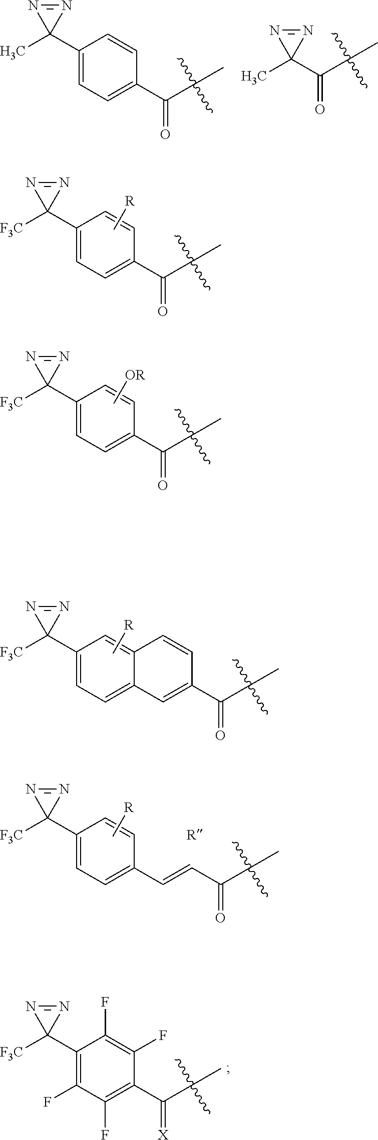
wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH3, C(O)CH₃, C(O)OH, and C(O)NH₂; 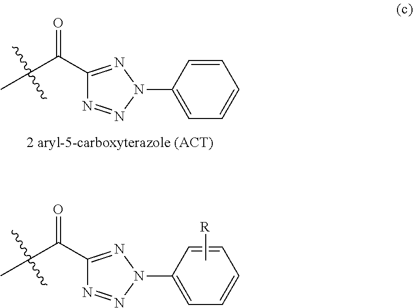
(2 aryl-5-carboxyterazole (ACT)); or an ACT derivative; wherein R is selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂; 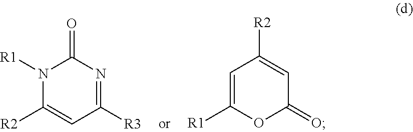
wherein one of R1-R3 is a linkage to the rest of the activatable moiety and the other two of R1-R3 are independently selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂
(e) a furanocoumarin selected from: 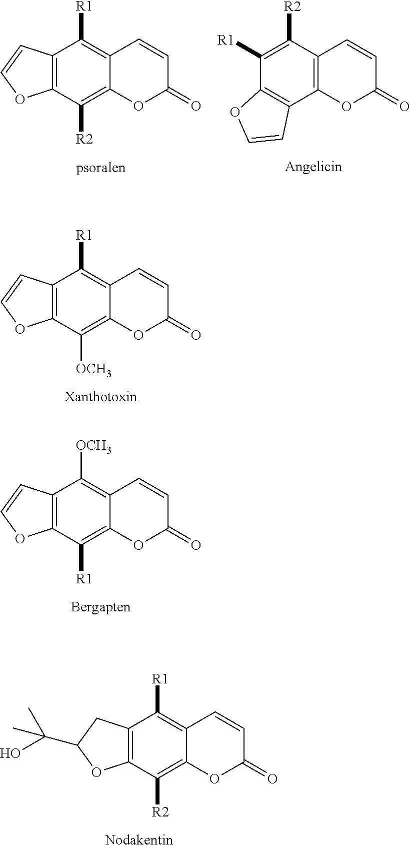
wherein one of R1-R2 is a linkage to the rest of the activatable moiety and the other R1-R2 are independently selected from H, Cl, F, Br, I, CH₃, OH, SH, NH₂, CN, CF₃, CCl₃, CH═CH₂, —CH₂—CH₃, —CH₂—OH, —CH₂NH₂, CH₂SH, CH₂Cl, CH₂Br, CH₂F, CHF₂, CH₂CN, CH₂CF₃, CH₂Cl₃, O—CH₃, C(O)CH₃, C(O)OH, and C(O)NH₂; 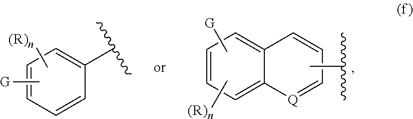
wherein
each n is independently 1, 2, 3, or 4;
each R is independently selected from hydrogen, halo, C₁-C₄ alkyl, C₂-C₄ alkenyl, hydroxy, mercapto, amino, cyano, C₁-C₄-alkoxy, halo-C₁-C₄-alkyl, hydroxy-C₁-C₄-alkyl, amino-C₁-C₄-alkyl, mercapto-C₁-C₄-alkyl, cyano-C₁-C₄-alkyl, —C(O)—C₁-C₄-alkyl, —C(O)OH, and —C(O)NH₂;
Q is CH or N; and
G is —N₃, —CH═CH—N₃, or 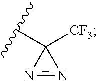 [SMILES: *C1(C(F)(F)F)N=N1]
and
(g) Z-A
Wherein A is: 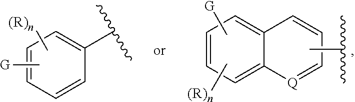
wherein each n is independently 1, 2, 3, or 4; each R is independently selected from hydrogen, halo, C₁-C₄ alkyl, C₂-C₄ alkenyl, hydroxy, mercapto, amino, cyano, C₁-C₄-alkoxy, halo-C₁-C₄-alkyl, hydroxy-C₁-C₄-alkyl, amino-C₁-C₄-alkyl, mercapto-C₁-C₄-alkyl, cyano-C₁-C₄-alkyl, —C(O)—C₁-C₄-alkyl, —C(O)OH, and —C(O)NH₂;
Q is CH or N; and G is —N₃, —CH═CH—N₃, or 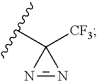
wherein Z is selected from —CR⁷═CR⁸—C(X)—, —C(X)—, and a bond, wherein R⁷ and R⁸ are each independently hydrogen or C₁-C₄ alkyl, and X is O or S.

52-58. (canceled)

59. The system of claim 46, wherein the functional group is selected from a capture agent, cleavable capture agent, fluorescent molecule, or click handle.

60. The system of claim 59, wherein the functional group is a fluorogenic fluorescent molecule selected from:

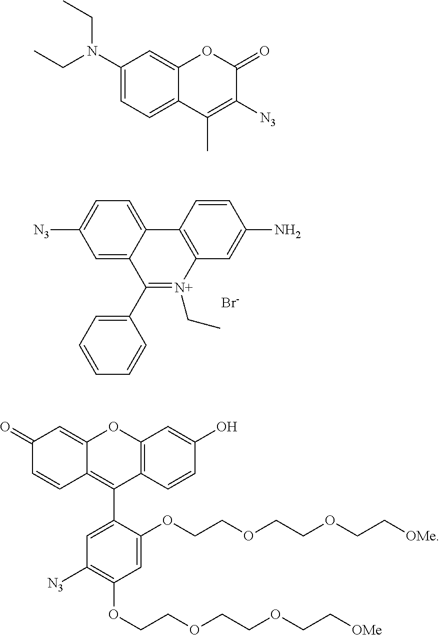 [SMILES: CCN(CC)c1ccc2c(C)c(N=[N+]=[N-])c(=O)oc2c1 ; CC[n+]1c(-c2ccccc2)c2cc(N=[N+]=[N-])ccc2c2ccc(N)cc21 ; [Br-] ; COCCOCCOCCOc1cc(OCCOCCOCCOC)c(-c2c3ccc(=O)cc-3oc3cc(O)ccc23)cc1N=[N+]=[N-]]

61. The system of claim 1, wherein the bioluminescent protein or component of the bioluminescent complex is fused to a first molecular entity and the photocatalyst or photosensitizer is tethered to a second molecular entity, wherein interaction of the first and second molecular entities places the bioluminescent protein or bioluminescent complex in sufficient proximity to the photocatalyst or photosensitizer such that light emitted by the luminophore upon interaction with the bioluminescent protein or bioluminescent complex activates the photocatalyst or photosensitizer.

62-88. (canceled)
